(19) United States
(12) Patent Application Publication
(10) Pub. No.: US 2018/0282348 A1
(43) Pub. Date: Oct. 4, 2018

(54) IMIDAZO[2,1-B]THIAZOLE AND 5,6-DIHYDROIMIDAZO[2,1-B]THIAZOLE DERIVATIVES USEFUL AS S100-INHIBITORS

(71) Applicant: Active Biotech AB, Lund (SE)

(72) Inventors: Ulf WELLMAR, Sodra Sandby (SE); Stephen EAST, Wallingford, Oxfordshire (GB); Marie BAINBRIDGE, Didcot, Oxfordshire (GB); Colin MACKINNON, Newport Pagnell, Buckinghamshire (GB); James CARR, Oxford, Oxfordshire (GB); Jonathan HARGRAVE, Bath, Bath and North East Somerset (GB)

(21) Appl. No.: 15/545,573

(22) PCT Filed: Dec. 4, 2015
(86) PCT No.: PCT/EP2015/078683

§ 371 Date: Jul. 21, 2017

(30) Foreign Application Priority Data
Feb. 3, 2015 (EP) 15153627.3

(51) Int. Cl. C07D 513/04; C07D 513/14

(52) U.S. Cl. CPC C07D 513/04; C07D 513/14

(57) ABSTRACT

A compound of formula (I) or a pharmaceutically acceptable salt thereof. The compound is useful for use in the treatment of cancer, an inflammatory disorder, an autoimmunity disorder or a neurodegenerative disorder.

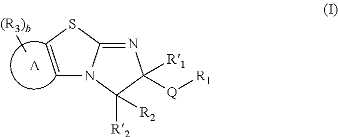

### FIELD OF THE INVENTION

The present invention relates to imidazo[2,1-b]thiazole and 5,6-dihydroimidazo[2,1-b]thiazole derivatives, pharmaceutical compositions of these derivatives and their use as medicaments. More particularly the invention relates to imidazo[2,1-b]thiazole and 5,6-dihydroimidazo[2,1-b]thiazole derivatives for use in the treatment of cancer, autoimmune disorders, inflammatory disorders and neurodegenerative disorders.

### BACKGROUND OF THE INVENTION

S100A9 belongs to the S100-family of calcium-binding proteins and has been recognized as an attractive novel therapeutic target for the treatment of e.g. autoimmunity, inflammatory disease, neurodegenerative disease and cancer. Other S100 proteins have distinct roles in many different biological processes and are connected to a number of diseases including cancer, cardiomyopathies, atherosclerosis, Alzheimer's disease and inflammatory diseases. Twenty-one of the human genes, including S100A9, are located at chromosomal region 1q21, which is frequently altered in tumors (Marenholz et al., 2004). Interestingly, although the primary sequence diverges between family members, the 3D-structures of the different proteins are very similar.

S100A9 is often co-expressed with S100A8, another member of the S100 protein family, and they are highly expressed in myeloid cells, such as neutrophils and monocytes, but can also be induced in other cells or tissues (Srikrishna 2012). They form non-covalent homo- and heterocomplexes that can be specifically released in response to cellular activation (Foell et al., 2007, Ryckman et al., 2003). S100A9 can functionally be described as a damage-associated molecular pattern (DAMP) molecule which is released in tissues and induces signaling by interacting with receptors such as RAGE and TLR4 (Foell et al., 2007). As for many other DAMP molecules, S100A9 also has intracellular roles in addition to its extracellular functions, e.g. by binding to the cytoskeleton and influencing cytoskeletal rearrangements and thereby cellular migration (Srikrishna 2012). A pro-inflammatory role for S100A9 is supported by elevated S100A9 serum levels in inflammatory diseases and by high concentrations of S100A9 at local sites of inflammation, for example in the synovial fluid of rheumatoid arthritis patients (Foell et al., 2004) or osteoarthritis patients (van Lent 2012) where high levels correlate with joint destruction. Also, preclinical studies with S100A9 knock-out mice show an involvement of S100A9 in many inflammatory processes including synovial activation and cartilage destruction during osteoarthritis (van Lent 2012). High levels of S100A9 have also been found in several forms of cancer and a high expression level has been shown to correlate with poor tumor differentiation in some of these cancer forms (Arai et al., 2008). Elevated S100A9 levels in pathological conditions of chronic inflammation as well as in cancer argue for a possible role in inflammation-associated carcinogenesis.

A role for S100A9 in the coupling between the immune system and cancer is also supported by studies showing that S100A8 and S100A9 are highly expressed in and important for the function of myeloid-derived suppressor cells (MDSCs) (Cheng et al., 2008, Sinha et al., 2008, Wang et al., 2013), a mixture of immature myeloid cells that suppress T- and NK-cell activation and promote angiogenesis and tumor growth. By interfering with S100A9-regulated accumulation of tumor infiltrating MDSCs, the balance between these processes may change in favor of an anti-angiogenic and less immune suppressive milieu with inhibited tumor progression. Furthermore, there are data suggesting a role for S100A9 in recruiting both inflammatory cells and tumor cells to metastatic sites (Hiratsuka et al., 2006, Acharyya et al. 2012, Hibino et al., 2013). Thus, blocking the function of S100A9 may provide a new approach to prevention of metastasis.

Although a number of possible biological functions of S100A9 have been proposed, the exact role of S100A9 in inflammation, in cancer and in other diseases is still unknown. Members of the S100 protein family have been reported to interact with the pro-inflammatory molecule RAGE and studies showed that S100A9 is the strongest RAGE binder within the S100 family in the presence of physiological levels of Ca²⁺ and Zn²⁺ (Bjirk et al. 2009). These studies further demonstrated that S100A9 interacts with toll-like receptor 4 (TLR4). As for the S100A9-RAGE interaction, the S100A9-TLR4 interaction appears to be strictly dependent on the presence of physiological levels of both Ca²⁺ and Zn²⁺. Another receptor for S100A9 that may be important in cancer is EMMPRIN (CD 147), this protein is expressed on different cell types and the S100A9-EMMPRIN interaction has been shown to be involved in melanoma metastasis (Hibino et al., 2013).

S100A8 and S100A9 proteins have predominantly been described as cytoplasmic proteins that are secreted from myeloid cells upon activation. It is generally believed that the major biological functions relevant to inflammation require the release of the S100 proteins to the extracellular space. In this model, extracellular S100A9 would bind to e.g. the pro-inflammatory receptors RAGE and TLR4 and result in an inflammatory response. This is supported by studies showing that S100A9 induces TNF production in human monocytes via TLR4 (Riva et al. 2012, Cesaro et al. 2012). Also, S100A9 in complex with S100A8 has shown growth promoting activity directly on tumors cells via RAGE signaling (Ghavami et al., 2008). S100A9 also exists in a membrane-associated form on monocytes (Bhardwaj et al., 1992). Membrane associated S100A9 opens up for the possibility of cell-cell or cell-ECM signaling involving S100A9.

The collected data suggest that S100A9 have important roles in inflammation, cancer growth, cancer metastasis and in their connections. Novel compounds that inhibit the activity of S100A9 in these processes, and thereby disturb the tumor microenvironment, would be attractive in treatment of cancer of different types.

Besides cancer, inflammation and autoimmunity, S100A9 has strong connections to neurodegenerative disease. S100A9 is upregulated in the brain in Alzheimer's disease (AD) patients and in mouse disease models (Shepherd et al., 2006, Ha et al., 2010). Furthermore, knock-down or deletion of S100A9 in mice models of AD inhibits cognition decline and plaque burden in the brain (Ha et al., 2010, Chang et al., 2012). A role for RAGE is also evident in AD where inhibition of RAGE reduces disease in a mouse AD model (Deane et al., 2013). Inhibition of S100A9 and its interactions represents a new promising approach for therapeutic intervention in AD and other neurodegenerative diseases.

WO 02/069965 (Transtech Pharma Inc) discloses certain benzimidazole derivatives, as modulators of the interaction between RAGE and its ligands for the management, treatment, control, or as an adjunct treatment for diseases in humans caused by RAGE, e.g. acute and chronic inflammation, the development of diabetic late complications such as increased vascular permeability, nephropathy, atherosclerosis, and retinopathy, the development of Alzheimer's disease, erectile dysfunction, and tumor invasion and metastasis.

A number of publications describe pharmacological effects (often low to moderate) of imidazo[2,1-b]thiazole derivatives, viz. 7-thia-2,5-diazatricyclo[6.4.0.0^{2,6}]dodeca-1(8),3,5,9,11-pentaene-4-carboxylic acids and imidazo[2,1-b][1,3]thiazole-6-carboxylic acids. Thus, analgesic and antiinflammotory effects were described by Palagiano 1995, Palagiano 1996, Abignente 1981 and Grandolini 1993. Antiallergic effect was reported by Ager 1988, anxiolytic effect by Clements-Jewery 1988 (inactive in flunitrazepam receptor binding) and SIRT1 activation by Vu et al 2009 (inactive). In addition, blood sugar reducing effect was reported in U.S. Pat. No. 4,137,320 and activity against hepatitis C was reported in WO2006008556. Other publications describe 7-thia-2,5-diazatricyclo[6.4.0.0^{2,6}]dodeca-1(8),3,5,9,11-pentaene-4-carboxylic acids and imidazo[2,1-b][1,3]thiazole-6-carboxylic derivatives as synthesis targets or synthetic intermediates with no pharmacological data e.g. Blackburn 2010, Herath 2010 and Patel 2009.

A number of imidazo[2,1-b]thiazole derivatives are commercially available, or have been disclosed in the literature, but have not hitherto been disclosed for use in therapy.

### SUMMARY OF THE INVENTION

A first aspect is a compound of formula (I)

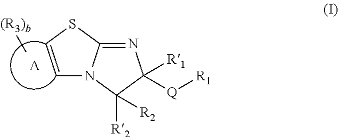

or a pharmaceutically acceptable salt thereof, wherein
b is an integer of from 0 to 4;
ring A is a 5- to 7-membered, aromatic or non-aromatic carbocycle or heterocycle;
Q is a direct bond, CH₂, CH(OH) or NH;
R₁ is R₄C(O), cyano, or tetrazolyl;
R₄ is H, R₅O, or NHR₆;
R₅ is H or C1-C6 alkyl;
R₆ is H, cyano, C1-C6 alkyl, or R₇S(O)₂;
R₇ is C1-C6 alkyl, C3-C6 cycloalkyl, R₅(CH₂)_{y}, or 5- or 6-membered aryl or heteroaryl, said aryl or heteroaryl optionally being substituted by one or more moieties independently selected from C1-C6 alkyl,
R₈ is R₉O, R₁₀R₁₁N or R₁₂OC(O);
R₉ is H or C1-C6 alkyl;
R₁₀ and R₁₁ are independently selected from H and C1-C6 alkyl, or R₁₀ and R₁₁, together with the nitrogen atom to which they are both attached, form a 4- to 6-membered ring;
R₁₂ is H or C1-C6 alkyl;
y is an integer from 1 to 4;
R₂ is H, C1-C6 alkyl, C2-C6 alkenyl, C3-C6 cycloalkyl, halogen, cyano, R₁₃R₁₄N(CH₂)_{d}, R₁₅O(CH₂)ₑ, R₁₆S(CH₂)_{f}, R₁₇C(O)(CH₂)_{g},

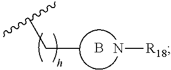

or
phenyl, optionally substituted by R₁₉O(CH₂)ᵢ;
R₁₃ is H, C1-C6 alkyl, R₂₀C(O), R₂₁S(O)₂, R₂₂O(CH₂)ⱼ, R₂₃R₂₄N(CH₂)ₖ, or benzyl, and
R₁₄ is H or C1-C6 alkyl; or
R₁₃ and R₁₄, together with the nitrogen atom to which they are both attached, form a 4- to 6-membered ring, said ring optionally being substituted by one or more substituents independently selected from oxo, halogen, C1-C6 alkyl, R₂₅C(O), R₂₆OC(O), and R₂₇O(CH₂)ₘ;
R₁₅ is H, C1-C6 alkyl or R₂₈C(O);
R₁₆ and R₁₇ are selected from H and C1-C6 alkyl;
R₁₈ is H, C1-C6 alkyl, R₂₉OC(O)(CH₂)ₙ, or R₃₀S(O)₂(CH₂)ₚ;
R₁₉, R₂₀, R₂₁, R₂₂, R₂₅, R₂₆, R₂₇, R₂₈, R₂₉ and R₃₀ are selected from H and C1-C6 alkyl;
R₂₃ and R₂₄ are independently selected from H and C1-C6 alkyl; or R₂₃ and R₂₄, together with the nitrogen atom to which they are both attached, form a 4- to 6-membered ring;
ring B is 4- to 6-membered, and saturated or unsaturated;
d, e, f, g, h, i, j, k, m, n, and p are integers of from 0 to 4;
R′₁ and R′₂ together form a bond; or
R′₁ is H, C1-C6 alkyl, C3-C6 carbocyclyl-(CH₂)_{q}, or R₃₁O(CH₂)ᵣ; and R′₂ is H;
R₃₁ is H or C1-C6 alkyl;
q and r are integers of from 0 to 4;
each R₃ is independently selected from C1-C6 alkyl, C3-C6 carbocyclyl, halogen, oxo, R₃₂O, R₃₃S, and R₃₄R₃₅N;
R₃₂ is H, C1-C6 alkyl, C3-C6 carbocyclyl-(CH₂)ₛ, or R₃₆R₃₇N(CH₂)ₜ; R₃₃ is H or C1-C6 alkyl;
R₃₄ and R₃₅ are independently selected from H and C1-C6 alkyl; or R₃₄ and R₃₅, together with the nitrogen atom to which they are both attached, form a 4- to 6-membered ring optionally substituted by one or more halogen;
R₃₆ and R₃₇ are independently selected from H and C1-C6 alkyl, or R₃₆ and R₃₇, together with the nitrogen atom to which they are both attached, form a 4- to 6-membered ring, optionally substituted by one or more halogen;
s and t are integers of from 0 to 4; and
two R₃ attached to adjacent atoms of ring A, together with the atoms to which they are attached, may form a 3- to 6 membered ring, said ring being optionally substituted by one or more C1-C6 alkyl; and
any alkyl, alkenyl and cycloalkyl is optionally substituted by one or more F;
provided that the compound is not
9,10-dichloro-7-thia-2,5-diazatricyclo[6.4.0.0^{2,6}]dodeca-1(8),3,5,9,11-pentaene-4-carboxylic acid,
10-(trifluoromethoxy)-7-thia-2,5-diazatricyclo[6.4.0.0^{2,6}]dodeca-1(8),3,5,9,11-pentaene-4-carboxylic acid,
9-chloro-7-thia-2,5-diazatricyclo[6.4.0.0^{2,6}]dodeca-1(8),3,5,9,11-pentaene-4-carboxylic acid,
10,11-dichloro-7-thia-2,5-diazatricyclo[6.4.0.0^{2,6}]dodeca-1(8),3,5,9,11-pentaene-4-carboxylic acid,
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-fluoro-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0²]dodeca-1(8),3,5,9,11-pentaene-4-carboxylic acid,
10,12-dichloro-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5-triene-4-carboxylic acid,
10-tert-butyl-7-thia-2,5-diazatricyclo[6.4.0.0² 6]dodeca-1(8),3,5-triene-4-carboxylic acid,
11-chloro-7-thia-2,5-diazatricyclo[6.4.0.0^{2,6}]dodeca-1(8),3,5,9,11-pentaene-4-carboxylic acid,
9-oxo-7-thia-2,5-diazatricyclo[6.4.0.0^{2,6}]dodeca-1(8),3,5-triene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0²]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
ethyl 2-{7-thia-2,5-diazatricyclo[6.4.0.0^{2,6}]dodeca-1(8),3,5,9,11-pentaen-4-yl}acetate,
ethyl 2-{10,11-dimethyl-7-thia-2,5-diazatricyclo[6.4.0.0^{2,6}]dodeca-1(8),3,5,9,11-pentaen-4-yl}acetate,
ethyl 2-{10-chloro-7-thia-2,5-diazatricyclo[6.4.0.0^{2,6}]dodeca-1(8),3,5,9,11-pentaen-4-yl}acetate,
ethyl 2-{11-methoxy-7-thia-2,5-diazatricyclo[6.4.0.0^{2,6}]dodeca-1(8),3,5,9,11-pentaen-4-yl}acetate,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
ethyl 7-thia-2,5-diazatricyclo[6.4.0.0^{2,6}]dodeca-1(8),3,5,9,11-pentaene-4-carboxylate,
ethyl 10,11-dimethyl-7-thia-2,5-diazatricyclo[6.4.0.0^{2,6}]dodeca-1(8),3,5,9,11-pentaene-4-carboxylate,
ethyl 10-methoxy-7-thia-2,5-diazatricyclo[6.4.0.0^{2,6}]dodeca-1(8),3,5,9,11-pentaene-4-carboxylate,
ethyl 12-methoxy-7-thia-2,5-diazatricyclo[6.4.0.0^{2,6}]dodeca-1(8),3,5,9,11-pentaene-4-carboxylate,
ethyl 10-ethyl-7-thia-2,5-diazatricyclo[6.4.0.0^{2,6}]dodeca-1(8),3,5,9,11-pentaene-4-carboxylate,
methyl 7-thia-2,5-diazatricyclo[6.4.0.0^{2,6}]dodeca-1(8),3,5,9,11-pentaene-4-carboxylate,
propyl 7-thia-2,5-diazatricyclo[6.4.0.0^{2,6}]dodeca-1(8),3,5,9,11-pentaene-4-carboxylate,
isopropyl 7-thia-2,5-diazatricyclo[6.4.0.0^{2,6}]dodeca-1(8),3,5,9,11-pentaene-4-carboxylate,
7-thia-2,5-diazatricyclo[6.4.0.02,6]dodeca-1(8),3,5,9,11-pentaene-4-carbaldehyde,
10-ethyl-7-thia-2,5-diazatricyclo[6.4.0.02,6]dodeca-1(8),3,5,9,11-pentaene-4-carbaldehyde,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1 (10),2,4,6,8,12,14-heptaen-13-yl}acetic acid,
ethyl 16-thia-11,14-diazatetracyclo[8.6.0.0^{2,7}.0^{11,15}]hexadeca-1 (10),2,4,6,8,12,14-heptaene-13-carboxylate,
ethyl 2-{16-thia-11,14-diazatetracyclo[8.6.0.0^{2,7}.0^{11,15}]hexadeca-1 (10),2,4,6,8,12,14-heptaen-13-yl}acetate,
ethyl 10-(trifluoromethyl)-7-thia-2,5-diazatricyclo[6.4.0.0^{2,6}]dodeca-1(8),3,5,9,11-pentaene-4-carboxylate,
ethyl 10-chloro-7-thia-2,5-diazatricyclo[6.4.0.0^{2,6}]dodeca-1(8),3,5,9,11-pentaene-4-carboxylate,
ethyl 10-bromo-7-thia-2,5-diazatricyclo[6.4.0.0^{2,6}]dodeca-1(8),3,5,9,11-pentaene-4-carboxylate,
ethyl 10-methyl-7-thia-2,5-diazatricyclo[6.4.0.0^{2,6}]dodeca-1(8),3,5,9,11-pentaene-4-carboxylate,
ethyl 12-methyl-7-thia-2,5-diazatricyclo[6.4.0.0^{2,6}]dodeca-1(8),3,5,9,11-pentaene-4-carboxylate,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid,
2-{12-methyl-7-thia-2,5-diazatricyclo[6.4.0.0^{2,6}]dodeca-1(8),3,5,9,11-pentaen-4-yl}acetic acid,
ethyl 2-{10-trifluoromethyl-7-thia-2,5-diazatricyclo[6.4.0.0^{2,6}]dodeca-1(8),3,5,9,11-pentaen-4-yl}acetate,
Ethyl 2-{10-bromo-7-thia-2,5-diazatricyclo[6.4.0.0^{2,6}]dodeca-1(8),3,5,9,11-pentaen-4-yl}acetate,
ethyl 2-{10-methyl-7-thia-2,5-diazatricyclo[6.4.0.0^{2,6}]dodeca-1(8),3,5,9,11-pentaen-4-yl}acetate, or
ethyl 2-{10-methyl-7-thia-2,5-diazatricyclo[6.4.0.0^{2,6}]dodeca-1(8),3,5,9,11-pentaen-4-yl}acetate.

The compounds of formula (I) as defined herein above are useful as inhibitors of interactions between S100A9 and interaction partners such as RAGE, TLR4 and EMMPRIN. Thus, a further aspect is a compound of formula (I) or a pharmaceutically acceptable salt thereof as defined herein above for use as an inhibitor of interactions of S100A9 and its interaction partners and for use in the treatment of disorders associated with functions of S100A9, e.g. inflammatory diseases, neurodegenerative diseases, autoimmune diseases and cancer.

A further aspect is a compound of formula (I)

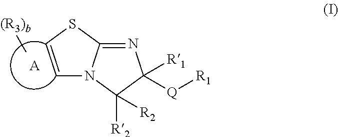

or a pharmaceutically acceptable salt thereof, wherein ring A, b, R₁, R₂, R′₁, R′₂, each R₃, and Q are as defined herein above, for use in therapy, e.g. for the treatment of a disorder selected from inflammatory diseases, neurodegenerative diseases, autoimmune diseases and cancer, provided that the compound is not
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0²′ ]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
ethyl 10-ethyl-7-thia-2,5-diazatricyclo[6.4.0.0^{2,6}]dodeca-1(8),3,5,9,11-pentaene-4-carboxylate,
methyl 7-thia-2,5-diazatricyclo[6.4.0.0^{2,6}]dodeca-1(8),3,5,9,11-pentaene-4-carboxylate,
propyl 7-thia-2,5-diazatricyclo[6.4.0.0^{2,6}]dodeca-1(8),3,5,9,11-pentaene-4-carboxylate,
isopropyl 7-thia-2,5-diazatricyclo[6.4.0.0^{2,6}]dodeca-1(8),3,5,9,11-pentaene-4-carboxylate,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1 (10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

According to a further aspect, a pharmaceutical composition is provided, comprising a compound of formula (I)

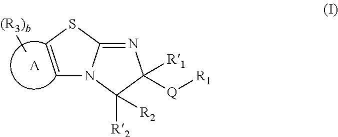

or a pharmaceutically acceptable salt thereof, wherein ring A, b, R₁, R₂, R′₁, R′₂, each R₃, and Q are as defined herein above, and optionally a pharmaceutically acceptable excipient, provided that the compound is not
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0² 6]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
ethyl 10-ethyl-7-thia-2,5-diazatricyclo[6.4.0.0^{2,6}]dodeca-1(8),3,5,9,11-pentaene-4-carboxylate,
methyl 7-thia-2,5-diazatricyclo[6.4.0.0^{2,6}]dodeca-1(8),3,5,9,11-pentaene-4-carboxylate,
propyl 7-thia-2,5-diazatricyclo[6.4.0.0^{2,6}]dodeca-1(8),3,5,9,11-pentaene-4-carboxylate,
isopropyl 7-thia-2,5-diazatricyclo[6.4.0.0^{2,6}]dodeca-1(8),3,5,9,11-pentaene-4-carboxylate,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1 (10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

The pharmaceutical composition of the invention is useful for the treatment of diseases selected from inflammatory diseases, autoimmune diseases, neurodegenerative diseases and cancer.

A further aspect is a compound of formula (I)

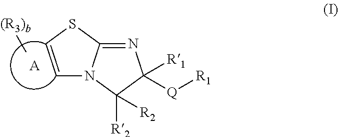

wherein ring A, b, R₁, R₂, R′₁, R′₂, each R₃, and Q are as defined herein above, for use in the treatment of a disorder selected from inflammatory diseases, neurodegenerative diseases, autoimmune diseases and cancer, provided that the compound is not
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0² 6]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1 (10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

A further aspect is a compound of formula (I)

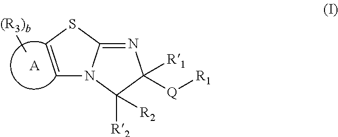

wherein ring A, b, R₁, R₂, R′₁, R′₂, each R₃, and Q are as defined herein above, for use in the treatment of a disorder selected from inflammatory diseases, neurodegenerative diseases, and cancer, e.g. for the treatment of inflammatory diseases, provided that the compound is not
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1 (10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

A further aspect is a compound of formula (I)

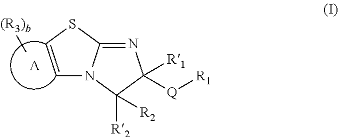

wherein ring A, b, R₁, R₂, R′₁, R′₂, each R₃, and Q are as defined herein above, for use in the treatment of a disorder selected from neurodegenerative diseases, autoimmune diseases and cancer, e.g. autoimmune diseases, provided that the compound is not
10,11-dimethyl-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid, or
12-methoxy-7-thia-2,5-diazatricyclo[6.4.0.0^{2,6}]dodeca-1(8),3,5,9,11-pentaene-4-carboxylic acid.

Still a further aspect is a compound of formula (I)

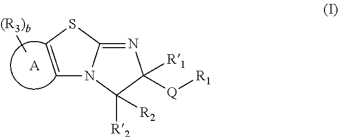

wherein ring A, b, R₁, R₂, R′₁, R′₂, each R₃, and Q are as defined herein above, for use in the treatment of a disorder selected from neurodegenerative diseases and cancer.

Still a further aspect is a compound of formula (I)

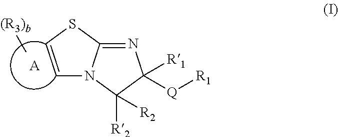

wherein ring A, b, R₁, R₂, R′₁, R′₂, each R₃, and Q are as defined herein above, for use in the treatment of a neurodegenerative disorder.

Still a further aspect is a compound of formula (I)

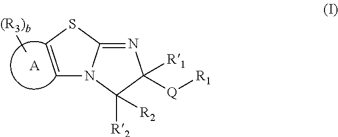

wherein ring A, b, R₁, R₂, R′₁, R′₂, each R₃, and Q are as defined herein above, for use in the treatment of cancer.

Another aspect is the use of a compound of formula (I) as defined hereinfor use in the treatment of a disorder selected from inflammatory diseases, neurodegenerative diseases, autoimmune diseases and cancer, or a pharmaceutically acceptable salt of such compound, in the manufacturing of a medicament for use in the treatment of any of said disorders, e.g. in the manufacturing of a medicament for use in the treatment of a disorder selected from neurodegenerative diseases, autoimmune diseases and cancer.

Still another aspect is a method of treatment of a disorder selected from inflammatory diseases, neurodegenerative diseases, autoimmune diseases and cancer, by administration of a compound of formula (I) as defined herein, or a pharmaceutically acceptable salt thereof, to a mammal in need of such treatment.

### BRIEF DESCRIPTION OF THE DRAWINGS

FIG. 1 is a schematic representation of an assay of the inhibition of the interaction between biotinylated human S100A9 and human RAGE-Fc using a small molecule S100A9 binder.

### DETAILED DESCRIPTION OF THE INVENTION

Some definitions of terms used herein are provided herein below. The listing is not exhaustive and it is noted that any term and expression used herein should be given its usual meaning, unless otherwise specified or clearly apparent from the context. Thus, for example, the term alkyl, either alone or as part of a radical, includes straight or branched chain alkyl of the general formula CₙH₂ₙ₊₁.

The term C1-C6 alkyl includes any alkyl group having 1, 2, 3, 4, 5 or 6 carbon atoms.

The term C1-C4 alkyl includes methyl, ethyl, n-propyl, isopropyl, n-butyl, isobutyl and tert-butyl.

The term C1-C3 alkyl includes methyl, ethyl, n-propyl and isopropyl.

The term cycloalkyl refers to a cyclic alkyl radical of the general formula CₙH₂ₙ₋₁.

The term C3-C6 cycloalkyl refers to cyclopropyl, cyclobutyl, cyclopentyl and cyclohexyl.

The term phenyl refers to a C₆H₅ radical of the formula

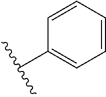

i.e. a 6-membered aryl.

The term benzyl refers to a radical of the formula

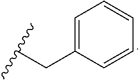

The term heterocycle (herein used synonymously with “heterocyclic ring”) refers to a saturated or unsaturated and aromatic or non-aromatic cyclic moiety containing at least one heteroatom in the ring.

The term carbocycle (herein used synonymously with “carbocyclic ring”) refers to a saturated or unsaturated and aromatic or non-aromatic cyclic moiety having only carbon atoms in the ring. For example, a cycloalkyl is a saturated carbocycle, a cycloalkenyl is an unsaturated carbocycle, and benzene is an aromatic carbocycle.

The term heteroaryl refers to a heterocyclyl that is aromatic.

The term halogen refers to F, Cl, Br and I, preferably F, Cl and Br.

The term hydroxy (OH) refers to a radical of the formula

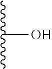

The term alkoxy refers to a radical of the formula RO, wherein R is alkyl.

The term RO refers to a radical of formula

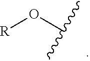

The term cyano, or CN, refers to a radical of the formula

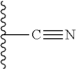

The term tetrazolyl refers to a radical of the formula

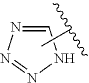

and any tautomer thereof, in particular it refers to a radical of the formula

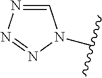

and any tautomer thereof.

A term of the type RC(O) refers to a moiety of formula

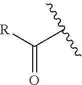

A term of the type RS refers to a radical of formula

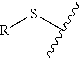

A term of the type RS(O)₂ refers to a radical of formula

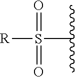

A term of the type ROC(O) refers to a radical of formula

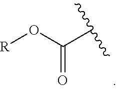

A term of the type RR′N (or NRR′) refers to a radical of formula

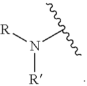

A term of the type R(CH₂)ₐ wherein a is an integer having a minimum value i and a maximum value ii, refers to a radical of the type

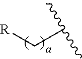

wherein a is an integer of from i to ii, and when i is 0, the radical is

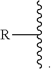

“Optional” or “optionally” means that the subsequently described event or circumstance may but need not occur, and that the description includes instances where the event or circumstance occurs and instances in which it does not.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term pharmaceutically acceptable salt of a compound refers to a salt that is pharmaceutically acceptable, as defined herein, and that possesses the desired pharmacological activity of the parent compound. Pharmaceutically acceptable salts include acid addition salts formed with inorganic acids, e.g. hydrochloric acid, hydrobromic acid, sulfuric acid, nitric acid, phosphoric acid; or formed with organic acids, e.g. acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid; or salts formed when an acidic proton present in the parent compound either is replaced by a metal ion, e.g., an alkali metal ion, an alkaline earth ion, or an aluminum ion; or coordinates with an organic or inorganic base. Acceptable organic bases include e.g. diethanolamine, ethanolamine, N-methylglucamine, triethanolamine, morpholine, and tromethamine. Acceptable inorganic bases include e.g. ammonia, aluminum hydroxide, calcium hydroxide, potassium hydroxide, sodium carbonate and sodium hydroxide.

Whenever a chiral carbon is present in a chemical structure, it is intended that all stereoisomers associated with that chiral carbon are encompassed by the structure, unless otherwise specified. Using the Cahn-Ingold-Prelog RS notational system, any asymmetric carbon atom may be present in the (R)- or (S)-configuration, and the compound may be present as a mixture of its stereoisomers, e.g. a racemic mixture, or one stereoisomer only.

Some of the compounds of the invention may exist in tautomeric forms. Any such tautomer is contemplated to be within the scope of the invention.

Also, in a compound of formula (I) as defined herein, any hydrogen atom may be replaced by a deuterium (²H), and any such deuterated compound of formula (I), comprising one or more deuteriums in place of the corresponding number of hydrogen atoms, is considered to be within the scope of the invention.

“Therapeutically effective amount” means an amount of a compound that, when administered to a subject for treating a disease state, is sufficient to effect such treatment for the disease state. The “therapeutically effective amount” will vary depending on the compound, the disease state being treated, the severity of the disease treated, the age and relative health of the subject, the route and form of administration, the judgment of the attending medical or veterinary practitioner, etc.

As used herein the terms “treatment” or “treating” is an approach for obtaining beneficial or desired results including clinical results. Beneficial or desired clinical results can include, but are not limited to, allevation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total) whether detectable or undetectable. The term can also mean prolonging survival as compared to expected survival without the treatment.

The term mammal refers to a human or any mammalian animal, e.g. a primate, a farm animal, a pet animal, or a laboratory animal. Examples of such animals are monkeys, cows, sheep, horses, pigs, dogs, cats, rabbits, mice, rats etc. Preferably, the mammal is a human.

The term cancer refers to any malignant growth or tumor caused by abnormal and uncontrolled cell division; it may spread to other parts of the body through the lymphatic system or the blood stream and includes both solid tumors and blood-borne tumors. Exemplary cancers include adrenocortical carcinoma, AIDS-related cancers, AIDS-related lymphoma, anal cancer, anorectal cancer, appendix cancer, childhood cerebellar astrocytoma, childhood cerebral astrocytoma, basal cell carcinoma, biliary cancer, extrahepatic bile duct cancer, intrahepatic bile duct cancer, urinary bladder cancer, bone and joint cancer, osteosarcoma and malignant fibrous histiocytoma, brain tumor, brain stem glioma, cerebellar astrocytoma, cerebral astrocytoma/malignant glioma, ependymoma, medulloblastoma, visual pathway and hypothalamic glioma, breast cancer, bronchial adenomas/carcinoids, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary syndrome, endometrial cancer, esophageal cancer, extracranial germ cell tumor, extragonadal germ cell tumor, eye cancer, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin's lymphoma, hypopharyngeal cancer, ocular cancer, Kaposi's sarcoma, renal cancer, laryngeal cancer, acute lymphoblastic leukemia, acute myeloid leukemia, hairy cell leukemia, lip and oral cavity cancer, lung cancer, non-small cell lung cancer, small cell lung cancer, non-Hodgkin's lymphoma, primary central nervous system lymphoma, Waldenstrom's macroglobulinemia, intraocular (eye) melanoma, Merkel cell carcinoma, malignant mesothelioma, metastatic squamous neck cancer, cancer of the tongue, multiple endocrine neoplasia syndrome, myelodysplastic syndromes, myelodysplastic/myeloproliferative diseases, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rhabdomyosarcoma, salivary gland cancer, Ewing's sarcoma family of tumors, soft tissue sarcoma, uterine cancer, uterine sarcoma, skin cancer (non-melanoma), skin cancer (melanoma), small intestine cancer, squamous cell carcinoma, testicular cancer, throat cancer, thymoma, thymoma and thymic carcinoma, thyroid cancer, transitional cell cancer of the renal pelvis and ureter and other urinary organs, gestational trophoblastic tumor, urethral cancer, vaginal cancer, vulvar cancer, and Wilm's tumor.

The term autoimmune disorder (or autoimmune disease) refers to any disorder arising from an inappropriate immune response of the body against substances and tissues normally present in the body (autoimmunity). Such response may be restricted to certain organs or involve a particular tissue in different places. Exemplary autoimmune disorders are acute disseminated encephalomyelitis (ADEM), Addison's disease, agammaglobulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease (one of two types of idiopathic inflammatory bowel disease “IBD”), Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis (or Idiopathic pulmonary fibrosis), gastritis, gastrointestinal pemphigoid, glomerulonephritis, Goodpasture's syndrome, Graves' disease, Guillain-Barré syndrome (GBS), Hashimoto's encephalopathy, Hashimoto's thyroiditis, Henoch-Schonlein purpura, herpes gestationis (aka gestational pemphigoid), Hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, Majeed syndrome, Ménière's disease, microscopic polyangiitis, mixed connective tissue disease, morphea, Mucha-Habermann disease (aka pityriasis lichenoides et varioliformis acuta), multiple sclerosis, myasthenia gravis, myositis, narcolepsy, neuromyelitis optica (also Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, pars planitis, pemphigus vulgaris, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome another form of APS, Schnitzler syndrome, Scleritis, Scleroderma, Serum Sickness, Sjögren's syndrome, spondyloarthropathy, stiff person syndrome, subacute bacterial endocarditis (SBE), Susac's syndrome, Sweet's syndrome, sympathetic ophthalmia, systemic lupus erythematosis, Takayasu's arteritis, temporal arteritis (also known as “giant cell arteritis”), thrombocytopenia, Tolosa-Hunt syndrome, transverse myelitis, ulcerative colitis (one of two types of idiopathic inflammatory bowel disease “IBD”), undifferentiated connective tissue disease different from mixed connective tissue disease, undifferentiated spondyloarthropathy, urticarial vasculitis, vasculitis, vitiligo, and Wegener's granulomatosis.

The term inflammatory disorder (or inflammatory disease) refers to a pathological state associated with inflammation, typically caused by leukocyte infiltration. The inflammatory disorder may be acute or chronic. Exemplary inflammatory disorders include inflammatory skin diseases, including, without limitation, psoriasis and atopic dermatitis, systemic scleroderma and sclerosis, responses associated with inflammatory bowel disease (IBD) (such as Crohn's disease and ulcerative colitis), ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms, cerebral edema secondary to stroke, cranial trauma, hypovolemic shock, asphyxia, adult respiratory distress syndrome, acute-lung injury, Behcet's Disease, dermatomyositis; polymyositis; multiple sclerosis (MS); dermatitis; meningitis; encephalitis; uveitis, osteoarthritis, lupus nephritis, autoimmune diseases such as rheumatoid arthritis (RA), Sjorgen's syndrome, vasculitis, diseases involving leukocyte diapedesis, central nervous system (CNS) inflammatory disorder, multiple organ injury syndrome secondary to septicemia or trauma, alcoholic hepatitis, bacterial pneumonia, antigen-antibody complex mediated diseases including glomerulonephritis, sepsis, sarcoidosis, immunopathologic responses to tissue or organ transplantation, inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis, etc.

The term neurogenerative disorder (or neurogenerative disease) refers to disorders associated with a progressive loss of structure or function of neurons affecting the structure or function of the brain, spinal cord or peripheral nervous system. Exemplary neurodegenerative disorders include mitochondrial encephalomyopathies and gut dysmotility syndromes, ataxia syndromes including Friedreich's ataxia and spinocerebellar ataxia (SCA), spinal cord injury, familial and sporadic amyotrophic lateral sclerosis (FALS and ALS, respectively), familial and sporadic Parkinson's disease, familial and sporadic Alzheimer's disease, Huntington's disease, olivopontocerebellar atrophy, multiple system atrophy, progressive supranuclear palsy, diffuse lewy body disease and synucleinopathies, Down Syndrome, corticodentatonigral degeneration, progressive familial myoclonic epilepsy, strionigral degeneration, torsion dystonia, familial tremor, Gilles de la Tourette syndrome, and Hallervorden-Spatz disease.

The term excipient refers to pharmaceutically acceptable chemicals, such as known to those of ordinary skill in the art of pharmacy to aid the administration of the medicinal agent. It a compound that is useful in preparing a pharmaceutical composition, generally safe, non-toxic and neither biologically nor otherwise undesirable, and includes excipients that are acceptable for veterinary use as well as human pharmaceutical use. Exemplary excipients include binders, surfactants, diluents, disintegrants, antiadherents, and lubricants.

According to a first aspect, a compound of formula (I)

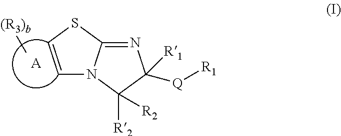

is provided, as defined herein.

In the compound of formula (I), ring A is a 5- to 7-membered, aromatic or non-aromatic carbocycle or heterocycle.

In some embodiments, ring A is 5- or 6-membered. In some other embodiments, ring A is 6- or 7-membered. In some embodiments, ring A is 6-membered. In some other embodiments, ring A is 5-membered. In still other embodiments, ring A is 7-membered.

Ring A may be either aromatic or non-aromatic. In some embodiments, when ring A is aromatic, ring A is 6-membered. In some embodiments, when ring A is aromatic, said ring is benzene.

Ring A may be either carbocyclic or heterocyclic. In some embodiments, ring A is carbocyclic. In some other embodiments, ring A is heterocyclic.

In some embodiments, when ring A is non-aromatic, said ring is mono-unsaturated e.g. a ring of formula

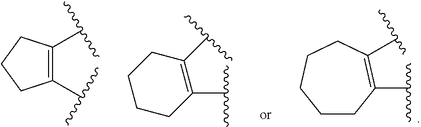 [SMILES: *C1=C(*)CCC1 ; *C1=C(*)CCCC1 ; *C1=C(*)CCCCC1]

In some other embodiments, when ring A is non-aromatic, said ring is di-unsaturated.

In some embodiments, ring A is a 5- to 7-membered, aromatic or non-aromatic carbocycle, or a 5- to 7-membered, non-aromatic heterocycle.

In some embodiments, ring A is a 5- to 7-membered, aromatic or non-aromatic carbocycle, or a 6-membered, non-aromatic heterocycle, e.g. a 6-membered, non-aromatic heterocycle containing one heteroatom in the ring.

When ring A is a heterocycle, said heterocycle may contain one or more heteroatoms in the ring. In some embodiments, when ring A is a heterocycle, e.g. a 6-membered, non-aromatic heterocycle, said heterocycle contains an oxygen atom in the ring. In some embodiments, e.g. when ring A is a 6-membered, non-aromatic heterocycle, ring A contains one heteroatom in the ring. In some of these embodiments, the heteroatom is oxygen.

In some embodiments, ring A is selected from

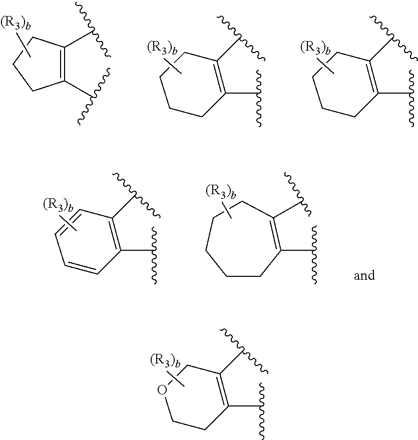

wherein R₃ and b are as defined herein.

In some particular embodiments, the compound of formula (I) more particularly is as represented by formula (Ia)

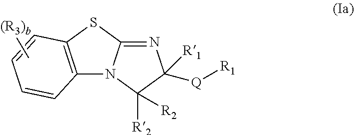

wherein b, R₁, R₂, R′₁, R′₂, each R₃ and Q are as defined herein.

In some other particular embodiments, the compound of formula (I) more particularly is as represented by formula (Ib)

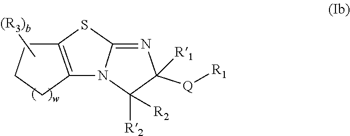

wherein b, R₁, R₂, R′₁, R′₂, each R₃ and Q are as defined herein and w is an integer of from 1 to 3, e.g. an integer of from 1 to 2, in particular w is 2.

In a compound of formula (I) as defined herein, the moiety Q is a direct bond, CH₂, CH(OH) or NH. In some embodiments, Q is CH₂, NH or CHOH, e.g. Q is CH₂ or CHOH. In some embodiments, Q is a direct bond, CH₂, or CH(OH). In some other embodiments, Q is a direct bond or CH₂, i.e. the compound may be represented by formula (Ic)

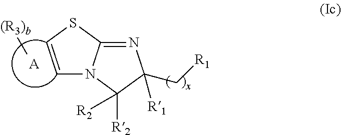

wherein ring A, b, R₁, R₂, R′₁, R′₂, and each R₃ are as defined herein, and x is 0 or 1.

In some particular embodiments, Q is a direct bond, i.e. x in formula (Ic) is 0. In some other embodiments, Q is CH₂, i.e. x in formula (Ic) is 1.

The moiety R₁ is R₄C(O), cyano, or tetrazolyl. In some embodiments, R₁ is R₄C(O) or tetrazolyl. In some other embodiments, R₁ is R₄C(O) or cyano. In some particular embodiments, R₁ is R₄C(O), i.e. the compound may be represented by formula (Id)

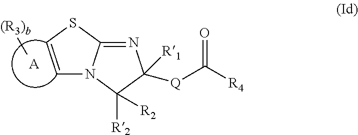

wherein ring A, b, R₂, R′₁, R′₂, each R₃, R₄ and Q are as defined herein.

In the moiety R₄C(O), R₄ is H, R₅O, or NHR₆. In some embodiments, R₄ is R₅O or NHR₆. In some particular embodiments, R₄ is R₅O, i.e. the compound of formula (I) is as represented by formula (Ie)

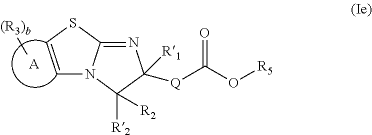

wherein ring A, b, R₂, R′₁, R′₂, each R₃, R₅ and Q are as defined herein.

In a compound of formula (Ie), the moiety R₅ is H or C1-C6 alkyl. When R₅ is C1-C6 alkyl, said alkyl more particularly may be selected from C1-C5 alkyl, or C1-C4 alkyl, or C1-C3 alkyl. In some embodiments, when R₅ is C1-C6 alkyl, said alkyl more particularly is methyl, ethyl or tert-butyl, e.g. R₅ is selected from methyl and ethyl, or R₅ is ethyl. In some embodiments R₅ is H or C1-C4 alkyl, e.g. H or C1-C3 alkyl, e.g. H, methyl or ethyl; or H or ethyl.

In some embodiments, R₅ is C1-C6 alkyl. In some other embodiments, R₅ is H, i.e. the compound of formula (I) more particularly is as represented by formula (If)

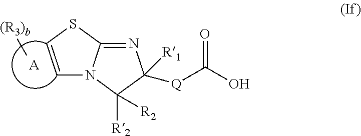

wherein ring A, b, R₂, R′₁, R′₂, each R₃, and Q are as defined herein.

In some embodiments, R₄ is NHR₆, i.e. the compound may be represented by formula (Ig)

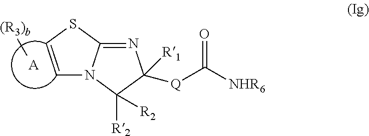

wherein ring A, b, R₂, R′₁, R′₂, each R₃, R₆ and Q are as defined herein.

In a compound of formula (Ig), the moiety R₆ is H, cyano, C1-C6 alkyl, or R₇S(O)₂. In some embodiments, R₆ is H, cyano, or R₇S(O)₂. In some embodiments, R₆ is cyano or R₇S(O)₂. In some embodiments, R₆ is H or R₇S(O)₂. In still other embodiments, R₆ is R₇S(O)₂, i.e. the compound may be represented by formula (Ih)

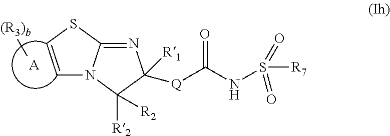

wherein ring A, b, R₂, R′₁, R′₂, each R₃, R₇ and Q are as defined herein.

In a compound of formula (Ih), R₇ is C1-C6 alkyl, C3-C6 cycloalkyl, R₈(CH₂)y, or 5- or 6-membered aryl or heteroaryl, said aryl or heteroaryl optionally being substituted by one or more moieties independently selected from C1-C6 alkyl. In some embodiments, R₇ is C1-C6 alkyl or C3-C6 cycloalkyl, e.g. R₇ is C1-C6 alkyl. In some other embodiments, R₇ is optionally substituted 5- or 6-membered aryl or heteroaryl. In still other embodiments, R₇ is C1-C6 alkyl, C3-C6 cycloalkyl, or R₈(CH₂)_{y}, e.g. R₇ is R₈(CH₂)_{y}.

When R₇ is C1-C6 alkyl, it more particularly may be selected from C1-C4 alkyl, or from C1-C3 alkyl, e.g. from methyl or ethyl.

When R₇ is C3-C6 cycloalkyl, it more particularly may be selected from C3-C5 cycloalkyl, or C3-C4 cycloalkyl, e.g. cyclopropyl.

When R₇ is optionally substituted 5- or 6-membered aryl or heteroaryl, said aryl or heteroaryl e.g. may be phenyl or 5- or 6-membered heteroaryl containing 1, 2, 3 or 4 heteroatoms in the ring, e.g. 1, 2 or 3 heteroatoms in the ring, and any substituent e.g. may be selected from C1-C4 alkyl, or from C1-C3 alkyl, e.g. any substituent is methyl. For example, when R₇ is optionally substituted 5- or 6-membered aryl or heteroaryl, R₇ may be optionally substituted phenyl or isoxazolyl, e.g. optionally substituted phenyl or isoxazol-4-yl. In some particular embodiments, when R₇ is optionally substituted 5- or 6-membered aryl or heteroaryl, R₇ is phenyl or 3,5-dimethyloxazol-4-yl, e.g. R₇ is phenyl. In the moiety R₈(CH₂)_{y}, y is an integer of from 1 to 4. In some embodiments, y is an integer of from 1 to 3, e.g. y is 2 or 3. In some other embodiments, y is an integer of from 2 to 4, e.g. y is 3.

The moiety R₈ is R₉O, R₁₀R₁₁N or R₁₂OC(O). In some embodiments, R₈ is R₉O or R₁₀R₁₁N. In some particular embodiments, R₈ is R₉O. In some other particular embodiments, R₈ is R₁₀R₁₁N. Thus, in some embodiments, R₇ is R₉O(CH₂)_{y}, wherein y is as defined herein above, e.g. y is 2 or 3. In some other embodiments, R₇ is R₁₀R₁₁N(CH₂)_{y}, wherein y is as defined herein above, e.g. y is 2 or 3, in particular 3. In still other embodiments, R₇ is R₁₂OC(O)(CH₂)_{y}, wherein y is as defined herein above, e.g. y is 2 or 3, in particular 2.

In the moiety R₉O, R₉ is H or C1-C6 alkyl, e.g. H or C1-C4 alkyl, or H or C1-C3 alkyl, in particular H or methyl. In some embodiments, R₉ is H. In some other embodiments, R₉ is as defined herein above, but is not H.

In the moiety R₁₀R₁₁N, R₁₀ and R₁₁ are independently selected from H and C1-C6 alkyl, or R₁₀ and R₁₁, together with the nitrogen atom to which they are both attached, form a 4- to 6-membered ring.

In some embodiments, when R₁₀ and R₁₁ are selected from H or C1-C6 alkyl, they more particularly are selected from C1-C6 alkyl, e.g. from C1-C4 alkyl, or from C1-C3 alkyl, e.g. R₁₀ and R₁₁ may both be ethyl. In some embodiments, when R₁₀ and R₁₁ are selected from H or C1-C6 alkyl, they more particularly are selected from H and C1-C4 alkyl, or from H and C1-C3 alkyl.

In some embodiments, when R₈ is R₁₀R₁₁N, R₁₀ and R₁₁, together with the nitrogen atom to which they are both attached, form a 4- to 6-membered ring.

In some embodiments, when R₁₀ and R₁₁, together with the nitrogen atom to which they are both attached, form a 4- to 6-membered ring, the ring more particularly is a 5- to 6-membered ring, or a 6-membered ring. Said ring optionally contains one or more further heteroatoms, e.g. one or more further heteroatoms selected from N, O and S, or from N and O. The ring may be saturated or unsaturated and heteroaromatic or non-aromatic. For example, in some embodiments, the ring is non-aromatic, e.g. non-aromatic and saturated, e.g. the ring is morpholino.

In the moiety R₁₂OC(O), R₁₂ is H or C1-C6 alkyl. In some embodiments, R₁₂ is H or C1-C4 alkyl, e.g. H or C1-C3 alkyl, in particular H or methyl. In some embodiments, R₁₂ is as defined herein above, but is not H.

In some embodiments, when R₇ is R₈(CH₂)_{y}, R₈ is a moiety selected from hydroxy, methoxy, diethylamino, morpholino and methoxycarbonyl.

In a compound of formula (I), the moiety R₂ is H, C1-C6 alkyl, C2-C6 alkenyl, C3-C6 cycloalkyl, halogen, cyano, R₁₃R₁₄N(CH₂)_{d}, R₁₅O(CH₂)ₑ, R₁₆S(CH₂)_{f}, R₁₇C(O)(CH₂)_{g},

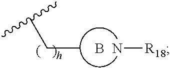

or
phenyl, optionally substituted by R₁₉O(CH₂)ᵢ.

When R₂ is C1-C6 alkyl, said alkyl e.g. may be selected from C1-C4 alkyl, or C1-C3 alkyl, e.g. methyl. As noted herein above, any alkyl group in a compound of formula (I) may be substituted by one or more F. Thus, in some embodiments, when R₂ is C1-C6 alkyl, R₂ more particularly is selected from methyl or trifluoromethyl.

When R₂ is C2-C6 alkenyl, said alkenyl e.g. may be selected from C2-C4 alkenyl, or from C2-C3 alkenyl, e.g. R₂ may be prop-1-en-2-yl.

When R₂ is C3-C6 cycloalkyl, said cycloalkyl e.g. may be C3-C5 cycloalkyl, or C3-C4 cycloalkyl, e.g. cyclopropyl.

When R₂ is halogen, said halogen e.g. may be Cl, Br or I.

In some embodiments, R₂ is H, C1-C6 alkyl, C2-C6 alkenyl, C3-C6 cycloalkyl, or halogen, e.g. R₂ is H, C1-C6 alkyl, C2-C6 alkenyl, or C3-C6 cycloalkyl, or R₂ is H, C1-C6 alkyl, or C3-C6 cycloalkyl, or R₂ is H or C1-C6 alkyl.

In some embodiments, R₂ is H, R₁₃R₁₄N(CH₂)_{d}, or

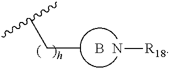

In some embodiments, R₂ is H or R₁₃R₁₄N(CH₂)_{d}. In some other embodiments, R₂ is R₁₃R₁₄N(CH₂)_{d} or

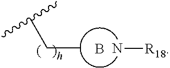

In some other embodiments, R₂ is H or

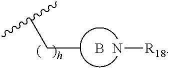

In some embodiments, R₂ is H, i.e. the compound may be represented by formula (Ij)

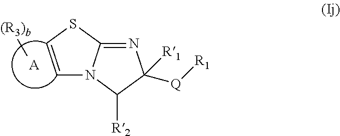

wherein ring A, b, R₁, R′₁, R′₂, each R₃ and Q are as defined herein.

In some embodiments, R₂ is R₁₃R₁₄N(CH₂)_{d}; i.e. the compound may be represented by formula (Ik)

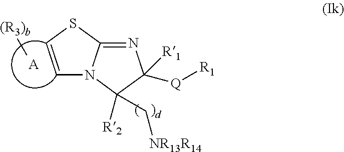

wherein ring A, b, R₁, R′₁, R′₂, each R₃, R₁₃, R₁₄, d and Q are as defined herein.

In a compound of formula (Ik), d is an integer of from 0 to 4, e.g. from 0 to 3, or from 0 to 2; e.g. d is 0 or 1. In some embodiments, d is 0. In some other embodiments, d is 1.

In the moiety R₁₃R₁₄N(CH₂)_{d}, R₁₃ is H, C1-C6 alkyl, R₂₀C(O), R₂₁S(O)₂, R₂₂O(CH₂)ⱼ, R₂₃R₂₄N(CH₂)ₖ, or benzyl, and R₁₄ is H or C1-C6 alkyl; or R₁₃ and R₁₄, together with the nitrogen atom to which they are both attached, form a 4- to 6-membered ring, said ring optionally being substituted by one or more substituents independently selected from oxo, halogen, C1-C6 alkyl, R₂₅C(O), R₂₆OC(O), and R₂₇O(CH₂)ₘ.

In some embodiments, of a compound of formula (Ik), R₁₃ is H, C1-C6 alkyl, R₂₀C(O), R₂₁S(O)₂, R₂₂O(CH₂)ⱼ, R₂₃R₂₄N(CH₂)ₖ, or benzyl, and R₁₄ is H or C1-C6 alkyl.

In some embodiments, when R₁₃ is H, C1-C6 alkyl, R₂₀C(O), R₂₁S(O)₂, R₂₂O(CH₂)ⱼ, R₂₃R₂₄N(CH₂)ₖ, or benzyl; R₁₃ more particularly is C1-C6 alkyl, R₂₀C(O), R₂₁S(O)₂, R₂₂O(CH₂)ⱼ, R₂₃R₂₄N(CH₂)ₖ, or benzyl; e.g. R₁₃ is C1-C6 alkyl, R₂₀C(O), R₂₁S(O)₂, R₂₂O(CH₂)ⱼ, or R₂₃R₂₄N(CH₂)ₖ, or R₁₃ is C1-C6 alkyl. When R₁₃ is C1-C6 alkyl, it e.g. may be C1-C4 alkyl, or C1-C3 alkyl, e.g. methyl.

When R₁₃ is R₂₀C(O), the moiety R₂₀ is H or C1-C6 alkyl, e.g. R₂₀ is H or C1-C4 alkyl, or H or C1-C3 alkyl, such as H or methyl. In some embodiments, R₂₀ is C1-C6 alkyl, e.g. C1-C4 alkyl, or C1-C3 alkyl, in particular methyl.

When R₁₃ is R₂₁S(O)₂, the moiety R₂₁ is H or C1-C6 alkyl, e.g. R₂₁ is H or C1-C4 alkyl, or H or C1-C3 alkyl, such as H or methyl. In some embodiments, R₂₁ is C1-C6 alkyl, e.g. C1-C4 alkyl, or C1-C3 alkyl, in particular methyl.

When R₁₃ is R₂₂O(CH₂)ⱼ, j is an integer of from 0 to 4, e.g. from 0 to 3, or from 0 to 2; and R₂₂ is H or C1-C6 alkyl, e.g. R₂₂ is H or C1-C4 alkyl, or H or C1-C3 alkyl, such as H or methyl. In some embodiments, R₂₂ is C1-C6 alkyl, e.g. C1-C4 alkyl, or C1-C3 alkyl, in particular methyl.

When R₁₃ is R₂₃R₂₄N(CH₂)ₖ, k is an integer of from 0 to 4, e.g. from 1 to 4, or from 1 to 3, e.g. k is 2; and R₂₃ and R₂₄ are independently selected from H and C1-C6 alkyl; or R₂₃ and R₂₄, together with the nitrogen atom to which they are both attached, form a 4- to 6-membered ring. In some embodiments, R₂₃ and R₂₄ are independently selected from C1-C6 alkyl; or R₂₃ and R₂₄, together with the nitrogen atom to which they are both attached, form a 4- to 6-membered ring. In some embodiments, R₂₃ and R₂₄, together with the nitrogen atom to which they are both attached, form a 4- to 6-membered ring.

When either of R₂₃ and R₂₄ is C1-C6 alkyl, said alkyl more e.g. may be selected from C1-C4 alkyl, or from C1-C3 alkyl.

When R₂₃ and R₂₄, together with the nitrogen atom to which they are both attached, form a 4- to 6-membered ring, said ring e.g. may be 5- to 6-membered, or 5-membered. Any such ring in particular may be non-aromatic and saturated, and optionally contain one or more further heteroatoms. In some embodiments, the ring contains no further heteroatoms, e.g. the ring is a 4- to 6-membered saturated ring containing no further heteroatoms, such as a pyrrolidinyl.

In some embodiments, when R₁₄ is H or C1-C6 alkyl, R₁₄ more particularly is H or C1-C4 alkyl, or H or C1-C3 alkyl, such as H or methyl. In some embodiments, R₁₄ is H. In some embodiments, R₁₄ is as defined herein above, but is different from H, e.g. R₁₄ is methyl.

In some embodiments of a compound of formula (Ik), R₁₃ and R₁₄, together with the nitrogen atom to which they are both attached, form a 4- to 6-membered ring, e.g. a 5- or 6-membered ring, said ring optionally being substituted by one or more substituents independently selected from oxo, halogen, C1-C6 alkyl, R₂₅C(O), R₂₆OC(O), and R₂₇O(CH₂)ₘ.

In the moiety R₂₅C(O), R₂₅ is H or C1-C6 alkyl, e.g. H or C1-C4 alkyl, or H or C1-C3 alkyl, such as H or methyl. In some embodiments, R₂₅ is C1-C6 alkyl, e.g. C1-C4 alkyl, or C1-C3 alkyl, in particular methyl.

In the moiety R₂₆OC(O), R₂₆ is H or C1-C6 alkyl, e.g. H or C1-C4 alkyl, or H or C1-C3 alkyl, such as H or methyl. In some embodiments, R₂₆ is C1-C6 alkyl, e.g. C1-C5 alkyl, or C1-C4 alkyl, such as tert-butyl.

In the moiety R₂₇O(CH₂)ₘ, m is an integer of from 0 to 4, e.g. from 1 to 4, or from 1 to 3, e.g. m is 2; and R₂₇ is H or C1-C6 alkyl, e.g. H or C1-C4 alkyl, or H or C1-C3 alkyl, such as H or methyl, e.g. H. In some embodiments, R₂₇ is C1-C6 alkyl, e.g. C1-C4 alkyl, or C1-C3 alkyl, in particular methyl.

When R₁₃ and R₁₄, together with the nitrogen atom to which they are both attached, form a 4- to 6-membered ring, said ring e.g. may be non-aromatic, e.g. the ring may be a saturated ring. The ring optionally may contain one or more further heteroatoms, e.g. one or more further heteroatoms selected from N and O. In some embodiments, when R₁₃ and R₁₄, together with the nitrogen atom to which they are both attached, form a 4- to 6-membered ring, said ring optionally contains no or at most one further heteroatom, said heteroatom being selected from N and O. In some embodiments, when R₁₃ and R₁₄, together with the nitrogen atom to which they are both attached, form an optionally substituted 4- to 6-membered ring, said ring is selected from azetidinyl, pyrrolidinyl, piperidinyl, morpholinyl and piperazin-1-yl.

In some particular embodiments, when R₁₃ and R₁₄, together with the nitrogen atom to which they are both attached, form a 4- to 6-membered ring containing a further nitrogen in the ring, this further nitrogen may be substituted by C1-C6 alkyl, R₂₅C(O), R₂₆OC(O), and R₂₇O(CH₂)ₘ. For example, in some embodiments, the ring is piperazinyl, optionally substituted in 4-position (i.e. at the further ring nitrogen) with a substituent selected from C1-C6 alkyl, R₂₅C(O), R₂₆OC(O), and R₂₇O(CH₂)ₘ as defined herein above.

In some embodiments, R₂ is

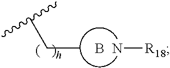

i.e. the compound of formula (I) more particularly is as represented by formula (Im)

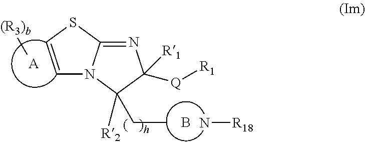

wherein ring A, b, R₁, R′₁, R′₂, each R₃, Q, h, ring B and R₁₈ are as defined herein.

In a compound of formula (Im), h is an integer of from 0 to 4, e.g. from 0 to 3, or from 0 to 2, e.g. h is 0 or 1.

In a compound of formula (Im), ring B is 4- to 6-membered, and saturated or unsaturated, e.g. saturated or mono-unsaturated. In some embodiments, ring B is selected from azetidinyl, pyrrolidinyl, piperidinyl, and tetrahydropyridinyl, e.g. from azetidinyl, piperidinyl, and tetrahydropyridinyl. In some embodiments, ring B is 4-membered, e.g. ring B is azetidinyl. In some other embodiments, ring B is 6-membered, e.g. ring B is piperidinyl or tetrahydropyridinyl.

In a compound of formula (Im), the moiety R₁₈ is H, C1-C6 alkyl, R₂₉OC(O)(CH₂)n, or R₃₀S(O)₂(CH₂)ₚ. In some embodiments, R₁₈ is H or C1-C6 alkyl, e.g. H or C1-C4 alkyl, or H or C1-C3 alkyl, such as H or methyl, e.g. H. In some other embodiments, R₁₈ is C1-C6 alkyl, R₂₉OC(O)(CH₂)n, or R₃₀S(O)₂(CH₂)ₚ, e.g. R₁₈ is R₂₉OC(O)(CH₂)n, or R₃₀S(O)₂(CH₂)ₚ, or R₁₈ is R₂₉OC(O)(CH₂)n.

In the moiety R₂₉OC(O)(CH₂)n, n is an integer of from 0 to 4, e.g. from 0 to 3, or from 0 to 2; e.g. n is 0 or 1, in particular n is 0; and R₂₉ is H or C1-C6 alkyl, e.g. H or C1-C5 alkyl, or H or C1-C4 alkyl. In some embodiments, R₂₉ is C1-C6 alkyl, e.g. C1-C5 alkyl, or C1-C4 alkyl; e.g. R₂₉ is tert-butyl.

In the moiety R₃₀S(O)₂(CH₂)ₚ, p is an integer of from 0 to 4, e.g. from 0 to 3, or from 0 to 2; e.g. p is 0 or 1, in particular p is 0; and R₉₉ is H or C1-C6 alkyl, e.g. R₃₀ is H or C1-C4 alkyl, or H or C1-C3 alkyl, such as H or methyl. In some embodiments, R₃₀ is C1-C6 alkyl, e.g. C1-C4 alkyl, or C1-C3 alkyl, such as methyl.

In some embodiments, R₂ is R₁₅O(CH₂)ₑ. In the moiety R₁₅O(CH₂)ₑ, e is an integer of from 0 to 4, e.g. from 0 to 3, or from 1 to 3, e.g. e is 1 or 2; or e is 1; and R₁₅ is H, C1-C6 alkyl or R₂₈C(O). In some embodiments, R₁₅ is H or C1-C6 alkyl, e.g. H or C1-C4 alkyl, or H or C1-C3 alkyl, such as H or methyl, in particular H. In some other embodiments, R₁₅ is H or R₂₈C(O). In some embodiments, R₁₅ is R₂₈C(O).

In R₂₈C(O), the moiety R₂₈ is H or C1-C6 alkyl, e.g. R₂₈ is H or C1-C4 alkyl, or H or C1-C3 alkyl, such as H or methyl. In some embodiments, R₂₈ is C1-C6 alkyl, e.g. C1-C4 alkyl, or C1-C3 alkyl, such as methyl.

In some embodiments, R₂ is R₁₆S(CH₂)_{f}. In the moiety R₁₆S(CH₂)_{f}, f is an integer of from 0 to 4, e.g. from 0 to 3, or from 0 to 2; e.g. f is 0 or 1, in particular f is 0; and R₁₆ is H or C1-C6 alkyl. In some embodiments, R₁₆ is H or C1-C6 alkyl, e.g. H or C1-C4 alkyl, or H or C1-C3 alkyl, such as H or methyl. In some embodiments, R₁₆ is C1-C6 alkyl, e.g. C1-C4 alkyl, or C1-C3 alkyl, such as methyl.

In some embodiments, R₂ is R₁₇C(O)(CH₂)_{g}. In the moiety R₁₇C(O)(CH₂)_{g}, g is an integer of from 0 to 4, e.g. from 0 to 3, or from 0 to 2; e.g. g is 0 or 1, in particular g is 0; and R₁₇ is H or C1-C6 alkyl. In some embodiments, R₁₇ is H or C1-C4 alkyl, or H or C1-C3 alkyl, such as H or methyl. In some embodiments, R₁₇ is C1-C6 alkyl, e.g. C1-C4 alkyl, or C1-C3 alkyl, such as methyl.

In some embodiments, R₂ is phenyl, optionally substituted by R₁₉O(CH₂)ᵢ, e.g. phenyl substituted by one moiety R₁₉O(CH₂)ᵢ, which moiety is attached in either ortho, meta or para position, e.g. in ortho or para position, in particular in para position on the phenyl ring. In the moiety R₁₉O(CH₂)ᵢ, i is an integer of from 0 to 4, e.g. from 0 to 3, or from 0 to 2; e.g. i is 0 or 1, in particular i is 0; and R₁₉ is H or C1-C6 alkyl. In some embodiments, R₁₉ is H or C1-C4 alkyl, or H or C1-C3 alkyl, such as H or methyl. In some embodiments, R₁₆ is C1-C6 alkyl, e.g. C1-C4 alkyl, or C1-C3 alkyl, such as methyl. In some embodiments, when R₂ is phenyl, optionally substituted by R₁₉O(CH₂), R₂ is 2-methoxyphenyl or 4-methoxyphenyl.

In a compound of formula (I), R′₁ and R′₂ together form a bond; or R′₁ is H, C1-C6 alkyl, C3-C6 carbocyclyl-(CH₂)_{q}, or R₃₁O(CH₂)ᵣ; and R′₂ is H.

In some embodiments, R′₁ and R′₂ together form a bond, i.e. the compound of formula (I) may be represented by formula (In)

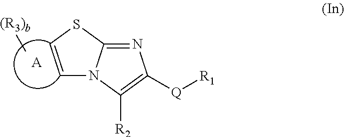

wherein ring A, b, R₁, R₂, each R₃, and Q are as defined herein.

In some embodiments of a compound of formula (In), Q is a direct bond, R₁ is R₄CO, and R₄ is R₅O or R₆NH.

In some other embodiments of a compound of formula (In), Q is a direct bond, R₁ is R₄CO, R₄ is R₅O or R₆NH, and R₂ is H or R₁₃R₁₄N(CH₂)_{d}.

In some other particular embodiments of a compound of formula (In), b is an integer of from 1 to 4,

ring A is a 5- or 6-membered carbocycle, Q is a direct bond, R₁ is R₄CO, and R₄ is R₅O or R₆NH.

In some embodiments of a compound of formula (In), b is an integer of from 1 to 4, ring A is a 6-membered carbocycle, Q is a direct bond, R₁ is R₄CO, R₄ is R₅O or R₆NH, and R₂ is H or R₁₃R₁₄N(CH₂)_{d}.

In some other embodiments of a compound of formula (In), b is an integer of from 1 to 4, ring A is a 6-membered carbocycle, Q is a direct bond, R₁ is R₄CO, R₄ is R₅O, and R₂ is H or R₁₃R₁₄N(CH₂)_{d}.

In still other embodiments of a compound of formula (In), b is an integer of from 1 to 4, ring A is a 6-membered carbocycle, Q is a direct bond, R₁ is R₄CO, R₄ is R₅O, and R₂ is H.

In some other embodiments of a compound of formula (In), b is an integer of from 1 to 4, ring A is a 6-membered carbocycle, Q is a direct bond, R₁ is R₄CO, R₄ is R₆NH, R₂ is H.

In some other embodiments, R′₁ is H, C1-C6 alkyl, C3-C6 carbocyclyl-(CH₂)_{q}, or R₃₁O(CH₂)ᵣ; and R′₂ is H, i.e. the compound of formula (I) may be represented by formula (Io)

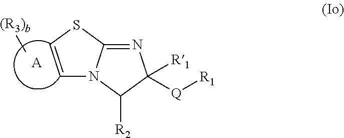

wherein ring A, b, R₁, R₂, R′, each R₃, and Q are as defined herein.

In some embodiments of a compound of formula (Io), ring A is benzene, R₂ is H, In some embodiments of a compound of formula (Io), R′₁ is H, C1-C6 alkyl, or C3-C6 carbocyclyl-(CH₂)_{q}; e.g. R′₁ is H, C1-C6 alkyl, non-aromatic C3-C6 carbocyclyl-(CH₂)_{q} or phenyl-(CH₂)_{q}. In some embodiments, R′₁ is H, C1-C6 alkyl, or C3-C6 cycloalkyl-(CH₂)_{q}, e.g. R′₁ is H or C1-C6 alkyl. In some embodiments, R′₁ is H.

When R′₁ is C1-C6 alkyl, said alkyl more particularly may be C1-C5 alkyl, or C1-C4 alkyl, e.g. methyl or isobutyl. In some embodiments, when R′₁ is C1-C6 alkyl, said alkyl is methyl. In some embodiments, R′, is selected from H, methyl and isobutyl, in particular from H and methyl.

In the moiety C3-C6 carbocyclyl-(CH₂)_{q}, q is an integer of from 0 to 4, e.g. q is an integer of from 1 to 4, or from 1 to 3, e.g. q is 1 or 2, e.g. q is 1; and the carbocyclyl e.g. is C3-C6 cycloalkyl or phenyl, e.g. C3-C5 cycloalkyl or phenyl, or C3-C4 cycloalkyl or phenyl, such as cyclopropyl or phenyl. In some embodiments, q is an integer of from 1 to 3 and the carbocyclyl is C3-C6 cycloalkyl or phenyl, e.g. C3-C5 cycloalkyl or phenyl, or C3-C4 cycloalkyl or phenyl. In some embodiments, q is 1, and the carbocyclyl is C3-C6 cycloalkyl or phenyl, e.g. C3-C5 cycloalkyl or phenyl, or C3-C4 cycloalkyl or phenyl, e.g. cyclopropyl or phenyl.

In some embodiments, R′₁ is R₃₁O(CH₂)ᵣ. In the moiety R₃₁O(CH₂)ᵣ, r is an integer of from 0 to 4, e.g. r is an integer of from 1 to 4, or r is an integer of from 1 to 3, e.g. r is 1 or 2, e.g. r is 1; and R₃₁ is H or C1-C6 alkyl, e.g. R₃₁ is H or C1-C4 alkyl, or H or C1-C3 alkyl, such as H or methyl. In some embodiments, R₃₁ is H. In some embodiments, the moiety R₃₁O(CH₂)ᵣ is a moiety of formula HO(CH₂)ᵣ, wherein r is as defined herein.

In some embodiments of a compound of formula (Io), R′₁ is selected from H, methyl, isobutyl, cyclopropylmethyl, benzyl and hydroxymethyl.

In a compound of formula (I), b, indicating the number of moieties R₃ attached to ring A, is an integer of from 0 to 4. In some embodiments b is 0, 1, 2 or 3. In some other embodiments, b is 0, 1 or 2. In some other embodiments, b is 0 or 1. In some embodiments, b is 0. In some embodiments, b is 1. In some embodiments, b is 2. In some other embodiments, b is an integer of from 1 to 4. In some embodiments, b is 1, 2 or 3. In some embodiments, b is 1 or 2. In some other embodiments, b is an integer of from 2 to 4, e.g. b is 2, or b is 4. In some embodiments, when ring A is benzene, b is not 0.

In some embodiments of a compound of formula (Ia), b is an integer of from 1 to 4, and one R₃ is in meta position (position 10 on the benzene ring, i.e. ring A), i.e. the compound is as represented by formula (Ip)

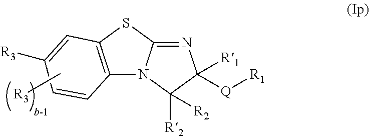

wherein R₁, R₂, R′₁, R′₂, each R₃, and Q are as defined herein and b is an integer of from 1 to 4, e.g. b is 1, 2 or 3, in particular b is 1 or 2, e.g. b is 2.

In some embodiments of a compound of formula (Ia), b is an integer of from 1 to 4, and one R₃ is in ortho position (position 9 on the benzene ring, i.e. ring A), i.e. the compound is as represented by formula (Iq)

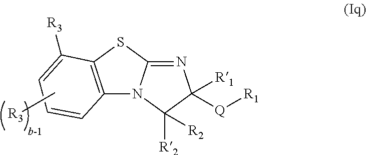

wherein R₁, R₂, R′₁, R′₂, each R₃, and Q are as defined herein and b is an integer of from 1 to 4, e.g. b is 1, 2 or 3, in particular b is 1 or 2.

In some embodiments of a compound of formula (Ia), the compound is as represented by formula (Ir)

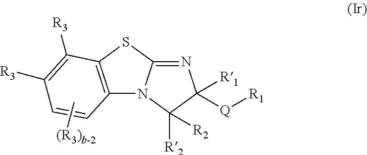

wherein R₁, R₂, R′₁, R′₂, each R₃, and Q are as defined herein and b is an integer of from 2 to 4, e.g. b is 2 or 3, in particular b is 2.

In some other embodiments, when b is 4, the compound of formula (I) more particularly is represented by formula (Is)

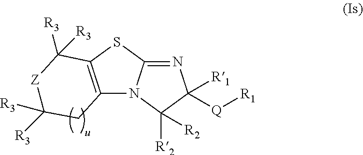

wherein R₁, R₂, R′₁, R′₂, each R₃, and Q are as defined herein, Z is a direct bond, CH₂ or a heteroatom, such as 0; and u is an integer of from 0 to 2, e.g. u is 1.

In these embodiments, when Z is a direct bond, u is 1 or 2, e.g. u is 1; and when Z is CH₂ or O, u is 0, 1 or 2. Preferably, u is 1. In some embodiments, u is 1 and Z is CH₂ or a direct bond; in particular u is 1 and Z is CH₂.

In some other embodiments, e.g. when the compound of formula (I) is a compound of formula (Ib), the compound more particularly is represented by formula (It)

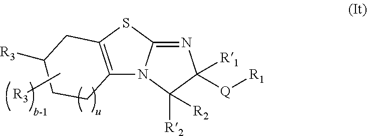

wherein R₁, R₂, R′₁, R′₂, each R₃, and Q are as defined herein, u is an integer of from 0 to 2, e.g. u is 0 or 1, or u is 1, and b is an integer of from 1 to 4, e.g. b is 1, 2 or 3, or b is 1 or 2, e.g. b is 1.

In a compound of formula (I), each moiety R₃ is independently selected from C1-C6 alkyl, C3-C6 carbocyclyl, halogen, oxo, R₃₂O, R₃₃S, and R₃₄R₃₅N and, when b is 2, 3 or 4, two R₃ attached to adjacent atoms of ring A, together with the atoms to which they are attached, may form a 3- to 6 membered ring, said ring being optionally substituted by one or more C1-C6 alkyl.

In some embodiments, each moiety R₃ is independently selected from C1-C6 alkyl, C3-C6 carbocyclyl, halogen, oxo, R₃₂O, R₃₃S, and R₃₄R₃₅N.

In some of these embodiments, e.g. in some embodiments wherein ring A is (hetero)aromatic, e.g. the compound is as represented by formula (Ia), in particular by formula (Ip), formula (Iq) or formula (Ir), each moiety R₃ is independently selected from C1-C6 alkyl, C3-C6 carbocyclyl, halogen, R₃₂O, R₃₃S, and R₃₄R₃₅N. In some of these embodiments, each R₃ is independently selected from C1-C6 alkyl, halogen, R₃₂O, R₃₃S, and R₃₄R₃₅N; or from C1-C6 alkyl, C3-C6 carbocyclyl, halogen, R₃₂O, and R₃₄R₃₅N; e.g. from C1-C6 alkyl, halogen, R₃₂O, and R₃₄R₃₅N; or from C1-C6 alkyl, halogen, and R₃₂O, in particular from halogen. In some of these embodiments, at least one R₃ is halogen.

In some other of these embodiments, e.g. in some embodiments wherein ring A is non-aromatic, e.g. the compound is as represented by formula (Ib), in particular by formula (It), or the compound is as represented by formula (Is), each moiety R₃ is independently selected from C1-C6 alkyl, C3-C6 carbocyclyl, halogen and R₃₂O; e.g. from C1-C6 alkyl.

In some embodiments, e.g. in embodiments as represented by formula (Is), when a moiety R₃ is C1-C6 alkyl, said alkyl more particularly may be C1-C4 alkyl, or C1-C3 alkyl, in particular methyl. In some embodiments represented by formula (Is), each R₃ is methyl.

In some other embodiments, e.g. in embodiments as represented by formula (It) wherein b is 1, when R₃ is C1-C6 alkyl, said alkyl more particularly is C2-C5 alkyl, or C3-C5 alkyl, e.g. tert-butyl.

In some embodiments when a moiety R₃ is C3-C6 carbocyclyl, said carbocyclyl more particularly is C3-C6 cycloalkyl or phenyl, e.g. C3-C5 cycloalkyl or phenyl, or C3-C4 cycloalkyl or phenyl, such as cyclopropyl or phenyl. In some embodiments, e.g. wherein the compound is as represented by formula (Ia), when any R₃ is a C3-C6 carbocyclyl, said carbocyclyl is non-aromatic, e.g. it is a cycloalkyl as mentioned herein above. In some other embodiments, e.g. wherein the compound is as represented by formula (Ib), in particular a compound as represented by formula (It), when R₃ is C3-C6 carbocyclyl, said carbocyclyl more particularly is phenyl.

When any R₃ is halogen, said halogen more particularly may be selected from F, Cl, and Br, e.g. from F and Cl, in particular Cl.

When any R₃ is R₃₂O, the moiety R₃₂ is H, C1-C6 alkyl, C3-C6 carbocyclyl-(CH₂)ₛ, or R₃₆R₃₇N(CH₂)ₜ. In some embodiments, R₃₂ is H or C1-C6 alkyl, e.g. R₃₂ is C1-C6 alkyl. In some other embodiments, R₃₂ is C1-C6 alkyl, C3-C6 carbocyclyl-(CH₂)ₛ, or R₃₆R₃₇N(CH₂)ₜ, e.g. R₃₂ is C3-C6 carbocyclyl-(CH₂)ₛ, or R₃₆R₃₇N(CH₂)ₜ. In some embodiments, R₃₂ is R₃₆R₃₇N(CH₂)ₜ; in some other embodiments, R₃₂ is C1-C6 alkyl or C3-C6 carbocyclyl-(CH₂)ₛ, e.g. R₃₂ is C3-C6 carbocyclyl-(CH₂)ₛ. In still other embodiments, R₃₂ is H.

When R₃₂ is C1-C6 alkyl, said alkyl e.g. may be selected from C1-C4 alkyl, or from C1-C3 alkyl, e.g. R₃₂ may be methyl.

When R₃₂ is C1-C6 alkyl, said alkyl e.g. may be selected from C1-C4 alkyl, or from C1-C3 alkyl, e.g. R₃₂ may be methyl. As noted herein, any alkyl may be substituted by one or more F. Therefore, in some embodiments, R₃₂ is methyl or trifluoromethyl.

When R₃₂ is C3-C6 carbocyclyl-(CH₂)ₛ, s is an integer of from 0 to 4, e.g. from 0 to 3, or from 0 to 2, e.g. s is 0 or 1; and the C3-C6 carbocyclyl e.g. is C4-C6 carbocyclyl, or C5-C6 carbocyclyl. In some embodiments, the carbocyclyl is C3-C6 cycloalkyl or phenyl, e.g. C4-C6 cycloalkyl or phenyl, such as cyclopentyl or phenyl. In some embodiments, any C3-C6 carbocyclyl-(CH₂)ₛ is selected from, C3-C6 cycloalkyl and benzyl, e.g. from cyclopentyl and benzyl.

When R₃₂ is R₃₆R₃₇N(CH₂)ₜ, t is an integer of from 0 to 4, e.g. from 1 to 4, or from 2 to 4, e.g. t is 2 or 3, in particular 2; and R₃₆ and R₃₇ are independently selected from H and C1-C6 alkyl, e.g. from H and C1-C3 alkyl, or from C1-C3 alkyl; or R₃₆ and R₃₇, together with the nitrogen atom to which they are both attached, form a 4- to 6-membered ring, or a 5- to 6-membered ring; optionally substituted by one or more halogen. In some embodiments, R₃₆ and R₃₇, together with the nitrogen atom to which they are both attached, form a 4- to 6-membered ring, e.g. morpholino.

When any R₃ is R₃₃S, R₃₃ is H or C1-C6 alkyl; in particular C1-C6 alkyl. When R₃₃ is C1-C6 alkyl, said alkyl e.g. may be selected from C1-C4 alkyl, or from C1-C3 alkyl, e.g. R₃₃ may be methyl. As noted herein, any alkyl may be substituted by one or more F. Therefore, in some embodiments, R₃₃ is methyl or trifluoromethyl.

When any R₃ is R₃₄R₃₅N, R₃₄ and R₃₅ are independently selected from H and C1-C6 alkyl, e.g. from C1-C6 alkyl, or from C1-C3 alkyl; or R₃₄ and R₃₅, together with the nitrogen atom to which they are both attached, form a 4- to 6-membered ring optionally substituted by one or more halogen. In some embodiments, R₃₄ and R₃₅, together with the nitrogen atom to which they are both attached, form a 4- to 6-membered ring optionally substituted by one or more halogen. In some embodiments, said ring is selected from azetidinyl, pyrrolidinyl, piperidinyl or morpholinyl. In some embodiments, any halogen attached to the ring is F.

In some other embodiments, e.g. when ring A is non-aromatic, two R₃ attached to adjacent atoms of ring A, together with the atoms to which they are attached, may form a 3- to 6 membered ring, said ring being optionally substituted by one or more C1-C6 alkyl, e.g. one or more C1-C4 alkyl, or one or more C1-C3 alkyl, or one or more methyl. Said 3- to 6 membered ring may be non-aromatic or aromatic, and heterocyclic or carbocyclic. In some embodiments, the ring formed by the two R₃ is a non-aromatic or aromatic, 3- to 6 membered carbocycle, e.g. a non-aromatic 3- to 6 membered carbocycle, e.g. a non-aromatic 3- to 5-membered carbocycle, or a non-aromatic 3- to 4-membered carbocycle, e.g. the ring is cyclopropane; or the ring is benzene. In some embodiments, when the ring is a non-aromatic carbocycle, said carbocycle is a cycloalkane. In some embodiments, the ring is a cycloalkane. For example, in some embodiments, when two R₃ attached to adjacent atoms of ring A, together with the atoms to which they are attached, form a 3- to 6 membered ring, the compound of the invention is as represented by formula (Iu)

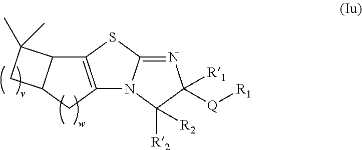

wherein R₁, R₂, R′₁, R′₂, and Q are as defined herein, v is an integer of from 0 to 3, e.g. from 0 to 2, or from 0 to 1, e.g. v is 0; and w is an integer of from 1 to 3, e.g. w is 1 or 2, and in particular w is 1.

In some embodiments, when two R₃ attached to adjacent atoms of ring A, together with the atoms to which they are attached, form a 3- to 6 membered ring, the compound of the invention is as represented by formula (Iv)

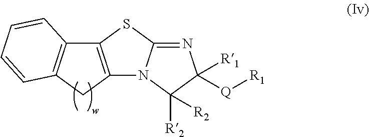

wherein R₁, R₂, R′₁, R′₂, Q are as defined herein, and w is an integer of from 1 to 3, e.g. w is 1 or 2, and in particular w is 2.

In some embodiments, two R₃ attached to adjacent atoms of ring A and, together with the atoms to which they are attached, form a benzene ring only when ring A is non-aromatic, e.g. only when ring A is not benzene.

It goes without saying that compounds of the invention may at the same be represented by more than one of the above formulas, or by formulas that combine specific features thereof. For example, a compound of formula (Ia) may also be a compound of formula (Ic), and may therefore herein be referred to as a compound of formula (Iac)

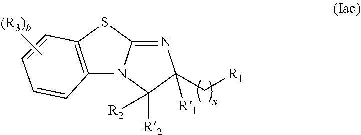

wherein b, R₁, R₂, R′₁, R′₂, each R₃ and x are as defined herein.

Likewise, a compound of formula (Iac) may also be a compound of formula (Id), i.e. represented by formula (Iacd)

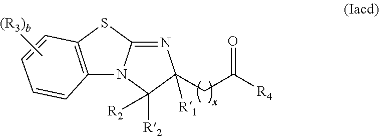

wherein b, R₂, R′₁, R′₂, each R₃, R₄ and x are as defined herein.

Likewise, a compound of formula (Iacd) may also be a compound of formula (In), i.e. represented by formula (Iacdn)

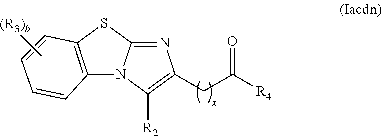

wherein b, R₂, each R₃, R₄ and x are as defined herein.

Likewise, a compound of formula (Iacn) may also be a compound of formula (Ie), i.e. represented by formula (Iacen)

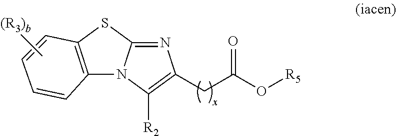

wherein b, R₂, each R₃, R₅ and x are as defined herein.

Numerous other embodiments exist within the scope of formula (I). For example, in some embodiments, a compound of formula (Ib) also is a compound of formula (Ic), and may be represented by formula (Ibc)

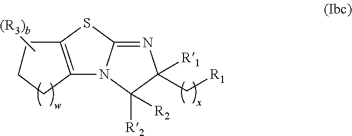

wherein b, w, R₁, R₂, R′₁, R′₂, each R₃, and x are as defined herein.

In some embodiments, a compound of formula (Ibc) also is a compound of formula (In), and may be represented by formula (Ibcn)

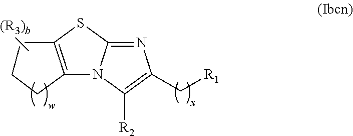

wherein b, w, R₁, R₂, each R₃, and x are as defined herein.

In some embodiments, a compound of formula (Ibcn) also is a compound of formula (Id) and may be represented by formula (Ibcdn)

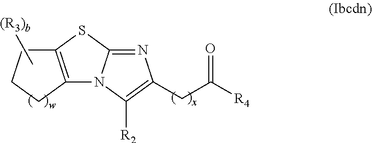

wherein b, w, R₂, each R₃, R₄ and x are as defined herein.

In some further embodiments, a compound of formula (Ic) also is a compound of formula (Id), and also is compound of formula (In), i.e. the compound is as represented by formula (Icdn)

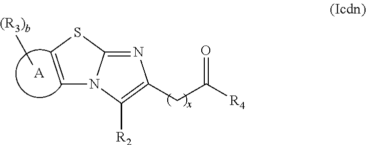

wherein ring A, b, R₂, each R₃, R₄ and x are as defined herein.

In some embodiments of a compound of formula (Ic), e.g. a compound of formula (Icd), or a compound of formula (Icdn)

ring A is a 5- to 7-membered, aromatic or non-aromatic carbocycle;
R₂ is H, halogen or R₁₃NH;
R₁₃ is C1-C6 alkyl or benzyl;
each R₃ is independently selected from C1-C6 alkyl, halogen, phenyl, and R₃₂O,
each R₃₂ is independently selected from H and C1-C6 alkyl; or
two R₃ are attached to adjacent carbon atoms of ring A and, together with the atoms to
which they are attached, form a benzene ring;
R₄ is OH, NC—NH, or R₇(S(O)₂NH;
R₇ is C1-C6 alkyl, C3-C6 cycloalkyl, or R₈(CH₂)_{y};
R₈ is R₉O, R₁₀R₁₁N, or R₁₂OC(O);
R₉ is H or C1-C6 alkyl;
R₁₀ is H or C1-C6 alkyl;
R₁₁ is H or C1-C6 alkyl; or
R₁₀ and R₁₁, together with the nitrogen atom to which they are both attached, form a 5- or 6-membered heterocyclic ring;
R₁₂ is H or C1-C6 alkyl;
y is an integer of from 1 to 4; and
any alkyl or cycloalkyl is optionally substituted by one or more F.

In some of the above embodiments of compound of formula (Icdn), R₄ is OH or R₇S(O)₂NH. In some embodiments, R₄ is OH.

In some of the above embodiments of a compound of formula (Icdn), R₄ is R₇S(O)₂NH the moiety R₇S(O)₂NH, R₇ is C3-C6 cycloalkyl, e.g. C3-C5 cyloalkyl, or C3-C4 cycloalkyl, such as cyclopropyl, or C1-C6 alkyl, e.g. C1-C4 alkyl, or C1-C3 alkyl, wherein any cycloalkyl or alkyl is optionally substituted by one or more F, or R₇ is R₈(CH₂)_{y}. In some embodiments, R₇ is C1-C6 alkyl, e.g. C1-C4 alkyl, or C1-C3 alkyl, wherein any alkyl optionally is substituted by one or more F, or R₇ is R₈(CH₂)_{y}. In some embodiments, R₇ is C3-C6 cycloalkyl, e.g. C3-C5 cyloalkyl, or C3-C4 cycloalkyl, such as cyclopropyl. In some embodiments, R₇ is alkyl, which alkyl is unsubstituted or substituted by one or more F. In some embodiments, when R₇ is alkyl, said alkyl is unsubstituted.

In some of the above embodiments of a compound of formula (Icdn), R₈ is R₉O or R₁₀R₁₁N. In some embodiments, R₈ is R₉O. In some other embodiments, R₈ is R₁₀R₁₁N.

In some of the above embodiments of a compound of formula (Icdn), R₈ is R₉O and the moiety R₉ is H or C1-C3 alkyl, e.g. H or methyl. In some embodiments of a compound of formula (Icdn), R₉ is H. In some other embodiments of a compound of formula (Icdn), R₉ is C1-C6 alkyl, e.g. C1-C3 alkyl, in particular methyl.

In some of the above embodiments of a compound of formula (Icdn), R₈ is R₁₀R₁₁N and R₁₀ and R₁₁ are independently selected from H and C1-C6 alkyl. In some other embodiments, R₈ is R₁₀R₁₁N and R₁₀ and R₁₁, together with the nitrogen atom to which they are both attached, form a 5- or 6-membered heterocyclic ring. In some embodiments of a compound of formula (Icdn), R₈ is R₁₀R₁₁N and the moieties R₁₀ and R₁₁ are independently selected from C1-C6 alkyl, e.g. C1-C3 alkyl; or R₁₀ and R₁₁, together with the nitrogen atom to which they are both attached, form a 5- or 6-membered heterocyclic ring, e.g. a 6-membered heterocyclic ring.

In some of the above embodiments of a compound of formula (Icdn), R₈ is R₁₂OC(O). In some of these embodiments, R₁₂ is H or C1-C3 alkyl, or H or methyl. In some embodiments, R₁₂ is H. In some other of these embodiments, R₁₂ is C1-C6 alkyl, e.g. C1-C3 alkyl, in particular methyl.

In some of the above embodiments of a compound of formula (Icdn), when R₄ is R₇S(O)₂NH, R₄ more specifically is selected from

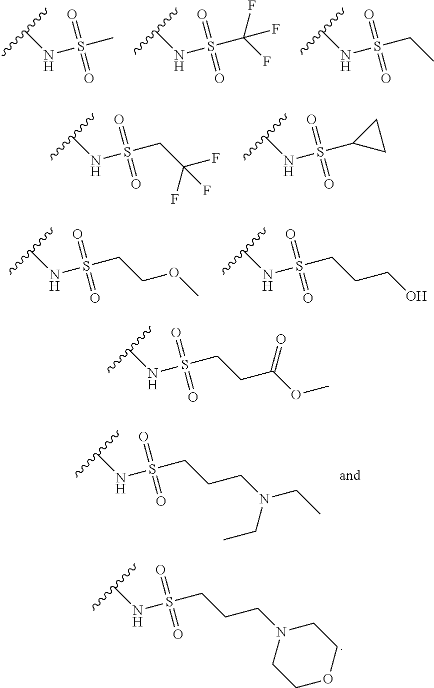

In some of the above embodiments of a compound of formula (Icdn), R₂ is H or halogen. In some other embodiments, R₂ is H or R₁₃NH. In some particular embodiments, R₂ is H.

In some other of the above embodiments of a compound of formula (Icdn), R₂ is R₁₃NH.

In a compound of formula (Icdn), ring A is a 5- to 7-membered, aromatic or non-aromatic carbocycle. In some embodiments, ring A is benzene. In some of these embodiments, b is an integer of from 0 to 3, or from 0 to 2, e.g. b is 1 or 2. In some particular embodiments, b is 1. In some other particular embodiments, b is 2.

As noted herein above, any alkyl moiety in a compound of formula (I) may be substituted by one or more F. Thus, for example, in some embodiments, when R₃₂ is alkyl, said alkyl is substituted by one or more F. In some particular embodiments, R₃₂ is selected from H, CH₃ and CF₃, e.g. from CH₃ and CF₃.

In some embodiments of a compound of formula (I), e.g. in some embodiments of a compound of formula (Icdn), R₃ is R₃₂O, wherein R₃₂ is as defined herein above, e.g. R₃₂ is CH₃ or CF₃, in particular CF₃.

In some embodiments of a compound of formula (I) e.g. in some embodiments of a compound of formula (Icdn), one R₃ is phenyl.

In some embodiments, the compound is as represented by formula (Iacen), and

x is 0 or 1; e.g. x is 0;
b is an integer of from 0 to 4; e.g. b is an integer of from 0 to 3; or b is 1 or 2;
R₂ is H, halogen or R₁₃NH; e.g. R₂ is H;
R₁₃ is C1-C6 alkyl or benzyl;
each R₃ is independently selected from C1-C6 alkyl, halogen, phenyl, and R₃₂O; e.g. from C1-C6 alkyl, halogen, and R₃₂O;
each R₃₂ is independently selected from H and C1-C6 alkyl;
or two R₃ are attached to adjacent carbon atoms of the benzene ring and, together with the atoms to which they are attached, form a benzene ring;
R₄ is OH, NC—NH, or R₇S(O)₂NH; e.g. R₄ is OH, or R₇S(O)₂NH;
R₇ is C3-C6 cycloalkyl C1-C6 alkyl, or R₈(CH₂)_{y};
R₈ is R₉O, R₁₀R₁₁N, or R₁₂OC(O);
R₉ is H or C1-C6 alkyl;
R₁₀ is H or C1-C6 alkyl; or R₁₀ is C1-C6 alkyl;
R₁₁ is H or C1-C6 alkyl; or R₁₁ is C1-C6 alkyl; or
R₁₀ and R₁₁, together with the nitrogen atom to which they are both attached, form a 5- or 6-membered heterocyclic ring;
R₁₂ is H or C1-C6 alkyl; or R₁₂ is C1-C6 alkyl;
y is an integer of from 1 to 4; and
any alkyl or cycloalkyl is optionally substituted by one or more F.

In some embodiments of a compound of formula (Icdn), e.g. in some of the above embodiments represented by formula (Iacen), each R₃ is independently selected from C1-C6 alkyl, halogen, and R₃₂O.

In some embodiments of a compound of formula (Icdn), e.g. in some of the above embodiments represented by formula (Iacen), R₁₀ is H or C1-C6 alkyl; e.g. R₁₀ is C1-C6 alkyl; and R₁₁ is H or C1-C6 alkyl; e.g. R₁₁ is C1-C6 alkyl.

In some embodiments of a compound of formula (Icdn), e.g. in some of the above embodiments represented by formula (Iacen), any C1-C6 alkyl more specifically is selected from C1-C4 alkyl, e.g. C1-C3 alkyl.

In some embodiments of a compound of formula (Icdn), e.g. in some of the above embodiments represented by formula (Iacen), x is 0 or 1; b is 1 or 2; R₄ is OH, or R₇S(O)₂NH; R₇ is C3-C6 cycloalkyl or C1-C6 alkyl, or R₈(CH₂)_{y}; R₈ is R₉O, R₁₀R₁₁N, or R₁₂OC(O); R₉ is H or C1-C6 alkyl; R₁₀ is C1-C6 alkyl; R₁₁ is C1-C6 alkyl; R₁₂ is C1-C6 alkyl; R₂ is H; each R₃ is independently selected from C1-C6 alkyl, halogen, and R₃₂O; each R₃₂ is independently selected from H and C1-C6 alkyl; and any alkyl is optionally substituted by one or more F.

In some embodiments of a compound of formula (Icdn), e.g. in some of the above embodiments represented by formula (Iacen), x is 0 or 1; b is 1 or 2; R₄ is OH, or R₇S(O)₂NH; R₂ is H; each R₃ is independently selected from CH₃, CF₃, F, Cl, Br, CH₃O, and CF₃O; and R₇ is cyclopropyl, CH₃, CF₃, CH₃CH₂, CF₃CH₂, HOCH₂CH₂, CH₃OCH₂CH₂, CH₃OC(O)CH₂CH₂, or (CH₃CH₂)₂NCH₂CH₂CH₂.

In some embodiments of a compound of formula (Icdn), e.g. in some of the above embodiments represented by formula (Iacen), x is 0; b is 1 or 2; R₄ is OH; R₂ is H; and each R₃ is independently selected from CH₃, CF₃, F, Cl, Br, CH₃O, and CF₃O.

In some embodiments of a compound of formula (Icdn), e.g. in some of the above embodiments represented by formula (Iacen), x is 1; b is 1 or 2; R₄ is OH; R₂ is H; and each R₃ is independently selected from CH₃, CF₃, F, Cl, Br, CH₃O, and CF₃O.

In some embodiments of a compound of formula (Icdn), e.g. in some of the above embodiments represented by formula (Iacen), x is 0; b is 1 or 2; R₄ is R₇S(O)₂NH; R₂ is H; each R₃ is independently selected from CH₃, CF₃, F, Cl, Br, CH₃O, and CF₃O; and R₇ is cyclopropyl, CH₃, CF₃, CH₃CH₂, CF₃CH₂, HOCH₂CH₂, CH₃OCH₂CH₂, CH₃OC(O)CH₂CH₂, or (CH₃CH₂)₂NCH₂CH₂CH₂.

In some embodiments of a compound of formula (Icdn), e.g. in some of the above embodiments represented by formula (Iacen), x is 1; b is 1 or 2; R₄ is R₇S(O)₂NH; R₂ is H; each R₃ is independently selected from CH₃, CF₃, F, Cl, Br, CH₃O, and CF₃O; and R₇ is cyclopropyl, CH₃, CF₃, CH₃CH₂, CF₃CH₂, HOCH₂CH₂, CH₃OCH₂CH₂, CH₃OC(O)CH₂CH₂, or (CH₃CH₂)₂NCH₂CH₂CH₂.

In some embodiments of a compound of formula (Icdn), e.g. in some of the above embodiments represented by formula (Iacen), when any R₃ is halogen selected from F, Cl and Br, said halogen more particularly may be selected from F and Cl.

In some other some of the above embodiments of a compound of formula (Icdn), ring A is 5, 6- or 7-membered carbocyclyl, e.g. 5- or 6-membered carbocyclyl, in particular 6-membered, mono-unsaturated carbocyclyl; i.e. the compound is a compound of formula (Ibcdn), and

x is 0 or 1; or x is 0;
b is an integer of from 0 to 4;
z is 1, 2 or 3; e.g. z is 1 or 2; or z is 2;
R₂ is H, halogen or R₁₃NH;
R₁₃ is C1-C6 alkyl or benzyl;
each R₃ is independently selected from C1-C6 alkyl, halogen, phenyl, and R₃₂O; e.g. each R₃ is independently selected from C1-C6 alkyl, phenyl, and R₃₂O; or each R₃ is independently selected from C1-C6 alkyl and R₃₂O; or each R₃ is independently selected from C1-C6 alkyl;
each R₃₂ is independently selected from H and C1-C6 alkyl; e.g. from H and methyl, e.g. each R₃₂ is H;
or two R₃ are attached to adjacent carbon atoms of ring A and, together with the atoms to which they are attached, form a benzene ring;
R₄ is OH, NC—NH, or R₇S(O)₂NH; preferably R₄ is OH or R₇S(O)₂NH;
R₇ is C3-C6 cycloalkyl or C1-C6 alkyl, or R₈(CH₂)_{y}; e.g. R₇ is C1-C6 alkyl or R₈(CH₂)_{y};
R₈ is R₉O, R₁₀R₁₁N, or R₁₂OC(O); e.g. R₈ is R₁₀R₁₁N;
R₉ is H or C1-C6 alkyl;
R₁₀ is H or C1-C6 alkyl;
R₁₁ is H or C1-C6 alkyl; or
R₁₀ and R₁₁, together with the nitrogen atom to which they are both attached, form a 5- or 6-membered heterocyclic ring;
R₁₂ is H or C1-C6 alkyl;
y is an integer of from 1 to 4; and
any alkyl or cycloalkyl is optionally substituted by one or more F.

In some of the above embodiments of a compound of formula (Icdn), e.g. in embodiments represented by formula (Ibcdn), R₄ is R₇S(O)₂NH wherein R₇ is C1-C6 alkyl, in particular C1-C3 alkyl, or R₈(CH₂)_{y}, wherein y e.g. is an integer of from 1 to 3.

In some of the above embodiments of a compound of formula (Icdn), e.g. in embodiments represented by formula (Ibcdn), R₄ is R₇S(O)₂NH, R₇ is C1-C6 alkyl, in particular C1-C3 alkyl, or R₈(CH₂)_{y}, wherein y e.g. is an integer of from 1 to 3, and R₈ is R₁₀R₁₁N.

In some of the above embodiments of a compound of formula (Icdn), e.g. in embodiments represented by formula (Ibcdn), R₄ is R₇S(O)₂NH, R₇ is C1-C6 alkyl, in particular R₈(CH₂)_{y}, wherein y e.g. is an integer of from 1 to 3, in particular from 2 to 3, and R₈ is R₁₀R₁₁N. In some of these embodiments, R₈ is diethylamino or morpholino.

In some of the above embodiments of a compound of formula (Icdn), e.g. in embodiments represented by formula (Ibcdn), R₄ is R₇S(O)₂NH wherein R₇ is C1-C6 alkyl, e.g. C1-C3 alkyl, optionally substituted by one or more F. In some embodiments, R₇ is methyl or ethyl, optionally substituted by one or more F, e.g. R₇ is CH₃ or CF₃CH₂.

In some of the above embodiments of a compound of formula (Icdn), e.g. in embodiments represented by formula (Ibcdn), when b is an integer of from 1 to 4, each R₃ is independently selected from C1-C6 alkyl, phenyl and R₃₂O; e.g. each R₃ is independently selected from C1-C6 alkyl and R₃₂O; or each R₃ is independently selected from C1-C6 alkyl.

In some embodiments of a compound of formula (Icdn), e.g. in embodiments represented by formula (Ibdn), any R₃ is methyl.

In some of the above embodiments of a compound of formula (Icdn), e.g. in embodiments represented by formula (Ibcdn), b is 1. In some of these embodiments, R₃ is C1-C6 alkyl, such as methyl or tert-butyl.

In some of the above embodiments of a compound of formula (Icdn), e.g. in embodiments represented by formula (Ibcdn), e.g. in embodiments where w is 3, the integer b is 0.

It should be realized that, unless the contrary is apparent from the context or specified, any reference herein to a compound of formula (I) also should be construed as a reference to a compound of any of the embodiments thereof, e.g. as illustrated in any of the formulas herein above. Furthermore, it should be realized that unless mutually exclusive or incompatible, the various features of the embodiments may be freely combined to give rise to further embodiments within the scope of formula (I). Thus, for example, in some embodiments, a compound of formula (Ia) also is a compound of formula (Id) (i.e. a compound of formula (Iad), e.g. a compound of formula (Ie) (i.e. a compound of formula (Iae)), a compound of formula (If) (i.e. a compound of formula (Iaf)) or a compound of formula (Ig) (i.e. a compound of formula (Iag)). Likewise in some embodiments, the compound of formula (I) is a compound of formula (Iah), or of formula (Iaj), (Iak), (Iam), (Ian), or (Iao). In some further embodiments, the compound of formula (I) is a compound of formula (Ibd), (Ibe), (Ibf), (Ibg), (Ibh), (Ibj), (Ibk), (Ibm), (Ibn) or (Ibo). In some other embodiments, using the nomenclature outlined herein above, the compound of formula (I) is a compound of formula (Icd), (Ice), (Icf), (Icg), (Ich), (Icj), (Ick), (Icm), (Icn), (Ico), (Icp), (Icq), (Icr), (Ics), (Ict), (Icu), (Icv), (Idj), (Idk), (Idm), (Idn), (Ido), (Idp), (Idq), (Idr), (Ids), (Idt), (Idu), (Idv), (Iej), (Iek), (Iem), (Ien), (Ieo), (Iep), (Ieq), (Ier), (Ies), (Iet), (Ieu), (Iev), (Ifj), (Ifk), (Ifm), (Ifn), (Ifo), (Ifp), (Ifq), (Ifr), (Ifs), (Ift), (Ifu), (Ifv), (Igj), (Igk), (Igm), (Ign), (Igo), (Igp), (Igq), (Igr), (Igs), (Igt), (Igu), (Igv), (Ihj), (Ihk), (Ihm), (Ihn), (Iho), (Ihp), (Ihq), (Ihr), (Ihs), (Iht), (Ihu), (Ihv), (Ijn), (Ijo), (Ijp), (Ijq), (Ijr), (Ijs), (Ijt), (Iju), (Ijv), (Ikn), (Iko), (Ikp), (Ikq), (Ikr), (Iks), (Ikt), (Iku), (Ikv), (Imn), (Imo), (Imp), (Imq), (Imr), (Ims), (Imt), (Imu), (Imv), (Inp), (Inq), (Inr), (Ins), (Int), (Inu), (Inv), (Iop), (Ioq), (Ior), (Ios), (Iot), (Iou) or (Iov).

Further embodiments are e.g. a compound as represented by formula (Imn) that is also a compound as represented by formula (Icd), i.e. a compound having a formula which, using the above outlined nomenclature, may be referred to as formula (Icdmn)

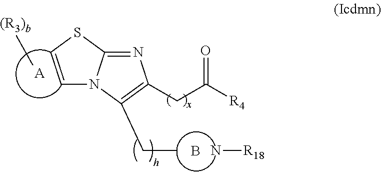

wherein ring A, b, each R₃, R₄, h, x, ring B and R₁₈ are as defined herein.

In some embodiments, the compound of formula (Icdmn) also is a compound of formula (Ib), i.e. a compound of formula (Ibcdmn)

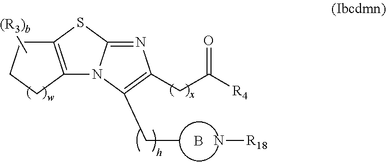

wherein w, b, each R₃, R₄, h, x, ring B and R₁₈ are as defined herein. In some of these embodiments, x is 0.

In some other embodiments the compound of formula (Icdmn) also is a compound of formula (Is), i.e. a compound of formula (Icdmns)

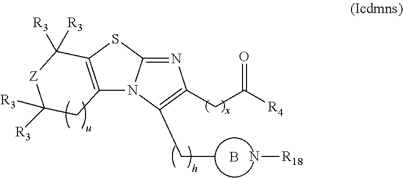

wherein Z, each R₃, R₄, h, x, ring B and R₁₈ are as defined herein.

In still other embodiments, the compound of formula (Icdmn) also is a compound of formula (Ia), i.e. a compound of formula (Iacdmn),

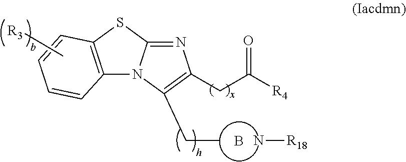

wherein b, each R₃, R₄, h, x, ring B and R₁₈ are as defined herein. In some of these embodiments, the compound also is a compound of formula (Ip), or a compound of formula (q), or a compound of formula (Ir). In some of these embodiments, the compound more particularly is a compound of formula (Ie), e.g. a compound of formula (If), in particular a compound of formula (Iep) or (Ifp). In some of these embodiments, x is 0.

In some other embodiments, a compound of formula (Ikn) also is a compound (Icd), i.e. a compound of formula (Icdkn)

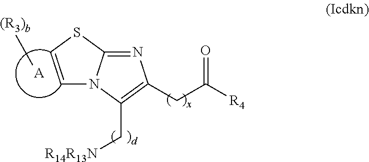

wherein ring A, b, each R₃, R₄, d, x, R₁₃ and R₁₄ are as defined herein.

In some of those embodiments, the compound of formula (Icdkn) also is a compound as represented by formula (Ib), e.g. by formula (It). In some other embodiments, the compound of formula (Icdkn) also is a compound of formula (Is). In some other of those embodiments, the compound of formula (Icdkn) also is as represented by formula (Ia). In some of these embodiments, x is 0.

In some other embodiments, a compound of formula (Iac) also is a compound of formula (Ido), i.e. a compound as represented by formula (Iacdo)

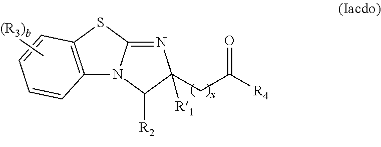

wherein b, R₂, R′₁, each R₃, R₄, and x are as defined herein. In some embodiments, the compound of formula (Iacdo) also is a compound of formula (Ij), i.e. wherein R₂ is H. In some of these embodiments, the compound of formula (Iacdo) or formula (Iacdjo), more particularly is a compound of formula (Iaceo), i.e.a compound wherein R₄ is R₅O. In some embodiments, the compound of formula (Iacdo) or (Iaceo) also is a compound of formula (Ip) or (Iq), or a compound of formula (Ijp) or (Ijq). In some of these embodiments, x is 0.

In some embodiments of a compound of formula (Iacen), in particular wherein R₂ is H, b is not 0.

In some embodiments of a compound of formula (Iacen), in particular wherein R₂ is H, b is an integer of from 1 to 4 and one R₃ is attached in position 9 on ring A.

In some embodiments of a compound of formula (Iacen), in particular wherein R₂ is H, b is not 0, and when b is 1, R₃ is not attached in position 12 on ring A.

In some other embodiments of a compound of formula (Iacen), in particular wherein R₂ is H, b is not 0, and when b is 1 and R₃ is attached in position 10 on ring A, R₃ is not halogen, methyl, methoxy, trifluoromethyl or ethyl.

In some other embodiments of a compound of formula (Iacen), in particular wherein R₂ is H, b is not 0, and when b is 1 and R₃ is attached in position 10 on ring A, R₃ is not halogen, C1-C6 alkyl optionally substituted by F, or C1-C6 alkoxy, optionally substituted by F.

In some other embodiments of a compound of formula (Iacen), in particular wherein R₂ is H, b is not 0, and when b is 1, R₃ is not attached in position 10 on ring A.

In some other embodiments of a compound of formula (Iacen), in particular wherein R₂ is H, b is not 0, and when b is 1, and R₃ is halogen, C1-C6 alkyl or C1-C6 alkoxy, R₃ is not attached in position 10 or 12 on ring A.

In some other embodiments of a compound of formula (Iacen), in particular wherein R₂ is H, b is not 0, and when b is 1, R₃ is not attached in position 10 or 12 on ring A.

In some other embodiments of a compound of formula (Iacen), in particular wherein R₂ is H, b is not 0, and when b is 1, R₃ is not attached in position 10 or 12 on ring A, and when b is 2, ring A is not 10,11-dialkylsubstituted.

In some other embodiments of a compound of formula (Iacen), in particular wherein R₂ is H, b is not 0, and when b is 1 and R₃ is halogen, C1-C6 alkyl or C1-C6 alkoxy, R₃ is not attached in position 10 or 12 on ring A, and when b is 2 and each R₃ is halogen, C1-C6 alkyl or C1-C6 alkoxy, ring A is not 10,11-disubstituted.

In some embodiments of a compound of formula (Iacen), in particular wherein R₂ is H, b is not 0 or 1, and when b is 2, one R₃ is in position 9 on ring A.

In some embodiments of a compound of formula (Iacen), in particular wherein R₂ is H, b is not 0, and when b is 1 or 2, one R₃ is in position 9 on ring A.

In some embodiments of a compound of formula (I), e.g. when ring A is benzene, Q is a direct bond or (CH₂), R₁ is R₅OC(O), and R′₁ and R′₂ form a bond, b is not 0.

In some other embodiments of a compound of formula (I), e.g. when ring A is benzene, Q is a direct bond or (CH₂), R₁ is R₅OC(O), and R′, and R′₂ form a bond, b is an integer of from 1 to 4 and one R₃ is attached in position 9 on ring A.

In some other embodiments of a compound of formula (I), e.g. when ring A is benzene, Q is a direct bond or (CH₂), R₁ is R₅OC(O), and R′, and R′₂ form a bond, b is not 0, and when b is 1, R₃ is not attached in position 12 on ring A.

In some other embodiments of a compound of formula (I), e.g. when ring A is benzene, Q is a direct bond or (CH₂), R₁ is R₅OC(O), and R′₁ and R′₂ form a bond, b is not 0, and when b is 1 and R₃ is attached in position 10 on ring A, R₃ is not halogen, methyl, methoxy, trifluoromethyl or ethyl.

In some other embodiments of a compound of formula (I), e.g. when ring A is benzene, Q is a direct bond or (CH₂), R₁ is R₅OC(O), and R′, and R′₂ form a bond, b is not 0, and when b is 1 and R₃ is attached in position 10 on ring A, R₃ is not halogen, C1-C6 alkyl optionally substituted by F, or C1-C6 alkoxy, optionally substituted by F.

In some other embodiments of a compound of formula (I), e.g. when ring A is benzene, Q is a direct bond or (CH₂), R₁ is R₅OC(O), and R′₁ and R′₂ form a bond, b is not 0, and when b is 1, R₃ is not attached in position 10 on ring A.

In some other embodiments of a compound of formula (I), when ring A is benzene, Q is a direct bond or (CH₂), R₁ is R₅OC(O), and R′₁ and R′₂ form a bond, b is not 0, and when b is 1, and R₃ is halogen, C1-C6 alkyl or C1-C6 alkoxy, R₃ is not attached in position 10 or 12 on ring A.

In some other embodiments of a compound of formula (I), e.g. when ring A is benzene, Q is a direct bond or (CH₂), R₁ is R₅OC(O), and R′, and R′₂ form a bond, b is not 0, and when b is 1, R₃ is not attached in position 10 or 12 on ring A.

In some other embodiments of a compound of formula (I), e.g. when ring A is benzene, Q is a direct bond or (CH₂), R₁ is R₅OC(O), and R′, and R′₂ form a bond, b is not 0, and when b is 1, R₃ is not attached in position 10 or 12 on ring A, and when b is 2 and each R₃ is methyl, ring A is not 10,11-disubstituted.

In some other embodiments of a compound of formula (I), when ring A is benzene, Q is a direct bond or (CH₂), R₁ is R₅OC(O), and R′₁ and R′₂ form a bond, b is not 0, and when b is 1, R₃ is not attached in position 10 or 12 on ring A, and when b is 2 and each R₃ is C1-C6 alkyl, ring A is not 10,11-disubstituted.

In some other embodiments of a compound of formula (I), e.g. when ring A is benzene, Q is a direct bond or (CH₂), R₁ is R₅OC(O), and R′₁ and R′₂ form a bond, b is not 0, and when b is 1 and R₃ is halogen, C1-C6 alkyl or C1-C6 alkoxy, R₃ is not attached in position 10 or 12 on ring A, and when b is 2 and each R₃ is halogen, C1-C6 alkyl or C1-C6 alkoxy, ring A is not 10,11-disubstituted.

In some other embodiments of a compound of formula (I), e.g. when ring A is benzene, Q is a direct bond or (CH₂), R₁ is R₅OC(O), and R′₁ and R′₂ form a bond, b is not 0 or 1, and when b is 2, one R₃ is in position 9 on ring A.

In some other embodiments of a compound of formula (I), e.g. when ring A is benzene, Q is a direct bond or (CH₂), R₁ is R₅OC(O), and R′, and R′₂ form a bond, b is not 0, and when b is 1 or 2, one R₃ is in position 9 on ring A.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0, and when b is 1 and R₃ is attached in position 10 on ring A, R₃ is not halogen, C1-C6 alkyl optionally substituted by F, or C1-C6 alkoxy, optionally substituted by F.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0, and when b is 1, R₃ is not attached in position 10 on ring A.

In some other embodiments of a compound of formula (I), when ring A is benzene, Q is a direct bond or (CH₂), R₁ is HC(O), R₅OC(O) or tetrazolyl, and R′₁ and R′₂ form a bond, b is not 0, and when b is 1, and R₃ is halogen, C1-C6 alkyl or C1-C6 alkoxy, R₃ is not attached in position 10 or 12 on ring A.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0, and when b is 1, R₃ is not attached in position 10 or 12 on ring A.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0, and when b is 1, R₃ is not attached in position 10 or 12 on ring A, and when b is 2 and each R₃ is methyl, ring A is not 10,11-disubstituted.

In some other embodiments of a compound of formula (I), when ring A is benzene, Q is a direct bond or (CH₂), R₁ is HC(O), R₅OC(O) or tetrazolyl, and R′₁ and R′₂ form a bond, b is not 0, and when b is 1, R₃ is not attached in position 10 or 12 on ring A, and when b is 2 and each R₃ is C1-C6 alkyl, ring A is not 10,11-disubstituted.

In some other embodiments of a compound of formula (I), e.g. when ring A is benzene, Q is a direct bond or (CH₂), R₁ is HC(O), R₅OC(O) or tetrazolyl, and R′, and R′₂ form a bond, b is not 0, and when b is 1 and R₃ is halogen, C1-C6 alkyl or C1-C6 alkoxy, R₃ is not attached in position 10 or 12 on ring A, and when b is 2 and each R₃ is halogen, C1-C6 alkyl or C1-C6 alkoxy, ring A is not 10,11-disubstituted.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0 or 1, and when b is 2, one R₃ is in position 9 on ring A.

In some other embodiments of a compound of formula (I), e.g. when ring A is benzene, Q is a direct bond or (CH₂), R₁ is HC(O), R₅OC(O) or tetrazolyl, and R′₁ and R′₂ form a bond, b is not 0, and when b is 1 or 2, one R₃ is in position 9 on ring A.

Some compounds falling within the scope of formula (I) are commercially available or have been previously disclosed, but have not previously been disclosed for use in therapy.

Thus, a compound of formula (I) selected from 9,10-dichloro-7-thia-2,5-diazatricyclo[6.4.0.0^{2,6}]dodeca-1(8),3,5,9,11-pentaene-4-carboxylic acid,
10-(trifluoromethoxy)-7-thia-2,5-diazatricyclo[6.4.0.0^{2,6}]dodeca-1(8),3,5,9,11-pentaene-4-carboxylic acid,
9-chloro-7-thia-2,5-diazatricyclo[6.4.0.0^{2,6}]dodeca-1(8),3,5,9,11-pentaene-4-carboxylic acid,
10,11-dichloro-7-thia-2,5-diazatricyclo[6.4.0.0^{2,6}]dodeca-1(8),3,5,9,11-pentaene-4-carboxylic acid,
10-fluoro-7-thia-2,5-diazatricyclo[6.4.0.0^{2,6}]dodeca-1(8),3,5,9,11-pentaene-4-carboxylic acid,
10,12-dichloro-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5-triene-4-carboxylic acid,
10-tert-butyl-7-thia-2,5-diazatricyclo[6.4.0.0² 6]dodeca-1(8),3,5-triene-4-carboxylic acid
ethyl 10,11-dimethyl-7-thia-2,5-diazatricyclo[6.4.0.0^{2,6}]dodeca-1(8),3,5,9,11-pentaene-4-carboxylate,
ethyl 10-methoxy-7-thia-2,5-diazatricyclo[6.4.0.0^{2,6}]dodeca-1(8),3,5,9,11-pentaene-4-carboxylate,
ethyl 12-methoxy-7-thia-2,5-diazatricyclo[6.4.0.0^{2,6}]dodeca-1(8),3,5,9,11-pentaene-4-carboxylate,
ethyl 10-chloro-7-thia-2,5-diazatricyclo[6.4.0.0^{2,6}]dodeca-1(8),3,5,9,11-pentaene-4-carboxylate,
ethyl 10-methyl-7-thia-2,5-diazatricyclo[6.4.0.0^{2,6}]dodeca-1(8),3,5,9,11-pentaene-4-carboxylate,
ethyl 10-bromo-7-thia-2,5-diazatricyclo[6.4.0.0^{2,6}]dodeca-1(8),3,5,9,11-pentaene-4-carboxylate, and
ethyl 12-methyl-7-thia-2,5-diazatricyclo[6.4.0.0^{2,6}]dodeca-1(8),3,5,9,11-pentaene-4-carboxylate,
   is not claimed per se, but at least is claimed for use in therapy, e.g. for use in the treatment of cancer, an inflammatory disorder, an autoimmunity disorder or a neurodegenerative disorder.

Therefore, as far any of the above defined embodiments are drawn to a compound of formula (I) per se, the above listed compounds are implicitly disclaimed.

As indicated herein above, some compounds falling within the scope of formula (I) have been previously suggested for use in therapy, but not for use for use in the treatment of cancer, an inflammatory disorder, an autoimmunity disorder or a neurodegenerative disorder. Thus, in some embodiments, a compound of formula (I) as defined herein above but also selected from
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid
   is claimed herein for only the use in the treatment of cancer or a neurodegenerative disorder.

In some other embodiments, a compound of formula (I) as defined herein above but also selected from
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
   is claimed herein for only the use in the treatment of cancer, an autoimmunity disorder or a neurodegenerative disorder.

In still other embodiments, a compound of formula (I) selected from
12-chloro-7-thia-2,5-diazatricyclo[6.4.0.0^{2,6}]dodeca-1(8),3,5,9,11-pentaene-4-carboxylic acid, and
10-methoxy-7-thia-2,5-diazatricyclo[6.4.0.0^{2,6}]dodeca-1(8),3,5,9,11-pentaene-4-carboxylic acid
   is claimed herein for only the use in the treatment of an inflammatory disorder, cancer, an auto immunity disorder or a neurodegenerative disorder.

Therefore, as far any of the above defined embodiments are drawn to a compound of formula (I), or to any embodiment thereof, per se or to its first medical use in therapy, the above identified compounds are implicitly disclaimed.

Studies have shown efficacy of the compounds of the invention in vitro and in vivo in mice and, although the compounds have been developed toward S100A9 inhibition, they can also show activity to other S100 proteins. The present invention therefore relates to compounds as defined herein, as S100 protein inhibitors, mainly as S100A9 inhibitors and to their use in treatment or prevention of S100-protein related diseases, in particular diseases related to the activity of S100A9 protein.

In particular, the present invention relates to the compounds of formula (I) as defined herein, to pharmaceutical compositions comprising said compounds, to the use of such compositions in the therapeutic treatment of conditions selected from in particular cancer, but also automimmune diseases, inflammatory diseases and neurodegenerative diseases, to a method of treatment of such conditions, and to said compounds for use in the treatment of conditions selected from in particular cancer, but also automimmune diseases, inflammatory diseases and neurodegenerative diseases, as well as the use of said compounds in the manufacture of pharmaceutical compositions for the treatment of such conditions.

The present invention includes pharmaceutical compositions comprising at least one compound according to formula (I), or an individual isomer, racemic or non-racemic mixture of isomers or a pharmaceutically acceptable salt thereof, together with at least one pharmaceutically acceptable excipient, e.g. a carrier, and optionally other therapeutic and/or prophylactic ingredients.

A pharmaceutical composition according to the invention may be for topical (local) or systemic administration, e.g. for enteral administration, such as rectal or oral administration, or for parenteral administration to a mammal (especially a human), and comprises a therapeutically effective amount of a compound according to the invention or a pharmaceutically acceptable salt thereof, as active ingredient, in association with a pharmaceutically acceptable excipient, e.g. a pharmaceutically acceptable carrier. The therapeutically effective amount of the active ingredient is as defined herein above and depends e.g. on the species of mammal, the body weight, the age, the individual condition, individual pharmacokinetic data, the disease to be treated and the mode of administration.

For enteral, e.g. oral, administration, the compounds of the invention may be formulated in a wide variety of dosage forms. The pharmaceutical compositions and dosage forms may comprise a compound or compounds of the present invention or pharmaceutically acceptable salt(s) thereof as the active component. The pharmaceutically acceptable carriers may be either solid or liquid. Solid form preparations include powders, tablets, pills, lozenges, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The formulation of the active compound may comprise an encapsulating material as carrier, providing a capsule in which the active component, with or without carriers, is surrounded by a carrier, which is in association with it.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Exemplary compositions for rectal administration include suppositories which can contain, for example, a suitable non-irritating excipient, such as cocoa butter, synthetic glyceride esters or polyethylene glycols, which are solid at ordinary temperatures, but liquefy and/or dissolve in the rectal cavity to release the drug.

The compounds of the invention also may be administered parenterally, e.g. by inhalation, injection or infusion, e.g. by intravenous, intraarterial, intraosseous, intramuscular, intracerebral, intracerebroventricular, intrasynovial, intrasternal, intrathecal, intralesional, intracranial, intratumoral, intracutaneous and subcutaneous injection or infusion.

Thus, for parenteral administration, the pharmaceutical compositions of the invention may be in the form of a sterile injectable or infusible preparation, for example, as a sterile aqueous or oleaginous suspension. This suspension may be formulated according to techniques known in the art using suitable dispersing or wetting agents (e.g., Tween 80), and suspending agents. The sterile injectable or infusible preparation may also be a sterile injectable or infusible solution or suspension in a non-toxic parenterally acceptable diluent or solvent. For example, the pharmaceutical composition may be a solution in 1,3-butanediol. Other examples of acceptable vehicles and solvents that may be employed in the compositions of the present invention include, but are not limited to, mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

Solutions for parenteral use also may contain suitable stabilizing agents, and if necessary, buffer substances. Suitable stabilizing agents include antioxidizing agents, such as sodium bisulfate, sodium sulfite or ascorbic acid, either alone or combined, citric acid and its salts and sodium EDTA. Parenteral solutions may also contain preservatives, such as benzalkonium chloride, methyl- or propyl-paraben, and cholorobutanol.

For inhalation or nasal administration, suitable pharmaceutical formulations are as particles, aerosols, powders, mists or droplets, e.g. with an average size of about 10 μm in diameter or less. For example, compositions for inhalation may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions of the invention also may be administered topically, to the skin or to a mucous membrane. For topical application, the pharmaceutical composition may be e.g. a lotion, a gel, a paste, a tincture, a transdermal patch, a gel for transmucosal delivery. The composition may be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition may be formulated as a suitable lotion or cream containing the active compound suspended or dissolved in a carrier. Suitable carriers include, but are not limited to, mineral oil, sorbitan monostearate, polysorbate 60, cetyl esters wax, cetaryl alcohol, 2-octyldodecanol, benzyl alcohol and water. The pharmaceutical compositions of this invention may also be topically applied to the lower intestinal tract by rectal suppository formulation or in a suitable enema formulation.

Suitable pharmaceutical excipients, e.g. carriers, and methods of preparing pharmaceutical dosage forms are described in Remington's Pharmaceutical Sciences, Mack Publishing Company, a standard reference text in art of drug formulation.

The pharmaceutical compositions may comprise from approximately 1% to approximately 95%, preferably from approximately 20% to approximately 90% of a compound of formula (I), together with at least one pharmaceutically acceptable excipient. In general, the compounds of the invention will be administered in a therapeutically effective amount by any of the accepted modes of administration for agents that serve similar utilities. Suitable daily dosages typically ranges from 1 to 1000 mg, e.g. 1-500 mg daily, or 1-50 mg daily, depending upon numerous factors such as the severity of the disease to be treated, the age and relative health of the patient, the potency of the compound used, the route and form of administration, and the indication towards which the administration is directed, etc. One of ordinary skill in the art of treating such diseases will be able, without undue experimentation and in reliance upon personal knowledge and the disclosure of this application, to ascertain a therapeutically effective amount of the compounds of the present invention for a given disease. Compounds of the invention may be administered as pharmaceutical formulations including those suitable for enteral or parenteral administration. The preferred manner of administration is generally oral using a convenient daily dosage regimen which can be adjusted according to the degree of affliction.

According to one aspect, the present invention relates to a method of treatment of a disease that responds to inhibition of a member of the S100 protein family, e.g. S100A9, e.g. a cancer, an autoimmune disease, an inflammatory disease, or a neurodegenerative disease, which method comprises administering a therapeutically effective amount of a compound of formula (I), or pharmaceutically acceptable salt thereof, to a warm-blooded animal, e.g., a human, in need of such treatment.

The preparation of compounds within the scope of formula (I) is well within the capacity of the person of ordinary skill in the art.

The following examples will enable a person skilled in the art to more clearly understand and practice the present invention. These examples, however, should not be considered as limiting the scope of the invention, but merely as being illustrative and representative thereof.

### EXAMPLES

Abbreviations Used

dba dibenzylidene acetone
DCM dichloromethane
DIBAL diisobutylaluminium hydride

### DIPEA N,N-diisopropylethylamine

DMA dimethylacetamide
DMAP 4-(dimethylamino)pyridine
DMAW 90 dichloromethane, methanol, acetic acid, water (90:18:3:2)
DMAW 120 dichloromethane, methanol, acetic acid, water (120:9:1.5:1)
DME 1,2-dimethoxyethylether
DMF N,N-dimethyl formamide
DMSO dimethyl sulfoxide
dppf 1,1 f-bis(diphenylphosphino)ferrocene
EDC N-[3-(dimethylamino)propyl]-N′-ethylcarbodiimide hydrochloride (1:1)
eq equivalent(s)
EtOAc ethyl acetate
EtOH ethanol
FCC flash column chromatography
h hour(s)
HPLC high performance liquid chromatography
IPA isopropanol
MeCN acetonitrile
MeOH methanol
min minute(s)
NBS 1-bromo-2,5-pyrrolidinedione
NMP 1-methylpyrrolidin-2-one
pTSA para-toluenesulfonic acid monohydrate
SCX strong cation exchange
TFA trifluoroacetic acid
THF tetrahydrofuran
TLC thin layer chromatography
TMSCl trimethylsilyl chloride
Tris 2-amino-2-(hydroxymethyl)propane-1,3-diol
Xantphos 4,5-bis(diphenylphosphino)-9,9-dimethylxanthene

General

Unless otherwise stated, any chiral targets or intermediates were prepared racemically.

All acetone, 1,4-dioxane, DMA, DME, DMF, THF, NMP and pyridine used was anhydrous.

All naming of molecules was performed using MarvinSketch 14.10.27.0

HPLC methods were as follows:

“Low pH Method” refers to HPLC purification using a mobile phase consisting of 0.1% formic acid in a gradient of 0-100% MeCN in water. The stationary phase consisted of a Waters Sunfire C18 column, 5 μm particle size, 19×100 mm.

“High pH method” refers to HPLC purification using a mobile phase consisting of 0.2% aqueous ammonia in a gradient of 5-100% MeCN in water. The stationary phase consisted of a Waters X-bridge C18 column, 10 μm particle size, 30×100 mm. “Neutral method” refers to HPLC purification using a mobile phase (without modifier) consisting of a gradient of 10-100% MeCN in water. The stationary phase consisted of a Waters Sunfire C18 column, 10 μm particle size, 30×100 mm.

Microwave reactions were carried out using a CEM Discover or Activent microwave apparatus.

### Intermediates

### (3,4-Dichloro-2-fluorophenyl)thiourea

A suspension of 3,4-dichloro-2-fluoroaniline (11.0 g, 61.1 mmol) and ammonium thiocyanate (6.15 g, 80.8 mmol) in 6M HCl (aq) (110 mL) was heated at 80° C. for 1.5 h with vigorous stirring. The cooled reaction mixture was diluted with water (600 mL) and the resulting precipitate collected by filtration, washed with water and dried under vacuum at 40° C. to afford the title compound as a yellow solid (2.45 g, 19% yield); m/z=238.8 (MH)⁺.

### 6,7-Dichloro-1,3-benzothiazol-2-amine

To a stirred solution of (3,4-dichloro-2-fluorophenyl)thiourea (85%, 5.94 g, 21.1 mmol) in NMP (120 mL), at room temperature under nitrogen, was added sodium hydride (60%, 1.27 g, 31.7 mmol). The reaction was heated at 130° C. for 3 h. The reaction was allowed to cool to room temperature and was made basic with 2M aqueous NH₃ and diluted with water (100 mL) and EtOAc (200 mL). The layers were separated. The organic layer was washed with water (3×100 mL), with vigorous shaking to remove NMP, washed with brine (100 mL), dried (Na₂SO₄), filtered and concentrated to give a solid which was triturated with DCM, affording the title compound as a beige solid (1.75 g, 38% yield); ¹H NMR (500 MHz, DMSO-d6) δ 7.29 (d, 1H), 7.43 (d, 1H), 7.84 (s, 2H).

### 2-Chloro-3-fluoro-6-nitrobenzene-1-thiol

Sodium sulfide (7.26 g, 93.0 mmol) was added to a stirred solution of 2-chloro-1,3-difluoro-4-nitrobenzene (5.00 g, 25.8 mmol) in DMSO (100 mL). The reaction was allowed to stir at room temperature for 18 h then the reaction mixture was diluted with water (500 mL), and acidified to pH 1-2 by the addition of 50% aqueous HCl. The mixture was then extracted with EtOAc (3×120 mL). The combined organic extracts were washed with brine (50 mL), dried over sodium sulfate, filtered and the filtrate was concentrated in vacuo. The residue was purified by FCC over silica (eluent: heptane:EtOAc 1:0 to 85:15) affording the title compound as fine yellow needles (1.98 g, 35% yield); ¹H NMR (500 MHz, Chloroform-d) δ 7.10 (dd, 1H), 8.26 (dd, 1H).

### 6-Amino-2-chloro-3-fluorobenzene-1-thiol

SnCl₂.H₂O (8.57 g, 38.0 mmol) was added portionwise to a stirred solution of 2-chloro-3-fluoro-6-nitrobenzene-1-thiol (1.97 g, 9.49 mmol) in EtOH (25 mL). The resulting mixture was allowed to stir for 1 min then concentrated HCl (8.2 mL) was added slowly. The reaction was stirred at 85° C. for 1.5 h. The reaction was then allowed to stand at room temperature for 18 h. The reaction mixture was brought to pH˜10 by the addition of 30% aqueous NaOH, the resulting suspension was filtered over glass fibre filter paper and the filter pad washed with EtOAc (3×100 mL). The combined filtrates were separated and the organic phase was dried over Na₂SO₄, the mixture filtered and the filtrate concentrated in vacuo to afford a bright yellow solid. This material was triturated with EtOAc/heptane and filtered. The solid collected was dried in vacuo to afford the title compound as a bright yellow solid (900 mg, 48% yield); m/z=177.9 (MH)⁺.

### 7-Chloro-6-fluoro-1,3-benzothiazol-2-amine

A solution of cyanogen bromide (373 mg, 3.52 mmol) in EtOH (2 mL) was added to a solution of 6-amino-2-chloro-3-fluorobenzene-1-thiol (500 mg, 2.81 mmol) in an EtOH:water mixture (9:1, 5 mL) in a pressure tube. The reaction was sealed and stirred at 70° C. for 4 h. The reaction was allowed to cool to room temperature then it was diluted with water (30 mL) and the aqueous phase extracted with EtOAc (3×30 mL). The combined organic extracts were washed with brine (20 mL), dried over Na₂SO₄, filtered and the filtrate was concentrated in vacuo. The residue obtained was purified by FCC over silica (eluent: heptane:EtOAc 1:0 to 6:4) to afford the title compound (122 mg, 21% yield) as an off white solid; ¹H NMR (500 MHz, DMSO-d6) δ 7.17-7.34 (m, 2H), 7.73 (s, 2H).

### 3-Chloro-2-fluoro-6-nitrobenzene-1-thiol

The procedure to prepare 2-chloro-3-fluoro-6-nitrobenzene-1-thiol was used except that 1,3-dichloro-2-fluoro-4-nitrobenzene was substituted for 2-chloro-1,3-difluoro-4-nitrobenzene (20% yield); ¹H NMR (500 MHz, Chloroform-d) δ 7.58 (dd, 1H), 7.65 (dd, 1H).

### 6-Amino-3-chloro-2-fluorobenzene-1-thiol

The procedure to prepare 6-amino-2-chloro-3-fluorobenzene-1-thiol was used except that 3-chloro-2-fluoro-6-nitrobenzene-1-thiol was substituted for 2-chloro-3-fluoro-6-nitrobenzene-1-thiol (52% yield); ¹H NMR (500 MHz, DMSO-d6) δ 6.68 (s, 2H), 7.63 (d, 1H), 7.76 (t, 1H).

### 6-Chloro-7-fluoro-1,3-benzothiazol-2-amine

The procedure to prepare 7-chloro-6-fluoro-1,3-benzothiazol-2-amine was used except that 6-amino-3-chloro-2-fluorobenzene-1-thiol was substituted for 6-amino-2-chloro-3-fluorobenzene-1-thiol (40% yield); ¹H NMR (500 MHz, DMSO-d6) δ 7.18 (d, 1H), 7.31-7.43 (m, 1H), 7.88 (s, 2H).

### 5,6-Dichloro-1,3-benzothiazol-2-amine

To a solution of (3,4-dichlorophenyl)thiourea (1.00 g, 4.52 mmol) in concentrated sulfuric acid (2.3 mL) was added ammonium bromide (438 mg, 4.52 mmol) and the solution heated at 100° C. for 45 min. The reaction was allowed to cool to room temperature, then ice/water (34 mL) was added and the solution basified with NH₃(aq). The resulting precipitate was sonicated and then collected by filtration to afford the title compound as a 50% mixture with the 6,7 dichloro isomer which was taken into the next stage without further purification (1.30 g, 48% purity, 31% yield); m/z=218.8 (MH)⁺.

### 6-Bromo-1,3-benzothiazol-2-amine

The procedure to prepare ethyl 5,6-dichloro-1,3-benzothiazol-2-amine was used except that (4-bromophenyl)thiourea was substituted for (3,4-dichlorophenyl)thiourea (39% yield); ¹H NMR (500 MHz, DMSO-d6) δ 7.41 (d, 1H), 7.50 (m, 2H), 9.77 (s, 1H).

### 6-(Trifluoromethyl)-1,3-benzothiazol-2-amine

The procedure to prepare ethyl 5,6-dichloro-1,3-benzothiazol-2-amine was used except that (4-(trifluoromethyl)phenyl)thiourea was substituted for (3,4-dichlorophenyl)thiourea (22% yield); ¹H NMR (500 MHz, DMSO-d6) δ 7.40 (s, 1H), 7.65 (d, 1H), 7.72 (d, 1H), 8.05 (s, 1H), 10.02 (s, 1H).

### 6-Methoxy-1,3-benzothiazol-2-amine

The procedure to prepare ethyl 5,6-dichloro-1,3-benzothiazol-2-amine was used except that (4-methoxy)thiourea was substituted for (3,4-dichlorophenyl)thiourea (29% yield); ¹H NMR (500 MHz, DMSO-d6) δ 3.73 (s, 3H), 6.81 (dd, 1H), 7.23 (m, 3H), 7.30 (d, 1H).

### Methyl 3-sulfamoylpropanoate

Methyl 3-(chlorosulfonyl)propanoate (437 μL, 2.68 mmol) was added dropwise to a stirred solution of 7M NH₃ in MeOH (8 mL) at 0° C. The reaction was allowed to warm to room temperature and stirred for 2 h. The reaction was concentrated in vacuo and the residue was extracted with hot diethyl ether (6×5 mL). The combined extracts were filtered and the filtrate was concentrated in vacuo to afford the title compound as a colourless oil (82 mg 18% yield); ¹H NMR (500 MHz, Chloroform-d) δ 2.89 (t, 2H), 3.47 (t, 2H), 3.74 (s, 3H), 4.83 (s, 2H).

### 5,5,7,7-Tetramethyl-4,5,6,7-tetrahydro-1,3-benzothiazol-2-amine

To a solution of 3,3,5,5-tetramethylcyclohexanone (3.59 g, 23.3 mmol) in EtOH (70 mL) was added thiourea (2.66 g, 34.9 mmol) and iodine (7.09 g, 27.9 mmol) and the mixture refluxed for 16 h. The EtOH was evaporated to afford a brown oil. This was partitioned between EtOAc (150 mL) and 2M NaOH (100 mL). The phases were separated and the organic phase was washed with 2M sodium hydrogensulfite (50 mL), brine (20 mL), dried (Na₂SO₄) and the mixture filtered. The filtrate evaporated to dryness to afford an orange oil which was successfully chromatographed on silica (eluent: 20-80% EtOAc in heptane) to afford the title compound as a yellow solid (1.93 g, 39% yield); ¹H NMR (500 MHz, DMSO-d6) δ 0.99 (s, 6H), 1.19 (s, 6H), 1.49 (s, 2H), 2.17 (s, 2H), 6.57 (s, 2H).

### 6,6-Dimethyl-4,5,6,7-tetrahydro-1,3-benzothiazol-2-amine

To a solution of 4,4-dimethylcyclohexan-1-one (1.00 g, 7.92 mmol) and EtOH (25 mL) was added thiourea (1.81 g, 23.8 mmol) and iodine (2.01 g, 7.92 mmol) and the mixture heated to reflux for 8 h. The reaction mixture was concentrated and the residue treated with ice-cold water (50 mL), basified with NH₃(aq) and extracted with EtOAc (3×30 mL). The combined organic extracts were dried (Na₂SO₄), filtered and concentrated to afford the title compound as an off-white solid (800 mg, 47% yield); ¹H NMR (400 MHz, DMSO-d6) δ 0.94 (s, 6H), 1.46 (t, 2H), 2.25 (t, 2H), 2.30-2.38 (m, 2H), 6.55 (s, 2H).

### 5,5,6,6-Tetramethyl-4H,5H,6H-cyclopenta[d][1,3]thiazol-2-amine

A solution of 3,3,4,4-tetramethylcyclopentan-1-one (available following a literature procedure, namely Aristoff, P. A.; Nelson, C. L., Organic Preparations and Procedures International, 1983, 15, 149-152, 5.00 g, 35.7 mmol), pyrrolidine (2.79 g, 39.2 mmol) and para-toluenesulfonic acid monohydrate (339 mg, 1.78 mmol) in cyclohexane (40 mL) was refluxed for 2 h. The solvent was removed and replaced by MeOH (10 mL) and sulfur (S₈, 1.14 g, 4.45 mmol) added followed by cyanamide (1.65 g, 39.2 mmol). The solution was refluxed for 2 h, cooled and filtered to remove unwanted particulates. The filtrate was evaporated and purified by silica chromatography (eluent: 50-100% EtOAc in heptane) to afford the title compound as a brown oil (2.88 g, 41% yield); ¹H NMR (500 MHz, DMSO-d6) δ 1.02 (2s, 2×6H), 2.35 (s, 2H), 6.68 (s, 2H).

### 5,5-Dimethyl-4,5,6,7-tetrahydro-1,3-benzothiazol-2-amine

The procedure to prepare 6,6-dimethyl-4,5,6,7-tetrahydro-1,3-benzothiazol-2-amine was used except that 3,3-dimethylcyclohexan-1-one was substituted for 4,4-dimethylcyclohexan-1-one and purification was carried out by recrystallization from DCM/heptane (30% yield); ¹H NMR (500 MHz, DMSO-d6) δ 0.94 (s, 6H), 1.48 (t, 2H), 2.18 (s, 2H), 2.47 (t, 2H), 6.57 (s, 2H).

### 2-Iodo-3,3-dimethylcyclohexan-1-one

Following a modification of a literature method (Sha, C. K. et al, Tetrahedron Lett., 2001, 42 (4), 683-685), to a stirring solution of copper(I) iodide (8.47 g, 44.5 mmol) in dry THF (50 mL) under N₂ was slowly added 3M bromo(methyl)magnesium (14.8 mL, 44.4 mmol) at 0° C. The reaction mixture was stirred at this temperature for 10 min then 2-iodo-3-methylcyclohex-2-en-1-one (prepared according to a literature procedure, namely Benhida R. et al, Tetrahedron Lett., 1998, 39, 6849-6852) (3.50 g, 14.8 mmol) was added. More THF (20 mL) was added to fully solubilise the reaction mixture, which was stirred for 90 min at 0-5° C. The reaction was then quenched with slow addition of saturated aqueous NH₄Cl (˜15 mL). The aqueous layer was extracted with Et₂O (3×50 mL). The combined organic extracts were washed with saturated NaHCO₃ (30 mL) and brine (20 mL), and then dried over Na₂SO₄. The mixture was filtered and the filtrate evaporated to dryness to yield a brown oil which was purified using FCC (eluent: 10% EtOAc in heptane) to yield the title compound as a yellow oil (1.50 g, 30% yield); m/z=252.9 (MH)⁺.

### 7,7-Dimethyl-4,5,6,7-tetrahydro-1,3-benzothiazol-2-amine

To a stirring solution of 2-iodo-3,3-dimethylcyclohexan-1-one (90%, 718 mg, 2.56 mmol) in EtOH (10 mL) under nitrogen was added thiourea (195 mg, 2.56 mmol), pyridine (0.210 mL, 2.56 mmol) and the mixture was refluxed for 2 h. The solution was cooled whereupon a yellow solid precipitated which was filtered off and discarded. The filtrate was taken up in EtOAc (20 mL) and washed with NaHCO₃(aq) (10 mL) and brine (10 mL). The organic layer was dried using Na₂SO₄, filtered and the filtrate concentrated in vacuo to yield a brown oil which was purified by FCC (eluent: 60% EtOAc in heptane) to yield the title compound as a brown oil (50 mg, 7% yield); ¹H NMR (250 MHz, Chloroform-d) δ 0.98 (s, 6H), 1.54 (t, 2H), 2.29 (s, 2H), 2.49 (t, 2H), 7.86 (s, 2H).

### N-(7-Hydroxy-7-methyl-4,5,6,7-tetrahydro-1,3-benzothiazol-2-yl)acetamide

To a suspension of N-(7-oxo-4,5,6,7-tetrahydro-1,3-benzothiazol-2-yl)acetamide (399 mg, 1.90 mmol) in THF (8 mL) at room temperature was added 3M methyl magnesium bromide in THF (1.27 mL, 3.80 mmol) and the mixture stirred 1 h. The resultant black mass was solubilised in EtOAc/3M HCl(aq) but the product did not extract, so the aqueous phase was made basic with concentrated NH₃(aq) and the mixture extracted with EtOAc (3×50 mL). The combined organic extracts were washed with brine (5 mL), dried (Na₂SO₄), the mixture filtered and the filtrate evaporated to dryness to afford the title compound as a yellow solid (360 mg, 78% yield); m/z=227.2 (MH)⁺.

### N-(7-Methyl-4,5,6,7-tetrahydro-1,3-benzothiazol-2-yl)acetamide

To a solution of N-(7-hydroxy-7-methyl-4,5,6,7-tetrahydro-1,3-benzothiazol-2-yl)acetamide (300 mg, 1.33 mmol) in a mixture in MeOH:TFA 9:1 (10 mL) was added 10% Pd/C (80 mg), the mixture degassed then stirred under a hydrogen atmosphere for 16 h. The mixture was filtered through Celite™ and evaporated. The residue was then chromatographed on silica (eluent: 20-60% EtOAc in heptane) to afford the title compound as a white solid (210 mg, 75% yield); ¹H NMR (500 MHz, Chloroform-d) δ 1.33 (d, 3H), 1.41-1.52 (m, 1H), 1.73-1.86 (m, 1H), 1.99-2.12 (m, 2H), 2.36 (s, 3H), 2.63-2.80 (m, 2H), 2.90-2.99 (m, 1H).

### 7-Methyl-4,5,6,7-tetrahydro-1,3-benzothiazol-2-amine

To N-(7-methyl-4,5,6,7-tetrahydro-1,3-benzothiazol-2-yl)acetamide (210 mg, 1 mmol) was added concentrated HCl (4 mL) and the solution heated at 100° C. for 2 h. The solvent was evaporated and the residue azeotroped from DCM to afford the title compound as a pink solid (172 mg, 100% yield); ¹H NMR (500 MHz, DMSO-d6) δ 1.14 (d, 3H), 1.30-1.42 (m, 1H), 1.59-1.72 (m, 1H), 1.86 (dd, 1H), 1.90-1.97 (m, 1H), 2.31-2.45 (m, 2H), 2.71-2.81 (m, 1H), 9.27 (s, 2H).

### 4,4,6,6-Tetramethyl-4H,6H,7H-pyrano[4,3-d][1,3]thiazol-2-amine

A solution of 2,2,6,6-tetramethyloxan-4-one (250 mg,1.60 mmol), pyrrolidine (125 mg, 1.76 mmol) and pTSA (15 mg, 0.08 mmol) in cyclohexane (2 mL) was refluxed for 2 h. The solvent was removed and replaced by MeOH (0.5 mL) and then sulfur was added (51 mg, 0.20 mmol) followed by cyanamide (74 mg, 1.76 mmol). The solution was refluxed for 2 h, cooled, diluted with EtOAc and filtered. The filtrate was adsorbed onto silica gel and purified by FCC (eluent: EtOAc:Heptane (0-100%) to afford the title compound as a yellow solid (210 mg, 62% yield); m/z=213.0 (MH)⁺.

### 6,6-Dimethylbicyclo[3.1.0]hexan-3-one

A solution of 6,6-dimethylbicyclo[3.1.0]hexan-3-ol (available via a literature procedure: US2008/318955 A1, 2008) (1.10 g, 8.72 mmol) in DCM (100 mL) was cooled to 0° C. and Dess Martin Periodinane (4.07 g, 9.59 mmol) was added portionwise. The resulting suspension was stirred at 0° C. for 1 h and then allowed to warm to room temperature over 3 h. After this time, the mixture was filtered and the resulting filtrate washed with 1M NaOH_{(aq)} (3×100 mL), water (100 mL) and brine (100 mL). The resulting solution was dried over MgSO₄ and evaporated to dryness. The residue was passed down a short silica plug, eluting with 0-50% EtOAc in heptane, to afford the title compound as a pale yellow oil (865 mg, 80% yield); ¹H NMR (250 MHz, Chloroform-d) δ 0.86 (s, 3H), 1.08 (s, 3H), 1.25-1.35 (m, 2H), 2.08-2.21 (m, 2H), 2.44-2.58 (m, 2H).

### 3,3-Dimethyl-9-thia-7-azatricyclo[4.3.0.0^{2,4}]nona-1 (6),7-dien-8-amine

The procedure to prepare 4,4,6,6-tetramethyl-4H,6H,7H-pyrano[4,3-d][1,3]thiazol-2-amine was used except that 6,6-dimethylbicyclo[3.1.0]hexan-3-one was substituted for 2,2,6,6-tetramethyloxan-4-one (8% yield); m/z=180.9 (MH)⁺.

### 4,7-Dichloro-1,3-benzothiazol-2-amine

To a stirred solution of (2,5-dichlorophenyl)thiourea (1.00 g, 4.52 mmol) in concentrated sulfuric acid (3 mL) was added portionwise ammonium bromide (439 mg, 4.52 mmol) over 20 min, and then the reaction mixture was heated at 100° C. for 30 min. The reaction mixture was cooled to room temperature and then poured onto ice water (30 mL), at which point a white precipitate was observed. The acidic solution was taken to pH 7 using aqueous ammonium hydroxide solution (˜5 mL). The solid was collected by filtration, washed with water and dried in vacuo to afford the title compound as a white solid (830 mg, 80% yield) m/z=218.9 (MH)⁺.

### [2-Fluoro-3-(trifluoromethoxy)phenyl]thiourea

To a solution of 2-fluoro-3-(trifluoromethoxy)aniline (5.00 g, 25.6 mmol) in acetone (75 mL) was added benzoyl isothiocyanate (4.54 mL, 32.0 mmol) and the reaction mixture heated at 60° C. for 3 h. Water (200 mL) was added to the reaction mixture and an orange precipitate formed. The solid was collected by filtration. The solid was added to 2M aqueous sodium hydroxide solution (100 mL, 200 mmol) and the mixture stirred at 90° C. for 30 min. The reaction mixture was cooled to room temperature and then neutralised using 6M HCl (aq) (˜20 mL). The resulting precipitate was collected by filtration. The solid was sonicated in MeCN and the remaining solid was filtered off and discarded. The filtrate was concentrated and triturated in DCM to afford the title compound as an off-white solid (800 mg, 12% yield); m/z=255.0 (MH)⁺.

### 7-(Trifluoromethoxy)-1,3-benzothiazol-2-amine

To a stirred suspension of sodium hydride (60% dispersion in oil, 139 mg, 3.46 mmol) in NMP (10 mL), under nitrogen was added [2-fluoro-3-(trifluoromethoxy)phenyl]thiourea (800 mg, 3.147 mmol). The suspension stirred at room temperature for 40 min, then heated to 85° C. for 1.5 h and at 95° C. for 3 h. Saturated aqueous sodium hydrogen carbonate solution (30 mL) followed by EtOAc (30 mL) were added to the reaction mixture and the layers were separated. The aqueous layer was extracted with EtOAc (3×20 mL) and the combined organic layers were then washed with water (3×20 mL), brine (30 mL), dried (MgSO₄), filtered and concentrated to afford the title compound as yellow solid (550 mg, 83% purity, 62% yield); m/z=235.0 (MH)⁺.

### 6,7-Difluoro-1,3-benzothiazol-2-amine

The procedure to prepare 7-(trifluoromethoxy)-1,3-benzothiazol-2-amine was used except that (2,3,4-trifluorophenyl)thiourea was substituted for [2-fluoro-3-(trifluoromethoxy)phenyl]thiourea (84% yield); m/z=187.0 (MH)⁺.

### 1-Benzoyl-3-[3-chloro-4-(trifluoromethoxy)phenyl]thiourea

To a stirred solution of 3-chloro-4-(trifluoromethoxy)aniline (5.00 g, 23.6 mmol) in acetone (150 mL) under nitrogen was added benzoyl isothiocyanate (3.98 mL, 29.5 mmol). The reaction was heated at 75° C. for 1.5 h. The cooled reaction mixture was poured into water (400 mL). The resulting precipitate was collected by filtration to give the title compound as a pale orange solid (9.0 g, quantitative yield); m/z=374.9 (MH)⁺.

### [3-Chloro-4-(trifluoromethoxy)phenyl]thiourea

A stirred suspension of 1-benzoyl-3-[3-chloro-4-(trifluoromethoxy)phenyl]thiourea (8.86 g, 23.6 mmol) in 2M NaOH (53 mL, 106 mmol) was heated at 80° C. for 1 h. On cooling to room temperature the reaction became heterogeneous and was filtered. The filtrate was poured onto ice containing 6M HCl_{(aq)} (25 mL). The pH was adjusted to pH 10 with concentrated NH_{3(aq)} with stirring. The resulting precipitate was collected by filtration and washed with water to give the title compound as a white solid (5.60 g, 88% yield); m/z=271.0 (MH)⁺.

### Mixture of 5-chloro-6-(trifluoromethoxy)-1,3-benzothiazol-2-amine and 7-chloro-6-(trifluoromethoxy)-1,3-benzothiazol-2-amine,1:1

The procedure to prepare 4,7-dichloro-1,3-benzothiazol-2-amine was used except that [3-chloro-4-(trifluoromethoxy)phenyl]thiourea was substituted for (2,5-dichlorophenyl)thiourea (88% combined yield, isomers not separated); m/z=269.0 (MH)⁺.

### (4-Chloro-2-fluoro-3-methoxyphenyl)thiourea

The procedure to prepare [2-fluoro-3-(trifluoromethoxy)phenyl]thiourea was used except that 4-chloro-2-fluoro-3-methoxyaniline was substituted for 2-fluoro-3-(trifluoromethoxy)aniline (82% yield; m/z=235.2 (MH)⁺.

### 6-Chloro-7-methoxy-1,3-benzothiazol-2-amine

The procedure to prepare 7-(trifluoromethoxy)-1,3-benzothiazol-2-amine was used except that (4-chloro-2-fluoro-3-methoxyphenyl)thiourea was substituted for [2-fluoro-3-(trifluoromethoxy)phenyl]thiourea (10% yield); m/z=215.0 (MH)⁺.

### 2,3-Dichloro-1-methoxy-4-nitrobenzene

To a stirred solution of 2,3-dichloro-1-fluoro-4-nitrobenzene (8.50 g, 40.5 mmol) in MeOH (85 mL) at room temperature was added a solution of sodium methoxide in MeOH (5.4M, 7.5 mL, 40.5 mmol) and the solution stirred at room temperature for 3 h. The solvent was evaporated, water was added (100 mL) and the suspension was filtered. The solid was washed with more water then dried in air to afford the title compound as a yellow solid (9.35 g, 94% yield); ¹H NMR (500 MHz, Chloroform-d) δ 4.04 (s, 3H), 6.95 (d, 1H), 7.92 (d, 1H).

### 2,3-Dichloro-4-methoxyaniline

A solution of 2,3-dichloro-1-methoxy-4-nitrobenzene (6.00 g, 27.0 mmol) and 10% Pd/C (600 mg) in EtOH (180 mL) was degassed under N₂ then stirred for 24 h at room temperature under a hydrogen atmosphere. The mixture was filtered through Celite and the filtrate evaporated to afford the title compound as a purple solid (6.80 g, quantitative yield); m/z=192.1 (MH)⁺.

### 1-Benzoyl-3-(2,3-dichloro-4-methoxyphenyl)thiourea

The procedure to prepare 1-benzoyl-3-[3-chloro-4-(trifluoromethoxy)phenyl]thiourea was used except that 2,3-dichloro-4-methoxyaniline was substituted for 3-chloro-4-(trifluoromethoxy)aniline (48% yield); ¹H NMR (500 MHz, DMSO-d6) δ 3.94 (s, 3H), 7.24 (d, 1H), 7.50-7.83 (m, 4H), 7.88-8.10 (m, 2H), 11.79 (s, 1H), 12.41 (s, 1H).

### (2,3-Dichloro-4-methoxyphenyl)thiourea

A stirred suspension of 1-benzoyl-3-(2,3-dichloro-4-methoxyphenyl)thiourea (4.50 g, 12.7 mmol) in 2M NaOH (30 mL) was heated at 80° C. for 2 h. The solution was cooled to room temperature, made acidic with 2M HCl_{(aq)} and filtered. The solid was then stirred with 2M ammonia solution and filtered. The resultant white solid was washed with water and dried in air to afford the title compound as a white solid (3.18 g, 86% purity, 68% yield); m/z=250.9 (MH)⁺.

### 7-Chloro-6-methoxy-1,3-benzothiazol-2-amine

To a stirred solution of (2,3-dichloro-4-methoxyphenyl)thiourea (86%, 2.55 g, 8.73 mmol) in NMP (30 mL), was added NaH (60% dispersion, 523 mg, 13.1 mmol). The reaction was stirred at room temperature for 5 min then heated at 130° C. for 3 h. The reaction mixture was allowed to cool to room temperature and diluted with water (80 mL) and EtOAc (120 mL). The layers were separated. The organic layer was washed with water (3×100 mL), with vigorous shaking to remove NMP, washed with brine (100 mL), dried (Na₂SO₄), filtered and concentrated. The residue was chromatographed on silica (eluent: 20-80% EtOAc in heptane) to afford the title compound as an off white solid (82% purity, 143 mg, 8% yield); m/z=215.0 (MH)⁺.

### 4-Fluoro-1,3-benzothiazol-2-amine

The procedure to prepare 7-(trifluoromethoxy)-1,3-benzothiazol-2-amine was used except that 1-(2,3-difluorophenyl)thiourea was substituted for [2-fluoro-3-(trifluoromethoxy)phenyl]thiourea and purification of the title compound by FCC on silica (eluent: 0-75% EtOAc in heptane) was performed (31% yield); m/z=169.2 (MH)⁺.

### 1-Benzoyl-3-[4-chloro-3-(trifluoromethoxy)phenyl]thiourea

The procedure to prepare 1-benzoyl-3-[3-chloro-4-(trifluoromethoxy)phenyl]thiourea was used except that 4-chloro-3-(trifluoromethoxy)aniline was substituted for 3-chloro-4-(trifluoromethoxy)aniline (quantitative yield); m/z=375.0 (MH)⁺.

### [4-Chloro-3-(trifluoromethoxy)phenyl]thiourea

The procedure to prepare [3-chloro-4-(trifluoromethoxy)phenyl]thiourea was used except that 1-benzoyl-3-[4-chloro-3-(trifluoromethoxy)phenyl]thiourea was substituted for 1-benzoyl-3-[3-chloro-4-(trifluoromethoxy)phenyl]thiourea (96% yield); m/z=270.9 (MH)⁺.

### 6-Chloro-5-(trifluoromethoxy)-1,3-benzothiazol-2-amine

The procedure to prepare 4,7-dichloro-1,3-benzothiazol-2-amine was used except that [4-chloro-3-(trifluoromethoxy)phenyl]thiourea was substituted for (2,5-dichlorophenyl)thiourea (81% yield); ¹H NMR (500 MHz, DMSO-d6) δ 7.41 (d, 1H), 7.85 (s, 2H), 8.01 (s, 1H).

### 1-Benzoyl-3-(4-bromo-3-chloro-2-fluorophenyl)thiourea

The procedure to prepare 1-benzoyl-3-[3-chloro-4-(trifluoromethoxy)phenyl]thiourea was followed except that 4-bromo-3-chloro-2-fluoroaniline was substituted for 3-chloro-4-(trifluoromethoxy)aniline (98% yield); m/z=387.0 (MH)⁺.

### (4-Bromo-3-chloro-2-fluorophenyl)thiourea

1-Benzoyl-3-(4-bromo-3-chloro-2-fluorophenyl)thiourea (17.6 g, 44.5 mmol) was suspended in 2M sodium hydroxide (150 mL, 0.300 mmol) and the reaction mixture stirred at 80° C. for 2 h. The reaction mixture was cooled, filtered, and the filtrate adjusted to ˜pH 7 using conc. HCl. The resulting precipitate was filtered off and washed with water, giving a grey solid. After aging, the filtrate was re-filtered, giving a second grey solid. The solids were combined and dried under reduced pressure to afford the title compound as a green-grey solid (11.7 g, 87%); m/z=282.8 (MH)⁺.

### 6-Bromo-7-chloro-1,3-benzothiazol-2-amine

The procedure to prepare 7-chloro-6-methoxy-1,3-benzothiazol-2-amine was followed except that (4-bromo-3-chloro-2-fluorophenyl)thiourea was substituted for (2,3-dichloro-4-methoxyphenyl)thiourea and the reaction temperature was 100° C. (heating for 30 min) (100% yield). m/z=262.9 (MH)⁺.

### 4-[2-(4,5-Dichloro-2-nitrophenoxy)ethyl]morpholine

To a stirred suspension of sodium hydride (60%, 210 mg, 5.24 mmol) in THF (10 mL) at 0° C. was added 2-(morpholin-4-yl)ethanol (665 μL, 5.48 mmol) dropwise. The reaction mixture was stirred at 0° C. for 10 min before the addition of 1,2-dichloro-4-fluoro-5-nitrobenzene (626 μL, 4.76 mmol) dropwise at 0° C. The reaction was stirred at 0° C. for 1 h. Water (20 mL) was added dropwise and the reaction was extracted with EtOAc (3×30 mL). The combined organic extracts were washed with water (20 mL), brine (20 mL), dried (Na₂SO₄), filtered and concentrated to afford the title compound as a red solid (1.60 g, quantitative yield); m/z=321.1 (MH)⁺.

### 4,5-Dichloro-2-[2-(morpholin-4-yl)ethoxy]aniline

To a stirred solution of 4-[2-(4,5-dichloro-2-nitrophenoxy)ethyl]morpholine (1.53 g, 4.76 mmol) in a mixture of water (10 mL), AcOH (2 mL) and EtOH (14 mL) at 80° C. was added iron powder (1.06 g, 19.1 mmol) in one portion. The reaction was heated at 80° C. for 30 min then allowed to cool to room temperature. The pH was adjusted to >pH 9 with ammonium hydroxide solution and the reaction was filtered through Celite™, rinsing with EtOAc. The filtrate was diluted with water (30 mL) and extracted with EtOAc (3×40 mL). The combined organic extracts were washed with brine (40 mL), dried (Na₂SO₄), filtered and the filtrate concentrated to afford the title compound as a beige oil (1.40 g, quantitative yield); m/z=291.1 (MH)⁺.

### 1-Benzoyl-3-{4,5-dichloro-2-[2-(morpholin-4-yl)ethoxy]phenyl}thiourea

The procedure to prepare 1-benzoyl-3-[3-chloro-4-(trifluoromethoxy)phenyl]thiourea was followed except that 4,5-dichloro-2-[2-(morpholin-4-yl)ethoxy]aniline was substituted for 3-chloro-4-(trifluoromethoxy)aniline (92% yield); m/z=454.1 (MH)⁺.

### {4,5-Dichloro-2-[2-(morpholin-4-yl)ethoxy]phenyl}thiourea

The procedure to prepare [3-chloro-4-(trifluoromethoxy)phenyl]thiourea was followed except that 1-benzoyl-3-{4,5-dichloro-2-[2-(morpholin-4-yl)ethoxy]phenyl}thiourea was substituted for 1-benzoyl-3-[3-chloro-4-(trifluoromethoxy)phenyl]thiourea (83% yield); m/z=350.0 (MH)⁺.

### 6,7-Dichloro-4-[2-(morpholin-4-yl)ethoxy]-1,3-benzothiazol-2-amine

The procedure to prepare 4,7-dichloro-1,3-benzothiazol-2-amine was used except that {4,5-dichloro-2-[2-(morpholin-4-yl)ethoxy]phenyl}thiourea was substituted for (2,5-dichlorophenyl)thiourea (100% yield); m/z=348.0 (MH)⁺.

### tert-Butyl N-{3,4-dichloro-5-[2-(morpholin-4-yl)ethoxy]phenyl}carbamate

To a stirred solution of tert-butyl N-(3,4-dichloro-5-hydroxyphenyl)carbamate (available via a literature method: WO2009/33581 A1, 2009) (4.10 g, 14.7 mmol) in DMF (82 mL) was added 4-(2-chloroethyl)morpholine hydrochloride (1:1) (3.02 g, 16.2 mmol) and K₂CO₃ (4.48 g, 32.4 mmol) and the mixture heated at 60° C. for 18 h. It was cooled, diluted with EtOAc (160 mL), washed with water (4×80 mL), dried (Na₂SO₄), filtered and concentrated. The residue was purified by FCC on silica (eluent: 0-10% MeOH in DCM) to afford the title compound as an orange oil (5.80 g, quantitative yield); m/z=391.5 (MH)⁺.

### 3,4-Dichloro-5-[2-(morpholin-4-yl)ethoxy]aniline

To a stirred solution of tert-butyl N-{3,4-dichloro-5-[2-(morpholin-4-yl)ethoxy]phenyl}carbamate (5.77 g, 14.8 mmol) in DCM (34 mL) was added TFA (11 mL). The solution was stirred at room temperature for 50 min and then concentrated. The residue was dissolved in saturated NaHCO_{3(aq)} (50 mL) and extracted with EtOAc (3×50 mL). The combined organic extracts were dried (Na₂SO₄), filtered and concentrated to give the title compound as a brown oil (4.50 g, 95% yield); m/z=291.0 (MH)⁺.

### 1-Benzoyl-3-{3,4-dichloro-5-[2-(morpholin-4-yl)ethoxy]phenyl}thiourea

The procedure to prepare 1-benzoyl-3-[3-chloro-4-(trifluoromethoxy)phenyl]thiourea was followed except that 3,4-dichloro-5-[2-(morpholin-4-yl)ethoxy]aniline was substituted for 3-chloro-4-(trifluoromethoxy)aniline (100% yield); m/z=454.1 (MH)⁺.

### {3,4-Dichloro-5-[2-(morpholin-4-yl)ethoxy]phenyl}thiourea

The procedure to prepare [3-chloro-4-(trifluoromethoxy)phenyl]thiourea was followed except that 1-benzoyl-3-{3,4-dichloro-5-[2-(morpholin-4-yl)ethoxy]phenyl}thiourea was substituted for 1-benzoyl-3-[3-chloro-4-(trifluoromethoxy)phenyl]thiourea (60% yield); m/z=350.0 (MH)⁺.

### 6,7-Dichloro-5-[2-(morpholin-4-yl)ethoxy]-1,3-benzothiazol-2-amine

The procedure to prepare 4,7-dichloro-1,3-benzothiazol-2-amine was used except that {3,4-dichloro-5-[2-(morpholin-4-yl)ethoxy]phenyl}thiourea was substituted for (2,5-dichlorophenyl)thiourea (92% yield); m/z=348.0 (MH)⁺.

### 4-[2-(2-Chloro-3-fluoro-5-nitrophenoxy)ethyl]morpholine

The procedure to prepare 4-[2-(4,5-dichloro-2-nitrophenoxy)ethyl]morpholine was used except that 2-chloro-1,3-difluoro-5-nitrobenzene was substituted for 1,2-dichloro-4-fluoro-5-nitrobenzene (75% yield); m/z=304.9 (MH)⁺.

### 4-Chloro-3-fluoro-5-[2-(morpholin-4-yl)ethoxy]aniline

The procedure to prepare 4,5-dichloro-2-[2-(morpholin-4-yl)ethoxy]aniline was used except that 4-[2-(2-chloro-3-fluoro-5-nitrophenoxy)ethyl]morpholine was substituted for 4-[2-(4,5-dichloro-2-nitrophenoxy)ethyl]morpholine (74% yield); m/z=275.0 (MH)⁺.

### {4-Chloro-3-fluoro-5-[2-(morpholin-4-yl)ethoxy]phenyl}thiourea

To a stirred solution of 4-chloro-3-fluoro-5-[2-(morpholin-4-yl)ethoxy]aniline (85% purity, 5.22 g, 16.1 mmol) in acetone (125 mL) under nitrogen was added benzoyl isothiocyanate (2.72 mL, 20.2 mmol). The reaction was stirred at room temperature for 30 min then filtered. The filtrate was concentrated to remove most of the acetone then sodium hydroxide pellets (13.0 g, 325 mmol) were added and the solution refluxed for 2 h. The cooled mixture was taken to pH 7 with 2M HCl_{(aq)} and the mixture extracted with DCM (3×100 mL). The combined organic extracts were dried (Na₂SO₄), filtered and the filtrate evaporated to dryness to afford the title compound as an orange oil (2.10 g, 64% purity, 25% yield); m/z=334.0 (MH)⁺.

### 6-Chloro-7-fluoro-5-[2-(morpholin-4-yl)ethoxy]-1,3-benzothiazol-2-amine

To a stirred solution of {4-chloro-3-fluoro-5-[2-(morpholin-4-yl)ethoxy]phenyl}thiourea (2.10 g, 64% purity, 4.03 mmol) in concentrated sulfuric acid (6.3 mL) was added solid ammonium bromide (394 mg, 4.03 mmol) and the mixture stirred at 80° C. for 1 h. The cooled reaction mixture was poured into water, made basic with concentrated ammonia solution and extracted with DCM (3×100 mL). The combined organic extracts were dried (Na₂SO₄), filtered and the filtrate evaporated to dryness. The residue was purified by FCC on silica (eluent 0-12% MeOH in EtOAc) to afford the title compound as a yellow solid (260 mg, 19% yield); m/z=332.0 (MH)⁺.

### Example 1

### 9,10-Dichloro-7-thia-2,5-diazatricyclo[6.4.0.0^{2,6}]dodeca-1(8),3,5,9,11-pentaene-4-carboxylic acid

To a solution of ethyl 9,10-dichloro-7-thia-2,5-diazatricyclo[6.4.0.0^{2,6}]dodeca-1(8),3,5,9,11-pentaene-4-carboxylate (600 mg, 1.90 mmol) in a THF:MeOH:H₂O mixture (8:1:1, 20 mL) was added lithium hydroxide monohydrate (399 mg, 9.52 mmol) and the reaction mixture stirred for 4 h before being concentrated. Water was added and the pH adjusted to 4 using 1M HCl, whereupon a precipitate was obtained which was filtered off and washed with cold water. The solid was dried in air to afford the title compound (250 mg, 85% purity, 39% yield); m/z=286.8 (MH)⁺; ¹H NMR (500 MHz, DMSO-d6) δ 7.89 (d, 1H), 8.18 (d, 1H), 9.01 (s, 1H), 12.81 (s, 1H).

### Example 2

### 9-Chloro-10-fluoro-7-thia-2,5-diazatricyclo[6.4.0.0^{2,6}]dodeca-1(8),3,5,9,11-pentaene-4-carboxylic acid

Ethyl 9-chloro-10-fluoro-7-thia-2,5-diazatricyclo[6.4.0.0^{2,6}]dodeca-1(8),3,5,9,11-pentaene-4-carboxylate (53 mg, 0.18 mmol) was suspended in EtOH (3 mL), 1M NaOH in water (1.51 mL, 1.51 mmol) was added and the mixture was stirred at 75° C. for 45 min. The reaction was allowed to cool to room temperature. The EtOH was removed in vacuo and the resulting white suspension was adjusted to pH 1 by the addition of 1M HCl. The white suspension was sonicated for 2 min then filtered over glass fibre filter paper. The filter pad was washed with 1M HCl (2×1 mL) then air dried. The white solid which was obtained was dried further under high vacuum to afford the title compound (24 mg, 48% yield) as a white solid; m/z=271.0 (MH)⁺; ¹H NMR (500 MHz, DMSO-d6) δ 7.64 (t, 1H), 8.13 (dd, 1H), 8.93 (s, 1H). Exchangeables not observed.

### Example 3

### 10-Chloro-9-fluoro-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10-chloro-9-fluoro-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (94% yield); m/z=270.9 (MH)⁺; ¹H NMR (500 MHz, DMSO-d6) δ 7.81-7.89 (m, 1H), 8.07 (d, 1H), 9.04 (s, 1H). Exchangeables not observed.

### Example 4

### 10-(Trifluoromethoxy)-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that 10-(trifluoromethoxy)-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (64% yield); m/z=302.8 (MH)⁺; ¹H NMR (500 MHz, DMSO-d6) δ 7.62 (dd, 1H), 8.22-8.32 (m, 2H), 8.97 (s, 1H), 12.71 (s, 1H).

### Example 5

### 9-Chloro-7-thia-2,5-diazatricyclo[6.4.0.0^{2,6}]dodeca-1(8),3,5,9,11-pentaene-4-carboxylic acid

The procedure to prepare 9,10-dichloro-7-thia-2,5-diazatricyclo[6.4.0.0^{2,6}]dodeca-1(8),3,5,9,11-pentaene-4-carboxylic acid was used except that ethyl 9-chloro-7-thia-2,5-diazatricyclo[6.4.0.0^{2,6}]dodeca-1(8),3,5,9,11-pentaene-4-carboxylate was substituted for ethyl 9,10-dichloro-7-thia-2,5-diazatricyclo[6.4.0.0^{2,6}]dodeca-1(8),3,5,9,11-pentaene-4-carboxylate (44% yield); m/z=253.1 (MH)⁺; ¹H NMR (400 MHz, DMSO-d6) δ 7.63 (d, 2H), 8.16 (s, 1H), 9.01 (s, 1H), 12.42 (s, 1H). Exchangeables not observed.

### Example 6

### 10,11-Dichloro-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10,11-dichloro-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (58% yield); m/z=286.9 (MH)⁺; ¹H NMR (500 MHz, DMSO-d6) δ 8.46 (s, 1H), 8.59 (s, 1H), 8.94 (s, 1H), 12.79 (s, 1H).

### Example 7

### 12-Chloro-7-thia-2,5-diazatricyclo[6.4.0.0^{2,6}]dodeca-1(8),3,5,9,11-pentaene-4-carboxylic acid

For the synthesis of 12-chloro-7-thia-2,5-diazatricyclo[6.4.0.0^{2,6}]dodeca-1(8),3,5,9,11-pentaene-4-carboxylic acid the procedure to prepare 9,10-dichloro-7-thia-2,5-diazatricyclo[6.4.0.0^{2,6}]dodeca-1(8),3,5,9,11-pentaene-4-carboxylic acid was used except that ethyl 12-chloro-7-thia-2,5-diazatricyclo[6.4.0.0^{2,6}]dodeca-1(8),3,5,9,11-pentaene-4-carboxylate was substituted for ethyl 9,10-dichloro-7-thia-2,5-diazatricyclo[6.4.0.0^{2,6}]dodeca-1(8),3,5,9,11-pentaene-4-carboxylate (31% yield); m/z=253.1 (MH)⁺; ¹H NMR (400 MHz, DMSO-d6) δ 7.48 (t, 1H), 7.67 (d, 1H), 8.07 (d, 1H), 8.80 (s, 1H), 12.86 (s, 1H).

### Example 8

### 10-Chloro-7-thia-2,5-diazatricyclo[6.4.0.0^{2,6}]dodeca-1(8),3,5,9,11-pentaene-4-carboxylic acid

Ethyl 10-chloro-7-thia-2,5-diazatricyclo[6.4.0.0^{2,6}]dodeca-1(8),3,5,9,11-pentaene-4-carboxylate (310 mg, 1.10 mmol) was dissolved in EtOH (10 mL) and water. NaOH (221 mg, 5.52 mmol) was added and the reaction mixture stirred at room temperature for 30 min. The mixture was then acidified with 6N HCl and the aqueous phase extracted with EtOAc (3×30 mL). The combined organic layers were dried over Na₂SO₄, the mixture filtered and the filtrate evaporated to afford the title compound as an off white solid (140 mg, 50% yield); m/z=253.1 (MH)⁺; ¹H NMR (400 MHz, DMSO-d6) δ 7.66 (dd, 1H), 8.19 (d, 1H), 8.25 (d, 1H), 9.0 (s, 1H). Exchangeables not observed.

### Example 9

### 10,11-Dimethyl-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10,11-dimethyl-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (43% yield); m/z=247.0 (MH)⁺; ¹H NMR (500 MHz, DMSO-d6) δ 2.34 (s, 3H), 2.36 (s, 3H), 7.79 (s, 1H), 7.97 (s, 1H), 8.85 (s, 1H), 12.62 (s, 1H).

### Example 10

### 10-Fluoro-7-thia-2,5-diazatricyclo[6.4.0.0^{2,6}]dodeca-1(8),3,5,9,11-pentaene-4-carboxylic acid

The procedure to prepare 10-chloro-7-thia-2,5-diazatricyclo[6.4.0.0^{2,6}]dodeca-1(8),3,5,9,11-pentaene-4-carboxylic acid was used except that ethyl 10-fluoro-7-thia-2,5-diazatricyclo[6.4.0.0^{2,6}]dodeca-1(8),3,5,9,11-pentaene-4-carboxylate was substituted for ethyl 10-chloro-7-thia-2,5-diazatricyclo[6.4.0.0^{2,6}]dodeca-1(8),3,5,9,11-pentaene-4-carboxylate (42% yield); m/z=237.1 (MH)⁺; ¹H NMR (400 MHz, DMSO-d6) δ 7.48 (td, 1H), 8.05 (dd, 1H), 8.20 (dd, 1H), 8.97 (s, 1H), 12.67 (s, 1H).

### Example 11

### 10-Methyl-7-thia-2,5-diazatricyclo[6.4.0.0^{2,6}]dodeca-1(8),3,5,9,11-pentaene-4-carboxylic acid

The procedure to prepare 10-chloro-7-thia-2,5-diazatricyclo[6.4.0.0^{2,6}]dodeca-1(8),3,5,9,11-pentaene-4-carboxylic acid was used except that ethyl 10-methyl-7-thia-2,5-diazatricyclo[6.4.0.0^{2,6}]dodeca-1(8),3,5,9,11-pentaene-4-carboxylate was substituted for ethyl 10-chloro-7-thia-2,5-diazatricyclo[6.4.0.0^{2,6}]dodeca-1(8),3,5,9,11-pentaene-4-carboxylate (27% yield); m/z=233.1 (MH)⁺; ¹H NMR (400 MHz, DMSO-d6) δ 2.43 (s, 3H), 7.39 (d, 1H), 7.85 (s, 1H), 8.04 (d, 1H), 8.93 (s, 1H), 12.65 (s, 1H).

### Example 12

### 11-Chloro-7-thia-2,5-diazatricyclo[6.4.0.0^{2,6}]dodeca-1(8),3,5,9,11-pentaene-4-carboxylic acid

The procedure to prepare 10-chloro-7-thia-2,5-diazatricyclo[6.4.0.0^{2,6}]dodeca-1(8),3,5,9,11-pentaene-4-carboxylic acid was used except that ethyl 11-chloro-7-thia-2,5-diazatricyclo[6.4.0.0^{2,6}]dodeca-1(8),3,5,9,11-pentaene-4-carboxylate was substituted for ethyl 10-chloro-7-thia-2,5-diazatricyclo[6.4.0.0^{2,6}]dodeca-1(8),3,5,9,11-pentaene-4-carboxylate (13% yield); m/z=253.2 (MH)⁺; ¹H NMR (400 MHz, DMSO-d6) δ 7.48 (dd, 1H), 8.02 (d, 1H), 8.32 (s, 1H), 8.54 (s, 1H). Exchangeables not observed.

### Example 13

### 10-Bromo-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10-bromo-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (89% yield); m/z=297 (MH)⁺; ¹H NMR (500 MHz, DMSO-d6) δ 7.77 (dd, 1H), 8.13 (d, 1H), 8.37 (d, 1H), 9.00 (s, 1H). Exchangeables not observed.

### Example 14

### 10-(Trifluoromethyl)-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10-(trifluoromethyl)-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (91% yield); m/z=287 (MH)⁺; ¹H NMR (500 MHz, DMSO-d6) δ 7.98 (d, 1H), 8.37 (d, 1H), 8.61 (s, 1H), 9.08 (s, 1H), 12.82 (s1H).

### Example 15

### 10-Methoxy-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 10-methoxy-7-thia-2,5-diazatricyclo[6.4.0.0^{2,6}]dodeca-1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (94% yield); m/z=249 (MH)⁺; ¹H NMR (500 MHz, DMSO-d6) δ 3.83 (s, 3H), 7.13 (dd, 1H), 7.67 (d, 1H), 8.05 (d, 1H), 8.89 (s, 1H), 12.62 (s, 1H).

### Example 16

### 10,12-Dichloro-7-thia-2,5-diazatricyclo[6.4.0.0^{2,6}]dodeca-1(8),3,5,9,11-pentaene-4-carboxylic acid

This compound was purchased from Kingchuk Chemicals (KC-005-71-2). m/z=287 (MH)⁺; ¹H NMR (500 MHz, DMSO-d6) δ 7.88 (d, 1H), 8.28 (d, 1H), 8.80 (s, 1H). Exchangeables not observed.

### Example 17

### 3-Bromo-10-(trifluoromethoxy)-7-thia-2,5-diazatricyclo[6.4.0.0^{2,6}]dodeca-1(8),3,5,9,11-pentaene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 3-bromo-10-(trifluoromethoxy)-7-thia-2,5-diazatricyclo[6.4.0.0^{2,6}]dodeca 1(8),3,5,9,11-pentaene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (78% yield); m/z=380.7 (MH)⁺; ¹H NMR (500 MHz, DMSO-d6) δ 7.65 (dd, 1H), 8.31 (d, 1H), 8.53 (d, 1H). Exchangeables not observed.

### Example 18

### 3-(tert-Butylamino)-10-(trifluoromethoxy)-7-thia-2,5-diazatricyclo[6.4.0.0^{2,6}]dodeca-1(8),3,5,9,11-pentaene-4-carboxylic acid

Ethyl 3-(tert-butylamino)-10-(trifluoromethoxy)-7-thia-2,5-diazatricyclo[6.4.0.0^{2,6}]dodeca-1(8),3,5,9,11-pentaene-4-carboxylate (90 mg, 0.18 mmol) and LiOH.H₂O (15 mg, 0.36 mmol) were stirred in MeOH/water (20 mL/1 mL) at 80° C. for 2 h. The heat was removed and more LiOH.H₂O (15 mg, 0.36 mmol) added and the reaction continued to stir at room temperature for 4 days. The crude reaction was concentrated and purified by low pH reverse phase HPLC to afford the title compound as a white solid (11 mg, 16% yield); m/z=374.1 (MH)⁺; ¹H NMR (500 MHz, DMSO-d6) δ 1.18 (s, 9H), 7.57 (d, 1H), 8.19 (d, 1H), 8.38 (d, 1H). Exchangeables not observed.

### Example 19

### 2-{9,10-Dichloro-7-thia-2,5-diazatricyclo[6.4.0.0^{2,6}]dodeca-1(8),3,5,9,11-pentaen-4-yl}acetic acid

To a solution of ethyl 2-{9,10-dichloro-7-thia-2,5-diazatricyclo[6.4.0.0^{2,6}]dodeca-1(8),3,5,9,11-pentaen-4-yl}acetate (65 mg, 0.20 mmol) in ethanol (5 mL) and water (1 mL) was added LiOH.H₂O (17 mg, 0.39 mmol). The resulting mixture was then heated at 75° C. for 1 h then evaporated to dryness. The residue was dissolved in water (20 mL) and then acidified with 1M HCl to ˜pH 3. The resulting white precipitate was filtered, washed with more water and dried in the vacuum oven to afford the title compound as a beige solid (51 mg, 86% yield); m/z=300.8 (MH)⁺; ¹H NMR (500 MHz, DMSO-d6) δ 3.65 (s, 2H), 7.84 (d, 1H), 8.08 (d, 1H), 8.20 (s, 1H), 12.42 (s, 1H).

### Example 20

### N-Methanesulfonyl-10-(trifluoromethoxy)-7-thia-2,5-diazatricyclo[6.4.0.0^{2,6}]dodeca-1(8),3,5,9,11-pentaene-4-carboxamide

To a stirred solution of 10-(trifluoromethoxy)-7-thia-2,5-diazatricyclo[6.4.0.0^{2,6}]dodeca-1(8),3,5,9,11-pentaene-4-carboxylic acid (200 mg, 0.66 mmol) in THF (2 mL) at room temperature was added carbonyl diimidazole (215 mg, 1.32 mmol). The mixture was stirred at room temperature for 30 min, heated at 55° C. for 30 min and allowed to cool. Methanesulfonamide (157 mg, 1.65 mmol) and DBU (249 μL, 1.65 mmol) were added and the mixture stirred at room temperature for 16 h. The reaction mixture was partitioned between EtOAc and water. The organic layer was dried (Na₂SO₄), the mixture filtered and the filtrate evaporated. The residue was purified by FCC on silica (eluent: DCM:methanol 98:2) to afford the title compound as a grey solid (210 mg, 82% yield); m/z=380.1 (MH)⁺; ¹H NMR (400 MHz, DMSO-d6) 3.34 (s, 3H), 7.65 (dd, 1H), 8.23-8.32 (m, 2H), 9.15 (s, 1H), 11.71 (s, 1H).

### Example 21

### 9,10-Dichloro-N-methanesulfonyl-7-thia-2,5-diazatricyclo[6.4.0.0^{2,6}]dodeca-1(8),3,5,9,11-pentaene-4-carboxamide

To a solution of 9,10-dichloro-7-thia-2,5-diazatricyclo[6.4.0.0^{2,6}]dodeca-1(8),3,5,9,11-pentaene-4-carboxylic acid (160 mg, 0.557 mmol) in a mixture of tert-butanol (1.5 mL) and DCM (1.5 mL) was added DMAP (204 mg, 1.67 mmol) and EDC (216 mg, 1.39 mmol). The reaction mixture was stirred for 15 min then methanesulfonamide (48 mg, 0.502 mmol) was added and the reaction mixture was stirred at room temperature overnight. More DCM (50 mL) was then added, the mixture was washed with 1N hydrochloric acid (3×25 mL), and the organic phase was dried over sodium sulfate and concentrated under reduced pressure to afford the title compound as a solid (150 mg, 31% purity, 23% yield). The material was combined with the crude product from a previous batch and further purified by FCC on silica (eluent: 4% MeOH in DCM). Product containing fractions were then triturated with DMSO and the resultant solid washed with water to afford the title compound in high purity; m/z=363.8 (MH)⁺; ¹H NMR (500 MHz, DMSO-d6) 2.89 (s, 3H), 7.86 (d, 1H), 8.18 (d, 1H), 8.69 (s, 1H). Exchangeables not observed.

### Example 22

### 9,10-Dichloro-N-(cyclopropanesulfonyl)-7-thia-2,5-diazatricyclo[6.4.0.0^{2,6}]dodeca-1(8),3,5,9,11-pentaene-4-carboxamide

To a stirred suspension of 9,10-dichloro-7-thia-2,5-diazatricyclo[6.4.0.0^{2,6}]dodeca-1(8),3,5,9,11-pentaene-4-carboxylic acid (90%, 126 mg, 0.39 mmol) in anhydrous DMF (5 mL) were added EDC (153 mg, 0.99 mmol) and DMAP (145 mg, 1.18 mmol). The mixture was stirred at room temp for 15 min before cyclopropanesulfonamide (48 mg, 0.39 mmol) was added. The reaction was sealed under nitrogen and stirred at room temp for 56 h. The reaction was treated with more EDC (153 mg, 0.99 mmol) and DMAP (145 mg, 1.18 mmol) and stirred at room temperature for a further 18 h. The reaction was quenched by the addition of 1M HCl (10 mL), then diluted with DCM (60 mL) and the phases were separated. The organic phase was washed with 1M HCl (2×10 mL), water (10 mL) and brine (20 mL) then concentrated in vacuo. The residue was dry loaded onto silica then purified by FCC over silica (eluent: DCM:MeOH 1:0 to 94:6) to afford the title compound as an off white solid (51 mg, 32% yield); m/z=389.8 (MH)⁺; ¹H NMR (500 MHz, DMSO-d6) 0.80-0.88 (m, 2H), 0.92-1.02 (m, 2H), 2.91-3.06 (m, 1H), 7.89 (d, 1H), 8.20 (d, 1H), 8.85 (s, 1H). Exchangeables not observed.

### Example 23

### 9,10-Dichloro-N-(2-methoxyethanesulfonyl)-7-thia-2,5-diazatricyclo[6.4.0.0^{2,6}]dodeca-1(8),3,5,9,11-pentaene-4-carboxamide

The procedure to prepare 9,10-dichloro-N-(cyclopropanesulfonyl)-7-thia-2,5-diazatricyclo[6.4.0.0^{2,6}]dodeca-1(8),3,5,9,11-pentaene-4-carboxamide was used except that 2-methoxyethanesulfonamide was substituted for cyclopropanesulfonamide (28% yield); m/z=407.8 (MH)⁺; ¹H NMR (500 MHz, DMSO-d6) δ 3.20 (s, 3H), 3.35-3.45 (m, 2H), 3.58-3.68 (m, 2H), 7.88 (d, 1H), 8.20 (d, 1H), 8.77 (s, 1H). Exchangeables not observed.

### Example 24

### 9,10-Dichloro-N-(ethanesulfonyl)-7-thia-2,5-diazatricyclo[6.4.0.0^{2,6}]dodeca-1(8),3,5,9,11-pentaene-4-carboxamide

The procedure to prepare 9,10-dichloro-N-(cyclopropanesulfonyl)-7-thia-2,5-diazatricyclo[6.4.0.0^{2,6}]dodeca-1(8),3,5,9,11-pentaene-4-carboxamide was used except that ethanesulfonamide was substituted for cyclopropanesulfonamide (29% yield); m/z=377.8 (MH)⁺; ¹H NMR (500 MHz, DMSO-d6) δ 1.15 (t, 3H), 3.10 (q, 2H), 7.88 (d, 1H), 8.20 (d, 1H), 8.74 (s, 1H). Exchangeables not observed.

### Example 25

### 9,10-Dichloro-N-(2,2,2-trifluoroethanesulfonyl)-7-thia-2,5-diazatricyclo[6.4.0.0^{2,6}]dodeca-1(8),3,5,9,11-pentaene-4-carboxamide

The procedure to prepare 9,10-dichloro-N-(cyclopropanesulfonyl)-7-thia-2,5-diazatricyclo[6.4.0.0^{2,6}]dodeca-1(8),3,5,9,11-pentaene-4-carboxamide was used except that 2,2,2-trifluoroethanesulfonamide was substituted for cyclopropanesulfonamide and no re-treatment with coupling reagents was deemed necessary (63% yield); m/z=431.8 (MH)⁺; ¹H NMR (500 MHz, DMSO-d6) δ 4.70-4.88 (m, 2H), 7.93 (d, 1H), 8.21 (d, 1H), 9.17 (s, 1H). Exchangeables not observed.

### Example 26

### 9,10-Dichloro-N-trifluoromethanesulfonyl-7-thia-2,5-diazatricyclo[6.4.0.0^{2,6}]dodeca-1(8),3,5,9,11-pentaene-4-carboxamide

To a stirred solution of 9,10-dichloro-7-thia-2,5-diazatricyclo[6.4.0.0^{2,6}]dodeca-1(8),3,5,9,11-pentaene-4-carboxylic acid (78 mg,0.24 mmol) in DMF (10 mL) were added EDC (1118 mg, 0.61 mmol) and DMAP (90 mg, 0.73 mmol). The mixture was stirred at room temperature for 10 min before trifluoromethanesulfonamide (38 mg, 0.24 mmol) was added. The mixture stirred for 61 h. The reaction mixture was concentrated in vacuo and diluted with EtOAc (50 mL). The organic phase was washed with water (2×40 mL), 1M HCl (40 mL) and brine (40 mL). The organic layer was dried over Na₂SO₄, filtered and concentrated in vacuo. The residue was purified by column chromatography (dry loaded onto silica, using 0-5% MeOH in DCM eluent) to afford an orange solid which was further purified via automated reverse phase HPLC (high pH method) to afford the title compound as a white solid (13 mg, 13% yield); m/z=417.9 (MH)⁺; 1H NMR (500 MHz, DMSO-d6) δ 6.89 (s, 1H), 7.85 (d, 1H), 8.21 (d, 1H), 8.73 (s, 1H).

### Example 27

### Methyl 3-[({9,10-dichloro-7-thia-2,5-diazatricyclo[6.4.0.0² 6]dodeca-1(8),3,5,9,11-pentaen-4-yl}formamido)sulfonyl]propanoate

A suspension of 9,10-dichloro-7-thia-2,5-diazatricyclo[6.4.0.0^{2,6}]dodeca-1(8),3,5,9,11-pentaene-4-carboxylic acid (85%, 110 mg, 0.33 mmol), EDC (126 mg, 0.81 mmol) and DMAP (119 mg, 0.98 mmol) was stirred in anhydrous DMF (5 mL) for 5 min before a solution of methyl 3-sulfamoylpropanoate (57 mg, 0.34 mmol) in DCM (1 mL) was added. The reaction was sealed under nitrogen and stirred at room temperature for 60 h. The reaction mixture was diluted with EtOAc (30 mL), washed with 1M HCl (5 mL), water (5 mL), brine (5 mL) then concentrated in vacuo. The residue was purified by FCC over silica (eluent: DCM:MeOH 98:2 to 92:8) to afford approximately 50 mg of product which was triturated with heptane:EtOAc:DCM 2:2:1 (2×4 mL). The residue was dried in vacuo to afford the title compound as a white solid (43 mg, 29% yield); m/z=435.8 (MH)⁺; ¹H NMR (250 MHz, DMSO-d6) δ 2.83 (t, 2H), 3.60 (s, 3H), 3.77 (t, 2H), 7.94 (d, 1H), 8.21 (d, 1H), 9.17 (s, 1H). Exchangeables not observed.

### Example 28

### 9,10-Dichloro-N-(3-hydroxypropanesulfonyl)-7-thia-2,5-diazatricyclo[6.4.0.0^{2,6}]dodeca-1(8),3,5,9,11-pentaene-4-carboxamide

A suspension of LiBH₄ (3 mg, 0.14 mmol) and methanol (6 μL, 0.14 mmol) in anhydrous dioxane (1 mL) was added to a suspension of methyl 3-[({9,10-dichloro-7-thia-2,5-diazatricyclo[6.4.0.0^{2,6}]dodeca-1(8),3,5,9,11-pentaen-4-yl}formamido)sulfonyl]propanoate (43 mg, 0.09 mmol) in anhydrous dioxane (2 mL). The reaction mixture was sealed under nitrogen and stirred at 80° C. for 2.5 h. The reaction was then allowed to cool to room temperature then stored in the fridge overnight. The reaction was stirred at 80° C. for 1 h. The reaction was retreated with LiBH₄ (3 mg, 0.14 mmol) and methanol (6 μL, 0.14 mmol) and then stirred at 80° C. for a further 60 min. The reaction was diluted with EtOAc (20 mL) and extracted with saturated aqueous NaHCO₃ (3×3 mL). The combined aqueous extracts were washed with DCM (3 mL) and then brought to pH 1 by the addition of 3N HCl (aq). The resulting suspension was washed with DCM (2×5 mL) then filtered over glass fibre filter paper. The solid residue was washed with water (5 mL) then dried under vacuum to afford the title compound as a white solid (20 mg, 50% yield); m/z=407.9 (MH)⁺; ¹H NMR (250 MHz, DMSO-d6) δ 1.85 (dt, 2H), 3.44-3.58 (m, 4H), 4.68 (s, 1H), 7.93 (d, 1H), 8.20 (d, 1H), 9.16 (s, 1H), 11.61 (s, 1H).

### Example 29

### 9,10-Dichloro-N-[3-(diethylamino)propanesulfonyl]-7-thia-2,5-diazatricyclo[6.4.0.0^{2,6}]dodeca-1(8),3,5,9,11-pentaene-4-carboxamide

To a solution of 9,10-dichloro-N-(3-chloropropanesulfonyl)-7-thia-2,5-diazatricyclo[6.4.0.0^{2,6}]dodeca-1(8),3,5,9,11-pentaene-4-carboxamide (80 mg, 0.19 mmol) in anhydrous dioxane (5 mL) in a pressure tube under nitrogen was added diethylamine (15 μL, 0.21 mmol), sodium iodide (3 mg, 0.02 mmol) and sodium carbonate (40 mg, 0.37 mmol). The tube was sealed and the resulting reaction mixture was stirred at 75° C. for 16 h. A further portion of diethylamine (15 μL, 0.21 mmol) was added, the tube re-sealed and the mixture stirred for a further 16 h at 100° C. The mixture was evaporated to dryness and suspended in chloroform (20 mL). The resulting suspension was filtered and the solid extracted with DCM (20 mL). The combined organic phases were evaporated to dryness and purified by automated reverse phase HPLC low pH method) to afford the title compound as a yellow solid (formate salt, 7 mg, 8% yield); m/z=462.9 (MH)⁺; ¹H NMR (500 MHz, Methanol-d4) δ 1.32 (t, 6H), 2.20-2.31 (m, 2H), 3.23-3.28 (m, 4H), 3.35-3.40 (m, 2H), 3.43-3.45 (m, 2H), 7.72 (d, 1H), 7.91 (d, 1H), 8.31 (s, 2H), 8.49 (s, 1H). Formic acid salt.

### Example 30

### 2-{9,10-Dichloro-7-thia-2,5-diazatricyclo[6.4.0.0^{2,6}]dodeca-1(8),3,5,9,11-pentaen-4-yl}-N-methanesulfonylacetamide

To a solution of 2-{9,10-dichloro-7-thia-2,5-diazatricyclo[6.4.0.0^{2,6}]dodeca-1(8),3,5,9,11-pentaen-4-yl}acetic acid (70 mg, 0.23 mmol) in anhydrous DMF (2 mL) was added EDC (111 mg, 0.58 mmol) and DMAP (85 mg, 0.7 mmol). The mixture was stirred at room temperature for 10 min under an inert atmosphere and then methanesulfonamide (33 mg, 0.35 mmol) was added. The resulting solution was stirred at ambient temperature for 16 h then heated at 50° C. for 6 h before being evaporated to dryness. The residue was dissolved in DCM (100 mL) and washed with water (3×25 mL) and 1M HCl (25 mL). The organic phase was dried over MgSO₄, the mixture filtered and the filtrate evaporated to dryness to afford the crude product as a black oil. This was purified by automated reverse phase HPLC (low pH method) to afford the title compound as a white solid (6 mg, 7% yield); m/z=378.0 (MH)⁺; ¹H NMR (500 MHz, Methanol-d4) δ 3.02 (s, 3H), 3.62 (d, 2H), 7.68 (d, 1H), 7.80 (d, 1H), 7.92 (s, 1H). Exchangeables not observed.

### Example 31

### 7-Thia-2,5-diazatricyclo[6.4.0.0^{2,6}]dodeca-1(8),3,5-triene-4-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 7-thia-2,5-diazatricyclo[6.4.0.0^{2,6}]dodeca-1(8),3,5-triene-4-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (92% yield); m/z=222.9 (MH)⁺; ¹H NMR (500 MHz, DMSO-d6) δ 1.72-1.97 (m, 4H), 2.65-2.73 (m, 4H), 8.27 (s, 1H), 12.45 (s, 1H).

### Example 32

### 9,9,11,11-Tetramethyl-7-thia-2,5-diazatricyclo[6.4.0.0^{2,6}]dodeca-1(8),3,5-triene-4-carboxylic acid

Ethyl 3-bromo-2-oxopropanoate (1.67 g, 8.56 mmol) was added dropwise to a solution of 5,5,7,7-tetramethyl-4,5,6,7-tetrahydro-1,3-benzothiazol-2-amine (1.50 g, 7.13 mmol) in NMP (40 mL) at 75° C. containing solid NaHCO₃ (1.20 g, 14.2 mmol) and the mixture stirred at this temperature for 30 min. The cooled reaction mixture was partitioned between EtOAc (200 mL) and water (100 mL). The phases were separated and the organic phase was washed with water (3×100 mL), brine (20 mL), dried (Na₂SO₄) and the mixture was filtered. The filtrate was evaporated to dryness to afford a brown oil which was purified by FCC on silica (eluent: 20-80% EtOAc in heptane) to afford the intermediate ethyl ester as a brown oil.

The ester was converted to the acid by refluxing in 6M HCl (10 mL) for 3 h followed by evaporation of the solvent to afford the title compound as a brown solid (555 mg, 26% yield); m/z=279.2 (MH)⁺; ¹H NMR (400 MHz, DMSO-d6) δ 1.07 (s, 6H), 1.30 (s, 6H), 1.66 (s, 2H), 2.53 (s, 2H), 8.25 (s, 1H), 12.45 (s, 1H).

### Example 33

### 10,10-Dimethyl-7-thia-2,5-diazatricyclo[6.4.0.0^{2,6}]dodeca-1(8),3,5-triene-4-carboxylic acid

To a solution of ethyl 10,10-dimethyl-7-thia-2,5-diazatricyclo[6.4.0.0^{2,6}]dodeca-1(8),3,5-triene-4-carboxylate (100 mg, 0.36 mmol) in MeOH (5 mL) was added LiOH.H₂O (30 mg, 0.72 mmol) in water (0.5 mL). The reaction was stirred overnight at room temperature. Water was added and the aqueous phase washed once with EtOAc. The aqueous layer was acidified with 1M HCl and extracted with EtOAc (3×20 mL). The combined organic layers were dried over sodium sulfate, filtered and the filtrate concentrated. The residue was recrystallized using DCM:n-pentane and dried in vacuo to afford the title compound as a light brown solid (20 mg, 20% yield); m/z=251.4 (MH)⁺; ¹H NMR (400 MHz, Chloroform-d) δ 1.10 (s, 6H), 1.73-1.80 (m, 2H), 2.52 (s, 2H), 2.72-2.80 (m, 2H), 8.15 (s, 1H). Exchangeables not observed.

### Example 34

### 10-Phenyl-7-thia-2,5-diazatricyclo[6.4.0.0^{2,6}]dodeca-1(8),3,5-triene-4-carboxylic acid

The procedure to prepare 10,10-dimethyl-7-thia-2,5-diazatricyclo[6.4.0.0^{2,6}]dodeca-1(8),3,5-triene-4-carboxylic acid was used except that ethyl 10-phenyl-7-thia-2,5-diazatricyclo[6.4.0.0^{2,6}]dodeca-1(8),3,5-triene-4-carboxylate was substituted for ethyl 10,10-dimethyl-7-thia-2,5-diazatricyclo[6.4.0.0^{2,6}]dodeca-1(8),3,5-triene-4-carboxylate (22% yield); m/z=299.2 (MH)⁺; ¹H NMR (400 MHz, Methanol-d4) δ 2.00-2.23 (m, 2H), 2.75-2.94 (m, 4H), 3.04-3.17 (m, 1H), 7.08-7.17 (m, 2H), 7.17-7.31 (m, 3H), 8.09 (s, 1H). Exchangeables not observed.

### Example 35

### 10-tert-Butyl-7-thia-2,5-diazatricyclo[6.4.0.0^{2,6}]dodeca-1(8),3,5-triene-4-carboxylic acid

A solution of ethyl 10-tert-butyl-7-thia-2,5-diazatricyclo[6.4.0.0^{2,6}]dodeca-1(8),3,5-triene-4-carboxylate (96%, 733 mg, 2.30 mmol) in 5M HCl (40 mL) was heated at 100° C. for 5 h. The reaction was concentrated in vacuo to leave a brown solid (600 mg). This was purified by automated reverse phase HPLC (low pH method) to afford the title compound as a grey solid (422 mg, 66% yield); m/z=279.4 (MH)⁺; ¹H NMR (500 MHz, DMSO-d6) δ 0.95 (s, 9H), 1.35-1.50 (m, 1H), 1.56-1.69 (m, 1H), 2.04-2.14 (m, 1H), 2.56-2.64 (m, 2H), 2.72 (dd, 1H), 2.87 (dd, 1H), 8.27 (s, 1H), 12.44 (s, 1H).

### Example 36

### 4,4,5,5-Tetramethyl-7-thia-1,9-diazatricyclo[6.3.0.0^{2,6}]undeca-2(6),8,10-triene-10-carboxylic acid

The procedure to prepare 10-tert-butyl-7-thia-2,5-diazatricyclo[6.4.0.0^{2,6}]dodeca-1(8),3,5-triene-4-carboxylic acid was used except that ethyl 4,4,5,5-tetramethyl-7-thia-1,9-diazatricyclo[6.3.0.0^{2,6}]undeca-2(6),8,10-triene-10-carboxylate was substituted for ethyl 10-tert-butyl-7-thia-2,5-diazatricyclo[6.4.0.0^{2,6}]dodeca-1(8),3,5-triene-4-carboxylate and the bulk of the product was not purified by HPLC (59% yield); m/z=265.4 (MH)⁺; ¹H NMR (400 MHz, DMSO-d6) δ 1.10 (s, 6H), 1.13 (s, 6H), 2.75 (s, 2H), 8.29 (s, 1H), 12.45 (s, 1H).

### Example 37

### 7-Thia-2,5-diazatricyclo[6.5.0.0^{2,6}]trideca-1(8),3,5-triene-4-carboxylic acid

The procedure to prepare 9,10-dichloro-7-thia-2,5-diazatricyclo[6.4.0.0^{2,6}]dodeca-1(8),3,5,9,11-pentaene-4-carboxylic acid was used except that ethyl 7-thia-2,5-diazatricyclo[6.5.0.0^{2,6}]trideca-1(8),3,5-triene-4-carboxylate was substituted for ethyl 9,10-dichloro-7-thia-2,5-diazatricyclo[6.4.0.0^{2,6}]dodeca-1(8),3,5,9,11-pentaene-4-carboxylate (35% yield); m/z=237.4 (MH)⁺; ¹H NMR (400 MHz, DMSO-d6) δ 1.67-1.85 (m, 6H), 2.72-2.76 (m, 2H), 2.88-2.95 (m, 2H), 8.32 (s, 1H), 12.45 (s, 1H).

### Example 38

### 11,11-Dimethyl-7-thia-2,5-diazatricyclo[6.4.0.0^{2,6}]dodeca-1(8),3,5-triene-4-carboxylic acid

Ethyl 11,11-dimethyl-7-thia-2,5-diazatricyclo[6.4.0.0^{2,6}]dodeca-1(8),3,5-triene-4-carboxylate (85%, 180 mg, 0.55 mmol), LiOH.H₂O (46 mg, 1.1 mmol), MeOH (4 mL) and water (4 mL) were charged to a round bottomed flask and stirred at 70° C. for a total of 4 h. The MeOH was removed in vacuo and the remaining aqueous phase was diluted with water (5 mL), washed with DCM (3×5 mL), then made acidic by the addition of saturated aqueous citric acid. The resulting suspension was extracted with DCM (4×10 mL). The combined extractions were washed with brine (10 mL), dried over anhydrous Na₂SO₄, filtered and the filtrate was concentrated in vacuo to afford the title compound as a light brown solid (120 mg, 83% yield); m/z=251.0 (MH)⁺; ¹H NMR (500 MHz, Chloroform-d) δ 1.11 (s, 6H), 1.71 (t, 2H), 2.44 (s, 2H), 2.73 (t, 2H), 7.96 (s, 1H). Exchangeables not observed.

### Example 39

### 9-Hydroxy-7-thia-2,5-diazatricyclo[6.4.0.0^{2,6}]dodeca-1(8),3,5-triene-4-carboxylic acid

To a stirred solution of ethyl 9-hydroxy-7-thia-2,5-diazatricyclo[6.4.0.0^{2,6}]dodeca-1(8),3,5-triene-4-carboxylate (65%, 175 mg, 0.43 mmol) in MeOH:water (4:1, 10 mL) under nitrogen was added 1M sodium hydroxide (1.28 mL, 1.28 mmol) and the mixture heated at 50° C. for 1 h. The reaction mixture was cooled down and solvent removed under reduced pressure. Water (1 mL) was added and the reaction mixture acidified to approximately pH 3-4 using 1M HCl. Excess solvent was removed under reduced pressure and the resulting brown residue was purified by automated reverse phase HPLC (low pH method) to afford the title compound as an orange solid (23 mg, 22% yield); m/z=239.1 (MH)⁺; ¹H NMR (500 MHz, DMSO-d6) δ 1.69-1.84 (m, 2H), 1.98-2.08 (m, 2H), 2.62-2.71 (m, 2H), 4.67 (s, 1H), 5.67 (s, 1H), 8.20 (s, 1H). 1 Exchangeable not observed.

### Example 40

### 10-tert-Butyl-3-(tert-butylamino)-7-thia-2,5-diazatricyclo[6.4.0.0^{2,6}]dodeca-1(8),3,5-triene-4-carboxylic acid

Ethyl 10-tert-butyl-3-(tert-butylamino)-7-thia-2,5-diazatricyclo[6.4.0.0^{2,6}]dodeca-1(8),3,5-triene-4-carboxylate (190 mg, 75%, 0.377 mmol) and LiOH.H₂O (31 mg, 0.755 mmol) were stirred in MeOH/water (5 mL/0.5 mL) at room temp for 16 h. Extra LiOH.H₂O (31 mg, 0.755 mmol) was added and the reaction heated at 40° C. for 2 days and then stirred at room temperature for 2 days. The crude reaction was concentrated and purified by automated reverse phase preparative HPLC (low pH method) to afford the title compound as an off white solid (19 mg, 14% yield); m/z=350.2 (MH)⁺; ¹H NMR (500 MHz, DMSO-d6) δ 0.92 (s, 9H), 1.07 (s, 9H), 1.33 (qd, 1H), 1.54-1.67 (m, 1H), 2.00-2.12 (m, 1H), 2.33-2.47 (m, 2H), 2.60-2.80 (m, 2H). Exchangeables not observed.

### Example 41

### 16-Thia-11,14-diazatetracyclo[8.6.0.0^{2,7}.0^{11,15}]hexadeca-1 (10),2(7),3,5,12,14-hexaene-13-carboxylic acid

The procedure to prepare 9-chloro-10-fluoro-7-thia-2,5-diazatricyclo[6.4.0.0^{2,6}]dodeca-1(8),3,5,9,11-pentaene-4-carboxylic acid was used except that ethyl 16-thia-11,14-diazatetracyclo[8.6.0.0^{2,7}.0^{11,15}]hexadeca-1(10),2(7),3,5,12,14-hexaene-13-carboxylate was substituted for ethyl 9-chloro-10-fluoro-7-thia-2,5-diazatricyclo[6.4.0.0^{2,6}]dodeca-1(8),3,5,9,11-pentaene-4-carboxylate (57% yield); m/z=271.0 (MH)⁺; ¹H NMR (500 MHz, DMSO-d6) δ 3.08-3.12 (m, 2H), 3.12-3.16 (m, 2H), 7.06-7.48 (m, 4H), 8.48 (s, 1H), 12.54 (s, 1H).

### Example 42

### 3-(Benzylamino)-10-tert-butyl-7-thia-2,5-diazatricyclo[6.4.0.0^{2,6}]dodeca-1(8),3,5-triene-4-carboxylic acid

The procedure to prepare 10-tert-butyl-3-(tert-butylamino)-7-thia-2,5-diazatricyclo[6.4.0.0^{2,6}]dodeca-1(8),3,5-triene-4-carboxylic acid was used except that ethyl 3-(benzylamino)-10-tert-butyl-7-thia-2,5-diazatricyclo[6.4.0.0^{2,6}]dodeca-1(8),3,5-triene-4-carboxylate was substituted for ethyl 10-tert-butyl-3-(tert-butylamino)-7-thia-2,5-diazatricyclo[6.4.0.0^{2,6}]dodeca-1(8),3,5-triene-4-carboxylate (17% yield); m/z=384.2 (MH)⁺; ¹H NMR (500 MHz, DMSO-d6) δ 0.92 (s, 9H), 1.38 (qd, 1H), 1.59 (td, 1H), 1.98-2.08 (m, 1H), 2.33-2.47 (m, 1H), 2.60-2.72 (m, 1H), 2.79-2.93 (m, 1H), 3.12-3.23 (m, 1H), 4.15-4.36 (m, 2H), 5.24 (s, 1H), 7.23-7.29 (m, 1H), 7.29-7.36 (m, 4H). 1 Exchangeable not observed.

### Example 43

### 9,9-Dimethyl-7-thia-2,5-diazatricyclo[6.4.0.0^{2,6}]dodeca-1(8),3,5-triene-4-carboxylic acid

To ethyl 9,9-dimethyl-7-thia-2,5-diazatricyclo[6.4.0.0^{2,6}]dodeca-1(8),3,5-triene-4-carboxylate (36 mg, 0.129 mmol) was added 6M HCl (5 mL) and the mixture heated at 100° C. for 2 h. The solution was concentrated in vacuo to yield a brown residue which was triturated using DCM to afford the title compound as a brown solid (24 mg, 73% yield); m/z=251.1 (MH)⁺; ¹H NMR (500 MHz, DMSO-d6) δ 1.05 (s, 6H), 1.65 (t, 2H), 2.52-2.54 (m, 2H), 2.71 (t, 2H), 8.29 (s, 1H). Exchangeables not observed.

### Example 44

### 9-Methyl-7-thia-2,5-diazatricyclo[6.4.0.0^{2,6}]dodeca-1(8),3,5-triene-4-carboxylic acid

To ethyl 9-methyl-7-thia-2,5-diazatricyclo[6.4.0.0^{2,6}]dodeca-1(8),3,5-triene-4-carboxylate (388 mg, 1.47 mmol) was added 6M HCl (20 mL) and the mixture heated at 100° C. for 2 h whereupon a brown solution formed. The solvent was evaporated and the residue azeotroped with DCM several times. The resulting yellow solid was then triturated with DCM to afford the title compound as a tan solid (296 mg, 83% yield); m/z=237.0 (MH)⁺; ¹H NMR (500 MHz, DMSO-d6) δ 1.24 (d, 3H), 1.42-1.56 (m, 1H), 1.71-1.86 (m, 1H), 1.92-2.11 (m, 2H), 2.66 (ddt, 1H), 2.70-2.78 (m, 1H), 2.90-3.03 (m, 1H), 8.34 (s, 1H). Exchangeables not observed.

### Example 45

### 2-{10-tert-Butyl-7-thia-2,5-diazatricyclo[6.4.0.0^{2,6}]dodeca-1(8),3,5-trien-4-yl}acetic acid

To a solution of 6-tert-butyl-4,5,6,7-tetrahydro-1,3-benzothiazol-2-amine (75%, 200 mg, 0.71 mmol) in acetone (5 mL) was added ethyl 4-bromo-3-oxobutanoate (0.300 mL, 2.14 mmol). The resulting solution was stirred at room temperature for 1 h then evaporated to dryness, re-dissolved in EtOH (5 mL) and refluxed at 75° C. for a further 3 h. The mixture was evaporated to dryness and the residue purified by silica chromatography (0-5% MeOH in DCM) to afford the intermediate ester which was dissolved in EtOH (3 mL) and water (1 mL) and treated with LiOH.H₂O (37 mg, 0.87 mmol). The resulting mixture was refluxed at 75° C. for 1 h then evaporated to dryness. The residue was dissolved in water (5 mL) and acidified with 1M HCl to pH 2. The aqueous phase was then extracted into DCM (2×25 mL). The combined organic phase was evaporated and purified by automated reverse phase HPLC (low pH method) to afford the title compound as a white solid (17 mg, 13% yield); m/z=293.0 (MH)⁺; ¹H NMR (500 MHz, Methanol-d4) δ 0.99 (s, 9H), 1.53 (qd, 1H), 1.61-1.70 (m, 1H), 2.14-2.24 (m, 1H), 2.46-2.65 (m, 2H), 2.76 (ddd, 2H), 3.67 (s, 2H), 7.44 (s, 1H). Exchangeables not observed.

### Example 46

### 2-{9,9,11,11-Tetramethyl-7-thia-2,5-diazatricyclo[6.4.0.0^{2,6}]dodeca-1(8),3,5-trien-4-yl}acetic acid

The procedure to prepare 2-{10-tert-butyl-7-thia-2,5-diazatricyclo[6.4.0.0^{2,6}]dodeca-1(8),3,5-trien-4-yl}acetic acid was used except that 5,5,7,7-tetramethyl-4,5,6,7-tetrahydro-1,3-benzothiazol-2-amine was substituted for 6-tert-butyl-4,5,6,7-tetrahydro-1,3-benzothiazol-2-amine (28% yield); m/z=293.4 (MH)⁺; ¹H NMR (500 MHz, DMSO-d6) δ 1.08 (s, 6H), 1.29 (s, 6H), 1.66 (s, 2H), 2.47 (s, 2H), 3.54 (s, 2H), 7.48 (s, 1H). Exchangeables not observed.

### Example 47

### 10-tert-Butyl-N-methanesulfonyl-7-thia-2,5-diazatricyclo[6.4.0.0^{2,6}]dodeca-1(8),3,5-triene-4-carboxamide

10-tert-Butyl-7-thia-2,5-diazatricyclo[6.4.0.0^{2,6}]dodeca-1(8),3,5-triene-4-carboxylic acid (201 mg, 0.72 mmol) was dissolved in DMF (6 mL). EDC (280 mg, 1.8 mmol) and DMAP (270 mg, 2.2 mmol) were added and the reaction mixture was stirred for 5 min. Methanesulfonamide (69 mg, 0.73 mmol) was then added and the reaction mixture was stirred at room temperature overnight then re-treated with EDC (279 mg, 1.8 mmol) and DMAP (263 mg, 2.15 mmol) and the reaction mixture was stirred for 5 min. More methanesulfonamide (69 mg, 0.73 mmol) was then added and the reaction mixture was stirred for a further 24 h before being diluted with EtOAc and water. The phases were separated and the aqueous phase washed with EtOAc (×3) before being acidified with 3M HCl (to take it to pH 1) and re-extracted with EtOAc (×3). The combined organic extracts were washed with brine and dried (Na₂SO₄), the mixture filtered and the filtrate evaporated to dryness. The residue was purified by automated reverse phase HPLC (low pH method) to afford the title compound as a white solid (26 mg, 10% yield); m/z=356.3 (MH)⁺; ¹H NMR (500 MHz, Methanol-d4) δ 1.01 (s, 9H), 1.50-1.63 (m, 1H), 1.65-1.76 (m, 1H), 2.11-2.28 (m, 1H), 2.48-2.61 (m, 1H), 2.61-2.73 (m, 1H), 2.73-2.92 (m, 2H), 3.34 (s, 3H), 8.22 (s, 1H). Exchangeables not observed.

### Example 48

### N-Methanesulfonyl-9-methyl-7-thia-2,5-diazatricyclo[6.4.0.0^{2,6}]dodeca-1(8),3,5-triene-4-carboxamide, Enantiomer 1

The procedure to prepare 10-tert-butyl-N-methanesulfonyl-7-thia-2,5-diazatricyclo[6.4.0.0^{2,6}]dodeca-1(8),3,5-triene-4-carboxamide was used except that racemic 9-methyl-7-thia-2,5-diazatricyclo[6.4.0.0^{2,6}]dodeca-1(8),3,5-triene-4-carboxylic acid was substituted for 10-tert-butyl-7-thia-2,5-diazatricyclo[6.4.0.0^{2,6}]dodeca-1(8),3,5-triene-4-carboxylic acid (270 mg, 69% yield of racemic acyl sulfonamide). The racemic mixture was resolved using chiral HPLC. The stationary phase used was a YMC AMY-C (20 mm×250 mm, 5 m) column. The mobile phase was heptane:isopropanol 70:30 containing some diethylamine, added as a modifier). 84 mg of Enantiomer 1 was recovered from the chiral purification; m/z=314.1 (MH)⁺; ¹H NMR (500 MHz, Methanol-d4) δ 1.29 (d, 3H), 1.49-1.63 (m, 1H), 1.79-1.95 (m, 1H), 2.03-2.20 (m, 2H), 2.60-2.76 (m, 2H), 2.93-3.00 (m, 1H), 3.11 (s, 3H), 7.95 (s, 1H). Exchangeables not observed.

### Example 49

### N-Methanesulfonyl-9-methyl-7-thia-2,5-diazatricyclo[6.4.0.0^{2,6}]dodeca-1(8),3,5-triene-4-carboxamide, Enantiomer 2

The procedure and chiral separation used to obtain Enantiomer 1 was used. Enantiomer 2 (75 mg) was recovered from the chiral purification; m/z=314.1 (MH)⁺; ¹H NMR (500 MHz, Methanol-d4) δ 1.29 (d, 3H), 1.46-1.62 (m, 1H), 1.77-1.96 (m, 1H), 2.03-2.16 (m, 2H), 2.61-2.77 (m, 2H), 2.91-3.00 (m, 1H), 3.11 (s, 3H), 7.94 (s, 1H). Exchangeables not observed.

### Example 50

### N-Methanesulfonyl-11,11-dimethyl-7-thia-2,5-diazatricyclo[6.4.0.0^{2,6}]dodeca-1(8),3,5-triene-4-carboxamide

The procedure to prepare 10-tert-butyl-N-methanesulfonyl-7-thia-2,5-diazatricyclo[6.4.0.0^{2,6}]dodeca-1(8),3,5-triene-4-carboxamide was used except that 11,11-dimethyl-7-thia-2,5-diazatricyclo[6.4.0.0^{2,6}]dodeca-1(8),3,5-triene-4-carboxylic acid was substituted for 10-tert-butyl-7-thia-2,5-diazatricyclo[6.4.0.0^{2,6}]dodeca-1(8),3,5-triene-4-carboxylic acid (20% yield); m/z=328.2 (MH)⁺; ¹H NMR (500 MHz, DMSO-d6) δ 1.05 (s, 6H), 1.65 (t, 2H), 2.53 (s, 2H), 2.67-2.78 (m, 2H), 3.32 (s, 3H), 8.46 (s, 1H). Exchangeables not observed.

### Example 51

### 10-tert-Butyl-N-[3-(morpholin-4-yl)propanesulfonyl]-7-thia-2,5-diazatricyclo[6.4.0.0^{2,6}]dodeca-1(8),3,5-triene-4-carboxamide

To a solution of 10-tert-butyl-N-(3-chloropropanesulfonyl)-7-thia-2,5-diazatricyclo[6.4.0.0^{2,6}]dodeca-1(8),3,5-triene-4-carboxamide (200 mg, 0.48 mmol, 50% purity) in anhydrous dioxane (5 mL) was added morpholine (45 μL, 0.53 mmol), sodium iodide (7 mg, 0.05 mmol) and sodium carbonate (101 mg, 0.96 mmol). The resulting solution was stirred at 75° C. for 16 h. The reaction mixture was evaporated to dryness, dissolved in DCM (50 mL) and washed with water (25 mL), 1M HCl (25 mL) and brine (25 mL). The organic layer was dried over MgSO₄ and evaporated to dryness to afford a brown oil which was purified by automated reverse phase HPLC (low pH method). This afforded the title compound as a white solid (53 mg, 24% yield); m/z=469.2 (MH)⁺; ¹H NMR (500 MHz, DMSO-d6) δ 0.94 (s, 9H), 1.45 (tq, 1H), 1.61 (td, 1H), 1.85 (p, 2H), 2.04-2.16 (m, 1H), 2.35-2.41 (m, 4H), 2.44 (t, 2H), 2.55-2.67 (m, 1H), 2.72 (dd, 1H), 2.85 (dd, 1H), 3.39-3.46 (m, 3H), 3.51-3.60 (m, 4H), 8.33 (s, 1H). Exchangeables not observed.

### Example 52

### N-Methanesulfonyl-4,4,5,5-tetramethyl-7-thia-1,9-diazatricyclo[6.3.0.0^{2,6}]undeca-2(6),8,10-triene-10-carboxamide

To a suspension of 4,4,5,5-tetramethyl-7-thia-1,9-diazatricyclo[6.3.0.0^{2,6}]undeca-2(6),8,10-triene-10-carboxylic acid (160 mg, 0.605 mmol) in DCM (10 mL) was added oxalyl chloride (230 mg, 1.82 mmol) and a drop of DMF and stirred for 15 min. The solvent was evaporated, more DCM was added (10 mL) followed by methanesulfonamide (69 mg, 0.726 mmol) and DIPEA (156 mg, 1.21 mmol). The mixture was stirred for 1 h. More 100 mg portions of DIPEA and methanesulfonamide were added and the mixture stirred for 6 h intervals until significant product had formed, as judged by LCMS. Brine was added (10 mL) and the phases separated. The organic phase was evaporated to dryness to afford a brown oil which was purified by automated preparative reverse phase HPLC (low pH method) to afford the title compound as an off white solid (37 mg, 18% yield); m/z=342.4 (MH)⁺; ¹H NMR (250 MHz, Methanol-d4) δ 1.21 (s, 6H), 1.24 (s, 6H), 2.81 (s, 2H), 3.37 (s, 3H), 8.25 (s, 1H). Exchangeables not observed.

### Example 53

### N-Methanesulfonyl-9,9,11,11-tetramethyl-7-thia-2,5-diazatricyclo[6.4.0.0^{2,6}]dodeca-1(8),3,5-triene-4-carboxamide

The procedure to prepare N-methanesulfonyl-4,4,5,5-tetramethyl-7-thia-1,9-diazatricyclo[6.3.0.0^{2,6}]undeca-2(6),8,10-triene-10-carboxamide was used except that 9,9,11,11-tetramethyl-7-thia-2,5-diazatricyclo[6.4.0.0^{2,6}]dodeca-1(8),3,5-triene-4-carboxylic acid was substituted for 4,4,5,5-tetramethyl-7-thia-1,9-diazatricyclo[6.3.0.0^{2,6}]undeca-2(6),8,10-triene-10-carboxylic acid and triethylamine substituted for DIPEA. No re-treatment with base and methanesulfonamide was necessary (22% yield); m/z=356.4 (MH)⁺; ¹H NMR (500 MHz, Methanol-d4) δ 1.17 (s, 6H), 1.40 (s, 6H), 1.77 (s, 2H), 2.56 (s, 2H), 3.36 (s, 3H), 8.24 (s, 1H). Exchangeables not observed.

### Example 54

### N-[3-(Diethylamino)propanesulfonyl]-9,9,11,11-tetramethyl-7-thia-2,5-diazatricyclo[6.4.0.0^{2,6}]dodeca-1(8),3,5-triene-4-carboxamide

To a suspension of 9,9,11,11-tetramethyl-7-thia-2,5-diazatricyclo[6.4.0.0^{2,6}]dodeca-1(8),3,5-triene-4-carboxylic acid (165 mg, 0.593 mmol) in DCM (10 mL) was added oxalyl chloride (226 mg, 1.78 mmol) and a drop of DMF and stirred for 15 min. The same amount again of oxalyl chloride and DMF was added and the mixture stirred until complete dissolution occurred. The solvents were evaporated then more DCM (10 mL) was added followed by 3-chloropropane-1-sulfonamide (140 mg, 0.889 mmol) and triethylamine (300 mg, 2.96 mmol). The mixture was stirred for 1 h then left to stand overnight. The solvent was evaporated, dioxane (4 mL) added followed by diethylamine (1.5 mL) and the mixture heated in a sealed tube at 100° C. for 5 h. The solvent was evaporated, DCM (30 mL) added followed by 2M HCl (20 mL). The phases were separated. The aqueous phase was washed with DCM (5 mL) and then evaporated. The residue was purified by low pH preparative HPLC to afford the title compound as the formate salt (15 mg, 6% yield); m/z=455.5 (MH)⁺; ¹H NMR (500 MHz, DMSO-d6) δ 1.03-1.14 (m, 12H), 1.31 (s, 6H), 1.68 (s, 2H), 1.85-1.96 (m, 2H), 2.81-3.00 (m, 6H), 3.13 (t, 2H), 7.88 (s, 1H), 8.17 (s, 1H). Formic acid salt. Exchangeables not observed. Assume 2 protons are under the water peak.

### Example 55

### 9,9,11,11-Tetramethyl-N-[3-(morpholin-4-yl)propanesulfonyl]-7-thia-2,5-diazatricyclo[6.4.0.0^{2,6}]dodeca-1(8),3,5-triene-4-carboxamide

The procedure to prepare N-[3-(diethylamino)propanesulfonyl]-9,9,11,11-tetramethyl-7-thia-2,5-diazatricyclo[6.4.0.0^{2,6}]dodeca-1(8),3,5-triene-4-carboxamide was used except that morpholine was substituted for diethylamine. In addition to low pH HPLC, the high pH HPLC method was used to provide further purification (32% yield); m/z=469.2 (MH)⁺; ¹H NMR (500 MHz, DMSO-d6) δ 1.09 (s, 6H), 1.32 (s, 6H), 1.69 (s, 2H), 1.85 (p, 2H), 2.35-2.42 (m, 4H), 2.45 (t, 2H), 2.54 (s, 2H), 3.54-3.58 (m, 4H), 8.31 (s, 1H). Exchangeables not observed. Assume 2 protons are under the water peak.

### Example 56

### 4,4,5,5-Tetramethyl-N-[3-(morpholin-4-yl)propanesulfonyl]-7-thia-1,9-diazatricyclo[6.3.0.0^{2,6}]undeca-2(6),8,10-triene-10-carboxamide

The procedure to prepare N-[3-(diethylamino)propanesulfonyl]-9,9,11,11-tetramethyl-7-thia-2,5-diazatricyclo[6.4.0.0^{2,6}]dodeca-1(8),3,5-triene-4-carboxamide was used except that 4,4,5,5-tetramethyl-7-thia-1,9-diazatricyclo[6.3.0.0^{2,6}]undeca-2(6),8,10-triene-10-carboxylic acid was substituted for 9,9,11,11-tetramethyl-7-thia-2,5-diazatricyclo[6.4.0.0^{2,6}]dodeca-1(8),3,5-triene-4-carboxylic acid and morpholine was substituted for diethylamine. The material obtained from HPLC purification was further purified by being passed down an SCX cartridge as a solution in DCM/MeOH and then eluting with 7M ammonia solution in methanol (22% yield); m/z=455.5 (MH)⁺; ¹H NMR (500 MHz, Methanol-d4) δ 1.19 (s, 6H), 1.20 (s, 6H), 2.04-2.19 (m, 2H), 2.70-2.87 (m, 8H), 3.39 (t, 2H), 3.70-3.81 (m, 4H), 7.96 (s, 1H). Exchangeables not observed.

### Example 57

### 11,11-Dimethyl-N-(2,2,2-trifluoroethanesulfonyl)-7-thia-2,5-diazatricyclo[6.4.0.0^{2,6}]dodeca-1(8),3,5-triene-4-carboxamide

The procedure to prepare 10-tert-butyl-N-methanesulfonyl-7-thia-2,5-diazatricyclo[6.4.0.0^{2,6}]dodeca-1(8),3,5-triene-4-carboxamide was used except that 11,11-dimethyl-7-thia-2,5-diazatricyclo[6.4.0.0² 6]dodeca-1(8),3,5-triene-4-carboxylic acid was substituted for 10-tert-butyl-7-thia-2,5-diazatricyclo[6.4.0.0^{2,6}]dodeca-1(8),3,5-triene-4-carboxylic acid and 2,2,2-trifluoroethanesulfonamide was substituted for methanesulfonamide (23% yield); m/z=396.0 (MH)⁺; ¹H NMR (500 MHz, DMSO-d6) δ 1.05 (s, 6H), 1.65 (t, 2H), 2.54 (s, 2H), 2.68-2.77 (m, 2H), 4.73 (q, 2H), 8.47 (s, 1H). Exchangeables not observed.

### Example 58

### 10-tert-Butyl-N-cyano-7-thia-2,5-diazatricyclo[6.4.0.0^{2,6}]dodeca-1(8),3,5-triene-4-carboxamide

To a solution of 10-tert-butyl-7-thia-2,5-diazatricyclo[6.4.0.0^{2,6}]dodeca-1(8),3,5-triene-4-carboxylic acid (90 mg, 0.323 mmol) and DCM (3 mL) was added oxalyl chloride (123 mg, 0.970 mmol) and DMF (1-2 drops) and the mixture stirred at room temperature for 30 min. The solvents were removed and the residue azeotroped twice with DCM. The residue was dissolved in DCM (3 mL) and cyanamide (14 mg, 0.323 mmol) and triethylamine (65 mg, 0.647 mmol) were added. The reaction was stirred for 40 min then it was diluted with DCM (40 mL) and washed with water (20 mL) and brine (20 mL). The organic phase was dried (Na₂SO₄), the mixture filtered and the filtrate concentrated. The crude product was purified by automated reverse phase HPLC (low pH method) to afford the title compound as a white solid (25 mg, 26% yield); m/z=303.0 (MH)⁺; ¹H NMR (500 MHz, DMSO-d6) δ 0.94 (s, 9H), 1.44 (qd, 1H), 1.61 (td, 1H), 2.10 (dd, 1H), 2.41-2.49 (m, 1H), 2.55-2.67 (m, 1H), 2.75 (dd, 1H), 2.88 (dd, 1H), 8.52 (s, 1H). Exchangeables not observed.

### Example 59

### Ethyl 9,10-dichloro-7-thia-2,5-diazatricyclo[6.4.0.0^{2,6}]dodeca-1(8),3,5,9,11-pentaene-4-carboxylate

To a stirred solution of 6,7-dichloro-1,3-benzothiazol-2-amine (1.75 g, 7.99 mmol) in DME (40 mL) was added ethyl 3-bromo-2-oxopropanoate (2.51 mL, 16.0 mmol) dropwise. The reaction was heated at 85° C. overnight then allowed to cool to room temperature. The reaction was diluted with ice/water (50 mL) and basified with NH₃ (aq). The resulting suspension was sonicated and the crude product was collected by filtration. The crude product was triturated in MeOH twice and filtered to afford the title compound as a beige solid (1.30 g, 52% yield); ¹H NMR (500 MHz, Chloroform-d) δ 1.44 (t, 3H), 4.44 (q, 2H), 7.53 (d, 1H), 7.59 (d, 1H), 8.32 (s, 1H).

### Example 60

### Ethyl 9-chloro-10-fluoro-7-thia-2,5-diazatricyclo[6.4.0.0^{2,6}]dodeca-1(8),3,5,9,11-pentaene-4-carboxylate

Ethyl 3-bromo-2-oxopropanoate (150 μL, 1.18 mmol) was added to a solution of 7-chloro-6-fluoro-1,3-benzothiazol-2-amine (120 mg, 0.59 mmol) in DME (3 mL) in a pressure tube. The reaction was sealed and stirred at 75° C. for 18 h. The reaction was allowed to cool to room temperature and re-treated with ethyl 3-bromo-2-oxopropanoate (50 μL, 0.39 mmol) then stirred at 80° C. for a further 6 h. The reaction was allowed to cool to room temperature then filtered over glass fibre filter paper and the filtrate was concentrated in vacuo. The residue was purified by FCC over silica (eluent: heptane:EtOAc 1:0 to 6:4). Product-containing fractions were further purified by silica FCC (eluent: DCM:MeOH 1:0 to 99:1). The residue obtained was triturated with DCM (3×2 mL) to afford the title compound as a white solid (58 mg, 33% yield); ¹H NMR (250 MHz, Chloroform-d) δ 1.38 (t, 3H), 4.37 (q, 2H), 7.18-7.34 (m, 1H), 7.51 (dd, 1H), 8.26 (s, 1H).

### Example 61

### Ethyl 10-chloro-9-fluoro-7-thia-2,5-diazatricyclo[6.4.0.0^{2,6}]dodeca-1(8),3,5,9,11-pentaene-4-carboxylate

6-Chloro-7-fluoro-1,3-benzothiazol-2-amine (90%, 430 mg, 1.91 mmol), ethyl 3-bromo-2-oxopropanoate (603 μL, 4.77 mmol) and DME (6 mL) were charged in a pressure tube. The reaction mixture was sealed and stirred at 75° C. for 2 h. The reaction mixture was allowed to cool to room temperature then diluted with MeCN (5 mL). The reaction was sealed and stirred at 75° C. for 18 h. The reaction was allowed to cool to room temperature then filtered over glass fibre filter paper and the filter pad was washed with MeCN (3×3 mL). The combined filtrates were concentrated and the residue was purified by FCC over silica (eluent: heptane:EtOAc constant gradient 1:0 to 2:7) to afford a solid which was suspended in hot isopropanol (4 mL), the solvent was allowed to cool to room temperature and the suspension was filtered. The filter pad was washed with isopropanol (2 mL) and the solid obtained was dried in vacuo to afford the title compound (97 mg, 18% yield) as a white solid; ¹H NMR (500 MHz, Chloroform-d) δ 1.46 (t, 3H), 4.47 (q, 2H), 7.47 (dd, 1H), 7.53-7.64 (m, 1H), 8.36 (s, 1H).

### Example 62

### Ethyl 10-(trifluoromethoxy)-7-thia-2,5-diazatricyclo[6.4.0.0^{2,6}]dodeca-1(8),3,5,9,11-pentaene-4-carboxylate

The procedure to prepare ethyl 9,10-dichloro-7-thia-2,5-diazatricyclo[6.4.0.0^{2,6}]dodeca-1(8),3,5,9,11-pentaene-4-carboxylate was used except that 6-(trifluoromethoxy)-1,3-benzothiazol-2-amine was substituted for 6,7-dichloro-1,3-benzothiazol-2-amine (48% yield); ¹H NMR (500 MHz, Chloroform-d) δ 1.46 (t, 3H), 4.46 (q, 2H), 7.40 (dd, 1H), 7.61-7.66 (m, 1H), 7.71 (d, 1H), 8.38 (s, 1H).

### Example 63

### Ethyl 9-chloro-7-thia-2,5-diazatricyclo[6.4.0.0^{2,6}]dodeca-1(8),3,5,9,11-pentaene-4-carboxylate

To a solution of 7-chloro-1,3-benzothiazol-2-amine (300 mg, 1.62 mmol) in DME (10 mL) was added ethyl 3-bromo-2-oxopropanoate (950 mg, 4.87 mmol) and the solution heated at 85° C. for 12 h, and then cooled to room temperature. Water was added to the reaction mixture and the aqueous phase extracted with EtOAc (3×30 mL). The combined organic extracts were dried on Na₂SO₄, the mixture filtered and the filtrate evaporated to dryness. The crude residue was purified by FCC (eluent: 10% EtOAc in hexanes) to afford the title compound as a brown solid (190 mg, 35% yield); ¹H NMR (400 MHz, Chloroform-d) δ 1.45 (t, 3H), 4.45 (q, 2H), 7.42-7.49 (m, 2H), 7.59 (dd, 1H), 8.34 (s, 1H).

### Example 64

### Ethyl 10,11-dichloro-7-thia-2,5-diazatricyclo[6.4.0.0^{2,6}]dodeca-1(8),3,5,9,11-pentaene-4-carboxylate

To a solution of 5,6-dichloro-1,3-benzothiazol-2-amine (1.18 g, 50%, containing 50% of the 6,7 dichloro isomer, 1.35 mmol) in DME (23 mL) was added ethyl 3-bromo-2-oxopropanoate (1.31 g, 80%, 5.39 mmol) and the solution heated at 85° C. overnight. The reaction was allowed to cool to room temperature and diluted with ice/water and basified with NH₃(aq). The resulting suspension was sonicated and the crude product was collected by filtration. The crude product was dried in the vacuum oven at room temperature giving a reddish brown solid (2.5 g). This was sonicated in 2:1 DMSO/MeOH (120 mL) and filtered. The solution was purified by automated reverse phase HPLC (neutral method) to afford the title compound as a white solid (110 mg, 13% yield); ¹H NMR (500 MHz, Chloroform-d) δ 1.44 (t, 3H), 4.44 (q, 2H), 7.79 (s, 1H), 7.83 (s, 1H), 8.31 (s, 1H).

### Example 65

### Ethyl 10-chloro-7-thia-2,5-diazatricyclo[6.4.0.0² 6]dodeca-1(8),3,5,9,11-pentaene-4-carboxylate

To a solution of 6-chloro-1,3-benzothiazol-2-amine (500 mg, 2.71 mmol) in acetonitrile (10 mL) was added ethyl 3-bromo-2-oxopropanoate (1.58 g, 8.10 mmol) and the solution heated at 85° C. for 12 h, and then cooled to room temperature. Water was added to the reaction mixture and the aqueous phase extracted with EtOAc (3×30 mL). The combined organic extracts were dried over Na₂SO₄, the mixture filtered and the filtrate evaporated. The residue was purified by FCC on silica (eluent: 2% MeOH in DCM) to afford the title compound as an off white solid (310 mg, 41% yield); ¹H NMR (400 MHz, Chloroform-d) δ 1.44 (t, 3H), 4.44 (q, 2H), 7.47 (dd, 1H), 7.60 (d, 1H), 7.73 (d, 1H), 8.34 (s, 1H).

### Example 66

### Ethyl 10,11-dimethyl-7-thia-2,5-diazatricyclo[6.4.0.0^{2,6}]dodeca-1(8),3,5,9,11-pentaene-4-carboxylate

To a stirred solution of 5,6-dimethyl-1,3-benzothiazol-2-amine (1.00 g, 5.61 mmol) in DME (13 mL) was added ethyl 3-bromo-2-oxopropanoate (1.93 g, 8.41 mmol) and the solution heated at 85° C. overnight. The reaction was poured onto ice/water (10 mL) and basified with NH₃ (aq). The resulting precipitate was collected by filtration and purified by FCC (eluent: 30% EtOAc in heptane), affording the title compound as a white solid (112 mg, 7% yield); ¹H NMR (500 MHz, Chloroform-d) δ 1.45 (t, 3H), 2.40 (s, 3H), 2.43 (s, 3H), 4.45 (q, 2H), 7.46 (s, 1H), 7.48 (s, 1H), 8.32 (s, 1H).

### Example 67

### Ethyl 10-fluoro-7-thia-2,5-diazatricyclo[6.4.0.0^{2,6}]dodeca-1(8),3,5,9,11-pentaene-4-carboxylate

The procedure to prepare ethyl 10-chloro-7-thia-2,5-diazatricyclo[6.4.0.0^{2,6}]dodeca-1(8),3,5,9,11-pentaene-4-carboxylate was used except that 6-fluoro-1,3-benzothiazol-2-amine was substituted for 6-chloro-1,3-benzothiazol-2-amine (48% yield); m/z=265.3 (MH)⁺.

### Example 68

### Ethyl 10-methyl-7-thia-2,5-diazatricyclo[6.4.0.0^{2,6}]dodeca-1(8),3,5,9,11-pentaene-4-carboxylate

The procedure to prepare ethyl 10-chloro-7-thia-2,5-diazatricyclo[6.4.0.0^{2,6}]dodeca-1(8),3,5,9,11-pentaene-4-carboxylate was used except that 6-methyl-1,3-benzothiazol-2-amine was substituted for 6-chloro-1,3-benzothiazol-2-amine (42% yield); m/z=261.3 (MH)⁺.

### Example 69

### Ethyl 11-chloro-7-thia-2,5-diazatricyclo[6.4.0.0² 6]dodeca-1(8),3,5,9,11-pentaene-4-carboxylate

The procedure to prepare ethyl 10-chloro-7-thia-2,5-diazatricyclo[6.4.0.0^{2,6}]dodeca-1(8),3,5,9,11-pentaene-4-carboxylate was used except that 5-chloro-1,3-benzothiazol-2-amine was substituted for 6-chloro-1,3-benzothiazol-2-amine (11% yield); m/z=281.1 (MH)⁺.

### Example 70

### Ethyl 10-bromo-7-thia-2,5-diazatricyclo[6.4.0.0^{2,6}]dodeca-1(8),3,5,9,11-pentaene-4-carboxylate

The procedure to prepare ethyl 9,10-dichloro-7-thia-2,5-diazatricyclo[6.4.0.0^{2,6}]dodeca-1(8),3,5,9,11-pentaene-4-carboxylate was used except that 6-bromo-1,3-benzothiazol-2-amine was substituted for 6,7-dichloro-1,3-benzothiazol-2-amine (36% yield); ¹H NMR (500 MHz, DMSO-d6) δ 1.14 (t, 3H), 4.30 (q, 2H), 7.77 (dd, 1H), 8.13 (d, 1H), 8.36 (d, 1H), 9.05 (s, 1H).

### Example 71

### Ethyl 10-trifluoromethoxy-7-thia-2,5-diazatricyclo[6.4.0.0^{2,6}]dodeca-1(8),3,5,9,11-pentaene-4-carboxylate

The procedure to prepare ethyl 9,10-dichloro-7-thia-2,5-diazatricyclo[6.4.0.0^{2,6}]dodeca-1(8),3,5,9,11-pentaene-4-carboxylate was used except that 6-(trifluoromethyl)-1,3-benzothiazol-2-amine was substituted for 6,7-dichloro-1,3-benzothiazol-2-amine (37% yield); ¹H NMR (500 MHz, Chloroform-d) δ 1.47 (t, 3H), 4.47 (q, 2H), 7.80 (m, 2H), 8.05 (s, 1H), 8.44 (s, 1H).

### Example 72

### Ethyl 10-methoxy-7-thia-2,5-diazatricyclo[6.4.0.0^{2,6}]dodeca-1(8),3,5,9,11-pentaene-4-carboxylate

The procedure to prepare ethyl 9,10-dichloro-7-thia-2,5-diazatricyclo[6.4.0.0^{2,6}]dodeca-1(8),3,5,9,11-pentaene-4-carboxylate was used except that 6-methoxy-1,3-benzothiazol-2-amine was substituted for 6,7-dichloro-1,3-benzothiazol-2-amine (23% yield); ¹H NMR (500 MHz, Chloroform-d) δ 1.45 (t, 3H), 3.90 (s, 3H), 4.44 (q, 2H), 7.04 (dd, 1H), 7.23 (d, 1H), 7.57 (d, 1H), 8.30 (s, 1H).

### Example 73

### Ethyl 3-bromo-10-(trifluoromethoxy)-7-thia-2,5-diazatricyclo[6.4.0.0^{2,6}]dodeca-1(8),3,5,9,11-pentaene-4-carboxylate

To a stirred solution of 10-(trifluoromethoxy)-7-thia-2,5-diazatricyclo[6.4.0.0^{2,6}]dodeca-1(8),3,5,9,11-pentaene-4-carboxylate (100 mg, 0.303 mmol) in EtOH (1 mL) at 0° C. was added NBS (59 mg, 0.333 mmol). The reaction mixture was stirred at this temperature for 30 min and then allowed to warm to room temperature overnight. A precipitate formed which was collected by filtration, dissolved in DCM (20 mL) and washed with saturated NaHCO₃(aq) (15 mL), and brine (15 mL), dried (Na₂SO₄), filtered and concentrated. The residue was purified by FCC (eluent: 20% EtOAc in heptane) to afford the title compound as a white solid (80 mg, 65% yield); m/z=408.8 (MH)⁺.

### Example 74

### Ethyl 3-(tert-butylamino)-10-(trifluoromethoxy)-7-thia-2,5-diazatricyclo[6.4.0.0^{2,6}]dodeca-1(8),3,5,9,11-pentaene-4-carboxylate

A mixture of 6-(trifluoromethoxy)-1,3-benzothiazol-2-amine (120 mg, 0.512 mmol) and ethyl oxoacetate (104 mg, 50% in toluene, 0.512 mmol) in acetonitrile (7 mL) was heated at reflux for 2 h whereupon a solution formed. The reaction mixture was cooled down to room temperature, evaporated to dryness and the solid residue was suspended in toluene and evaporated to dryness to ensure complete removal of water. The residue was suspended in acetonitrile (5 mL) and treated with a solution of trimethylchlorosilane (56 mg, 0.512 mmol) in a minimum volume (0.5-1.0 mL) of DCM. The mixture was stirred at room temperature for 30 min and then treated with a solution of tert-butyl isocyanide (43 mg, 0.512 mmol). The reaction mixture was heated at 70° C. overnight and cooled to room temperature. The solvent was evaporated and the residue purified by FCC on silica (eluent: 0-100% EtOAc in heptane) to afford the title compound (90 mg at 58% purity, 26% yield); ¹H NMR (500 MHz, Chloroform-d) δ 1.28 (s, 9H), 1.46 (t, 3H), 4.43 (q, 2H), 7.31 (d, 1H), 7.55 (s, 1H), 8.38 (d, 1H).

### Example 75

### Ethyl 2-{9,10-dichloro-7-thia-2,5-diazatricyclo[6.4.0.0^{2,6}]dodeca-1(8),3,5,9,11-pentaen-4-yl}acetate

To a solution of 6,7-dichloro-1,3-benzothiazol-2-amine (400 mg, 1.83 mmol) in anhydrous DME (25 mL) was added ethyl 4-bromo-3-oxobutanoate (0.250 mL, 1.83 mmol). The resulting mixture was refluxed at 80° C. for 5 h then evaporated to dryness and triturated in DCM (30 mL). The resulting solid was removed by filtration. The filtrate was evaporated to dryness and purified by silica chromatography (eluent: 0-50% EtOAc in heptane) to afford the title compound as a yellow oil (80 mg, 13% yield); ¹H NMR (500 MHz, DMSO-d6) δ 1.20 (t, 3H), 3.74 (s, 2H), 4.11 (q, 2H), 7.85 (d, 1H), 8.08 (d, 1H), 8.22 (s, 1H).

### Example 76

### Ethyl 7-thia-2,5-diazatricyclo[6.4.0.0^{2,6}]dodeca-1(8),3,5-triene-4-carboxylate

To a solution of 4,5,6,7-tetrahydro-1,3-benzothiazol-2-amine (690 mg, 4.47 mmol) in DMF (7 mL) in a sealable tube were added triethylamine (453 mg, 4.47 mmol) and ethyl 3-bromo-2-oxopropanoate (1.54 g, 85% purity, 6.71 mmol). The tube was sealed and the mixture stirred at 140° C. overnight. The cooled reaction mixture was diluted with water (40 mL). The resulting suspension was extracted with EtOAc (40 mL). The organic layer was washed with water (3×30 mL), brine (30 mL) and then dried (Na₂SO₄), filtered and concentrated. Partial purification was achieved using FCC (eluent: 30% EtOAc in heptane). Purification was achieved using automated reverse phase HPLC (neutral method), affording the title compound as a white solid (90 mg, 8% yield); ¹H NMR (500 MHz, Chloroform-d) δ 1.41 (t, 3H), 1.89-2.02 (m, 4H), 2.60-2.75 (m, 4H), 4.40 (q, 2H), 7.91 (s, 1H).

### Example 77

### Ethyl 10,10-dimethyl-7-thia-2,5-diazatricyclo[6.4.0.0^{2,6}]dodeca-1(8),3,5-triene-4-carboxylate

6,6-Dimethyl-4,5,6,7-tetrahydro-1,3-benzothiazol-2-amine (500 mg, 2.74 mmol) was treated with excess HCl in dioxane and stirred at room temperature for 30 min. Diethyl ether was added, a precipitate formed and the solvent was decanted to leave the HCl salt of the amine, which was dried in vacuo. To this salt was added EtOH (25 mL) and NaOMe (440 mg, 8.23 mmol) and the mixture was stirred at room temperature for 30 min. To this was added ethyl 3-bromo-2-oxopropanoate (1.28 g, 6.58 mmol) and the mixture stirred at room temperature for 2 h, then it was refluxed for 16 h. The solvent was removed and the residue purified by FCC (eluent: 100% DCM) to afford the title compound as a yellow oil (100 mg, 10% yield); m/z=279.1 (MH)⁺.

### Example 78

### Ethyl 10-phenyl-7-thia-2,5-diazatricyclo[6.4.0.0^{2,6}]dodeca-1(8),3,5-triene-4-carboxylate

6-Phenyl-4,5,6,7-tetrahydro-1,3-benzothiazol-2-amine (500 mg, 2.17 mmol) was dissolved in NMP (5 mL) followed by addition of ethyl 3-bromo-2-oxopropanoate (1.06 g, 5.43 mmol) and heated to 180° C. in the microwave for 2 h. The reaction mixture was allowed to cool to room temperature and the reaction mixture purified directly by FCC (eluent: DCM:MeOH 98:2), to obtain the title compound as an off white solid (51 mg, 5% yield); m/z=327.4 (MH)⁺.

### Example 79

### Ethyl 10-tert-butyl-7-thia-2,5-diazatricyclo[6.4.0.0^{2,6}]dodeca-1(8),3,5-triene-4-carboxylate

To a heated solution of 6-tert-butyl-4,5,6,7-tetrahydro-1,3-benzothiazol-2-amine (10.0 g, 46.6 mmol) and NaHCO₃ (7.83 g, 93.2 mmol) in NMP (90 mL) at 75° C. was added ethyl 3-bromo-2-oxopropanoate (6.00 mL, 47.5 mmol). The reaction mixture was heated for 40 min. The cooled reaction mixture was diluted with EtOAc (150 mL), brine (100 mL) and water (200 mL). After vigorous shaking the organic layer was separated and washed with water (3×100 mL). The organic layer was then washed with brine, dried with Na₂SO₄ and the mixture filtered. The filtrate was concentrated in vacuo to give the crude product as a brown oil. The crude product was purified twice using FCC on silica (eluent: 20-40% EtOAc in heptane then 0-40% EtOAc in heptane) to afford the title compound as a brown gum (733 mg, 5% yield); ¹H NMR (500 MHz, DMSO-d6) δ 0.94 (s, 9H), 1.29 (t, 3H), 1.36-1.52 (m, 1H), 1.54-1.67 (m, 1H), 2.04-2.17 (m, 1H), 2.42-2.48 (m, 1H), 2.55-2.68 (m, 1H), 2.68-2.78 (m, 1H), 2.82-2.95 (m, 1H), 4.26 (q, 2H), 8.34 (s, 1H).

### Example 80

### Ethyl 4,4,5,5-tetramethyl-7-thia-1,9-diazatricyclo[6.3.0.0^{2,6}]undeca-2(6),8,10-triene-10-carboxylate

The procedure to prepare ethyl 10-tert-butyl-7-thia-2,5-diazatricyclo[6.4.0.0^{2,6}]dodeca-1(8),3,5-triene-4-carboxylate was used except that 5,5,6,6-tetramethyl-4H,5H,6H-cyclopenta[d][1,3]thiazol-2-amine was substituted for 6-tert-butyl-4,5,6,7-tetrahydro-1,3-benzothiazol-2-amine (58% yield); ¹H NMR (500 MHz, Chloroform-d) δ 1.20 (2s, 2×6H), 1.43 (t, 3H), 2.71 (s, 2H), 4.42 (q, 2H), 7.92 (s, 1H).

### Example 81

### Ethyl 7-thia-2,5-diazatricyclo[6.5.0.0^{2,6}]trideca-1(8),3,5-triene-4-carboxylate

To a solution of 4H,5H,6H,7H,8H-cyclohepta[d][1,3]thiazol-2-amine (90%, 600 mg, 3.56 mmol) in DME (12 mL) was added ethyl 3-bromo-2-oxopropanoate (1.2 mL, 9.52 mmol) at room temperature and the reaction heated at 90° C. for 16 h. The solvent was evaporated, water was added to the remaining residue and the pH adjusted to ˜8. The aqueous phase was extracted with EtOAc. The organic layer was dried (Na₂SO₄), filtered and evaporated to dryness. The residue was purified twice; first by FCC (eluent: 1-40% ethyl acetate in n-hexane) and then by preparative TLC (eluent: 3% methanol in DCM) to afford the title compound as a red solid (22 mg, 3% yield); m/z=265.4 (MH)⁺.

### Example 82

### Ethyl 11,11-dimethyl-7-thia-2,5-diazatricyclo[6.4.0.0^{2,6}]dodeca-1(8),3,5-triene-4-carboxylate

Ethyl 3-bromo-2-oxopropanoate (561 μL, 4.44 mmol) was added to a solution of 5,5-dimethyl-4,5,6,7-tetrahydro-1,3-benzothiazol-2-amine (540 mg, 2.96 mmol) in anhydrous NMP (6 mL) in a pressure tube. The reaction was sealed under nitrogen and stirred at 75-80° C. for 60 min. The reaction was allowed to cool to room temperature before being diluted with EtOAc (50 mL). The organics were washed with water (4×10 mL), brine (10 mL), dried over Na₂SO₄, filtered and the filtrate was concentrated in vacuo. The residue was purified by FCC over silica (eluent: heptane:EtOAc 1:0 to 6:4) to afford (180 mg, 19% yield) of the title compound as a brown solid; ¹H NMR (250 MHz, Chloroform-d) δ 1.11 (s, 6H), 1.41 (t, 3H), 1.70 (t, 2H), 2.42 (t, 2H), 2.64-2.79 (m, 2H), 4.40 (q, 2H), 7.89 (s, 1H).

### Example 83

### Ethyl 9-oxo-7-thia-2,5-diazatricyclo[6.4.0.0.0^{2,6}]dodeca-1(8),3,5-triene-4-carboxylate

Ethyl 3-bromo-2-oxopropanoate (3.85 mL, 30.5 mmol) was added dropwise to a solution of 2-amino-4,5,6,7-tetrahydro-1,3-benzothiazol-7-one (4.27 g, 25.4 mmol) in anhydrous DMF (90 mL) and stirred at 75-80° C. for 60 min. The solution was cooled to room temperature and about half of the solvent was removed under reduced pressure. The remaining solution was diluted with EtOAc (100 mL) and washed with water (5×100 mL). The organic layer was concentrated in vacuo to yield a yellow residue which was chromatographed on silica (eluent: 50 to 90% EtOAc in heptane) to afford the title compound as a yellow solid (2.60 g, 74% purity, 29% yield); ¹H NMR (250 MHz, DMSO-d6) δ 1.31 (t, 3H), 2.23 (p, 2H), 2.57-2.70 (m, 2H), 3.12 (t, 2H), 4.30 (q, 2H), 8.69 (s, 1H).

### Example 84

### Ethyl 9-hydroxy-7-thia-2,5-diazatricyclo[6.4.0.0^{2,6}]dodeca-1(8),3,5-triene-4-carboxylate

To a stirring solution of ethyl 9-oxo-7-thia-2,5-diazatricyclo[6.4.0.0.0^{2,6}]dodeca-1(8),3,5-triene-4-carboxylate (74%, 256 mg, 0.72 mmol) in MeOH (10 mL) at 0° C. under nitrogen was added NaBH₄ (20 mg, 0.54 mmol) and stirred for 1 h. More NaBH₄ (7 mg, 0.18 mmol) was added and the mixture stirred for 20 min whereupon a clear solution formed. The solvent was evaporated, DCM added followed by 0.5M HCl. The phases were separated and the organic phase was washed with brine (20 mL), dried (Na₂SO₄), the mixture filtered and the filtrate concentrated in vacuo to afford the title compound as yellow solid (174 mg, 65% purity, 59% yield); m/z=267.0 (MH)⁺.

### Example 85

### Ethyl 10-tert-butyl-3-(tert-butylamino)-7-thia-2,5-diazatricyclo[6.4.0.0^{2,6}]dodeca-1(8),3,5-triene-4-carboxylate

To a solution of 6-tert-butyl-4,5,6,7-tetrahydro-1,3-benzothiazol-2-amine (500 mg, 2.38 mmol) in toluene (10 mL) was added ethyl oxoacetate (50% solution in toluene, 485 mg, 2.38 mmol) and the mixture stirred at room temperature for 2 h with solid sodium sulfate (1.70 g, 11.9 mol). The reaction mixture was filtered and the solvent evaporated. The residue was suspended in acetonitrile (10 mL) and treated with a solution of TMSCl (258 mg, 2.38 mmol). The mixture was stirred at ambient temperature for 30 min and then treated with tert-butyl isocyanide (198 mg, 2.38 mmol). The reaction mixture was stirred at 70° C. overnight and cooled to room temperature, and then the solvent was evaporated. The residue was purified by FCC on silica (eluent: 0-10% dichloromethane in MeOH) to afford the title compound as a brown solid (190 mg, 75% purity, 16% yield); m/z=378.0 (MH)⁺.

### Example 86

### Ethyl 16-thia-11,14-diazatetracyclo[8.6.0.0^{2,7}.0^{11,15}]hexadeca-1(10),2(7),3,5,12,14-hexaene-13-carboxylate

Ethyl 3-bromo-2-oxopropanoate (760 μL, 5.44 mmol) was added to a stirred solution of 4H,5H-naphtho[2,1-d][1,3]thiazol-2-amine (1.00 g, 4.94 mmol) in DME (38 mL) and heated at 80° C. for 45 min. The reaction was allowed to cool to room temperature, diluted with ice/water (30 mL) and then neutralised with concentrated NH₃ (aq). The mixture was evaporated to dryness then the residue was diluted with EtOAc (75 mL) and water (75 mL). The layers were separated and the aqueous was extracted with EtOAc (3×75 mL). The combined organic extracts were dried (Na₂SO₄), filtered and the filtrate concentrated to give the crude product. The crude product was sonicated in EtOAc and the resulting suspension was filtered. The filtrate was concentrated, sonicated in DCM and the mixture filtered. The filtrate was concentrated to give the title compound as an orange solid (1.0 g, 53% purity, 36% yield);

A portion of the material was purified by automated reverse phase HPLC (low pH method) to provide an analytical sample of the title compound; ¹H NMR (250 MHz, DMSO-d6) δ 1.30 (t, 3H), 3.13 (s, 4H), 4.28 (q, 2H), 7.14-7.41 (m, 4H), 8.57 (s, 1H).

### Example 87

### Ethyl 3-(benzylamino)-10-tert-butyl-7-thia-2,5-diazatricyclo[6.4.0.0^{2,6}]dodeca-1(8),3,5-triene-4-carboxylate

The procedure to prepare ethyl 10-tert-butyl-3-(tert-butylamino)-7-thia-2,5-diazatricyclo[6.4.0.0^{2,6}]dodeca-1(8),3,5-triene-4-carboxylate was used except that (isocyanomethyl)benzene was substituted for tert-butyl isocyanide (33% yield); m/z=412.2 (MH)⁺.

### Example 88

### Ethyl 9,9-dimethyl-7-thia-2,5-diazatricyclo[6.4.0.0^{2,6}]dodeca-1(8),3,5-triene-4-carboxylate

The procedure to prepare ethyl 10-tert-butyl-7-thia-2,5-diazatricyclo[6.4.0.0^{2,6}]dodeca-1(8),3,5-triene-4-carboxylate was used except that 7,7-dimethyl-4,5,6,7-tetrahydro-1,3-benzothiazol-2-amine was substituted for 6-tert-butyl-4,5,6,7-tetrahydro-1,3-benzothiazol-2-amine (24% yield); m/z=279.3 (MH)⁺.

### Example 89

### Ethyl 9-methyl-7-thia-2,5-diazatricyclo[6.4.0.0^{2,6}]dodeca-1(8),3,5-triene-4-carboxylate

The procedure to prepare ethyl 10-tert-butyl-7-thia-2,5-diazatricyclo[6.4.0.0^{2,6}]dodeca-1(8),3,5-triene-4-carboxylate was used except that 7-methyl-4,5,6,7-tetrahydro-1,3-benzothiazol-2-amine was substituted for 6-tert-butyl-4,5,6,7-tetrahydro-1,3-benzothiazol-2-amine (388 mg, 20% yield); m/z=265.0 (MH)⁺.

### Example 90

### 10-tert-Butyl-N-(3-chloropropanesulfonyl)-7-thia-2,5-diazatricyclo[6.4.0.0^{2,6}]dodeca-1(8),3,5-triene-4-carboxamide

To a solution of 10-tert-butyl-7-thia-2,5-diazatricyclo[6.4.0.0^{2,6}]dodeca-1(8),3,5-triene-4-carboxylic acid (222 mg, 0.80 mmol) in anhydrous DMF (20 mL) was added EDC (382 mg, 1.99 mmol) followed by DMAP (292 mg, 2.39 mmol) under an inert atmosphere. The reaction was stirred at ambient temperature for 10 min and then 3-chloropropane-1-sulfonamide (188 mg, 1.20 mmol) was added. The resulting solution was then stirred at 40° C. for 3 h under an inert atmosphere. The mixture was evaporated to dryness, dissolved in DCM (25 mL), washed with water (3×25 mL), 1M HCl(aq) (25 mL) and brine (25 mL). The organic phase was dried over MgSO₄ and evaporated to dryness to afford a yellow solid. This was partially purified by silica chromatography (eluent: 0-5% MeOH in DCM) providing the title compound as a yellow solid (211 mg, 50% purity, 32% yield); m/z=418.0 (MH)⁺.

### Example 91

### 9,10-Dichloro-N-(3-chloropropanesulfonyl)-7-thia-2,5-diazatricyclo[6.4.0.0^{2,6}]dodeca-1(8),3,5,9,11-pentaene-4-carboxamide

The procedure to prepare 10-tert-butyl-N-(3-chloropropanesulfonyl)-7-thia-2,5-diazatricyclo[6.4.0.0^{2,6}]dodeca-1(8),3,5-triene-4-carboxamide was used except that 9,10-dichloro-7-thia-2,5-diazatricyclo[6.4.0.0² 6]dodeca-1(12),3,5,8,10-pentaene-4-carboxylic acid was substituted for 10-tert-butyl-7-thia-2,5-diazatricyclo[6.4.0.0^{2,6}]dodeca-1(8),3,5-triene-4-carboxylic acid. Purification was by methanol trituration rather than FCC (45% yield); ¹H NMR (500 MHz, DMSO-d6) δ 2.11-2.21 (m, 2H), 3.60-3.68 (m, 2H), 3.78 (t, 2H), 7.93 (d, 1H), 8.20 (d, 1H), 9.16 (s, 1H), 11.92 (s, 1H).

### Example 92

### 9,9,11,11-Tetramethyl-7-thia-2,5-diazatricyclo[6.4.0.0^{2,6}]dodeca-1(8),3,5-triene-4-carbaldehyde

A stirred solution of ethyl 9,9,11,11-tetramethyl-7-thia-2,5-diazatricyclo[6.4.0.0^{2,6}dodeca-1(8),3,5-triene-4-carboxylate (90% purity, 555 mg, 1.63 mmol) in toluene (10 mL) was cooled to −78° C. under nitrogen. DIBAL in toluene (2.7 mL, 1.2M, 3.2 mmol) was added dropwise over 30 min. The reaction mixture was then stirred at −78° C. for 30 min and then quenched at −78° C. by the dropwise addition of 50% EtOH in water (1 mL), followed by 2M NaOH_{(aq)} (1 mL) and water (1 mL). The reaction mixture was allowed to warm to room temperature over 30 min, diluted with 2M NaOH_{(aq)} and extracted with toluene. The organic extracts were combined and washed with brine, dried (MgSO₄), filtered and the filtrate evaporated to dryness. The residue was purified using FCC on silica (eluent: 40-60% EtOAc in heptane) to afford the title compound as a yellow oil (251 mg, 55% yield); m/z=263.4 (MH)⁺.

### Example 93

### Methyl 2-hydroxy-2-{9,9,11,11-tetramethyl-7-thia-2,5-diazatricyclo[6.4.0.0^{2,6}]dodeca-1(8),3,5-trien-4-yl}acetate

A solution of tert-butyl isocyanide (123 μL, 1.09 mmol) in DCM (1 mL) was added to a solution of 9,9,11,11-tetramethyl-7-thia-2,5-diazatricyclo[6.4.0.0^{2,6}]dodeca-1(8),3,5-triene-4-carbaldehyde (251 mg, 0.91 mmol), pyridine 1-oxide (1 drop) and tetrachlorosilane (114 μL, 1 mmol) in DCM (1 mL) at −78° C. over 30 min. After being stirred for 3 h, anhydrous MeOH (2.5 mL) was added dropwise over 30 min at −78° C. The reaction was stirred for a further 15 min at −78° C. The mixture was then transferred dropwise to an ice cold solution of NaHCO_{3(aq)}. The reaction mixture was allowed to warm to room temperature and was stirred overnight. The mixture was extracted with DCM (×4). The combined organic extracts were washed with brine and dried (MgSO₄). Filtration and evaporation of the filtrate afforded the crude title compound as a yellow oil (232 mg, 50% purity, 40% yield); m/z=323.0 (MH)⁺.

### Example 94

### Ethyl 9,9,11,11-tetramethyl-10-oxa-7-thia-2,5-diazatricyclo[6.4.0.0^{2,6}]dodeca-1(8),3,5-triene-4-carboxylate

Ethyl 3-bromo-2-oxopropanoate (57 μL, 0.45 mmol) was added to a solution of 4,4,6,6-tetramethyl-4H,6H,7H-pyrano[4,3-d][1,3]thiazol-2-amine (48 mg, 0.26 mmol) in NMP (0.38 mL) under nitrogen. The reaction was stirred at 75° C. for 1 h then allowed to cool to room temperature before being diluted with EtOAc and saturated aqueous sodium hydrogen carbonate solution was added. The aqueous layer was removed and the organic layer was washed extensively with brine, dried (MgSO₄), filtered and concentrated. The crude product was purified by FCC, eluent: 0 to 100% EtOAc in heptane, to afford the title compound as a yellow oil (19 mg, 27% yield); m/z=309.3 (MH)⁺.

### Example 95

### Ethyl 2-{9,10-dichloro-7-thia-2,5-diazatricyclo[6.4.0.0^{2,6}]dodeca-1(8),3,5,9,11-pentaen-4-yl}-2-hydroxyacetate

To a stirred solution of ethyl 2-{9,10-dichloro-7-thia-2,5-diazatricyclo[6.4.0.0^{2,6}]dodeca-1(8),3,5,9,11-pentaen-4-yl}acetate (270 mg, 0.82 mmol) in THF (30 mL) at −78° C. was added 2M LDA in THF/hexane (533 μL, 1.07 mmol) dropwise. The reaction was stirred at −78° C. for 1 h before it was added, via syringe, to a solution of iodine (312 mg, 1.23 mmol) in THF (20 mL) at −78° C. The reaction was stirred at −78° C. for 1 h before quenching the reaction with saturated NH₄Cl_{(aq)} (20 mL). The mixture was allowed to warm to room temperature and was diluted with DCM (100 mL). The iodine was decolourised with saturated Na₂S₂O_{3(aq)} and the pH was adjusted to pH 1 with 1M HCl. The phases were separated and the organic layer was washed with water (50 mL), dried (Na₂SO₄), filtered and concentrated. Purification by FCC on silica (eluent: 0-4% MeOH in DCM) afforded the title compound as a yellow oil (52 mg, 18% yield); m/z=344.8 (MH)⁺.

### Example 96

### Ethyl 3-bromo-10-tert-butyl-7-thia-2,5-diazatricyclo[6.4.0.0^{2,6}]dodeca-1(8),3,5-triene-4-carboxylate

To a solution of ethyl 10-tert-butyl-7-thia-2,5-diazatricyclo[6.4.0.0^{2,6}]dodeca-1(8),3,5-triene-4-carboxylate (462 mg, 1.51 mmol) in EtOH (10 mL) at 0° C. was added a suspension of NBS (295 mg, 1.66 mmol) in EtOH (5 mL) dropwise. The reaction mixture was allowed to warm to room temperature over 1 h. After this time the reaction mixture was concentrated and the residual material was partitioned between EtOAc and saturated aqueous sodium hydrogen carbonate solution. The aqueous layer was removed and the organic layer was washed with saturated brine, dried (MgSO₄), filtered and concentrated. The residue was purified by FCC on silica (eluent: 0-20% EtOAc in heptane) to afford the title compound as a yellow solid (561 mg, 97% yield) m/z=384.9 (MH)⁺.

### Example 97

### Ethyl 10-tert-butyl-3-(4-methoxyphenyl)-7-thia-2,5-diazatricyclo[6.4.0.0^{2,6}]dodeca-1(8),3,5-triene-4-carboxylate

In a microwave vial a mixture of ethyl 3-bromo-10-tert-butyl-7-thia-2,5-diazatricyclo[6.4.0.0^{2,6}]dodeca-1(8),3,5-triene-4-carboxylate (30 mg, 0.078 mmol), Pd(PPh₃)₄(9 mg, 0.008 mmol), (4-methoxyphenyl)boronic acid (14 mg, 0.093 mmol) in 1,4-dioxane (0.4 mL) and 2M Na₂CO₃ (0.155 mL, 0.31 mmol) was degassed for 5 min before the vial was capped and heated at 110° C. for 30 min. After this time the reaction mixture was diluted with EtOAc and water, and passed through a pad of Celite. The phases were separated. The organic layer was washed with saturated brine, dried (MgSO₄), filtered and concentrated. The residual material was purified by FCC on silica, (eluent: 0-20% EtOAc in heptane) to afford the title compound as a pale yellow solid (28 mg, 87% yield); m/z=313.4 (MH)⁺.

### Example 98

### Ethyl 10,10-dimethyl-7-thia-2,5-diazatetracyclo[6.4.0.0^{2,6}.0^{9,11}]dodeca-1(8),3,5-triene-4-carboxylate

To a solution of 3,3-dimethyl-9-thia-7-azatricyclo[4.3.0.0^{2,4}]nona-1(6),7-dien-8-amine (80% purity, 300 mg, 1.33 mmol) in NMP (10 mL) was added NaHCO₃ (246 mg, 2.93 mmol). The resulting solution was heated to 90° C. and then ethyl 3-bromo-2-oxopropanoate (337 μL, 2.66 mmol) was added dropwise. The resulting solution was stirred at 90° C. for a further 30 min. The reaction mixture was cooled to room temperature and then partitioned between EtOAc (150 mL) and water (150 mL). The phases were separated and the organic phase was washed further with water (3×100 mL), brine (2×100 mL) and then dried (Na₂SO₄), filtered and concentrated to afford the title compound as a brown oil which was used in the next step without further purification (745 mg, estimated 50% purity); m/z=276.9 (MH)⁺.

### Example 99

### Ethyl 3-amino-10-tert-butyl-7-thia-2,5-diazatricyclo[6.4.0.0^{2,6}]dodeca-1(8),3,5-triene-4-carboxylate

5M HCl (20 mL) was added to ethyl 3-(benzylamino)-10-tert-butyl-7-thia-2,5-diazatricyclo[6.4.0.0^{2,6}]dodeca-1(8),3,5-triene-4-carboxylate (200 mg, 0.49 mmol) and refluxed for 3 h. The cooled reaction mixture was washed with EtOAc (20 mL) and the aqueous phase concentrated to yield an orange solid which was purified by FCC on silica (eluent: 0 to 100% EtOAc in heptane then 10% MeOH in DCM) to afford the title compound as a brown solid (140 mg, 72% purity); m/z=322.4 (MH)⁺.

### Example 100

### Ethyl 10-tert-butyl-3-acetamido-7-thia-2,5-diazatricyclo[6.4.0.0^{2,6}]dodeca-1(8),3,5-triene-4-carboxylate

Acetic anhydride (1 mL) was added to ethyl 3-amino-10-tert-butyl-7-thia-2,5-diazatricyclo[6.4.0.0^{2,6}]dodeca-1(8),3,5-triene-4-]carboxylate (72% purity, 55 mg, 0.12 mmol). The reaction was stirred at room temperature for 18 h and then heated at 40° C. for 16 h. The reaction mixture was concentrated to yield the title compound as a brown solid which was used in the next step without further purification (40 mg, 18% purity, 16% yield); m/z=364.0 (MH)⁺.

### Example 101

### Ethyl 10-tert-butyl-3-methyl-7-thia-2,5-diazatricyclo[6.4.0.0^{2,6}]dodeca-1(8),3,5-triene-4-carboxylate

Ethyl 3-bromo-2-oxobutanoate (198 mg, 0.951 mmol) was added to a solution of 6-tert-butyl-4,5,6,7-tetrahydro-1,3-benzothiazol-2-amine (100 mg, 0.48 mmol) in NMP (0.8 mL). The reaction was sealed under nitrogen and stirred at 75° C. for 1 h. The reaction was allowed to cool to room temperature before being diluted with EtOAc and saturated aqueous sodium hydrogen carbonate solution. The aqueous layer was removed and the organic layer was washed extensively with saturated brine (×4), dried (MgSO₄), filtered and concentrated. Purification of the residual material by FCC on silica (eluent: 0-100% EtOAc in heptane) afforded the title compound as a red oil (39 mg, 26% yield); m/z=321.4 (MH)⁺.

### Example 102

### Ethyl 10-tert-butyl-3-methanesulfonamido-7-thia-2,5-diazatricyclo[6.4.0.0^{2,6}]dodeca-1(8),3,5-triene-4-carboxylate

A pressure tube was charged with ethyl 3-bromo-10-tert-butyl-7-thia-2,5-diazatricyclo[6.4.0.026]dodeca-1(8),3,5-triene-4-carboxylate (100 mg, 0.260 mmol), methanesulfonamide (30 mg, 0.31 mmol), Pd₂(dba)₃ (24 mg, 0.026 mmol), Xantphos (30 mg, 0.052 mmol) and caesium carbonate (118 mg, 0.363 mmol) and 1,4-dioxane (0.5 mL). The reaction was degassed with nitrogen for 10 min. The tube was sealed and stirred at 95° C. for 18 h. The mixture was cooled and re-treated with methanesulfonamide (30 mg, 0.31 mmol), Pd₂(dba)₃ (24 mg, 0.026 mmol), Xantphos (30 mg, 0.052 mmol) and caesium carbonate (118 mg, 0.363 mmol) and 1,4-dioxane (0.4 mL). The reaction was degassed, sealed and stirred at 95° C. for a further 24 h. The mixture was allowed to cool down and then diluted with EtOAc (15 mL), washed with water (10 mL) and brine (10 mL). The organic layer was dried over magnesium sulfate, filtered and concentrated. The crude product was purified using FCC on silica (eluent: 25-30% EtOAc in heptane) to yield the title compound as an orange solid (30 mg, 27% yield); m/z=400.4 (MH)⁺.

### Example 103

### Ethyl 9,12-dichloro-7-thia-2,5-diazatricyclo[6.4.0.0^{2,6}]dodeca-1(8),3,5,9,11-pentaene-4-carboxylate

To a solution of 4,7-dichloro-1,3-benzothiazol-2-amine (800 mg, 3.65 mmol) in DME (2 mL) was added dropwise ethyl 3-bromo-2-oxopropanoate (0.632 mL, 4.02 mmol). The reaction mixture stirred at 85° C. for 5 h then at room temperature overnight. Additional ethyl 3-bromo-2-oxopropanoate (1.90 mL, 12.1 mmol) was added and the reaction mixture heated at 85° C. for 5 h. The reaction mixture was cooled to room temperature, diluted with ice/water (30 mL) and basified with concentrated ammonia (5 mL). The resulting solid was collected by filtration, dried in air and purified by FCC on silica (eluent: 0-100% EtOAc in heptane) to afford the title compound as a brown solid (200 mg, 58% purity, 10% yield); m/z=314.9 (MH)⁺.

### Example 104

### [Ethyl 9-(trifluoromethoxy)-7-thia-2,5-diazatricyclo[6.4.0.0^{2,6}]dodeca-1(8),3,5,9,11-pentaene-4-carboxylate

The procedure to prepare ethyl 9,12-dichloro-7-thia-2,5-diazatricyclo[6.4.0.0^{2,6}]dodeca-1(8),3,5,9,11-pentaene-4-carboxylate was used except that 7-(trifluoromethoxy)-1,3-benzothiazol-2-amine was substituted for 4,7-dichloro-1,3-benzothiazol-2-amine (47% yield); m/z=331.3 (MH)⁺.

### Example 105

### Ethyl 3-iodo-9,9,11,11-tetramethyl-7-thia-2,5-diazatricyclo[6.4.0.0^{2,6}]dodeca-1(8),3,5-triene-4-carboxylate

To a solution of ethyl 9,9,11,11-tetramethyl-7-thia-2,5-diazatricyclo[6.4.0.0^{2,6}]dodeca-1(8),3,5-triene-4-carboxylate (310 mg, 0.951 mmol) in EtOH (5 mL) was added dropwise a suspension of N-iodosuccinimide (257 mg, 1.14 mmol) in EtOH (2 mL) and the reaction mixture stirred at room temperature for 1 h. Additional N-iodosuccinimide (257 mg, 1.14 mmol) was added and the reaction mixture stirred for 18 h and concentrated. The residual material was then partitioned between EtOAc (20 mL) and saturated aqueous sodium hydrogen carbonate solution (20 mL). The layers were separated. The organic layer was washed with saturated aqueous sodium hydrogen carbonate solution (20 mL) and brine (2×20 mL), dried (MgSO₄), filtered and concentrated to afford the title compound as a brown oil (370 mg, 84% yield); m/z=432.9 (MH)⁺.

### Example 106

### 10-tert-Butyl-7-thia-2,5-diazatricyclo[6.4.0.0^{2,6}]dodeca-1(8),3,5-triene-4-carboxamide

A solution of 10-tert-butyl-7-thia-2,5-diazatricyclo[6.4.0.0^{2,6}]dodeca-1(8),3,5-triene-4-carboxylic acid (172 mg, 0.530 mmol) and thionyl chloride (225 μL, 2.63 mmol) in toluene (5 mL) was heated at reflux for 2 h. After this time the reaction mixture was allowed to cool to room temperature and concentrated, azeotroping (×2) with toluene. The residual material was dissolved in DCM and ammonium hydroxide (2.1 mL, 21 mmol) was added. The mixture stirred at room temperature for 72 h. The reaction mixture was concentrated and partitioned between EtOAc and saturated sodium hydrogen carbonate solution. The insoluble material was collected by filtration. The organic layer was separated, washed with saturated brine, dried (MgSO₄), filtered and concentrated to afford the title compound as a pale tan solid. This material was combined with the insoluble material to afford the title compound (142 mg, 98% yield); m/z=278.1 (MH)⁺.

### Example 107

### 10-tert-Butyl-7-thia-2,5-diazatricyclo[6.4.0.0^{2,6}]dodeca-1(8),3,5-triene-4-carbonitrile

To a solution of 10-tert-butyl-7-thia-2,5-diazatricyclo[6.4.0.0^{2,6}]dodeca-1(8),3,5-triene-4-carboxamide (142 mg, 0.43 mmol) and pyridine (104 μL, 1.29 mmol) in THF (1.4 mL) at 0° C. was added trifluoroacetic anhydride (92 μL, 0.64 mmol). The resulting solution was allowed to warm to room temperature over 16 h. After this time the reaction mixture was partitioned between saturated aqueous sodium hydrogen carbonate solution and EtOAc. The organic layer was washed with saturated brine, dried (MgSO₄), filtered and concentrated. The residual material was dissolved in DCM, adsorbed onto silica gel and purified by FCC on silica, eluent: 0-100% EtOAc in heptane, to afford the title compound as a yellow solid (45 mg, 40% yield); m/z=260.1 (MH)⁺.

### Example 108

### Ethyl 10-[(trifluoromethyl)sulfanyl]-7-thia-2,5-diazatricyclo[6.4.0.0^{2,6}]dodeca-1(8),3,5,9,11-pentaene-4-carboxylate

Ethyl 3-bromo-2-oxopropanoate (252 μL, 2.00 mmol) was added to a solution of 6-[(trifluoromethyl)sulfanyl]-1,3-benzothiazol-2-amine (250 mg, 1.00 mmol) in NMP (1.7 mL). The reaction was sealed under nitrogen and stirred at 75° C. for 1 h and allowed to cool to room temperature over 60 h. The reaction mixture was diluted with EtOAc and saturated aqueous sodium hydrogen carbonate solution. The aqueous layer was removed and the organic layer was washed extensively with saturated brine (×4), dried (MgSO₄), filtered and concentrated. Purification of the residual material by FCC on silica (eluent: 0-100% EtOAc in heptane) afforded the title compound as a yellow solid (131 mg, 38% yield); m/z=347.0 (MH)⁺.

### Example 109

### Ethyl 10-(methylsulfanyl)-7-thia-2,5-diazatricyclo[6.4.0.0^{2,6}]dodeca-1(8),3,5,9,11-pentaene-4-carboxylate

The procedure to prepare ethyl 10-[(trifluoromethyl)sulfanyl]-7-thia-2,5-diazatricyclo[6.4.0.0^{2,6}]dodeca-1(8),3,5,9,11-pentaene-4-carboxylate was used except that 6-methylsulfanyl-1,3-benzothiazol-2-amine was substituted for 6-[(trifluoromethyl)sulfanyl]-1,3-benzothiazol-2-amine (29% yield); m/z=293.0 (MH)⁺.

### Example 110

### Ethyl 9,11-dichloro-7-thia-2,5-diazatricyclo[6.4.0.0^{2,6}]dodeca-1(8),3,5,9,11-pentaene-4-carboxylate

The procedure to prepare ethyl 9,12-dichloro-7-thia-2,5-diazatricyclo[6.4.0.0^{2,6}]dodeca-1(8),3,5,9,11-pentaene-4-carboxylate was used except that 5,7-dichloro-1,3-benzothiazol-2-amine was substituted for 4,7-dichloro-1,3-benzothiazol-2-amine (24% yield); m/z=314.9 (MH)⁺.

### Example 111

### Ethyl 10-tert-butyl-7-thia-2,5-diazatricyclo[6.4.0.0^{2,6}]dodeca-1(8),3,5,9,11-pentaene-4-carboxylate

The procedure to prepare ethyl 10-[(trifluoromethyl)sulfanyl]-7-thia-2,5-diazatricyclo[6.4.0.0^{2,6}]dodeca-1(8),3,5,9,11-pentaene-4-carboxylate was used except that 6-tert-butyl-1,3-benzothiazol-2-amine was substituted for 6-[(trifluoromethyl)sulfanyl]-1,3-benzothiazol-2-amine (21% yield); m/z=303.1 (MH)⁺.

### Example 112

### Ethyl 3-chloro-9,9,11,11-tetramethyl-7-thia-2,5-diazatricyclo[6.4.0.0^{2,6}]dodeca-1(8),3,5-triene-4-carboxylate

To a solution of ethyl 9,9,11,11-tetramethyl-7-thia-2,5-diazatricyclo[6.4.0.0^{2,6}]dodeca-1(8),3,5-triene-4-carboxylate (78 mg, 0.26 mmol) in EtOH (3 mL) at 0° C. was added N-chlorosuccinimide (34 mg, 0.26 mmol). The reaction mixture was stirred at room temperature for 21 h. The reaction mixture was re-cooled to 0° C. and additional N-chlorosuccinimide (34 mg, 0.26 mmol) was added. Stirring was continued at room temperature for 60 h. The reaction mixture was concentrated and the residue thus obtained partitioned between EtOAc (30 mL) and saturated aqueous sodium hydrogen carbonate solution (30 mL). The aqueous layer was removed and the organic layer was washed with saturated brine (30 mL), dried (MgSO₄), filtered and concentrated to yield a brown residue which was purified by FCC on silica, eluent: 0 to 100% EtOAc in heptane, to afford the title compound as a white solid (25 mg, 25% yield); m/z=341.4 (MH)⁺.

### Example 113

### Ethyl 10-hydroxy-7-thia-2,5-diazatricyclo[6.4.0.0^{2,6}]dodeca-1(8),3,5,9,11-pentaene-4-carboxylate

To a solution of 2-amino-1,3-benzothiazol-6-ol (1.10 g, 6.62 mmol) in DMA (25 mL) was added ethyl 3-bromo-2-oxopropanoate (1.25 mL, 9.93 mmol). The resulting solution was stirred at 100° C. for 3 h under an inert atmosphere. The cooled mixture was poured into ice water (˜150 mL) and the resulting mixture filtered and the solid washed with more water. This was triturated in hot MeOH and filtered again. The collected solid was dried under vacuum to afford the title compound as a brown solid (600 mg, 35% yield); m/z=263.0 (MH)⁺.

### Example 114

### Ethyl 10-(benzyloxy)-7-thia-2,5-diazatricyclo[6.4.0.0^{2,6}]dodeca-1(8),3,5,9,11-pentaene-4-carboxylate

To a solution of ethyl 10-hydroxy-7-thia-2,5-diazatricyclo[6.4.0.0^{2,6}]dodeca-1(8),3,5,9,11-pentaene-4-carboxylate (150 mg, 0.57 mmol) in DMA (5 mL) was added (bromomethyl)benzene (102 μL, 0.860 mmol) followed by K₂CO₃ (158 mg, 1.14 mmol).

The solution was stirred at 75° C. for 2 h and then poured into water (20 mL). The resulting mixture extracted with EtOAc (3×50 mL). The combined organic extracts were washed with brine (2×50 mL), dried over MgSO₄ and evaporated to dryness. The resulting crude oil was purified by FCC on silica (eluent: 5-75% EtOAc in heptane) to afford the title compound as a yellow solid (137 mg, 68% yield); m/z=353.0 (MH)⁺.

### Example 115

### Ethyl 9,10-difluoro-7-thia-2,5-diazatricyclo[6.4.0.0^{2,6}]dodeca-1(8),3,5,9,11-pentaene-4-carboxylate

The procedure to prepare ethyl 9,12-dichloro-7-thia-2,5-diazatricyclo[6.4.0.0^{2,6}]dodeca-1(8),3,5,9,11-pentaene-4-carboxylate was used except that 6,7-difluoro-1,3-benzothiazol-2-amine was substituted for 4,7-dichloro-1,3-benzothiazol-2-amine (92% yield); m/z=282.9 (MH)⁺.

### Example 116

### Ethyl 10-(cyclopentyloxy)-7-thia-2,5-diazatricyclo[6.4.0.0^{2,6}]dodeca-1(8),3,5,9,11-pentaene-4-carboxylate

The procedure to prepare ethyl 10-(benzyloxy)-7-thia-2,5-diazatricyclo[6.4.0.0^{2,6}]dodeca-1(8),3,5,9,11-pentaene-4-carboxylate was used except that iodocyclopentane was substituted for (bromomethyl)benzene (58% yield); m/z=331.4 (MH)⁺.

### Example 117

### Ethyl 9-bromo-7-thia-2,5-diazatricyclo[6.4.0.0^{2,6}]dodeca-1(8),3,5,9,11-pentaene-4-carboxylate

To a solution of 7-bromo-1,3-benzothiazol-2-amine (1.00 g, 4.36 mmol) in DME (20 mL) was added ethyl 3-bromo-2-oxopropanoate (1.70 g, 8.73 mmol). The resulting mixture was stirred at 80° C. under nitrogen for 2 h then poured into water (100 mL) and neutralised with aqueous ammonia to ˜pH 7. The resulting mixture was filtered and the solid dried under vacuum. The collected solid was then triturated in hot MeOH and filtered to afford the title compound as a beige solid (254 mg, 18% yield); m/z=324.9 (MH)⁺.

### Example 118

### Ethyl 11-chloro-10-(trifluoromethoxy)-7-thia-2,5-diazatricyclo[6.4.0.0^{2,6}]dodeca-1(8),3,5,9,11-pentaene-4-carboxylate

To a stirred solution of a mixture of 5-chloro-6-(trifluoromethoxy)-1,3-benzothiazol-2-amine and 7-chloro-6-(trifluoromethoxy)-1,3-benzothiazol-2-amine (1:1, 2.50 g, 4.65 mmol) in DME (75 mL) was added ethyl 3-bromo-2-oxopropanoate (2.93 mL, 18.6 mmol) dropwise. The reaction was heated at 85° C. for 20 h. The reaction was allowed to cool to room temperature, diluted with ice/water (30 vol) and neutralised with concentrated NH_{3(aq)}. The resulting precipitate was collected by filtration then purified by automated reverse phase preparative HPLC (low pH method) to afford the title compound as a yellow solid (80 mg, 4%); ¹H NMR (250 MHz, DMSO-d6) δ 1.32 (t, 3H), 4.31 (q, 2H), 8.48 (d, 1H), 8.66 (s, 1H), 9.04 (s, 1H).

### Example 119

### Ethyl 9-chloro-10-(trifluoromethoxy)-7-thia-2,5-diazatricyclo[6.4.0.0^{2,6}]dodeca-1(8),3,5,9,11-pentaene-4-carboxylate

The procedure to prepare ethyl 11-chloro-10-(trifluoromethoxy)-7-thia-2,5-diazatricyclo[6.4.0.0^{2,6}]dodeca-1(8),3,5,9,11-pentaene-4-carboxylate was used, with the title compound also eluting from the preparative HPLC column (120 mg, 6% yield); ¹H NMR (250 MHz, DMSO-d6) δ 1.32 (t, 3H), 4.31 (q, 2H), 7.89 (dd, 1H), 8.31 (d, 1H), 9.14 (s, 1H).

### Example 120

### Ethyl 12-methyl-7-thia-2,5-diazatricyclo[6.4.0.0^{2,6}]dodeca-1(8),3,5,9,11-pentaene-4-carboxylate

The procedure to prepare ethyl 10-hydroxy-7-thia-2,5-diazatricyclo[6.4.0.0^{2,6}]dodeca-1(8),3,5,9,11-pentaene-4-carboxylate was used except that 4-methyl-1,3-benzothiazol-2-amine was substituted for 2-amino-1,3-benzothiazol-6-ol (7% yield); m/z=261.0 (MH)⁺.

### Example 121

### Ethyl 12-methoxy-7-thia-2,5-diazatricyclo[6.4.0.0^{2,6}]dodeca-1(8),3,5,9,11-pentaene-4-carboxylate

The procedure to prepare ethyl 10-hydroxy-7-thia-2,5-diazatricyclo[6.4.0.0^{2,6}]dodeca-1(8),3,5,9,11-pentaene-4-carboxylate was used except that 4-methoxy-1,3-benzothiazol-2-amine was substituted for 2-amino-1,3-benzothiazol-6-ol and purification was by FCC (eluent 0-60% EtOAc in heptane) rather than by trituration (6% yield); m/z=276.3 (MH)⁺.

### Example 122

### Ethyl 10-chloro-9-methoxy-7-thia-2,5-diazatricyclo[6.4.0.0^{2,6}]dodeca-1(8),3,5,9,11-pentaene-4-carboxylate

To a solution of 6-chloro-7-methoxy-1,3-benzothiazol-2-amine (300 mg, 1.40 mmol) in DME (10 mL) was added dropwise ethyl 3-bromo-2-oxopropanoate (0.88 mL, 5.59 mmol) and the reaction mixture stirred at 75° C. After 18 h the reaction mixture was cooled to room temperature and then ice/water (50 mL) was added. The suspension was neutralised using ammonium hydroxide (˜5 mL) and filtered. The solid was washed with water (10 mL), then MeOH. The washings were concentrated and triturated in MeOH, yielding a second crop of solid which was isolated by filtration. The solids were combined, affording the title compound as a tan solid (70 mg, 16% yield); m/z=310.9 (MH)⁺.

### Example 123

### Ethyl 9-fluoro-7-thia-2,5-diazatricyclo[6.4.0.0^{2,6}]dodeca-1(8),3,5,9,11-pentaene-4-carboxylate

The procedure to prepare ethyl 10-hydroxy-7-thia-2,5-diazatricyclo[6.4.0.0^{2,6}]dodeca-1(8),3,5,9,11-pentaene-4-carboxylate was used except that 4-fluoro-1,3-benzothiazol-2-amine was substituted for 2-amino-1,3-benzothiazol-6-ol (26% yield); m/z=264.9 (MH)⁺.

### Example 124

### Ethyl 3-methanesulfonamido-9,9,11,11-tetramethyl-7-thia-2,5-diazatricyclo[6.4.0.0^{2,6}]dodeca1(8),3,5-triene-4-carboxylate

A pressure tube was charged with ethyl 3-iodo-9,9,11,11-tetramethyl-7-thia-2,5-diazatricyclo[6.4.0.0^{2,6}]dodeca-1(8),3,5-triene-4-carboxylate (50 mg, 0.12 mmol), methanesulfonamide (22 mg, 0.23 mmol), Pd₂(dba)₃ (11 mg, 0.012 mmol), Xantphos (7 mg, 0.012 mmol) and caesium carbonate (53 mg, 0.16 mmol) and 1,4-dioxane (0.6 mL). The suspension was degassed with nitrogen for 10 min. The tube was sealed and the reaction was heated at 95° C. for 18 h. The reaction mixture was cooled and re-treated with identical amounts of methanesulfonamide, Pd₂(dba)₃, Xantphos and caesium carbonate. The reaction was degassed and heated at 95° C. for a further 24 h. The reaction mixture was allowed to cool and was then filtered through Celite™ washing with MeOH (50 mL). The combined filtrates were then concentrated. The residue was taken up in EtOAc (15 mL), washed with water (10 mL) and brine (10 mL), dried over MgSO₄, filtered and concentrated. The crude product was purified using FCC on silica (eluent: 25-30% EtOAc in heptane) to yield the title compound as a yellow solid (46 mg, 65% yield); m/z=400.1 (MH)⁺.

### Example 125

### Ethyl 9-(pyrrolidin-1-yl)-7-thia-2,5-diazatricyclo[6.4.0.0^{2,6}]dodeca-1(8),3,5,9,11-pentaene-4-carboxylate

Ethyl 9-bromo-7-thia-2,5-diazatricyclo[6.4.0.0^{2,6}]dodeca-1(8),3,5,9,11-pentaene-4-carboxylate (100 mg, 0.308 mmol), pyrrolidine (51 μL, 0.62 mmol), Pd₂(dba)₃ (28 mg, 0.031 mmol), Xantphos (18 mg, 0.031 mmol), caesium carbonate (140 mg, 0.431 mmol) and 1,4-dioxane (2 mL) were added to a pressure tube. The resulting suspension was degassed with nitrogen before being sealed and heated to 95° C. for 16 h. The solvent was removed under reduced pressure and the residue was partitioned between EtOAc (10 mL) and 0.5M HCl_{(aq)} (10 mL). The organic layer was reserved and the aqueous layer extracted with EtOAc (15 mL). The organic fractions were combined, washed with water (2×15 mL) and brine (20 mL) and dried over magnesium sulfate. The solvent was removed under reduced pressure and the residue purified by automated reverse-phase HPLC (low pH method) to afford the title compound as a yellow solid (41 mg, 71% purity, 30% yield); m/z=316.0 (MH)⁺.

### Example 126

### Ethyl 2-{9-chloro-10-fluoro-7-thia-2,5-diazatricyclo[6.4.0.0^{2,6}]dodeca-1(8),3,5,9,11-pentaen-4-yl}acetate

To a stirred solution of 7-chloro-6-fluoro-1,3-benzothiazol-2-amine (300 mg, 1.48 mmol) in 1,2-dimethoxyethane (30 mL) at 100° C. was added ethyl 4-bromo-3-oxobutanoate (464 mg, 2.21 mmol and the solution stirred for 16 h. Additional ethyl 4-bromo-3-oxobutanoate (200 mg, 0.952 mmol) was added and heating continued for 1 h. The solvent was evaporated and the residue chromatographed on silica (15-80% EtOAc in heptane, starting material and product co-eluted). More starting material (143 mg) eluted after a column strip with 0-10% MeOH in DCM. The recovered 7-chloro-6-fluoro-1,3-benzothiazol-2-amine (143 mg, 0.704 mmol) was resubmitted to the above cyclisation conditions using more ethyl 4-bromo-3-oxobutanoate (200 mg, 0.952 mmol) and heated for 2 h. After evaporation, the combined residues were purified by automated reverse phase HPLC (low pH method) to afford the title compound as a yellow solid (107 mg, 22% yield); m/z=312.9 (MH)⁺.

### Example 127

### Ethyl 3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylate

Aqueous hydrogen peroxide (28%, 72 μL, 0.58 mmol) was added dropwise to a stirred solution of ethyl 9,9,11,11-tetramethyl-7-thia-2,5-diazatricyclo[6.4.0.0^{2,6}]dodeca-1(8),3,5-triene-4-carboxylate (200 mg, 0.653 mmol), concentrated sulfuric acid (35 μL, 0.65 mmol), tert-butyl 3-iodoazetidine-1-carboxylate (370 mg, 1.31 mmol) and iron(II) sulfate heptahydrate (136 mg, 0.490 mmol) in DMSO (7 mL) at room temperature. After 30 min, additional iron(II) sulfate heptahydrate (136 mg, 0.490 mmol) and aqueous hydrogen peroxide (28%, 72 μL, 0.58 mmol) were added, and the mixture was stirred at room temperature for 30 min. After 30 min, iron(II) sulfate heptahydrate (136 mg, 0.490 mmol) and aqueous hydrogen peroxide (28%, 72 μL, 0.58 mmol) were added, and the mixture was stirred at room temperature for 2 h. The reaction mixture was quenched with saturated NaHCO₃ and was diluted with EtOAc (100 mL). The phases were separated and the organic layer was washed with water (5×35 mL) and then brine (10 mL), dried over Na₂SO₄, filtered and concentrated. The residue was purified using FCC on silica (eluent: 0-100% EtOAc in heptane) to afford the title compound as an off white solid (165 mg, 88% purity, 48% yield); m/z=462.1 (MH)⁺.

### Example 128

### Ethyl 9-chloro-10-fluoro-3-(trifluoromethyl)-7-thia-2,5-diazatricyclo[6.4.0.0^{2,6}]dodeca-1(8),3,5,9,11-pentaene-4-carboxylate

To a stirred solution of ethyl 9-chloro-10-fluoro-7-thia-2,5-diazatricyclo[6.4.0.0^{2,6}dodeca-1(8),3,5,9,11-pentaene-4-carboxylate (50 mg, 0.17 mmol) in DMSO (2 mL) and water (0.5 mL) was added sodium trifluoromethanesulfinate (157 mg, 1.00 mmol). The reaction was cooled to 0° C. and tert-butyl hydroperoxide (229 μL, 1.67 mmol) was added dropwise. The reaction was stirred at room temperature for 66 h, then more sodium trifluoromethanesulfinate (157 mg, 1.00 mmol) was added. The reaction was cooled to 0° C. and more tert-butyl hydroperoxide (229 μL, 1.67 mmol) was added dropwise. The reaction was stirred at room temperature for 48 h then the reaction mixture was recharged with the previously described amounts of sodium trifluoromethanesulfinate and tert-butyl hydroperoxide, followed by stirring for 48 h. A final recharge with the previously described amounts of sodium trifluoromethanesulfinate and tert-butyl hydroperoxide was performed and the mixture stirred for another 48 h. The reaction was then diluted with EtOAc (20 mL) and washed with water (4×15 mL). The organic phase was dried (Na₂SO₄), filtered and concentrated. The residue was purified by FCC (eluent: 0-30% EtOAc in heptane) to afford the title compound as a white solid (10 mg, 16% yield); m/z=366.9 (MH)⁺.

### Example 129

### Ethyl 10-tert-butyl-7-thia-2,5-diazatricyclo[6.4.0.0^{2,6}]dodeca-1(8),5,9,11-tetraene-4-carboxylate

To a solution of 6-tert-butyl-1,3-benzothiazol-2-amine (300 mg, 1.45 mmol) in EtOH (3 mL) was added ethyl 2-bromoprop-2-enoate, (available via a literature procedure: J. Org. Chem., 1999, 64, 7618-7621), (312 mg, 1.74 mmol), triethylamine (405 μL, 2.91 mmol) and hydroquinone (32 mg, 0.29 mmol). The reaction mixture was stirred in a pressure tube at 85° C. for 2 h then the reaction mixture was evaporated to dryness, the residue dissolved in EtOAc (30 mL) and the solution washed with water (25 mL) and brine (25 mL). The organic layer was dried over MgSO₄, filtered and evaporated to dryness. The residue was purified by automated reverse phase HPLC (low pH method) to afford the title compound as a purple oil (236 mg, 70% purity, 37% yield); m/z=305.6 (MH)⁺.

### Example 130

### Ethyl 9-chloro-10-fluoro-7-thia-2,5-diazatricyclo[6.4.0.0^{2,6}]dodeca-1(8),5,9,11-tetraene-4-carboxylate

Benzyl 2-bromoprop-2-enoate (available by a literature procedure: WO2010/127856 A1, 2010) (1.54 g, 5.86 mmol), 7-chloro-6-fluoro-1,3-benzothiazol-2-amine (1.00 g, 4.89 mmol), hydroquinone (108 mg, 0.977 mmol) and EtOH (10 mL) were added to a pressure tube and the resulting suspension heated to 85° C. for 1 h. Triethylamine (1.36 mL, 9.77 mmol) was added and the resulting solution was stirred at 85° C. for 15 h. The solvent was removed under reduced pressure and the resulting oil partitioned between EtOAc (20 mL) and 1M HCl (20 mL). The organic layer was reserved. The aqueous layer was adjusted to ˜pH 7-8 using saturated sodium bicarbonate and extracted with more EtOAc (2×15 mL). The organic fractions were combined, washed with water (2×15 mL) and saturated brine (25 mL) and dried over MgSO₄. The solvent was removed under reduced pressure and the residue purified by FCC on silica, eluent: 0-5% MeOH in DCM. Further purification was achieved using FCC on reverse-phase silica, eluent: 0-100% MeCN in water (+0.1% formic acid in both), to afford the title compound as a pink oil (354 mg, 90% purity, 22% yield); m/z=301.0 (MH)⁺.

### Example 131

### Methyl 9-chloro-10-fluoro-7-thia-2,5-diazatricyclo[6.4.0.0^{2,6}]dodeca-1(8),3,5,9,11-pentaene-4-carboxylate

7-Chloro-6-fluoro-1,3-benzothiazol-2-amine (641 mg, 3.13 mmol), benzyl 2-bromoprop-2-enoate (83%, 1.00 g, 3.44 mmol) and MeOH (8 mL) were added to a pressure tube and the resulting solution heated to 70° C. for 1 h. Triethylamine (0.87 mL, 6.30 mmol) was added and the solution heated to 70° C. for a further 2 h. More benzyl 2-bromoprop-2-enoate (83%, 0.50 g, 1.72 mmol) was added and the solution stirred at 70° C. for 2 h. More benzyl 2-bromoprop-2-enoate (83%, 0.5 g, 1.72 mmol) was added. The solution was stirred at 70° C. for a further 6 h. The reaction mixture was concentrated under reduced pressure and partitioned between EtOAc (20 mL) and 1M HCl (20 mL) and the phases separated. The aqueous layer was adjusted to pH 7 using saturated sodium bicarbonate and filtered, to afford a solid. The solid was purified by automated reverse phase HPLC (low pH method) to afford the title compound as a pink solid (113 mg, 12% yield); m/z=287.1 (MH)⁺.

### Example 132

### Ethyl 3-bromo-9,9,11,11-tetramethyl-7-thia-2,5-diazatricyclo[6.4.0.0^{2,6}]dodeca-1(8),3,5-triene-4-carboxylate

To a solution of ethyl 9,9,11,11-tetramethyl-7-thia-2,5 diazatricyclo[6.4.0.0^{2,6}]dodeca-1(8),3,5-triene-4-carboxylate (511 mg, 1.67 mmol) in EtOH (10 mL) at 0° C. was added a suspension of NBS (327 mg, 1.83 mmol) in EtOH (7 mL) dropwise. The reaction mixture was allowed to warm to room temperature over 1 h. After this time the reaction mixture was concentrated. The residual material was partitioned between EtOAc and saturated aqueous sodium hydrogen carbonate solution. The aqueous layer was removed and the organic layer was washed with saturated brine, dried (MgSO₄), filtered and concentrated. The residue was purified by FCC on silica (eluent: 0-20% EtOAc in heptane) to afford the title compound as a pale yellow solid (360 mg, 56% yield); m/z=386.7 (MH)⁺.

### Example 133

### Ethyl 3-ethynyl-9,9,11,11-tetramethyl-7-thia-2,5-diazatricyclo[6.4.0.0^{2,6}]dodeca-1(8),3,5-triene-4-carboxylate

A vial was charged with ethyl 3-bromo-9,9,11,11-tetramethyl-7-thia-2,5-diazatricyclo[6.4.0.0^{2,6}]dodeca-1(8),3,5-triene-4-carboxylate (30 mg, 0.078 mmol), tributyl(ethynyl)stannane (75 μL, 0.19 mmol), Pd(PPh₃)₄(9 mg, 0.008 mmol) and DMF (0.4 mL). The reaction mixture was degassed for 5 min prior to heating at 80° C. for 4 h. The reaction mixture was allowed to cool, then partitioned between EtOAc and 1M aqueous hydrogen chloride solution and filtered through Celite. The phases were separated and the organic layer washed with saturated brine (×3), dried (MgSO₄), filtered and concentrated. The residue was purified by FCC on silica, eluent: 0-20% EtOAc in heptane, to afford the desired product as a purple oil (17 mg, 86% purity, 57% yield); m/z=331.1 (MH)⁺.

### Example 134

### Ethyl 10-chloro-11-(trifluoromethoxy)-7-thia-2,5-diazatricyclo[6.4.0.0^{2,6}]dodeca-1(8),3,5,9,11-pentaene-4-carboxylate

The procedure to prepare ethyl 11-chloro-10-(trifluoromethoxy)-7-thia-2,5-diazatricyclo[6.4.0.0^{2,6}]dodeca-1(8),3,5,9,11-pentaene-4-carboxylate was used except that 6-chloro-5-(trifluoromethoxy)-1,3-benzothiazol-2-amine was substituted for 5-chloro-6-(trifluoromethoxy)-1,3-benzothiazol-2-amine (94% yield); m/z=365.0 (MH)⁺.

### Example 135

### Ethyl 10-bromo-7-thia-2,5-diazatricyclo[6.4.0.0^{2,6}]dodeca-1(8),5,9,11-tetraene-4-carboxylate

The procedure to prepare ethyl 10-tert-butyl-7-thia-2,5-diazatricyclo[6.4.0.0^{2,6}]dodeca-1(8),5,9,11-tetraene-4-carboxylate was used except that 6-bromo-1,3-benzothiazol-2-amine was substituted for 6-tert-butyl-1,3-benzothiazol-2-amine (55% yield); m/z=327.0 (MH)⁺.

### Example 136

### Ethyl 3-bromo-9-chloro-10-fluoro-7-thia-2,5-diazatricyclo[6.4.0.0^{2,6}]dodeca-1(8),3,5,9,11-pentaene-4-carboxylate

A vial was charged with ethyl 9-chloro-10-fluoro-7-thia-2,5-diazatricyclo[6.4.0.0^{2,6}dodeca-1(8),3,5,9,11-pentaene-4-carboxylate (1.00 g, 3.35 mmol) and EtOH (22 mL). NBS (655 mg, 3.68 mmol) was added and the resulting suspension stirred for 5 h. The mixture was sonicated and the precipitate collected by filtration and rinsed with EtOH. The resultant solid was dissolved in DCM (20 mL) and washed with saturated NaHCO₃(aq) (15 mL), and brine (15 mL), dried (Na₂SO₄), filtered and concentrated. The residue was triturated in MeOH to give the title compound as a white solid (550 mg, 44% yield); m/z=376.9 (MH)⁺.

### Example 137

### Ethyl 9-chloro-10-fluoro-3-methanesulfonamido-7-thia-2,5-diazatricyclo[6.4.0.0^{2,6}]dodeca-1(8),3,5,9,11-pentaene-4-carboxylate

A sealed tube was charged with ethyl 3-bromo-9-chloro-10-fluoro-7-thia-2,5-diazatricyclo[6.4.0.0^{2,6}dodeca-1(8),3,5,9,11-pentaene-4-carboxylate (300 mg, 0.794 mmol), methanesulfonamide (76 mg, 0.79 mmol), Pd₂(dba)₃ (73 mg, 0.079 mmol), Xantphos (35 mg, 0.060 mmol), caesium carbonate (362 mg, 1.11 mmol) and 1,4-dioxane (4.5 mL). The suspension was degassed, sealed and then heated at 95° C. for 18 h. The reaction was re-treated with methanesulfonamide (76 mg, 0.79 mmol), Pd₂(dba)₃ (73 mg, 0.079 mmol), Xantphos (35 mg, 0.060 mmol), caesium carbonate (362 mg, 1.11 mmol) and degassed. The reaction was heated at 95° C. for 24 h. The reaction was retreated with methanesulfonamide (76 mg, 0.79 mmol), Pd₂(dba)₃ (73 mg, 0.079 mmol), Xantphos (35 mg, 0.060 mmol), caesium carbonate (362 mg, 1.11 mmol) and degassed. The reaction was heated at 95° C. for another 24 h. The mixture was allowed to cool, diluted with EtOAc, sonicated and the solution decanted (50 mL). The solution was discarded and the solid remaining in the tube was treated with MeOH and sonicated. The resulting suspension was filtered (×2) to remove palladium residues and the filtrate evaporated to dryness. The residue was purified by automated reverse phase HPLC (low pH method) to afford the title compound as a white solid (46 mg, 15% yield); m/z=392.0 (MH)⁺.

### Example 138

### Ethyl 9,9,11,11-tetramethyl-3-(morpholin-4-yl)-7-thia-2,5-diazatricyclo[6.4.0.0^{2,6}]dodeca-1(8),3,5-triene-4-carboxylate

A vial was charged with ethyl 3-bromo-9,9,11,11-tetramethyl-7-thia-2,5-diazatricyclo[6.4.0.0^{2,6}]dodeca-1(8),3,5-triene-4-carboxylate (50 mg, 0.13 mmol), Pd₂(dba)₃ (7.5 mg, 0.01 mmol), Cs₂CO₃ (59 mg, 0.18 mmol), Xantphos (15 mg, 0.026 mmol) and morpholine (28 μL, 0.32 mmol), then 1,4-dioxane (0.65 mL). The reaction mixture was degassed for 5 min prior to heating at 100° C. for 16 h. The reaction mixture was allowed to cool, partitioned between EtOAc and 1M aqueous HCl and filtered through Celite™. The phases were separated and the organic layer was washed with saturated aqueous brine (×3), dried (MgSO₄), filtered and concentrated. The residue was purified by FCC on silica, eluent: 0-30% EtOAc in heptane, to afford the title compound as a pale yellow oil (30 mg, 58% yield); m/z=392.6 (MH)⁺.

### Example 139

### Ethyl 9-chloro-10-fluoro-3-methyl-7-thia-2,5-diazatricyclo[6.4.0.0^{2,6}]dodeca-1(8),3,5,9,11-pentaene-4-carboxylate

To a stirred solution of 7-chloro-6-fluoro-1,3-benzothiazol-2-amine (400 mg, 1.97 mmol) in MeOH (1 mL) was added ethyl 3-bromo-2-oxobutanoate (339 μL, 2.17 mmol) and the solution was heated at 65° C. for 18 h. The reaction was allowed to cool to room temperature and a solid formed which was collected by filtration. To the solid was added DMSO (1 mL) and MeOH (0.5 mL) and the suspension was sonicated. The solid was collected by filtration affording the title compound as a yellow solid (28 mg, 5% yield); m/z=313.0 (MH)⁺.

### Example 140

### Ethyl 9-chloro-10-fluoro-7-thia-2,5-diazatricyclo[6.4.0.0^{2,6}]dodeca-1(8),5,9,11-tetraene-4-carboxylate

7-Chloro-6-fluoro-1,3-benzothiazol-2-amine (91%, 780 mg, 3.45 mmol), ethyl 2-bromoprop-2-enoate (1.05 g, 4.14 mmol), triethylamine (0.96 mL, 6.9 mmol), hydroquinone (76 mg, 0.69 mmol) and EtOH (8 mL) were added to a pressure tube and the resulting suspension stirred at 85° C. for 4 h. The reaction mixture was concentrated under reduced pressure and partitioned between EtOAc (20 mL) and water (20 mL). The organic layer was taken and the aqueous layer extracted with EtOAc (15 mL). The organic layers were combined, washed with water (15 ml) and saturated brine (15 mL) and dried over MgSO₄. After filtration, the solvent was removed under reduced pressure. The residue was suspended in DCM and filtered. The filtrate was evaporated and purified via acidic reverse-phase FCC, eluent: 15-40% MeCN in water (+0.1% formic acid), to afford the title compound as a pink oil (388 mg, 37% yield); m/z=301.0 (MH)⁺.

### Example 141

### Ethyl (4S)-9-chloro-10-fluoro-7-thia-2,5-diazatricyclo[6.4.0.0^{2,6}]dodeca-1(8),5,9,11-tetraene-4-carboxylate and ethyl (4R)-9-chloro-10-fluoro-7-thia-2,5-diazatricyclo[6.4.0.0^{2,6}]dodeca-1(8),5,9,11-tetraene-4-carboxylate (configurations arbitrarily assigned)

Racemic ethyl 9-chloro-10-fluoro-7-thia-2,5-diazatricyclo[6.4.0.0^{2,6}]dodeca-1(8),5,9,11-tetraene-4-carboxylate (166 mg, 0.55 mmol) was resolved using SFC on a Lux C-4 column to afford the title compounds (48 mg, 29% yield; 59 mg, 36% yield);

### Example 142

### Ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate

A vial was charged with ethyl 3-bromo-9-chloro-10-fluoro-7-thia-2,5-diazatricyclo[6.4.0.0^{2,6}dodeca-1(8),3,5,9,11-pentaene-4-carboxylate (100 mg, 0.265 mmol), Pd₂(dba)₃ (15 mg, 0.026 mmol), Cs₂CO₃ (121 mg, 0.371 mmol), Xantphos (31 mg, 0.053 mmol), morpholine (57 μL, 0.66 mmol) and degassed 1,4-dioxane (1.5 mL). The vial was flushed with nitrogen, sealed and stirred at 100° C. for 18 h. The reaction mixture was allowed to cool and was diluted with EtOAc (10 mL) and water (10 mL). The phases were separated and the aqueous phase was extracted with EtOAc (2×10 mL). The combined organic extracts were washed with brine (15 mL), dried (Na₂SO₄), the mixture filtered and the filtrate concentrated. The crude residue was dissolved in DMSO (1 mL) and MeOH (0.5 mL) and sonicated. Two drops of water were added. The resulting suspension was sonicated and the precipitate was collected by filtration to afford the title compound as an orange solid (34 mg, 46% purity, 15% yield); m/z=384.1 (MH)⁺.

### Example 143

### Ethyl 10-bromo-9-chloro-7-thia-2,5-diazatricyclo[6.4.0.0^{2,6}]dodeca-1(8),5,9,11-tetraene-4-carboxylate

Ethyl 2-bromoprop-2-enoate (85%, 4.79 g, 22.8 mmol), 6-bromo-7-chloro-1,3-benzothiazol-2-amine (5.00 g, 19.0 mmol), triethylamine (5.3 mL, 38 mmol), hydroquinone (209 mg, 1.90 mmol) and EtOH (50 mL) were added to a pressure tube. The reaction mixture was heated to 85° C. for 5 h. The solvent was removed under reduced pressure and the resulting solid stirred with diethyl ether and the solid removed by filtration. The filtrate was concentrated under reduced pressure and purified by FCC using reverse-phase silica, eluent: 0-100% MeCN in water (+0.1% formic acid), to afford the title compound as a pink gum (1.07 g, 15% yield); m/z=360.9 (MH)⁺.

### Example 144

### Ethyl 9,10-dichloro-12-[2-(morpholin-4-yl)ethoxy]-7-thia-2,5-diazatricyclo[6.4.0.0^{2,6}]dodeca-1(8),3,5,9,11-pentaene-4-carboxylate

To a stirred solution of 6,7-dichloro-4-[2-(morpholin-4-yl)ethoxy]-1,3-benzothiazol-2-amine (900 mg, 2.58 mmol) in DMA (22 mL) was added ethyl 3-bromo-2-oxopropanoate (610 μL, 3.88 mmol). The reaction was heated at 85° C. for 3 h then the reaction was concentrated. The residue was dissolved in EtOAc (100 mL) and washed with water (75 mL). The aqueous layer was extracted with EtOAc (3×75 mL). The combined organic extracts were washed with brine (100 mL), dried (Na₂SO₄), filtered and concentrated to afford the title compound as a brown solid (1.20 g, 36% purity, 38% yield); m/z=444.1 (MH)⁺.

### Example 145

### Ethyl 10-tert-butyl-3-(morpholin-4-ylmethyl)-7-thia-2,5-diazatricyclo[6.4.0.0^{2,6}]dodeca-1(8),3,5-triene-4-carboxylate

In a microwave vial was stirred a solution of morpholine (26 mg, 0.29 mmol) and formaldehyde (37% aqueous solution; 24 mg, 0.29 mmol) in AcOH (1 mL) at 4° C. for 1 h. After this time, ethyl 10-tert-butyl-7-thia-2,5-diazatricyclo[6.4.0.0^{2,6}]dodeca-1(8),3,5-triene-4-carboxylate (30 mg, 0.098 mmol) was added and the reaction vial was capped and heated at 120° C. under microwave irradiation for 20 min. The reaction media was basified by addition of 5M aqueous NaOH solution, and diluted with EtOAc. The organic layer was separated, washed with saturated aqueous sodium hydrogen carbonate solution, then brine, dried (MgSO₄), filtered and the filtrate concentrated in vacuo. The residual material was purified by FCC on silica, eluent: 0-40% EtOAc in heptane, to afford the title compound as a colourless solid (27 mg, 66% yield); m/z=406.2 (MH)⁺.

### Example 146

### Ethyl 10-bromo-9-chloro-7-thia-2,5-diazatricyclo[6.4.0.0^{2,6}]dodeca-1(8),3,5,9,11-pentaene-4-carboxylate

The procedure to prepare ethyl 10-hydroxy-7-thia-2,5-diazatricyclo[6.4.0.0^{2,6}]dodeca-1(8),3,5,9,11-pentaene-4-carboxylate was followed except that 6-bromo-7-chloro-1,3-benzothiazol-2-amine was substituted for 2-amino-1,3-benzothiazol-6-ol (59% yield); m/z=360.8 (MH)⁺.

### Example 147

### Ethyl 10-tert-butyl-3-[(2-oxopiperidin-1-yl)methyl]-7-thia-2,5-diazatricyclo[6.4.0.0^{2,6}]dodeca-1(8),3,5-triene-4-carboxylate

In a microwave vial was added 1-(hydroxymethyl)piperidin-2-one (available via a literature procedure: J. Med. Chem., 1995, 38, 4198-4210) (25 mg, 0.20 mmol), ethyl 10-tert-butyl-7-thia-2,5-diazatricyclo[6.4.0.0^{2,6}]dodeca-1(8),3,5-triene-4-carboxylate (30 mg, 0.098 mmol), conc. sulfuric acid (1 drop) and AcOH (1 mL). The mixture was heated at 120° C. under microwave irradiation for 80 min. After this time, the reaction mixture was basified with aqueous 1M NaOH and extracted with EtOAc (×2). The organic layers were combined, washed with brine, dried (MgSO₄), filtered and the filtrate concentrated. The residual material was purified by FCC on silica (eluent: 0-100% EtOAc in heptane) to afford the title compound as a pale yellow oil (33 mg, 75% yield); m/z=418.2 (MH)⁺.

### Example 148

### Ethyl 3,9,9,11,11-pentamethyl-7-thia-2,5-diazatricyclo[6.4.0.0^{2,6}]dodeca-1(8),3,5-triene-4-carboxylate

Ethyl 3-bromo-2-oxobutanoate (198 mg, 0.951 mmol) was added to a solution of 5,5,7,7-tetramethyl-4,5,6,7-tetrahydro-1,3-benzothiazol-2-amine (100 mg, 0.475 mmol) in NMP (0.8 mL). The reaction was sealed under nitrogen and stirred at 75° C. for 1 h. The reaction was allowed to cool to room temperature and was aged for 16 h before being diluted with EtOAc and saturated aqueous sodium hydrogen carbonate solution. The aqueous layer was removed and the organic layer was washed extensively with brine, dried (MgSO₄), filtered and the filtrate concentrated. This residual material was purified by FCC on silica (eluent: 0-100% EtOAc in heptane) to afford the title compound as a dark red oil (84 mg, 66% purity, 36% yield); m/z=321.6 (MH)⁺.

### Example 149

### Ethyl 9,9,11,11-tetramethyl-3-(morpholin-4-ylmethyl)-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that ethyl 9,9,11,11-tetramethyl-7-thia-2,5-diazatricyclo[6.4.0.0^{2,6}]dodeca-1(8),3,5-triene-4-carboxylate was substituted for ethyl 10-tert-butyl-7-thia-2,5-diazatricyclo[6.4.0.0^{2,6}]dodeca-1(8),3,5-triene-4-carboxylate (52% yield); m/z=406.2 (MH)⁺.

### Example 150

### Ethyl 3-(2-methoxyphenyl)-9,9,11,11-tetramethyl-7-thia-2,5-diazatricyclo[6.4.0.0^{2,6}]dodeca-1(8),3,5-triene-4-carboxylate

In a pressure tube was degassed a mixture of ethyl 3-bromo-9,9,11,11-tetramethyl-7-thia-2,5-diazatricyclo[6.4.0.0^{2,6}]dodeca-1(8),3,5-triene-4-carboxylate (100 mg, 0.260 mmol), (2-methoxyphenyl)boronic acid (47 mg, 0.026 mmol), 2M Na₂CO_{3(aq)} (519 μL, 1.04 mmol) and palladium tetrakistriphenylphospine (30 mg, 0.03 mmol) in 1,4-dioxane (1.3 mL) for 10 min. The tube was sealed and heated at 110° C. for 18 h. The reaction mixture was diluted with EtOAc and H₂O, passed through a pad of Celite™. The organic layer was separated, washed with saturated brine, dried (MgSO₄), filtered and the filtrate concentrated. The residual material was purified by FCC on silica (eluent: 0-20% EtOAc in heptane) to afford the title compound as a pale yellow solid (80 mg, 73% yield); m/z=413.7 (MH)⁺.

### Example 151

### Ethyl 9-chloro-10-(pyrrolidin-1-yl)-7-thia-2,5-diazatricyclo[6.4.0.0^{2,6}]dodeca-1(8),3,5,9,11-pentaene-4-carboxylate

The procedure to prepare ethyl 9-(pyrrolidin-1-yl)-7-thia-2,5-diazatricyclo[6.4.0.0^{2,6}]dodeca-1(8),3,5,9,11-pentaene-4-carboxylate was used except that ethyl 10-bromo-9-chloro-7-thia-2,5-diazatricyclo[6.4.0.0^{2,6}]dodeca-1(8),3,5,9,11-pentaene-4-carboxylate was substituted for ethyl 9-bromo-7-thia-2,5-diazatricyclo[6.4.0.0^{2,6}]dodeca-1(8),3,5,9,11-pentaene-4-carboxylate (23% yield); m/z=350.0 (MH)⁺.

### Example 152

### Ethyl 10-tert-butyl-3-{[(2-methoxyethyl)(methyl)amino]methyl}-7-thia-2,5-diazatricyclo[6.4.0.0^{2,6}]dodeca-1(8),3,5-triene-4-carboxylate

A solution of (2-methoxyethyl)dimethylamine (13 mg, 0.15 mmol) and formaldehyde (37% aqueous solution; 12 mg, 0.15 mmol) in AcOH (0.5 mL) was stirred in a microwave vial for 1 h at room temperature. After this time ethyl 10-tert-butyl-7-thia-2,5-diazatricyclo[6.4.0.0^{2,6}]dodeca-1(8),3,5-triene-4-carboxylate (15 mg, 0.049 mmol) was added and the reaction vial was sealed and heated at 120° C. under microwave irradiation for 20 min. The reaction was re-treated twice with the same initial quantities of (2-methoxyethyl)dimethylamine (13 mg, 0.15 mmol) and formaldehyde (12 mg, 0.15 mmol) and heated at 120° C. in the microwave for 20 min after each re-treatment. After this time the reaction mixture was basified with aqueous 1M sodium hydroxide solution and extracted with 20% IPA/DCM (×2). The organic layers were combined, washed with saturated brine, dried (MgSO₄), filtered and concentrated. The residual material was purified by FCC on silica (eluent: 0-100% EtOAc in heptane) to afford the title compound as a pale yellow oil (15 mg, 60% yield); m/z=408.2 (MH)⁺.

### Example 153

### Ethyl 9-chloro-10-cyclopropyl-7-thia-2,5-diazatricyclo[6.4.0.0^{2,6}]dodeca-1(8),3,5,9,11-pentaene-4-carboxylate

To a solution of ethyl 10-bromo-9-chloro-7-thia-2,5-diazatricyclo[6.4.0.0^{2,6}]dodeca-1(8),3,5,9,11-pentaene-4-carboxylate (250 mg, 0.695 mmol), cyclopropylboronic acid (119 mg, 1.39 mmol) and Cs₂CO₃ (340 mg, 1.04 mmol) in 1,4-dioxane (5 mL) was added Pd(PPh₃)₄(0.080 g, 0.070 mmol). The resulting mixture was degassed with nitrogen and then stirred at 100° C. for 16 h. The mixture was then diluted with EtOAc (50 mL) and washed with water (25 mL), 1M HCl (25 mL) and brine (25 mL). The organic layer was dried over MgSO₄, filtered and the filtrate evaporated to dryness. The crude product was purified by automated reverse phase HPLC (low pH method) to afford the title compound as a light yellow solid (79 mg, 35%); m/z=321.1 (MH)⁺.

### Example 154

### Ethyl 3-(4-methoxyphenyl)-9,9,11,11-tetramethyl-7-thia-2,5-diazatricyclo[6.4.0.0^{2,6}]dodeca-1(8),3,5-triene-4-carboxylate

The procedure to prepare ethyl 3-(2-methoxyphenyl)-9,9,11,11-tetramethyl-7-thia-2,5-diazatricyclo[6.4.0.0^{2,6}]dodeca-1(8),3,5-triene-4-carboxylate was used except that (4-methoxyphenyl)boronic acid was substituted for (2-methoxyphenyl)boronic acid (75% yield); m/z=413.2 (MH)⁺.

### Example 155

### Ethyl 10-tert-butyl-3-(acetamidomethyl)-7-thia-2,5-diazatricyclo[6.4.0.0^{2,6}]dodeca-1(8),3,5-triene-4-carboxylate

In a pressure tube was added N-(hydroxymethyl)acetamide (35 mg, 0.39 mmol), ethyl 10-tert-butyl-7-thia-2,5-diazatricyclo[6.4.0.0^{2,6}]dodeca-1(8),3,5-triene-4-carboxylate (30 mg, 0.098 mmol), sulfuric acid (1 drop) and AcOH (1 mL). This mixture was heated at 120° C. under thermal conditions for 16 h. The reaction mixture was allowed to cool to room temperature and more N-(hydroxymethyl)acetamide (35 mg, 0.39 mmol) was added, with heating was continued for 20 h. The reaction mixture was basified with aqueous 1M sodium hydroxide solution and extracted with EtOAc (×2). The combined organic layers were washed with brine, dried (MgSO₄), filtered and the filtrate concentrated. The residue was purified by FCC on silica (eluent: 0-100% EtOAc in heptane) to afford the title compound as a pale yellow oil (13 mg, 32% yield); m/z=378.3 (MH)⁺.

### Example 156

### Ethyl 3-[(acetyloxy)methyl]-10-tert-butyl-7-thia-2,5-diazatricyclo[6.4.0.0^{2,6}]dodeca-1(8),3,5-triene-4-carboxylate

In a microwave vial was stirred a solution of tert-butylmethylamine (51 mg, 0.59 mmol) and formaldehyde (37% aqueous solution; 48 mg, 0.60 mmol) in AcOH (1 mL) for 1 h at room temperature. After this time ethyl 10-tert-butyl-7-thia-2,5-diazatricyclo[6.4.0.0^{2,6}]dodeca-1(8),3,5-triene-4-carboxylate (30 mg, 0.098 mmol) was added and the reaction vial was capped and heated at 120° C. under microwave irradiation for 20 min. More tert-butylmethylamine (51 mg, 0.59 mmol) and formaldehyde (37% aqueous solution; 48 mg, 0.60 mmol) were added, with the reaction vial capped and heated at 120° C. under thermal conditions for 16 h. After this time, the reaction mixture was basified with aqueous 1M sodium hydroxide solution and extracted with EtOAc (×2). The combined organic extracts were washed with brine, dried (MgSO₄), filtered and the filtrate concentrated. The residue was purified by FCC on silica (eluent: 0-100% EtOAc in heptane) to afford the title compound as a pale yellow solid (40 mg, 92% purity, 99% yield); m/z=379.6 (MH)⁺.

### Example 157

### Ethyl 10-tert-butyl-3-{[methoxy(methyl)amino]methyl}-7-thia-2,5-diazatricyclo[6.4.0.0^{2,6}]dodeca-1(8),3,5-triene-4-carboxylate

The procedure to prepare ethyl 3-[(acetyloxy)methyl]-10-tert-butyl-7-thia-2,5-diazatricyclo[6.4.0.0^{2,6}]dodeca-1(8),3,5-triene-4-carboxylate was used except that N,O-dimethyl hydroxylamine hydrochloride was substituted for tert-butylmethylamine and no further heating after microwave irradiation was performed. Extraction was by 20% IPA in DCM rather than EtOAc (63% yield); m/z=380.6 (MH)⁺.

### Example 158

### Ethyl 3-cyclopropyl-9,9,11,11-tetramethyl-7-thia-2,5-diazatricyclo[6.4.0.0^{2,6}]dodeca-1(8),3,5-triene-4-carboxylate

Cyclopropylboronic acid (31 mg, 0.36 mmol) was added to a mixture of ethyl 3-bromo-9,9,11,11-tetramethyl-7-thia-2,5-diazatricyclo[6.4.0.0^{2,6}]dodeca-1(8),3,5-triene-4-carboxylate (100 mg, 0.26 mmol), Pd(OAc)₂ (5.8 mg, 0.026 mmol), tricyclohexylphosphonium tetrafluoroborate (9.6 mg, 0.026 mmol) and tripotassium phosphate (193 mg, 0.908 mmol) in toluene (1.3 mL) and water (0.2 mL). The resulting mixture was heated to 120° C. under microwave conditions for 1 h. The mixture was retreated twice with the same initial amounts of tricyclohexylphosphonium tetrafluoroborate, Pd(OAc)₂ and cyclopropylboronic acid and heated for 1 h at 120° C. under microwave conditions after each retreatment. The mixture was dissolved in EtOAc (25 mL) and washed with water (10 mL) and saturated aqueous NH₄Cl (10 mL). The aqueous washings were back-extracted with EtOAc (20 mL). The combined organic phases were washed with brine (10 mL), dried over Na₂SO₄, filtered and the filtrate concentrated. The residue was purified by FCC on silica (eluent: 20-25% EtOAc in heptane) to afford the title compound as an off white solid (35 mg, 66% purity, 26% yield); m/z=347.2 (MH)⁺.

### Example 159

### tert-Butyl 9-chloro-10-fluoro-7-thia-2,5-diazatricyclo[6.4.0.0^{2,6}]dodeca-1(8),5,9,11-tetraene-4-carboxylate

To a stirred solution of 7-chloro-6-fluoro-1,3-benzothiazol-2-amine (2.07 g, 10.2 mmol) in EtOH (25 mL) was added tert-butyl 2-bromoprop-2-enoate (available via a literature method: Eur. J. Inorg. Chem., 2006, 18, 3622-3626) (2.54 g, 12.3 mmol), triethylamine (2.85 mL, 20.4 mmol) and hydroquinone (113 mg, 1.02 mmol). The reaction mixture was heated to 85° C. for 24 h. The solvent was removed under reduced pressure and the residue was purified by reverse-phase FCC, eluent: 15-40% MeCN in water (+0.1% formic acid), to afford the title compound as a pink gum (713 mg, 85% purity, 18% yield); m/z=329.1 (MH)⁺.

### Example 160

### Ethyl 9-chloro-10-(morpholin-4-yl)-7-thia-2,5-diazatricyclo[6.4.0.0^{2,6}]dodeca-1(8),3,5,9,11-pentaene-4-carboxylate

To a solution of ethyl 10-bromo-9-chloro-7-thia-2,5-diazatricyclo[6.4.0.0^{2,6}]dodeca-1(8),3,5,9,11-pentaene-4-carboxylate (250 mg, 0.695 mmol) in 1,4-dioxane (2 mL) in a pressure tube was added (1E,4E)-1,5-diphenylpenta-1,4-dien-3-one bis((1Z,4E)-1,5-diphenylpenta-1,4-dien-3-one) dipalladium (64 mg, 0.070 mmol), (9,9-dimethyl-9H-xanthene-4,5-diyl)bis(diphenylphosphane) (40 mg, 0.070 mmol) and Cs₂CO₃ (340 mg, 1.04 mmol). The resulting solution was de-gassed with nitrogen for 10 min and then morpholine (120 μL, 1.39 mmol) was added. The mixture was then stirred at 100° C. for 16 h, cooled and poured into EtOAc (25 mL). The organic layer was washed with 1M HCl (25 mL), water (25 mL) and brine (25 mL), dried over MgSO₄, the mixture filtered and the filtrate evaporated to dryness. The residue was purified by FCC on silica (eluent: 0-10% EtOAc in heptane) to afford the title compound as a cream solid (91 mg, 36% yield); m/z=366.1 (MH)⁺.

### Example 161

### Ethyl 9,10-dichloro-11-[2-(morpholin-4-yl)ethoxy]-7-thia-2,5-diazatricyclo[6.4.0.0^{2,6}]dodeca-1(8),3,5,9,11-pentaene-4-carboxylate

To a stirred solution of 6,7-dichloro-5-[2-(morpholin-4-yl)ethoxy]-1,3-benzothiazol-2-amine (1.00 g, 2.87 mmol) in DME (30 mL) was added ethyl 3-bromo-2-oxopropanoate (1.81 mL, 11.5 mmol). The reaction mixture was heated at 85° C. then poured onto ice/water (35 mL) and neutralised with 2M ammonia solution. The resulting precipitate, which contained product, was collected by filtration. The aqueous filtrate was extracted with EtOAc (3×100 mL). The combined organic extracts were dried (Na₂SO₄), filtered and the filtrate concentrated. The resulting residue was combined with the previous precipitate and purified by automated reverse phase HPLC (low pH method). The resulting solid was triturated in MeOH to give the title compound as a cream solid (85 mg, 7% yield); m/z=444.1 (MH)⁺.

### Example 162

### Ethyl 9-chloro-10-(piperidin-1-yl)-7-thia-2,5-diazatricyclo[6.4.0.0^{2,6}]dodeca-1(8),3,5,9,11-pentaene-4-carboxylate

Ethyl 10-bromo-9-chloro-7-thia-2,5-diazatricyclo[6.4.0.0^{2,6}]dodeca-1(8),3,5,9,11-pentaene-4-carboxylate (250 mg, 0.695 mmol), piperidine (137 μL, 1.39 mmol), Pd₂(dba)₃ (64 mg, 0.070 mmol), Xantphos (40 mg, 0.070 mmol), caesium carbonate (317 mg, 0.973 mmol) and 1,4-dioxane (2.5 mL) were added to a pressure tube and the resulting suspension degassed, before being sealed and heated to 95° C. for 16 h. The cooled reaction mixture was diluted with EtOAc (10 mL) and water (10 mL) and filtered. The phases of the filtrate were separated and the organic layer reserved. The aqueous layer was re-extracted with EtOAc (15 mL). The organic fractions were combined, washed with saturated brine (20 mL) and the aqueous layer re-extracted with EtOAc (15 mL). The organic fractions were combined, dried over MgSO₄ and the solvent removed under reduced pressure. The residue was purified by FCC on silica (eluent: 0-60% EtOAc in heptane) to afford the title compound as a yellow solid (160 mg, 90% purity, 57% yield); m/z=364.1 (MH)⁺.

### Example 163

### Ethyl 3-(3,3-difluoroazetidin-1-yl)-9,9,11,11-tetramethyl-7-thia-2,5-diazatricyclo[6.4.0.0^{2,6}]dodeca-1(8),3,5-triene-4-carboxylate

The procedure to prepare ethyl 9,9,11,11-tetramethyl-3-(morpholin-4-yl)-7-thia-2,5-diazatricyclo[6.4.0.0^{2,6}]dodeca-1(8),3,5-triene-4-carboxylate was used except for 3,3-difluoroazetidine hydrochloride was substituted for morpholine (50% yield); m/z=398.5 (MH)⁺.

### Example 164

### Ethyl 3-{1-[(tert-butoxy)carbonyl]-1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylate

In a microwave vial was added ethyl 3-bromo-9,9,11,11-tetramethyl-7-thia-2,5-diazatricyclo[6.4.0.0^{2,6}]dodeca-1(8),3,5-triene-4-carboxylate (120 mg, 0.311 mmol), tert-butyl 4-(4,4,5,5-tetramethyl-1,3,2-dioxaborolan-2-yl)-3,6-dihydropyridine-1(2H)-carboxylate (106 mg, 0.343 mmol), Pd(dppf)Cl₂.DCM (51 mg, 0.062 mmol) and tripotassium phosphate (132 mg, 0.623 mmol) in DMF/water (0.38 mL/20 μL) and the reaction mixture heated at 120° C. for 2 h. It was then cooled, taken up in EtOAc (30 mL) and washed with water (15 mL) and then saturated ammonium chloride solution (15 mL). The aqueous layer was further extracted using EtOAc (20 mL). The organic layers were combined, dried over Na₂SO₄, filtered and the filtrate concentrated. The residue was purified using FCC on silica (eluent: 0 to 100% EtOAc in heptane) to afford the title compound as a yellow solid (110 mg, 69% yield); m/z=388.3 (MH)⁺.

### Example 165

### Ethyl 3-({4-[(tert-butoxy)carbonyl]piperazin-1-yl}methyl)-10-tert-butyl-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that N-Boc piperazine was substituted for morpholine and 1M NaOH_{(aq)} was utilised in place of 5M NaOH solution (49% yield); m/z=505.40 (MH)⁺.

### Example 166

### Ethyl 10-tert-butyl-3-{[4-(2-hydroxyethyl)piperazin-1-yl]methyl}-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that 2-hydroxyethylpiperazine was substituted for morpholine and 1M NaOH_{(aq)} was utilised in place of 5M NaOH_{(aq)} (60% yield); m/z=449.3 (MH)⁺.

### Example 167

### Ethyl 10-tert-butyl-3-{[4-(2-methoxyethyl)piperazin-1-yl]methyl}-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that 2-methoxyethylpiperazine was substituted for morpholine and 1M NaOH_{(aq)} was utilised in place of 5M NaOH_{(aq)} (77% yield); m/z=463.4 (MH)⁺.

### Example 168

### Ethyl 10-tert-butyl-3-[(4-methylpiperazin-1-yl)methyl]-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that N-methyl piperazine was substituted for morpholine and 1M NaOH_{(aq)} was utilised in place of 5M NaOH_{(aq)} (86% yield); m/z=419.3 (MH)⁺.

### Example 169

### Ethyl 3-[(4-acetylpiperazin-1-yl)methyl]-10-tert-butyl-7-thia-2,5-diazatricyclo[6.4.0.0^{2,6}]dodeca-1(8),3,5-triene-4-carboxylate

The procedure to prepare ethyl 10-tert-butyl-3-(morpholin-4-ylmethyl)-7-thia-2,5-diazatricyclo[6.4.0.0^{2,6}]dodeca-1(8),3,5-triene-4-carboxylate was used except that N-acetyl piperazine was substituted for morpholine and 1M NaOH_{(aq)} was utilised in place of 5M NaOH_{(aq)} (85% yield); m/z=447.3 (MH)⁺.

### Example 170

### Ethyl 9-chloro-10-fluoro-3-{[2-(pyrrolidin-1-yl)ethyl]amino}-7-thia-2,5-diazatricyclo[6.4.0.0^{2,6}]dodeca-1(8),3,5,9,11-pentaene-4-carboxylate

The procedure to prepare ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate was used except that 2-(pyrrolidin-1-yl)ethan-1-amine substituted for morpholine. Additionally, the filtrate from the precipitation was purified by automated reverse phase HPLC (low pH method) (17% yield); m/z=411.2 (MH)⁺.

### Example 171

### Ethyl 3-(dimethylamino)-9,9,11,11-tetramethyl-7-thia-2,5-diazatricyclo[6.4.0.0^{2,6}]dodeca-1(8),3,5-triene-4-carboxylate

The procedure to prepare ethyl 9,9,11,11-tetramethyl-3-(morpholin-4-yl)-7-thia-2,5-diazatricyclo[6.4.0.0^{2,6}]dodeca-1(8),3,5-triene-4-carboxylate was used except that dimethylamine hydrochloride (4 eq) was substituted for morpholine and Cs₂CO₃ (5 eq) was used. The title compound was afforded as a pale brown solid (24% yield); m/z=350.5 (MH)⁺.

### Example 172

### Ethyl 3-{1-[(tert-butoxy)carbonyl]azetidin-3-yl}-9-chloro-10-fluoro-7-thia-2,5-diazatricyclo[6.4.0.0^{2,6}]dodeca-1(8),3,5-triene-4-carboxylate

To a stirred suspension of ethyl 9-chloro-10-fluoro-7-thia-2,5-diazatricyclo[6.4.0.0^{2,6}]dodeca-1(8),3,5,9,11-pentaene-4-carboxylate (300 mg, 1.00 mmol) was added 18.7M sulfuric acid (107 μL, 2.00 mmol) and iron(2+) sulfate hydrate (1:1:7) (84 mg, 0.301 mmol). 50% Hydrogen peroxide in water (171 μL, 3.01 mmol) was then added dropwise. After 2 minutes, iron(2+) sulfate hydrate (1:1:7) (84 mg, 0.301 mmol) was added. The reaction was then stirred for 30 min before the dropwise addition of 50% hydrogen peroxide in water (171 μL, 3.01 mmol) followed by the addition of iron(2+) sulfate hydrate (1:1:7) (84 mg, 0.301 mmol). After 15 min, the reaction mixture was filtered. The filtrate was quenched by the addition of 0.2M NaOH_{(aq)} (50 mL) and the resulting precipitate collected by filtration. This material was dissolved in DMSO/MeOH (2:1; 1.5 mL/100 mg), sonicated and filtered. The filtrate was purified by automated reverse phase HPLC (low pH method) to give the title compound as a white solid (32 mg, 4% yield); m/z=454.1 (MH)⁺.

### Example 173

### 3-{1-[(tert-Butoxy)carbonyl]azetidin-3-yl}-9-chloro-10-fluoro-7-thia-2,5-diazatricyclo[6.4.0.0^{2,6}]dodeca-1(8),3,5-triene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 3-{1-[(tert-butoxy)carbonyl]azetidin-3-yl}-9-chloro-10-fluoro-7-thia-2,5-diazatricyclo[6.4.0.0^{2,6}]dodeca-1(8),3,5-triene-4-carboxylate was substituted for ethyl 9,11-dichloro-7-thia-2,5-diazatricyclo[6.4.0.0^{2,6}]dodeca-1(8),3,5,9,11-pentaene-4-carboxylate (71% yield); m/z=369.9 (MH)⁺.

### Example 174

### Ethyl 3-{4-[(tert-butoxy)carbonyl]piperazin-1-yl}-9,9,11,11-tetramethyl-7-thia-2,5-diazatricyclo[6.4.0.0^{2,6}]dodeca-1(8),3,5-triene-4-carboxylate

The procedure to prepare ethyl 9,9,11,11-tetramethyl-3-(morpholin-4-yl)-7-thia-2,5-diazatricyclo[6.4.0.0^{2,6}]dodeca-1(8),3,5-triene-4-carboxylate was used except that N-Boc-piperazine was substituted for morpholine (13% yield); m/z=491.9 (MH)⁺.

### Example 175

### Ethyl 9,9,11,11-tetramethyl-3-(piperazin-1-yl)-7-thia-2,5-diazatricyclo[6.4.0.0^{2,6}]dodeca-1(8),3,5-triene-4-carboxylate

To a solution of ethyl 3-{1-[(tert-butoxy)carbonyl]piperazin-1-yl}-9,9,11,11-tetramethyl-7-thia-2,5-diazatricyclo[6.4.0.0^{2,6}]dodeca-1(8),3,5-triene-4-carboxylate (70% purity, 42 mg, 0.060 mmol) in MeOH (0.6 mL) at 0° C. was added AcCl (10 μL), with the resulting solution allowed to warm to room temperature over 1 h. The solution was re-cooled to 0° C. and further AcCl (20 μL) was added, with the reaction mixture allowed to warm to room temperature over 2 h then concentrated and azeotroped with toluene (×2) to afford the title compound as a pale red solid (41 mg, 44% purity, 77% yield); m/z=391.1 (MH)⁺.

### Example 176

### Ethyl 3-(1,2,3,6-tetrahydropyridin-4-yl)-9,9,11,11-tetramethyl-7-thia-2,5-diazatricyclo[6.4.0.0^{2,6}]dodeca-1(8),3,5-triene-4-carboxylate

The procedure to prepare ethyl 9,9,11,11-tetramethyl-3-(piperazin-1-yl)-7-thia-2,5-diazatricyclo[6.4.0.0^{2,6}]dodeca-1(8),3,5-triene-4-carboxylate was utilised except that ethyl 3-{1-[(tert-butoxy)carbonyl]-1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylate was substituted for ethyl 3-{1-[(tert-butoxy)carbonyl]piperazin-1-yl}-9,9,11,11-tetramethyl-7-thia-2,5-diazatricyclo[6.4.0.0^{2,6}]dodeca-1(8),3,5-triene-4-carboxylate (89% yield); m/z=388.1 (MH)⁺.

### Example 177

### Ethyl 3-(4-acetylpiperazin-1-yl)-9,9,11,11-tetramethyl-7-thia-2,5-diazatricyclo[6.4.0.0^{2,6}]dodeca-1(8),3,5-triene-4-carboxylate

The procedure to prepare ethyl 9,9,11,11-tetramethyl-3-(morpholin-4-yl)-7-thia-2,5-diazatricyclo[6.4.0.0^{2,6}]dodeca-1(8),3,5-triene-4-carboxylate was used except that acetylpiperazine (2.5 eq) was utilised in place of morpholine (60% yield); m/z=433.8 (MH)⁺.

### Example 178

### Ethyl 9,10-dichloro-11-[2-(morpholin-4-yl)ethoxy]-7-thia-2,5-diazatricyclo[6.4.0.0^{2,6}]dodeca-1(8),5,9,11-tetraene-4-carboxylate

The procedure to prepare ethyl 10-bromo-9-chloro-7-thia-2,5-diazatricyclo[6.4.0.0^{2,6}]dodeca-1(8),5,9,11-tetraene-4-carboxylate was used except that the reaction mixture was heated for 4 h after which the reaction mixture was concentrated. The residue was dissolved in EtOAc (25 mL) and was washed with water (25 mL) and brine (25 mL), dried (Na₂SO₄), filtered and concentrated. The crude product was purified by automated reverse phase HPLC (low pH method) to give the title compound as a pale pink gel (7% yield); m/z=446.1 (MH)⁺.

### Example 179

### Ethyl 9,9,11,11-tetramethyl-3-(prop-1-en-2-yl)-7-thia-2,5-diazatricyclo[6.4.0.0^{2,6}]dodeca-1(8),3,5-triene-4-carboxylate

Potassium isopropenyl trifluoroborate (138 mg, 0.934 mmol) was added to a mixture of MeCN (4.5 mL) and water (0.4 mL) containing ethyl 3-bromo-9,9,11,11-tetramethyl-7-thia-2,5-diazatricyclo[6.4.0.0^{2,6}]dodeca-1(8),3,5-triene-4-carboxylate (400 mg, 1.04 mmol), Pd(OAc)₂ (23 mg, 0.010 mmol), tricyclohexylphosphonium tetrafluoroborate (38 mg, 0.10 mmol) and caesium carbonate (676 mg, 2.08 mmol). The resulting mixture was degassed with nitrogen, then heated to 100° C. in a sealed tube for 1 h. The cooled reaction mixture was taken up in EtOAc (25 mL) and washed with water (10 mL) and brine (10 mL). The organic phase was dried over Na₂SO₄, filtered and concentrated. The residue was purified by FCC on silica (eluent: 20-80% EtOAc in heptane) to yield the title compound as a colourless oil (107 mg, 84% purity, 50% yield); m/z=347.6 (MH)⁺.

### Example 180

### Ethyl 9,9,11,11-tetramethyl-3-(4-methylpiperazin-1-yl)-7-thia-2,5-diazatricyclo[6.4.0.0^{2,6}]dodeca-1(8),3,5-triene-4-carboxylate

The procedure to prepare ethyl 9,9,11,11-tetramethyl-3-(morpholin-4-yl)-7-thia-2,5-diazatricyclo[6.4.0.0^{2,6}]dodeca-1(8),3,5-triene-4-carboxylate was used except that 1-methylpiperazine (2.5 eq) was utilised in place of morpholine (25% yield); m/z=405.7 (MH)⁺.

### Example 181

### Ethyl 3-(azetidin-3-yl)-9-chloro-10-fluoro-7-thia-2,5-diazatricyclo[6.4.0.0^{2,6}]dodeca-1(8),3,5,9,11-pentaene-4-carboxylate

To a suspension of ethyl 3-{1-[(tert-butoxy)carbonyl]azetidin-3-yl}-9-chloro-10-fluoro-7-thia-2,5-diazatricyclo[6.4.0.0^{2,6}]dodeca-1(8),3,5-triene-4-carboxylate (29% purity, 10.0 g, 6.4 mmol) in DCM (100 mL) was added TFA (20 mL). The reaction was stirred at room temperature for 6 h then concentrated. The residue was dissolved in the minimum volume of DCM/MeOH (1:1) then purified by filtration through SCX silica. After loading the solution, the column was washed with DCM then MeOH then DCM then MeOH. Product was eluted with 7M NH₃ in MeOH. The ammonia solution was then concentrated to afford the title compound as a brown solid (2.47 g, 86% LCMS purity, 93% yield); m/z=354.0 (MH)⁺.

### Example 182

### Ethyl 9-chloro-10-fluoro-3-(1-methylazetidin-3-yl)-7-thia-2,5-diazatricyclo[6.4.0.0^{2,6}]dodeca-1(8),3,5,9,11-pentaene-4-carboxylate

To a suspension of ethyl 3-(azetidin-3-yl)-9-chloro-10-fluoro-7-thia-2,5-diazatricyclo[6.4.0.0^{2,6}]dodeca-1(8),3,5,9,11-pentaene-4-carboxylate (86% purity, 500 mg, 1.22 mmol) in DCM (20 mL) was added formaldehyde (455 μL, 6.08 mmol), followed by sodium triacetoxyborohydride (644 mg, 3.04 mmol). The reaction mixture was stirred at room temperature for 66 h then filtered. The filtrate was diluted with water (15 mL) and the aqueous layer extracted with DCM (2×20 mL). The combined organic extracts were dried (Na₂SO₄), filtered and concentrated. The residue was purified by automated reverse phase HPLC (low pH method) to afford the title compound as a yellow solid (30 mg, 7% yield); m/z=368.1 (MH)⁺.

### Example 183

### Ethyl 9-chloro-3-cyclopropyl-10-fluoro-7-thia-2,5-diazatricyclo[6.4.0.0^{2,6}]dodeca-1(8),3,5,9,11-pentaene-4-carboxylate

The procedure to prepare ethyl 3-cyclopropyl-9,9,11,11-tetramethyl-7-thia-2,5-diazatricyclo[6.4.0.0^{2,6}]dodeca-1(8),3,5-triene-4-carboxylate was used except that ethyl 3-bromo-9-chloro-10-fluoro-7-thia-2,5-diazatricyclo[6.4.0.0^{2,6}]dodeca-1(8),3,5,9,11-pentaene-4-carboxylate was substituted for ethyl 3-bromo-9,9,11,11-tetramethyl-7-thia-2,5-diazatricyclo[6.4.0.0^{2,6}]dodeca-1(8),3,5-triene-4-carboxylate (19% yield); m/z=339.0 (MH)⁺.

### Example 184

### Ethyl 9,9,11,11-tetramethyl-3-(methylsulfanyl)-7-thia-2,5-diazatricyclo[6.4.0.0^{2,6}]dodeca-1(8),3,5-triene-4-carboxylate

A suspension of ethyl 3-bromo-9,9,11,11-tetramethyl-7-thia-2,5-diazatricyclo[6.4.0.0^{2,6}]dodeca-1(8),3,5-triene-4-carboxylate (100 mg, 0.260 mmol), Pd₂(dba)₃ (47 mg, 0.052 mmol), Xantphos (31 mg, 0.052 mmol), sodium methanethiolate (37 mg, 0.52 mmol) and DIPEA (90 μL, 0.52 mmol) in 1,4-dioxane (1.3 mL) was degassed with nitrogen before being sealed and stirred at 90° C. for 16 h. The reaction mixture was allowed to cool to room temperature and was partitioned between EtOAc and saturated aqueous NaHCO₃. The organic layer was separated and washed with brine, dried (MgSO₄), filtered and the filtrate concentrated. The residual material was purified by FCC (eluent: 0-14% EtOAc in heptane) to afford the title compound as a pale yellow oil (33 mg, 81% purity, 29% yield); m/z=353.5 (MH)⁺.

### Example 185

### 9,10-Dichloro-7-thia-2,5-diazatricyclo[6.4.0.0^{2,6}]dodeca-1(8),5,9,11-tetraene-4-carboxylic acid

To a solution of 6,7-dichloro-1,3-benzothiazol-2-amine (500 mg, 2.28 mmol) and ethyl 2-bromoprop-2-enoate (817 mg, 4.56 mmol) in EtOH (15 mL) was added triethylamine (635 μL, 4.56 mmol) and hydroquinone (50 mg, 0.46 mmol). The resulting solution was stirred at 90° C. After 5 h of heating an additional portion of ethyl 2-bromoprop-2-enoate (817 mg, 4.56 mmol) was added. After a further 11 h, the reaction mixture was evaporated to dryness, and then stirred with DCM. The resulting mixture was filtered and the filtrate evaporated. The residue was purified by FCC on silica (eluent: 0-50% EtOAc in heptane) to afford the ethyl ester intermediate (70% purity, 155 mg). This was dissolved in EtOH (5 mL) and LiOH.H₂O (27 mg, 0.65 mmol) was added. The resulting solution was stirred at 50° C. for 30 min. The reaction mixture was evaporated to dryness, dissolved in water (20 mL) and acidified with 1M HCl_{(aq)} to pH 2. The resulting precipitate was extracted with DCM (3×50 mL). The aqueous layer was evaporated to dryness and submitted to automated reverse phase HPLC purification (low pH method) to afford the title compound as a white solid (25 mg, 27% yield); m/z=289.0 (MH)⁺; 1H NMR (500 MHz, Methanol-d4) δ 4.51 (dd, 1H), 4.60 (dd, 1H), 5.18 (dd, 1H), 7.29 (d, 1H), 7.66 (d, 1H). Exchangeable protons not observed.

### Example 186

### 2-Hydroxy-2-{9,9,11,11-tetramethyl-7-thia-2,5-diazatricyclo[6.4.0.0^{2,6}]dodeca-1(8),3,5-trien-4-yl}acetic acid

To a solution of methyl 2-hydroxy-2-{9,9,11,11-tetramethyl-7-thia-2,5-diazatricyclo[6.4.0.02′6]dodeca-1(8),3,5-trien-4-yl}acetate (50%, 232 mg, 0.36 mmol) in water (1 mL) and EtOH (5 mL) was added LiOH.H₂O (30 mg, 0.72 mmol). The resulting solution was then heated at 40° C. for 2 h. The reaction mixture was then concentrated to dryness, diluted with water and acidified to pH 1. The reaction mixture was diluted with DCM, the organic layer was separated and the aqueous layer washed with DCM. The aqueous layer was then concentrated. The crude product was purified by automated reverse phase HPLC (low pH method) to afford the title compound as a white solid (33 mg, 30% yield); m/z=309.3 (MH)⁺; 1H NMR (500 MHz, DMSO-d6) δ 1.09 (s, 6H), 1.30 (s, 6H), 1.67 (s, 2H), 2.49 (s, 2H), 4.99 (s, 1H), 5.58 (s, 1H), 7.57 (s, 1H). Exchangeable protons not observed.

### Example 187

### 9,9,11,11-Tetramethyl-10-oxa-7-thia-2,5-diazatricyclo[6.4.0.0^{2,6}]dodeca-1(8),3,5-triene-4-carboxylic acid

A solution of ethyl 9,9,11,11-tetramethyl-10-oxa-7-thia-2,5-diazatricyclo[6.4.0.0^{2,6}]dodeca-1(8),3,5-triene-4-carboxylate (19 mg, 0.06 mmol) and LiOH.H₂O (3 mg, 0.12 mmol) in EtOH:H₂O (1:1, 0.6 mL) was heated to 60° C. for 30 min. After this time the reaction mixture was concentrated, azeotroped with toluene (×3) and dissolved in DMSO for purification by automated reverse phase HPLC (low pH method) to afford the title compound as a white solid (8 mg, 46%); m/z=281.0 (MH)⁺; 1H NMR (500 MHz, DMSO-d6) δ 1.32 (s, 6H), 1.49 (s, 6H), 2.79 (s, 2H), 8.29 (s, 1H). Exchangeable protons not observed.

### Example 188

### 2-{9,10-Dichloro-7-thia-2,5-diazatricyclo[6.4.0.0^{2,6}]dodeca-1(8),3,5,9,11-pentaen-4-yl}-2-hydroxyacetic acid

To a stirred solution of ethyl 2-{9,10-dichloro-7-thia-2,5-diazatricyclo[6.4.0.0^{2,6}]dodeca-1(8),3,5,9,11-pentaen-4-yl}-2-hydroxyacetate (52 mg, 0.14 mmol) in EtOH (5 mL) and water (1 mL) was added LiOH.H₂O (17 mg, 0.42 mmol). The reaction was at heated at 75° C. After 1.5 h the reaction mixture was re-treated by the portionwise addition of LiOH.H₂O (29 mg, 0.69 mmol) and then NaOH (27 mg, 0.69 mmol) over 6 h. After heating for a total of 8 h, the reaction was concentrated, dissolved in water (20 mL) and washed with DCM (10 mL). The aqueous phase was acidified with 1M HCl to pH1 and concentrated. The residue was purified by automated reverse phase HPLC (low pH method) to give the title compound as a white solid (7 mg, 15% yield); m/z=317.0 (MH)⁺; 1H NMR (500 MHz, Methanol-d4) δ 6.76 (s, 1H), 9.27 (d, 1H), 9.41 (d, 1H), 9.64 (s, 1H). Exchangeable protons not observed.

### Example 189

### 1-{10-tert-Butyl-7-thia-2,5-diazatricyclo[6.4.0.0^{2,6}]dodeca-1(8),3,5-trien-4-yl}-3-methanesulfonylurea

Diphenyl phosphorazidate (88 μL, 0.41 mmol) was added to a mixture of 10-tert-butyl-7-thia-2,5-diazatricyclo[6.4.0.0^{2,6}}]dodeca-1(8),3,5-triene-4-carboxylic acid (95 mg, 0.34 mmol), methanesulfonamide (34 mg, 0.36 mmol) and K₂CO₃ (104 mg, 0.751 mmol) in 1,4-dioxane (5 mL). The mixture was sealed under nitrogen and stirred at 85° C. for 5.5 h. The reaction was cooled to room temperature, diluted with EtOAc (30 mL) and washed with 1M HCl (10 mL) and brine (10 mL). The organic phase was dried over Na₂SO₄, the mixture filtered and the filtrate was concentrated. The residue was purified by automated reverse-phase HPLC (low pH method) with further purification by filtration through a silica plug, eluting with 0-5% MeOH in DCM, to afford the title compound as a light brown solid (9 mg, 7% yield); m/z=371.3 (MH)⁺; 1H NMR (500 MHz, DMSO-d6) δ 0.93 (s, 9H), 1.41-1.45 (m, 1H), 1.56-1.60 (m, 1H), 2.06-2.09 (m, 1H), 2.43 (d, 1H), 2.52-2.61 (m, 1H), 2.68 (dd, 1H), 2.74-2.84 (m, 1H), 3.27 (s, 3H), 7.49 (s, 1H), 8.97 (s, 1H), 10.16 (s, 1H).

### Example 190

### 10-tert-Butyl-3-(4-methoxyphenyl)-7-thia-2,5-diazatricyclo[6.4.0.0^{2,6}]dodeca-1(8),3,5-triene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 10-tert-butyl-3-(4-methoxyphenyl)-7-thia-2,5-diazatricyclo[6.4.0.0^{2,6}]dodeca-1(8),3,5-triene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (57% yield); m/z=385.3 (MH)⁺; 1H NMR (500 MHz, Methanol-d4) δ 0.96 (s, 9H), 1.32 (m, 1H), 1.50-1.66 (m, 1H), 1.95 (d, 1H), 2.10 (dd, 1H), 2.14-2.28 (m, 1H), 2.41-2.63 (m, 1H), 2.76 (dd, 1H), 3.88 (s, 3H), 7.01 (d, 2H), 7.39 (d, 2H). Exchangeable protons not observed.

### Example 191

### 10,10-Dimethyl-7-thia-2,5-diazatetracyclo[6.4.0.0^{2,6}.0^{9,11}]dodeca-1(8),3,5-triene-4-carboxylic acid

To a solution of ethyl 10,10-dimethyl-7-thia-2,5-diazatetracyclo[6.4.0.0^{2,6}.0^{9,11}]dodeca-1(8),3,5-triene-4-carboxylate (50% purity, 745 mg, 1.35 mmol) in EtOH (25 mL) and water (5 mL) was added LiOH.H₂O (113 mg, 2.70 mmol). The resulting solution was stirred at 70° C. for 2 h. More LiOH.H₂O (113 mg, 2.70 mmol) was added and heated for a further 1 h at 70° C. The mixture was evaporated to dryness, re-dissolved in water (75 mL), acidified with 2M HCl to pH 2 then extracted with DCM (2×50 mL). The combined organic layers were dried over MgSO₄, filtered and concentrated. The crude product was purified by automated reverse phase HPLC (low pH method) affording the title compound as a white solid (108 mg, 32% yield); m/z=249.1 (MH)⁺; 1H NMR (500 MHz, DMSO-d6) δ 0.68 (s, 3H), 1.12 (s, 3H), 1.88 (t, 1H), 2.24-2.33 (m, 1H), 2.72 (d, 1H), 3.08 (dd, 1H), 8.23 (s, 1H), 12.50 (s, 1H).

### Example 192

### N-Methanesulfonyl-9,9,11,11-tetramethyl-10-oxa-7-thia-2,5-diazatricyclo[6.4.0.0^{2,6}]dodeca-1(8),3,5-triene-4-carboxamide

A mixture of 9,9,11,11-tetramethyl-10-oxa-7-thia-2,5-diazatricyclo[6.4.0.0^{2,6}]dodeca-1(8),3,5-triene-4-carboxylic acid (37 mg, 0.13 mmol), EDC (63 mg, 0.33 mmol) methanesulfonamide (13 mg, 0.13 mmol) and DMAP (48 mg, 0.40 mmol) in DMF (1.3 mL) was stirred at room temperature for 16 h. The previously described amounts of EDC, DMAP and methanesulfonamide were added, with stirring continued for a further 4 h. After this time, the reaction mixture was diluted with EtOAc and saturated aqueous sodium hydrogen carbonate solution added. The aqueous layer was removed and the organic layer was washed with saturated brine, dried (MgSO₄), filtered and concentrated. The residual material was dissolved in DMSO and purified by automated reverse phase HPLC (low pH method), affording the title compound as a white solid (16 mg, 34% yield); m/z=358.0 (MH)⁺; 1H NMR (500 MHz, DMSO-d6) δ 1.32 (s, 6H), 1.50 (s, 6H), 2.79 (s, 2H), 3.22 (s, 3H), 8.39 (s, 1H). Exchangeable protons not observed.

### Example 193

### N-Methanesulfonyl-10,10-dimethyl-7-thia-2,5-diazatetracyclo[6.4.0.0^{2,6}.0^{9,11}]dodeca-1(8),3,5-triene-4-carboxamide

To a solution of 10,10-dimethyl-7-thia-2,5-diazatetracyclo[6.4.0.0^{2,6}.0^{9,11}]dodeca-1(8),3,5-triene-4-carboxylic acid (60 mg, 0.24 mmol) in DCM (10 mL) was added 1 drop of DMF followed by oxalyl chloride (62 μL, 0.72 mmol) under nitrogen. The mixture was stirred at room temperature for 1 h and then evaporated to dryness. The resulting solid was immediately dissolved in DCM (10 mL) under nitrogen and methanesulfonamide (29 mg, 0.30 mmol) followed by DIPEA (86 μL, 0.48 mmol) was added and the mixture stirred at room temperature for 2 h. More methanesulfonamide (29 mg, 0.3 mmol) followed by DIPEA (86 μL, 0.48 mmol) was added and the mixture was stirred at room temperature for 18 h. The reaction mixture was washed with 1M HCl (2×25 mL) and water (2×25 mL). The combined organic layers were dried over MgSO₄, filtered and evaporated to dryness. The crude product was purified by automated reverse phase HPLC (low pH method) to afford the title compound as a yellow solid (29 mg, 37% yield); m/z=326.3 (MH)⁺; 1H NMR (500 MHz, Methanol-d4) δ 0.75 (s, 3H), 1.17 (s, 3H), 1.94 (t, 1H), 2.26 (dd, 1H), 2.78 (dd, 1H), 3.11 (dd, 1H), 3.34 (s, 3H), 8.20 (s, 1H). Exchangeable protons not observed.

### Example 194

### 10-tert-Butyl-3-acetamido-7-thia-2,5-diazatricyclo[6.4.0.0^{2,6}]dodeca-1(8),3,5-triene-4-carboxylic acid

To a stirred solution of ethyl 10-tert-butyl-3-acetamido-7-thia-2,5-diazatricyclo[6.4.0.0^{2,6}]dodeca-1(8),3,5-triene-4-carboxylate (18% purity, 41 mg, 0.11 mmol) in EtOH (3 mL) and water (0.6 mL) was added 2M sodium hydroxide (0.11 mL, 0.22 mmol) and the mixture stirred at room temperature for 3 h. A further charge of 2M sodium hydroxide (0.11 mL, 0.22 mmol) was added to the mixture and it was stirred at room temperature for 18 h then heated at 50° C. for 3 h. The reaction mixture was cooled and the EtOH removed in vacuo. The resulting residue was washed with EtOAc (15 mL). The aqueous phase was acidified to pH 1-2 and concentrated. The crude product was purified by automated reverse phase HPLC (low pH method) to yield the title compound as an off white solid (1.6 mg, 4.2% yield); m/z=336.1 (MH)⁺; 1H NMR (500 MHz, Methanol-d4) δ 1.00 (s, 9H), 1.48-1.52 (m, 1H), 1.61-1.65 (m, 1H), 2.14-2.18 (m, 1H), 2.20 (s, 3H), 2.49-2.58 (m, 1H), 2.64-2.81 (m, 2H), 2.92-3.04 (m, 1H), 8.45 (s, 1H). Formic acid salt. Exchangeable protons not observed.

### Example 195

### 10-tert-Butyl-3-methyl-7-thia-2,5-diazatricyclo[6.4.0.0^{2,6}]dodeca-1(8),3,5-triene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 10-tert-butyl-3-methyl-7-thia-2,5-diazatricyclo[6.4.0.0^{2,6}]dodeca-1(8),3,5-triene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (37% yield); m/z=293.0 (MH)⁺; 1H NMR (500 MHz, DMSO-d6) δ 0.94 (s, 9H), 1.41 (ddt, 1H), 1.52-1.58 (m, 1H), 2.05-2.10 (m, 1H), 2.42-2.48 (m, 1H), 2.62-2.71 (m, 1H), 2.77 (s, 3H), 2.83-2.91 (m, 1H), 3.15 (dd, 1H). Exchangeable protons not observed.

### Example 196

### 10-tert-Butyl-3-methanesulfonamido-7-thia-2,5-diazatricyclo[6.4.0.0^{2,6}]dodeca-1(8),3,5-triene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 10-tert-butyl-3-methanesulfonamido-7-thia-2,5-diazatricyclo[6.4.0.0^{2,6}]dodeca-1(8),3,5-triene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (24% yield); m/z=371.9 (MH)⁺; 1H NMR (500 MHz, Methanol-d4) δ 1.01 (s, 9H), 1.51 (dd, 1H), 1.60-1.70 (m, 1H), 2.20-2.30 (m, 1H), 2.52-2.60 (m, 1H), 2.75-2.80 (m, 1H), 2.92 (m, 1H), 3.12 (s, 3H). 1 Proton hidden. Exchangeable protons not observed.

### Example 197

### 9,12-Dichloro-7-thia-2,5-diazatricyclo[6.4.0.0^{2,6}]dodeca-1(8),3,5,9,11-pentaene-4-carboxylic acid

To a stirred suspension of ethyl 9,12-dichloro-7-thia-2,5-diazatricyclo[6.4.0.0^{2,6}]dodeca-1(8),3,5,9,11-pentaene-4-carboxylate (200 mg, 0.635 mmol) in EtOH:water (2:1, 3 mL) was added LiOH.H₂O (29 mg, 0.70 mmol). The resulting suspension stirred at 45° C. for 18 h. More LiOH.H₂O (29 mg, 0.70 mmol) was added and the reaction mixture heated at 65° C. for 2 h. The EtOH was evaporated under reduced pressure and the aqueous suspension was further diluted with water (3 mL), acidified to pH 3 using saturated aqueous citric acid (4 mL). The resulting precipitate was collected by filtration, washed with water and purified by automated reverse phase HPLC (low pH method) to afford to the title compound as a white solid (5 mg, 3% yield); m/z=286.8 (MH)⁺; 1H NMR (500 MHz, Methanol-d4) δ 7.57 (d, 1H), 7.67 (d, 1H), 8.96 (s, 1H). Exchangeable protons not observed.

### Example 198

### 9-(Trifluoromethoxy)-7-thia-2,5-diazatricyclo[6.4.0.0^{2,6}]dodeca-1(8),3,5,9,11-pentaene-4-carboxylic acid

The procedure to prepare 9,12-dichloro-7-thia-2,5-diazatricyclo[6.4.0.0^{2,6}]dodeca-1(8),3,5,9,11-pentaene-4-carboxylic acid was used except that ethyl 9-(trifluoromethoxy)-7-thia-2,5-diazatricyclo[6.4.0.0^{2,6}]dodeca-1(8),3,5,9,11-pentaene-4-carboxylate was substituted for ethyl 9,12-dichloro-7-thia-2,5-diazatricyclo[6.4.0.0^{2,6}]dodeca-1(8),3,5,9,11-pentaene-4-carboxylate (7% yield); m/z=303.0 (MH)⁺; 1H NMR (500 MHz, DMSO-d6) δ 7.58 (d, 1H), 7.73 (t, 1H), 8.22 (d, 1H), 9.03 (s, 1H). Exchangeable protons not observed.

### Example 199

### 3-Iodo-9,9,11,11-tetramethyl-7-thia-2,5-diazatricyclo[6.4.0.0^{2,6}]dodeca-1(8),3,5-triene-4-carboxylic acid

The procedure to prepare 9,12-dichloro-7-thia-2,5-diazatricyclo[6.4.0.0^{2,6}]dodeca-1(8),3,5,9,11-pentaene-4-carboxylic acid was used except that ethyl 3-iodo-9,9,11,11-tetramethyl-7-thia-2,5-diazatricyclo[6.4.0.0^{2,6}]dodeca-1(8),3,5-triene-4-carboxylate was substituted for ethyl 9,12-dichloro-7-thia-2,5-diazatricyclo[6.4.0.0^{2,6}]dodeca-1(8),3,5,9,11-pentaene-4-carboxylate (21% yield); m/z=405.0 (MH)⁺; 1H NMR (500 MHz, Methanol-d4) δ 1.16 (s, 6H), 1.37 (s, 6H), 1.73 (s, 2H), 3.07 (s, 2H). Exchangeable protons not observed.

### Example 200

### 10-tert-Butyl-4-(1H-1,2,3,4-tetrazol-5-yl)-7-thia-2,5-diazatricyclo[6.4.0.0^{2,6}]dodeca-1(8),3,5-triene

To a stirred suspension of 10-tert-butyl-7-thia-2,5-diazatricyclo[6.4.0.0^{2,6}]dodeca-1(8),3,5-triene-4-carbonitrile (45 mg, 0.17 mmol) and dibutyl(oxo)stannane (50 μL, 0.17 mmol) in xylene (0.85 mL) under nitrogen was added azido(trimethyl)silane (69 μL, 0.52 mmol). The resulting solution heated at 130° C. for 2 h. After this time the reaction mixture was diluted with MeOH (2 mL) and stirred at room temperature for a further 2 h. The reaction mixture was concentrated. The residue was diluted with diethyl ether (3 mL) and MeOH (3 mL). The resulting precipitate was isolated by filtration. The crude product was purified by automated reverse phase HPLC (low pH method) affording the title compound as a white solid (3 mg, 5% yield); m/z=303.1 (MH)⁺; 1H NMR (500 MHz, Methanol-d4) δ 1.03 (s, 9H), 1.55-1.65 (m, 1H), 1.66-1.77 (m, 1H), 2.25 (dd, 1H), 2.51-2.64 (m, 1H), 2.66-2.74 (m, 1H), 2.77-2.83 (m, 1H), 2.87-2.93 (m, 1H), 8.10 (s, 1H). Exchangeable protons not observed.

### Example 201

### 10-[(Trifluoromethyl)sulfanyl]-7-thia-2,5-diazatricyclo[6.4.0.0^{2,6}]dodeca-1(8),3,5,9,11-pentaene-4-carboxylic acid

A suspension of ethyl 10-[(trifluoromethyl)sulfanyl]-7-thia-2,5-diazatricyclo[6.4.0.0^{2,6}]dodeca-1(8),3,5,9,11-pentaene-4-carboxylate (48 mg, 0.16 mmol) and LiOH.H₂O (8 mg, 0.3 mmol in EtOH (1.6 mL) was heated at 60° C. for 1 h. The reaction mixture was diluted with water and EtOAc and the organic layer was discarded. The aqueous layer was acidified to pH 1 by addition of 1M HCl_{(aq)} and extracted with EtOAc. The organic layer was washed with brine, dried (MgSO₄), filtered and concentrated to afford the title compound as an off-white solid (29 mg, 63% yield); m/z=318.9 (MH)⁺;

1H NMR (500 MHz, DMSO-d6) δ 7.93 (dd, 1H), 8.30 (d, 1H), 8.57 (d, 1H), 9.03 (s, 1H). Exchangeable protons not observed.

### Example 202

### 10-(Methylsulfanyl)-7-thia-2,5-diazatricyclo[6.4.0.0^{2,6}]dodeca-1(8),3,5,9,11-pentaene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 10-(methylsulfanyl)-7-thia-2,5-diazatricyclo[6.4.0.0^{2,6}]dodeca-1(8),3,5,9,11-pentaene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (7% yield); m/z=265.0 (MH)⁺; 1H NMR (500 MHz, DMSO-d6) δ 2.57 (s, 3H), 7.47 (dd, 1H), 8.00 (d, 1H), 8.08 (d, 1H), 8.93 (s, 1H). Exchangeable protons not observed.

### Example 203

### 9,10-Dichloro-3-[(dimethylamino)methyl]-7-thia-2,5-diazatricyclo[6.4.0.0^{2,6}]dodeca-1(8),3,5,9,11-pentaene-4-carboxylic acid

To a solution of 9,10-dichloro-7-thia-2,5-diazatricyclo[6.4.0.0^{2,6}]dodeca-1(8),3,5,9,11-pentaene-4-carboxylic acid (250 mg, 0.871 mmol) in DMSO (3 mL) was added dimethyl(methylidene)azanium iodide (483 mg, 2.61 mmol). The resulting solution was stirred in a pressure tube at 130° C. for 2 h. The reaction mixture was poured into water (10 mL) and the resulting mixture filtered. The collected solid was purified by automated reverse phase HPLC (low pH method) to afford the title compound as a light brown solid (32 mg, 11% yield); m/z=344.0 (MH)⁺; 1H NMR (500 MHz, Methanol-d4) δ 2.86 (s, 6H), 7.76 (d, 1H), 8.11 (d, 1H). 2 Protons hidden. Exchangeable protons not observed.

### Example 204

### 9,11-Dichloro-7-thia-2,5-diazatricyclo[6.4.0.0^{2,6}]dodeca-1(8),3,5,9,11-pentaene-4-carboxylic acid

To a stirred suspension of ethyl 9,11-dichloro-7-thia-2,5-diazatricyclo[6.4.0.0^{2,6}]dodeca-1(8),3,5,9,11-pentaene-4-carboxylate (120 mg, 0.38 mmol) in EtOH (3.5 mL) was added 1M NaOH_{(aq)} (2.67 mL, 2.67 mmol). The reaction was heated at 50° C. for 50 min. The reaction was concentrated and the residue diluted with water (10 mL). The pH was adjusted to pH 2 with 1M HCl_{(aq)}. The resulting solid was collected by filtration and triturated in DCM to give the title compound as a white solid (78 mg, 71% yield); m/z=286.9 (MH)⁺; 1H NMR (500 MHz, DMSO-d6) δ 7.84 (d, 1H), 8.42 (d, 1H), 8.97 (s, 1H), 12.75 (s, 1H).

### Example 205

### 3-[(Dimethylamino)methyl]-9,9,11,11-tetramethyl-7-thia-2,5-diazatricyclo[6.4.0.0^{2,6}]dodeca-1(8),3,5-triene-4-carboxylic acid

To a solution of 9,9,11,11-tetramethyl-7-thia-2,5-diazatricyclo[6.4.0.0^{2,6}]dodeca-1(8),3,5-triene-4-carboxylic acid (92 mg, 0.33 mmol) in DMSO (4 mL) was added dimethyl(methylidene)azanium iodide (183 mg, 0.99 mmol). The resulting mixture was stirred at 130° C. for 2 h then cooled and water added (50 mL). The mixture was filtered and the filtrate evaporated to dryness. The residue was purified using automated reverse phase HPLC (low pH method) to yield the title compound as a yellow solid (15 mg, 12% yield); m/z=336.1 (MH)⁺; 1H NMR (500 MHz, DMSO-d6) δ 1.08 (s, 6H), 1.32 (s, 6H), 1.65 (s, 2H), 2.27 (s, 6H), 2.85 (s, 2H), 4.04 (s, 2H). Exchangeable protons not observed.

### Example 206

### 10-tert-Butyl-7-thia-2,5-diazatricyclo[6.4.0.0^{2,6}]dodeca-1(8),3,5,9,11-pentaene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 10-tert-butyl-7-thia-2,5-diazatricyclo[6.4.0.0^{2,6}]dodeca-1(8),3,5,9,11-pentaene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (22% yield); m/z=275.4 (MH)⁺; 1H NMR (500 MHz, DMSO-d6) δ 1.36 (s, 9H), 7.62 (dd, 1H), 7.98-8.15 (m, 2H), 8.92 (s, 1H), 12.62 (s, 1H).

### Example 207

### 3-Chloro-9,9,11,11-tetramethyl-7-thia-2,5-diazatricyclo[6.4.0.0^{2,6}]dodeca-1(8),3,5-triene-4-carboxylic acid

The procedure to prepare 10-[(trifluoromethyl)sulfanyl]-7-thia-2,5-diazatricyclo[6.4.0.0^{2,6}]dodeca-1(8),3,5,9,11-pentaene-4-carboxylic acid was used except that ethyl 3-chloro-9,9,11,11-tetramethyl-7-thia-2,5-diazatricyclo[6.4.0.0^{2,6}]dodeca-1(8),3,5-triene-4-carboxylate was substituted for ethyl 10-[(trifluoromethyl)sulfanyl]-7-thia-2,5-diazatricyclo[6.4.0.0^{2,6}]dodeca-1(8),3,5,9,11-pentaene-4-carboxylate (78% yield); m/z=313.0 (MH)⁺; 1H NMR (500 MHz, DMSO-d6) δ 1.09 (s, 6H), 1.31 (s, 6H), 1.65 (s, 2H), 2.83 (s, 2H). Exchangeable protons not observed.

### Example 208

### 10-(Benzyloxy)-7-thia-2,5-diazatricyclo[6.4.0.0^{2,6}]dodeca-1(8),3,5,9,11-pentaene-4-carboxylic acid

To a solution of ethyl 10-(benzyloxy)-7-thia-2,5-diazatricyclo[6.4.0.0^{2,6}]dodeca-1(8),3,5,9,11-pentaene-4-carboxylate (132 mg, 0.375 mmol) in EtOH (5 mL) and water (1 mL) was added LiOH.H₂O (31 mg, 0.75 mmol). The resulting mixture was stirred at 50° C. for 2 h then evaporated to dryness, the residue dissolved in water and the solution acidified with 1M HCl_{(aq)} to pH 2. The resulting precipitate was collected by filtration, washed with water and dried in the vacuum oven to afford the title compound as a white solid (81 mg, 67% yield); m/z=324.9 (MH)⁺; 1H NMR (500 MHz, DMSO-d6) δ 5.18 (s, 2H), 7.23 (dd, 1H), 7.35 (dd, 1H), 7.41 (t, 2H), 7.48 (d, 2H), 7.77 (d, 1H), 8.06 (d, 1H), 8.88 (s, 1H), 12.58 (s, 1H).

### Example 209

### 9,10-Difluoro-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 9,10-difluoro-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate and trituration was performed in MeOH and water rather than DCM (22% yield); m/z=254.9 (MH)⁺; 1H NMR (500 MHz, DMSO-d6) δ 7.76 (dt, 1H), 8.06 (ddd, 1H), 9.02 (s, 1H), 12.77 (s, 1H).

### Example 210

### 9-Chloro-3-[(dimethylamino)methyl]-10-fluoro-7-thia-2,5-diazatricyclo[6.4.0.0^{2,6}]dodeca-1(8),3,5,9,11-pentaene-4-carboxylic acid

The procedure to prepare 3-[(dimethylamino)methyl]-9,9,11,11-tetramethyl-7-thia-2,5-diazatricyclo[6.4.0.0^{2,6}]dodeca-1(8),3,5-triene-4-carboxylic acid was used except that 9-chloro-10-fluoro-7-thia-2,5-diazatricyclo[6.4.0.0^{2,6}]dodeca-1(8),3,5,9,11-pentaene-4-carboxylic acid was substituted for 9,9,11,11-tetramethyl-7-thia-2,5-diazatricyclo[6.4.0.0^{2,6}]dodeca-1(8),3,5-triene-4-carboxylic acid (8% yield); m/z=328.0 (MH)⁺; 1H NMR (500 MHz, DMSO-d6) δ 2.29 (s, 6H), 4.23 (s, 2H), 7.70 (t, 1H), 8.02 (dd, 1H). Exchangeable protons not observed.

### Example 211

### 10-(Cyclopentyloxy)-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 10-(cyclopentylmethoxy)-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (73% yield); m/z=302.9 (MH)⁺; 1H NMR (500 MHz, DMSO-d6) δ 1.55-1.67 (m, 2H), 1.67-1.81 (m, 4H), 1.91-2.00 (m, 2H), 4.81-4.94 (m, 1H), 7.10 (dd, 1H), 7.64 (d, 1H), 8.02 (d, 1H), 8.87 (s, 1H), 12.59 (s, 1H).

### Example 212

### 9-Bromo-7-thia-2,5-diazatricyclo[6.4.0.0^{2,6}]dodeca-1(8),3,5,9,11-pentaene-4-carboxylic acid

Ethyl 9-bromo-7-thia-2,5-diazatricyclo[6.4.0.0^{2,6}]dodeca-1(8),3,5,9,11-pentaene-4-carboxylate (100 mg, 0.308 mmol) was dissolved in EtOH (10 mL) and water (1 mL). LiOH.H₂O (39 mg, 0.92 mmol) was added and the reaction mixture was stirred at 50° C. for 1 h then evaporated to dryness. The residue was dissolved in water and acidified to pH 2 using 1M HCl_{(aq)}. The resulting mixture was extracted into EtOAc (3×25 mL). The combined organic extracts were dried over MgSO₄ and evaporated to dryness. The resulting residue was purified by automated reverse phase HPLC (low pH method) to afford the title compound as a white solid (63 mg, 12% yield); m/z=297.0 (MH)⁺; 1H NMR (500 MHz, DMSO-d6) δ 7.56 (t, 1H), 7.73 (dd, 1H), 8.15-8.27 (m, 1H), 8.99 (s, 1H), 12.72 (s, 1H).

### Example 213

### 11-Chloro-10-(trifluoromethoxy)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 11-chloro-10-(trifluoromethoxy)-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.02,6]dodeca-1(8),3,5,9,11-pentaene-4-carboxylate (99% yield); m/z=337.0 (MH)⁺; 1H NMR (500 MHz, DMSO-d6) δ 8.48 (d, 1H), 8.64 (s, 1H), 8.95 (s, 1H), 12.77 (s, 1H).

### Example 214

### 9-Chloro-10-(trifluoromethoxy)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 9-chloro-10-(trifluoromethoxy)-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.02,6]dodeca-1(8),3,5,9,11-pentaene-4-carboxylate (91% yield); m/z=336.9 (MH)⁺; 1H NMR (500 MHz, DMSO-d6) δ 7.88 (dd, 1H), 8.29 (d, 1H), 9.04 (s, 1H). Exchangeable protons not observed.

### Example 215

### 12-Methyl-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 12-methyl-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate. The product was triturated in MeOH (39% yield); m/z=233.1 (MH)⁺; 1H NMR (500 MHz, DMSO-d6) δ 2.77 (s, 3H), 7.37 (d, 2H), 7.85-7.90 (m, 1H), 8.62 (s, 1H). Exchangeable protons not observed.

### Example 216

### 12-Methoxy-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 12-methoxy-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (51% yield); m/z=249.0 (MH)⁺; 1H NMR (500 MHz, DMSO-d6) δ 4.07 (s, 3H), 7.23-7.25 (m, 1H), 7.44 (t, 1H), 7.60-7.62 (m, 1H), 8.52 (s, 1H), 12.67 (s, 1H).

### Example 217

### 10-Chloro-9-methoxy-7-thia-2,5-diazatricyclo[6.4.0.0^{2,6}]dodeca-1(8),3,5,9,11-pentaene-4-carboxylic acid

The procedure to prepare 9,12-dichloro-7-thia-2,5-diazatricyclo[6.4.0.0^{2,6}]dodeca-1(8),3,5,9,11-pentaene-4-carboxylic acid was used except that ethyl 10-chloro-9-methoxy-7-thia-2,5-diazatricyclo[6.4.0.0^{2,6}]dodeca-1(8),3,5,9,11-pentaene-4-carboxylate was substituted for ethyl 9,12-dichloro-7-thia-2,5-diazatricyclo[6.4.0.0^{2,6}]dodeca-1(8),3,5,9,11-pentaene-4-carboxylate. Preparative HPLC was not necessary (66% yield); m/z=282.9 (MH)⁺; 1H NMR (500 MHz, Methanol-d4) δ 4.07 (s, 3H), 7.61 (d, 1H), 7.74 (d, 1H), 8.68 (s, 1H). Exchangeable protons not observed.

### Example 218

### 9-Chloro-10-methoxy-7-thia-2,5-diazatricyclo[6.4.0.0^{2,6}]dodeca-1(8),3,5,9,11-pentaene-4-carboxylic acid

To a stirred solution of 7-chloro-6-methoxy-1,3-benzothiazol-2-amine (0.090 g, 0.419 mmol) in DME (6 mL) was added ethyl 3-bromo-2-oxopropanoate (264 μL, 1.68 mmol). The reaction was heated at 85° C. for 4 h. The DME was evaporated and MeOH/2M NaOH_{(aq)} were added. After stirring for 30 min the solvent was evaporated and the mixture partitioned between water and DCM. The DCM layer was discarded and the aqueous phase was made acidic with 2M HCl_{(aq)} then extracted with EtOAc (3×30 mL). On standing, the combined organic extracts formed a white precipitate which was filtered off and retained. The organic filtrate was dried (Na₂SO₄), filtered and evaporated and combined with the solid. This mixture was purified by automated reverse phase HPLC (low pH method) to afford the title compound as an off white solid (8 mg, 4% yield); m/z=282.9 (MH)⁺; 1H NMR (500 MHz, DMSO-d6) δ 3.97 (s, 3H), 7.42 (d, 1H), 8.11 (d, 1H), 8.75 (s, 1H). Exchangeable protons not observed.

### Example 219

### 9-Fluoro-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 9-fluoro-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (45% yield); m/z=237.0 (MH)⁺; 1H NMR (500 MHz, DMSO) δ 7.41-7.48 (m, 1H), 7.65 (td, 1H), 8.05 (dd, 1H), 9.03 (s, 1H), 12.78 (s, 1H).

### Example 220

### 9-Chloro-N-methanesulfonyl-10-(trifluoromethoxy)-7-thia-2,5-diazatricyclo[6.4.0.0^{2,6}]dodeca-1(8),3,5,9,11-pentaene-4-carboxamide

The procedure to prepare N-methanesulfonyl-10,10-dimethyl-7-thia-2,5-diazatetracyclo[6.4.0.0^{2,6}.0^{9′11}]dodeca-1(8),3,5-triene-4-carboxamide was used except that 9-chloro-10-(trifluoromethoxy)-7-thia-2,5-diazatricyclo[6.4.0.0^{2,6}]dodeca-1(8),3,5,9,11-pentaene-4-carboxylic acid was substituted for 10,10-dimethyl-7-thia-2,5-diazatetracyclo[6.4.0.0^{2,6}.0^{9′11}]dodeca-1(8),3,5-triene-4-carboxylic acid. Purification was by trituration in MeOH rather than by HPLC (25% yield); m/z=413.9 (MH)⁺; 1H NMR (500 MHz, DMSO-d6) δ 3.35 (s, 3H), 7.91 (dd, 1H), 8.30 (d, 1H), 9.20 (s, 1H), 11.71 (s, 1H).

### Example 221

### 3-Methanesulfonamido-9,9,11,11-tetramethyl-7-thia-2,5-diazatricyclo[6.4.0.0^{2,6}]dodeca-1(8),3,5-triene-4-carboxylic acid

The procedure to prepare 9,9,11,11-tetramethyl-10-oxa-7-thia-2,5-diazatricyclo[6.4.0.0^{2,6}]dodeca-1(8),3,5-triene-4-carboxylic acid was used except that ethyl 3-methanesulfonamido-9,9,11,11-tetramethyl-7-thia-2,5-diazatricyclo[6.4.0.0^{2,6}]dodeca1(8),3,5-triene-4-carboxylate was substituted for ethyl 9,9,11,11-tetramethyl-10-oxa-7-thia-2,5-diazatricyclo[6.4.0.0^{2,6}]dodeca-1(8),3,5-triene-4-carboxylate (24% yield); m/z=372.0 (MH)⁺; 1H NMR (500 MHz, DMSO-d6) δ 1.05 (s, 6H), 1.30 (s, 6H), 1.65 (s, 2H), 2.80 (s, 2H), 3.05 (s, 3H). Exchangeable protons not observed.

### Example 222

### 3-Bromo-9,9,11,11-tetramethyl-7-thia-2,5-diazatricyclo[6.4.0.0^{2,6}]dodeca-1(8),3,5-triene-4-carboxylic acid

To a solution of 9,9,11,11-tetramethyl-7-thia-2,5-diazatricyclo[6.4.0.0^{2,6}]dodeca-1(8),3,5-triene-4-carboxylic acid (250 mg, 0.898 mmol) in EtOH (10 mL) at 0° C. was added NBS (176 mg, 0.988 mmol) in three portions. The reaction mixture stirred at 0° C. for 1 h, then allowed to warm to room temperature and stirred for a further 1 h. The reaction mixture was concentrated and the residue partitioned between EtOAc (30 mL) and water (30 mL). The phases were separated and the aqueous layer was treated with 1M HCl (10 mL). The resultant mixture was filtered and air dried to afford the title compound as a white solid (195 mg, 61% yield); m/z=356.8 (MH)⁺; 1H NMR (250 MHz, Methanol-d4) δ 1.19 (s, 6H), 1.41 (s, 6H), 1.76 (s, 2H), 3.01 (s, 2H). Exchangeable protons not observed.

### Example 223

### 9-(Pyrrolidin-1-yl)-7-thia-2,5-diazatricyclo[6.4.0.0^{2,6}]dodeca-1(8),3,5,9,11-pentaene-4-carboxylic acid

To a stirred suspension of ethyl 9-(pyrrolidin-1-yl)-7-thia-2,5-diazatricyclo[6.4.0.0^{2,6}]dodeca-1(8),3,5,9,11-pentaene-4-carboxylate (71%, 41 mg, 0.092 mmol) in EtOH (2 mL) and water (2 mL) was added LiOH.H₂O (12 mg, 0.28 mmol). The resulting suspension stirred at room temperature for 2.5 days. The suspension was heated to 45° C. for 2 h then more LiOH.H₂O (6.0 mg, 0.14 mmol) was added and the resulting suspension heated to 45° C. for a further 4 h. The solvent was removed under reduced pressure and water (10 mL) was added. The resulting solution was washed with EtOAc (10 mL) and the aqueous layer subsequently acidified to pH 1 using 1M HCl_{(aq)}. EtOAc (15 mL) was added to the resulting suspension and the mixture filtered, affording the title compound as a pale-orange solid (14 mg, 53% yield); m/z=288.0 (MH)⁺; 1H NMR (500 MHz, DMSO-d6) δ 1.95-2.03 (m, 4H), 3.51-3.58 (m, 4H), 6.66 (d, 1H), 7.36 (t, 1H), 7.46 (d, 1H), 8.90 (s, 1H), 12.67 (s, 1H).

### Example 224

### 2-{9-Chloro-10-fluoro-7-thia-2,5-diazatricyclo[6.4.0.0^{2,6}]dodeca-1(8),3,5,9,11-pentaen-4-yl}-2-hydroxyacetic acid

To a solution of potassium hexamethyldisilazide (99 mg, 0.50 mmol) in dry THF (5 mL) at −78° C. under nitrogen was added a solution of ethyl 2-{9-chloro-10-fluoro-7-thia-2,5-diazatricyclo[6.4.0.0^{2,6}]dodeca-1(8),3,5,9,11-pentaen-4-yl}acetate (107 mg, 0.332 mmol) in THF dropwise. The mixture stirred for 15 min and then a solution of the 3-phenyl-2-(phenylsulfonyl)-1,2-oxaziridine (130 mg, 0.498 mmol) was added. The mixture was stirred for 20 min at −78° C. The solvent was evaporated and the mixture stirred in MeOH (10 mL) and 2M NaOH_{(aq)} (3 mL) for 1 h. The solvent was evaporated and the mixture was partitioned between DCM and water. The DCM was discarded and the aqueous layer made acidic with 2M HCl_{(aq)}. The mixture was then extracted with EtOAc (3×20 mL). The combined organic extracts were dried (Na₂SO₄), filtered and evaporated. The crude product was purified by automated reverse phase HPLC (low pH method) to afford the title compound as a white solid (16 mg, 16% yield); m/z=301.0 (MH)⁺; 1H NMR (500 MHz, DMSO-d6) δ 5.09 (s, 1H), 7.69 (t, 1H), 8.14 (dd, 1H), 8.31 (s, 1H). Exchangeable protons not observed.

### Example 225

### 3-{1-[(tert-Butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylic acid

To a stirred solution of ethyl 3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylate (160 mg, 0.35 mmol) in EtOH:H₂O (1:1; 5 mL) was added LiOH.H₂O (29 mg, 0.69 mmol) and heated at 60° C. for 1 h. The mixture was concentrated. The residue was then taken up in EtOAc (20 mL) and water (10 mL). The phases were separated. The organic layer was concentrated and purified using FCC on silica, eluent: DCM and DMAW 120, to afford the title compound as a red solid (20 mg, 98% purity, 13% yield); m/z=434.1 (MH)⁺; 1H NMR (500 MHz, Methanol-d4) δ 1.13 (s, 6H), 1.32 (s, 6H), 1.46 (s, 9H), 1.69 (s, 2H), 2.71 (s, 2H), 4.19 (s, 2H), 4.29-4.49 (m, 2H), 4.69 (s, 1H). Exchangeable protons not observed.

### Example 226

### 9-Chloro-10-fluoro-3-(trifluoromethyl)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 9-chloro-10-fluoro-3-(trifluoromethyl)-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate (43% yield); m/z=338.9 (MH)⁺; 1H NMR (500 MHz, DMSO-d6) δ 7.76 (t, 1H), 7.89 (dd, 1H), 13.80 (s, 1H).

### Example 227

### 10-tert-Butyl-7-thia-2,5-diazatricyclo[6.4.0.0^{2,6}]dodeca-1(8),5,9,11-tetraene-4-carboxylic acid

To a solution of ethyl 10-tert-butyl-7-thia-2,5-diazatricyclo[6.4.0.0^{2,6}]dodeca-1(8),5,9,11-tetraene-4-carboxylate (200 mg, 0.657 mmol) in EtOH (5 mL) and water (1 mL) was added LiOH.H₂O (55 mg, 1.3 mmol). The resulting solution was stirred at 50° C. for 1 h. The reaction mixture was evaporated to dryness and dissolved in water (2 mL). 1M HCl was added to adjust the pH to pH3. The resulting aqueous solution was then extracted with a mixture of IPA/CHCl₃ (3:1) (3×25 mL). The combined organic layers were evaporated to dryness to afford a colourless oil. MeCN (10 mL) was then added and a white precipitate formed. The mixture was filtered and the solid dried in vacuo to afford the title compound as a white solid (110 mg, 61% yield); m/z=277.1 (MH)⁺; 1H NMR (500 MHz, DMSO-d6) δ 1.26 (s, 9H), 4.02-4.12 (m, 2H), 5.02 (dd, 1H), 6.90 (d, 1H), 7.28 (dd, 1H), 7.62 (d, 1H). Exchangeable protons not observed.

### Example 228

### 9-Chloro-10-fluoro-7-thia-2,5-diazatricyclo[6.4.0.0^{2,6}]dodeca-1(8),5,9,11-tetraene-4-carboxylic acid

To a stirred suspension of ethyl 9-chloro-10-fluoro-7-thia-2,5-diazatricyclo[6.4.0.0^{2,6}]dodeca-1(8),5,9,11-tetraene-4-carboxylate (90%, 150 mg, 0.450 mmol) and methyl 9-chloro-10-fluoro-7-thia-2,5-diazatricyclo[6.4.0.0^{2,6}]dodeca-1(8),5,9,11-tetraene-4-carboxylate (102 mg, 0.352 mmol) in EtOH (4 mL) and water (4 mL) was added LiOH.H₂O (38 mg, 0.90 mmol). The resulting suspension was heated to 45° C. for 1 h. The solvent was removed under reduced pressure and water (5 mL) was added. The suspension was adjusted to pH 1-2 using 1M HCl_{(aq)}. The aqueous layer was washed with EtOAc (10 mL) and 1:1 IPA:CHCl₃ (2×5 mL). The organic washings contained impure product. The aqueous layer was then extracted using 1:1 IPA:CHCl₃ (2×10 mL). These latter organic extracts were combined, dried (MgSO₄), the mixture filtered and the filtrate evaporated under reduced pressure to afford the title compound as a pink solid (45 mg, 21% yield); m/z=273.0 (MH)⁺; 1H NMR (500 MHz, MeOD-d4) δ 4.55 (d, 2H), 5.26 (t, 1H), 7.28 (dd, 1H), 7.41 (t, 1H). Exchangeable protons not observed.

### Example 229

### 3-Ethynyl-9,9,11,11-tetramethyl-7-thia-2,5-diazatricyclo[6.4.0.0^{2,6}]dodeca-1(8),3,5-triene-4-carboxylic acid

A suspension of ethyl 3-ethynyl-9,9,11,11-tetramethyl-7-thia-2,5-diazatricyclo[6.4.0.0^{2,6}]dodeca-1(8),3,5-triene-4-carboxylate (17 mg, 0.05 mmol) and LiOH.H₂O (4 mg, 0.1 mmol) in a mixture of EtOH/H₂O [1:1 (v/v); 0.5 mL] was heated at 60° C. for 1 h. After this time, the reaction mixture was diluted with water and EtOAc, with the organic layer discarded and the aqueous layer acidified to pH 1 by addition of 1M HCl_{(aq)}. The aqueous layer was extracted with EtOAc. The organic layer was washed with saturated brine, dried (MgSO₄), the mixture filtered and the filtrate concentrated. The residue was purified by FCC, eluting with 0-35% DMAW 90 in DCM, to afford the title compound as a scarlet solid (8 mg, 51% yield); m/z=303.0 (MH)⁺; 1H NMR (250 MHz, DMSO-d6) δ 1.09 (s, 6H), 1.32 (s, 6H), 1.67 (s, 2H), 2.85 (s, 2H), 4.94 (s, 1H). Exchangeable protons not observed.

### Example 230

### 10-Chloro-11-(trifluoromethoxy)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 10-chloro-11-(trifluoromethoxy)-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate (12% yield); m/z=337.0 (MH)⁺; 1H NMR (500 MHz, DMSO-d6) δ 8.51 (s, 1H), 8.56 (s, 1H), 9.05 (s, 1H), 12.76 (s, 1H).

### Example 231

### 10-Bromo-7-thia-2,5-diazatricyclo[6.4.0.0^{2,6}]dodeca-1(8),5,9,11-tetraene-4-carboxylic acid

The procedure to prepare 9-chloro-10-fluoro-7-thia-2,5-diazatricyclo[6.4.0.0^{2,6}]dodeca-1(8),5,9,11-tetraene-4-carboxylic acid was used except that ethyl 10-bromo-7-thia-2,5-diazatricyclo[6.4.0.0^{2,6}]dodeca-1(8),5,9,11-tetraene-4-carboxylate was substituted for methyl 9-chloro-10-fluoro-7-thia-2,5-diazatricyclo[6.4.0.0^{2,6}]dodeca-1(8),5,9,11-tetraene-4-carboxylate (57% yield); m/z=298.9 (MH)⁺; 1H NMR (500 MHz, DMSO-d6) δ 4.60-4.68 (m, 2H), 5.42 (dd, 1H), 7.48 (d, 1H), 7.73 (dd, 1H), 8.22 (s, 1H). Exchangeable protons not observed.

### Example 232

### 9-Chloro-10-fluoro-3-methanesulfonamido-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was followed except that ethyl 9-chloro-10-fluoro-3-methanesulfonamido-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate. Trituration was in MeOH (56% yield); m/z=364.0 (MH)⁺; 1H NMR (500 MHz, DMSO-d6) δ 3.18 (s, 3H), 7.77 (t, 1H), 8.16 (dd, 1H). Exchangeable protons not observed.

### Example 233

### 9,9,11,11-Tetramethyl-3-(morpholin-4-yl)-7-thia-2,5-diazatricyclo[6.4.0.0^{2,6}]dodeca-1(8),3,5-triene-4-carboxylic acid

The procedure to prepare 3-ethynyl-9,9,11,11-tetramethyl-7-thia-2,5-diazatricyclo[6.4.0.0^{2,6}]dodeca-1(8),3,5-triene-4-carboxylic acid was followed except that ethyl 9,9,11,11-tetramethyl-3-(morpholin-4-yl)-7-thia-2,5-diazatricyclo[6.4.0.0^{2,6}]dodeca-1(8),3,5-triene-4-carboxylate was substituted for ethyl 3-ethynyl-9,9,11,11-tetramethyl-7-thia-2,5-diazatricyclo[6.4.0.0^{2,6}]dodeca-1(8),3,5-triene-4-carboxylate and purification was by low pH HPLC rather than FCC (27% yield); m/z=364.6 (MH)⁺; 1H NMR (500 MHz, DMSO-d6) δ 1.10 (s, 6H), 1.29 (s, 6H), 1.64 (s, 2H), 2.85 (s, 2H), 3.48-3.59 (m, 4H), 4.05-4.12 (m, 4H). Exchangeable protons not observed.

### Example 234

### 9-Chloro-10-fluoro-3-methyl-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was followed except that ethyl 9-chloro-10-fluoro-3-methyl-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate. No trituration was needed (72% yield); m/z=284.9 (MH)⁺; 1H NMR (500 MHz, DMSO-d6) δ 2.97 (s, 3H), 7.66 (t, 1H), 8.06 (dd, 1H), 12.65 (s, 1H).

### Example 235

### 3-(Azetidin-3-yl)-9,9,11,11-tetramethyl-7-thia-2,5-diazatricyclo[6.4.0.0^{2,6}]dodeca-1(8),3,5-triene-4-carboxylic acid

To a solution of 3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylic acid (70 mg, 0.16 mmol) in DCM (2 mL) under nitrogen was added TFA (0.1 mL) at 0° C. The reaction mixture was allowed to warm up to room temperature and stirred overnight for 18 h. It was then cooled to 0° C. and more TFA (0.3 mL) was added dropwise. The mixture was allowed to warm to room temperature and stirred for 6 h, then concentrated, followed by azeotroping with toluene, to afford the title compound (TFA salt) as a pink solid (50 mg, 69% yield); m/z=334.1 (MH)⁺; 1H NMR (500 MHz, DMSO-d6) 1.10 (s, 6H), 1.31 (s, 6H), 1.64 (s, 2H), 2.76 (s, 2H), 4.16-4.25 (m, 2H), 4.50 (p, 2H), 4.75 (p, 1H), 8.52 (s, 1H), 8.71 (s, 1H). TFA salt. Exchangeable protons not observed.

### Example 236

### (4S)-9-Chloro-10-fluoro-7-thia-2,5-diazatricyclo[6.4.0.0^{2,6}]dodeca-1(8),5,9,11-tetraene-4-carboxylic acid (Configuration Arbitrarily Assigned)

To a stirred suspension of ethyl (4S)-9-chloro-10-fluoro-7-thia-2,5-diazatricyclo[6.4.0.0^{2,6}]dodeca-1(8),5,9,11-tetraene-4-carboxylate (configuration arbitrarily assigned) (48 mg, 0.16 mmol) in EtOH (1 mL) and water (1 mL) was added LiOH.H₂O (6.3 mg, 0.16 mmol). The resulting suspension was stirred at room temperature for 1 h. More LiOH.H₂O (3 mg, 0.08 mmol) was added and the suspension stirred at room temperature for 16 h. The reaction mixture was acidified to ˜pH 2 using 1M HCl_{(aq)}. The aqueous layer was extracted with IPA:CHCl₃ (1:1, 4×5 mL). The organic fractions were combined, dried over MgSO₄ and the solvent removed under reduced pressure, to afford the title compound as a white solid (12 mg, 28% yield); m/z=273.0 (MH)⁺; 1H NMR (500 MHz, DMSO-d6) δ 4.13-4.26 (m, 2H), 5.08-5.17 (m, 1H), 7.03-7.10 (m, 1H), 7.40 (t, 1H). Exchangeable protons not observed.

### Example 237

### (4R)-9-Chloro-10-fluoro-7-thia-2,5-diazatricyclo[6.4.0.0^{2,6}]dodeca-1(8),5,9,11-tetraene-4-carboxylic acid (Configuration Arbitrarily Assigned)

The procedure to prepare (4S)-9-chloro-10-fluoro-7-thia-2,5-diazatricyclo[6.4.0.0^{2,6}]dodeca-1(8),5,9,11-tetraene-4-carboxylic acid was followed except that ethyl (4R)-9-chloro-10-fluoro-7-thia-2,5-diazatricyclo[6.4.0.0^{2,6}]dodeca-1(8),5,9,11-tetraene-4-carboxylate (configuration arbitrarily assigned) was substituted for ethyl (4S)-9-chloro-10-fluoro-7-thia-2,5-diazatricyclo[6.4.0.0^{2,6}]dodeca-1(8),5,9,11-tetraene-4-carboxylate (53% yield); m/z=273.0 (MH)⁺; 1H NMR (500 MHz, DMSO) δ 4.57-4.67 (m, 2H), 5.41 (dd, 1H), 7.52 (dd, 1H), 7.65 (t, 1H). Exchangeable protons not observed.

### Example 238

### 9-Chloro-10-fluoro-3-(morpholin-4-yl)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was followed except that ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate was substituted for ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate. In addition, trituration of the solid was carried out using DMSO/MeOH/water (54% yield); m/z=356.0 (MH)⁺; 1H NMR (500 MHz, DMSO-d6) δ 2.89 (d, 2H), 3.56-3.62 (m, 2H), 3.74-3.83 (m, 2H), 3.84-3.90 (m, 2H), 7.67 (t, 1H), 8.36 (dd, 1H), 12.79 (s, 1H).

### Example 239

### 3-Acetyl-9,9,11,11-tetramethyl-7-thia-2,5-diazatricyclo[6.4.0.0^{2,6}]dodeca-1(8),3,5-triene-4-carboxylic acid

Ethyl 3-bromo-9,9,11,11-tetramethyl-7-thia-2,5-diazatricyclo[6.4.0.0^{2,6}]dodeca-1(8),3,5-triene-4-carboxylate (200 mg, 0.503 mmol), 1-(ethenyloxy)butane (327 μL, 2.52 mmol), Pd(OAc)₂ (11 mg, 0.05 mmol), propane-1,3-diylbis(diphenylphosphane) (42 mg, 0.10 mmol), K₂CO₃ (90 mg, 0.65 mmol) and DMF (2 mL) were added to a pressure tube. The suspension was degassed, the tube sealed and then heated to 100° C. for 4 h. 1M HCl_{(aq)} (1 mL) was added and the reaction mixture was concentrated under reduced pressure. The residue was diluted with MeCN (1 mL) and 1M HCl_{(aq)} (1 mL). The resulting solution was stirred for 30 min at room temperature and then concentrated. The residue was extracted with EtOAc (2×15 mL) was added. The combined organic extracts were washed with water (20 mL) and brine (20 mL) and dried (MgSO₄), filtered and concentrated. To this residue was added EtOH (2 mL) and water (2 mL) and LiOH.H₂O (98 mg, 2.3 mmol). The resulting suspension was stirred at room temperature for 1 h. The reaction mixture was concentrated under reduced pressure and EtOAc (10 mL) and water (10 mL) added. The phases were separated and the aqueous layer acidified to ˜pH 2 using 1M HCl_{(aq)} and extracted with IPA:CHCl₃ (1:1, 4×10 mL). The combined organic extracts were washed with water (10 mL) and brine (10 mL) and dried (MgSO₄), filtered and concentrated. The residue was purified using FCC on reverse-phase silica, eluent: 0-100% MeCN in water (+0.1% formic acid). Further purified was performed via automated reverse phase HPLC (low pH method) to afford the title compound as an off-white solid (7.7 mg, 23% yield); m/z=321.1 (MH)⁺; 1H NMR (500 MHz, MeOD-d4) δ 1.09 (s, 6H), 1.39 (s, 6H), 1.71 (s, 2H), 2.39 (s, 2H), 2.73 (s, 3H). Exchangeable protons not observed.

### Example 240

### 10-Bromo-9-chloro-7-thia-2,5-diazatricyclo[6.4.0.0^{2,6}]dodeca-1(8),5,9,11-tetraene-4-carboxylic acid

To a stirred suspension of ethyl 10-bromo-9-chloro-7-thia-2,5-diazatricyclo[6.4.0.0^{2,6}]dodeca-1(8),5,9,11-tetraene-4-carboxylate (100 mg, 0.26 mmol) in EtOH (4 mL) and water (4 mL) was added LiOH.H₂O (22 mg, 0.52 mmol). The resulting suspension was stirred at room temperature for 2 h. The suspension was concentrated under reduced pressure and washed with EtOAc (10 mL). The aqueous layer was then adjusted to pH 2 using 1M HCl_{(aq)} and extracted with IPA:CHCl₃ (1:1, 3×5 mL). The combined organic extracts were washed with brine (5 mL), dried (MgSO₄), filtered and concentrated. The crude product was triturated in MeOH to afford the title compound as a pink solid (11 mg, 12% yield); m/z=332.9 (MH)⁺; 1H NMR (500 MHz, DMSO-d6) δ 4.06-4.16 (m, 2H), 5.08 (dd, 1H), 6.93 (d, 1H), 7.65 (d, 1H). Exchangeable protons not observed.

### Example 241

### 9,10-Dichloro-12-[2-(morpholin-4-yl)ethoxy]-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was followed except that ethyl 9,10-dichloro-12-[2-(morpholin-4-yl)ethoxy]-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate and purification was by preparative HPLC (low pH) rather than trituration (2% yield); m/z=416.0 (MH)⁺; 1H NMR (500 MHz, DMSO-d6) δ 2.81 (t, 2H), 3.60-3.63 (m, 4H), 4.43 (t, 2H), 7.67 (s, 1H), 8.22 (s, 1H), 8.69 (s, 1H). Formic acid salt. 4 Protons hidden. Exchangeable protons not observed.

### Example 242

### 10-tert-Butyl-3-(morpholin-4-ylmethyl)-7-thia-2,5-diazatricyclo[6.4.0.0^{2,6}]dodeca-1(8),3,5-triene-4-carboxylic acid

A suspension of ethyl 10-tert-butyl-3-(morpholin-4-ylmethyl)-7-thia-2,5-diazatricyclo[6.4.0.0^{2,6}]dodeca-1(8),3,5-triene-4-carboxylate (27 mg, 0.068 mmol) and LiOH.H₂O (6 mg, 0.13 mmol) in EtOH/H₂O [1:1 (v/v); 0.7 mL] was heated at 60° C. for 90 min. After this time, the reaction mixture was diluted with water and the aqueous layer acidified to pH 1 by addition of 1M HCl_{(aq)}. The aqueous layer was extracted with 20% IPA in DCM solution (×2). The combined organic extracts were washed with brine, dried (MgSO₄), filtered and concentrated to afford the title compound as an off-white solid (23 mg, 90% yield); m/z=378.1 (MH)⁺; 1H NMR (500 MHz, DMSO-d6) δ 0.95 (s, 9H), 1.38-1.50 (m, 1H), 1.53-1.63 (m, 1H), 2.08-2.11 (m, 1H), 2.70-2.78 (m, 1H), 2.88-2.95 (m, 1H), 3.66 (s, 4H), 4.38 (s, 2H). 6 Protons hidden. Exchangeable protons not observed.

### Example 243

### 10-Bromo-9-chloro-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was followed except that ethyl 10-bromo-9-chloro-7-thia-2,5-diazatricyclo[6.4.0.026]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate and final purification was achieved by trituration in MeOH (50% yield); m/z=330.7 (MH)⁺; 1H NMR (500 MHz, DMSO-d6) δ 8.01 (d, 1H), 8.11 (d, 1H), 9.01 (s, 1H), 12.79 (s, 1H).

### Example 244

### 10-tert-Butyl-3-[(2-oxopiperidin-1-yl)methyl]-7-thia-2,5-diazatricyclo[6.4.0.0^{2,6}]dodeca-1(8),3,5-triene-4-carboxylic acid

A suspension of ethyl 10-tert-butyl-3-[(2-oxopiperidin-1-yl)methyl]-7-thia-2,5-diazatricyclo[6.4.0.0^{2,6}]dodeca-1(8),3,5-triene-4-carboxylate (33 mg, 0.079 mmol) and LiOH.H₂O (7 mg, 0.2 mmol) in EtOH/H₂O [1:1 (v/v); 0.8 mL] was heated at 60° C. for 1 h. After this time, the reaction mixture was treated with water, 2M aqueous NaOH and EtOAc. The aqueous layer was retained and the organic layer extracted with more 2M aqueous NaOH_{(aq)}. The combined aqueous extracts were washed with EtOAc, acidified to pH 3 by addition of 4M HCl_{(aq)} and then extracted with EtOAc (×2). The combined organic extracts washed with brine, dried (MgSO₄), filtered and concentrated to afford the title compound as a tan solid (26 mg, 83% yield); m/z=390.1 (MH)⁺; 1H NMR (500 MHz, Methanol-d4) δ 0.99 (s, 9H), 1.45-1.55 (m, 1H), 1.59-1.67 (m, 1H), 1.71-1.81 (m, 4H), 2.12-2.18 (m, 1H), 2.40 (t, 2H), 2.55 (t, 1H), 2.63-2.72 (m, 1H), 2.76 (dd, 1H), 3.04 (dd, 1H), 3.21 (t, 2H), 5.20 (d, 1H), 5.33 (d, 1H). Exchangeable protons not observed.

### Example 245

### 3,9,9,11,11-Pentamethyl-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was followed except that ethyl 3,9,9,11,11-pentamethyl-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate (36% yield); m/z=293.1 (MH)⁺; 1H NMR (500 MHz, Methanol-d4) δ 1.19 (s, 6H), 1.40 (s, 6H), 1.74 (s, 2H), 2.84 (s, 2H), 2.89 (s, 3H). Exchangeable protons not observed.

### Example 246

### 9,9,11,11-Tetramethyl-3-(morpholin-4-ylmethyl)-7-thia-2,5-diazatricyclo[6.4.0.0^{2,6}]dodeca-1(8),3,5-triene-4-carboxylic acid

The procedure to prepare 10-tert-butyl-3-[(2-oxopiperidin-1-yl)methyl]-7-thia-2,5-diazatricyclo[6.4.0.0^{2,6}]dodeca-1(8),3,5-triene-4-carboxylic acid was followed except that ethyl 9,9,11,11-tetramethyl-3-(morpholin-4-ylmethyl)-7-thia-2,5-diazatricyclo[6.4.0.0^{2,6}]dodeca-1(8),3,5-triene-4-carboxylate was substituted for ethyl 10-tert-butyl-3-[(2-oxopiperidin-1-yl)methyl]-7-thia-2,5-diazatricyclo[6.4.0.0^{2,6}]dodeca-1(8),3,5-triene-4-carboxylate (56% yield); m/z=378.2 (MH)⁺; 1H NMR (500 MHz, DMSO-d6) δ 1.09 (s, 6H), 1.32 (s, 6H), 1.66 (s, 2H), 2.42 (s, 4H), 2.98 (s, 2H), 3.53 (s, 4H), 4.03 (s, 2H). Exchangeable protons not observed.

### Example 247

### 3-(2-Methoxyphenyl)-9,9,11,11-tetramethyl-7-thia-2,5-diazatricyclo[6.4.0.0^{2,6}]dodeca-1(8),3,5-triene-4-carboxylic acid

The procedure to prepare 10-tert-butyl-3-[(2-oxopiperidin-1-yl)methyl]-7-thia-2,5-diazatricyclo[6.4.0.0^{2,6}]dodeca-1(8),3,5-triene-4-carboxylic acid was followed except that ethyl 3-(2-methoxyphenyl)-9,9,11,11-tetramethyl-7-thia-2,5-diazatricyclo[6.4.0.0^{2,6}]dodeca-1(8),3,5-triene-4-carboxylate was substituted for ethyl 10-tert-butyl-3-[(2-oxopiperidin-1-yl)methyl]-7-thia-2,5-diazatricyclo[6.4.0.0^{2,6}]dodeca-1(8),3,5-triene-4-carboxylate (53% yield); m/z=385.4 (MH)⁺; 1H NMR (500 MHz, DMSO-d6) δ 0.82 (s, 3H), 0.89 (s, 3H), 1.27 (s, 3H), 1.31 (s, 3H), 1.56 (d, 2H), 1.71-1.86 (m, 2H), 3.71 (s, 3H), 7.00-7.03 (m, 1H), 7.10 (d, 1H), 7.31 (dd, 1H), 7.46-7.51 (m, 1H). Exchangeable protons not observed.

### Example 248

### 9-Chloro-10-(pyrrolidin-1-yl)-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was followed except that ethyl 9-chloro-10-(pyrrolidin-1-yl)-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (76% yield); m/z=322.0 (MH)⁺; 1H NMR (250 MHz, DMSO-d6) δ 1.86-2.02 (m, 4H), 3.42 (t, 4H), 7.14 (d, 1H), 7.97 (d, 1H), 8.86 (s, 1H), 12.61 (s, 1H).

### Example 249

### 9-Chloro-10-fluoro-4-(2-methylpropyl)-7-thia-2,5-diazatricyclo[6.4.0.0^{2,6}]dodeca-1(8),5,9,11-tetraene-4-carboxylic acid

To a solution of ethyl 9-chloro-10-fluoro-7-thia-2,5-diazatricyclo[6.4.0.0^{2,6}]dodeca-1(8),5,9,11-tetraene-4-carboxylate (250 mg, 0.831 mmol) and sodium 2-methylpropan-2-olate (80 mg, 0.83 mmol) in DMF (10 mL) was added 1-iodo-2-methylpropane (134 μL, 1.66 mmol). The resulting mixture was stirred at room temperature under nitrogen for 3 h. 1M HCl_{(aq)} (10 mL) was added and the mixture extracted with DCM (5×25 mL). The combined organic extracts were dried (Na₂SO₄), filtered and the filtrate evaporated to dryness. The crude product was purified by automated reverse phase HPLC (low pH method) to afford the title compound as the formic acid salt as a colourless oil which crystalized on standing (20 mg, 7% yield); m/z=329.4 (MH)⁺; 1H NMR (500 MHz, Methanol-d4) δ 1.00 (dd, 6H), 1.86 (dq, 1H), 2.01 (dd, 2H), 4.14 (d, 1H), 4.70 (d, 1H), 7.22 (dd, 1H), 7.36 (t, 1H). Exchangeable protons not observed.

### Example 250

### 9-Chloro-10-fluoro-4-methyl-7-thia-2,5-diazatricyclo[6.4.0.0^{2,6}]dodeca-1(8),5,9,11-tetraene-4-carboxylic acid

To a solution of ethyl 9-chloro-10-fluoro-7-thia-2,5-diazatricyclo[6.4.0.0^{2,6}]dodeca-1(8),5,9,11-tetraene-4-carboxylate (250 mg, 0.83 mmol) and sodium 2-methylpropan-2-olate (80 mg, 0.83 mmol) in DMF (10 mL) was added iodomethane (104 μL, 1.66 mmol). The resulting mixture was stirred at room temperature under nitrogen for 3 h. After this time, LiOH.H₂O (70 mg, 1.66 mmol) and water (1 mL) were added and the mixture stirred at 45° C. for 2 h. The mixture was evaporated to dryness and partitioned between DCM (25 mL) and 1M HCl_{(aq)} (25 mL). The organic layer was extracted with water. The combined aqueous extracts were evaporated to dryness and stirred in MeOH. The MeOH was decanted and evaporated to dryness. The residue purified by automated reverse phase HPLC (low pH method) to afford the title compound as a light pink solid (66 mg, 28% yield); m/z=287.0 (MH)⁺; 1H NMR (500 MHz, DMSO-d6) δ 1.50 (s, 3H), 3.76 (d, 1H), 4.29 (d, 1H), 6.96 (dd, 1H), 7.36 (dd, 1H). Exchangeable protons not observed.

### Example 251

### 10-tert-Butyl-3-{[(2-methoxyethyl)(methyl)amino]methyl}-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was followed except that ethyl 10-tert-butyl-3-{[(2-methoxyethyl)(methyl)amino]methyl}-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate (34% yield); m/z=380.6 (MH)⁺; 1H NMR (500 MHz, Methanol-d4) δ 0.90 (s, 9H), 1.19 (s, 3H), 1.39-1.59 (m, 2H), 2.11-2.20 (m, 1H), 2.39-2.51 (m, 1H), 2.65-2.75 (m, 2H), 2.71 (s, 3H), 2.99 (d, 1H), 3.16 (s, 2H), 3.57 (s, 2H), 4.56 (s, 2H). Exchangeable protons not observed.

### Example 252

### 9-Chloro-10-cyclopropyl-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was followed except that ethyl 9-chloro-10-cyclopropyl-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (85% yield); m/z=293.0 (MH)⁺; 1H NMR (500 MHz, DMSO-d6) δ 0.77-0.88 (m, 2H), 1.03-1.15 (m, 2H), 2.20-2.26 (m, 1H), 7.27 (d, 1H), 8.06 (d, 1H), 8.96 (s, 1H). Exchangeable protons not observed.

### Example 253

### 3-Bromo-10-tert-butyl-7-thia-2,5-diazatricyclo[6.4.0.0^{2,6}]dodeca-1(8),3,5-triene-4-carboxylic acid

The procedure to prepare 10-bromo-9-chloro-7-thia-2,5-diazatricyclo[6.4.0.0^{2,6}]dodeca-1(8),5,9,11-tetraene-4-carboxylic acid was used except that ethyl 3-bromo-10-tert-butyl-7-thia-2,5-diazatricyclo[6.4.0.0^{2,6}]dodeca-1(8),3,5-triene-4-carboxylate was substituted for ethyl 10-bromo-9-chloro-7-thia-2,5-diazatricyclo[6.4.0.0^{2,6}]dodeca-1(8),5,9,11-tetraene-4-carboxylate and purification was by silica chromatography (eluent: DCM/DMAW 120) rather than by trituration (89% yield); m/z=357.0 (MH)⁺; 1H NMR (500 MHz, Methanol-d4) δ 1.00 (s, 9H), 1.42-1.58 (m, 1H), 1.58-1.74 (m, 1H), 2.13-2.28 (m, 1H), 2.46-2.62 (m, 1H), 2.69-2.83 (m, 1H), 2.83-3.00 (m, 1H), 3.38-3.51 (m, 1H). Exchangeable protons not observed.

### Example 254

### 3-(4-Methoxyphenyl)-9,9,11,11-tetramethyl-7-thia-2,5-diazatricyclo[6.4.0.0^{2,6}]dodeca-1(8),3,5-triene-4-carboxylic acid

A solution of ethyl 3-(4-methoxyphenyl)-9,9,11,11-tetramethyl-7-thia-2,5-diazatricyclo[6.4.0.0^{2,6}]dodeca-1(8),3,5-triene-4-carboxylate (85 mg, 0.21 mmol) and LiOH.H₂O (26 mg, 0.62 mmol) in EtOH:H₂O (1:1, 2 mL) was warmed to 60° C. for 1 h. The reaction mixture was concentrated in vacuo and purified using automated reverse phase HPLC (low pH method) to afford the title compound as an off white solid (25% yield); m/z=385.2 (MH)⁺; 1H NMR (250 MHz, DMSO-d6) δ 0.85 (s, 6H), 1.29 (s, 6H), 1.57 (s, 2H), 1.82 (s, 2H), 3.82 (s, 3H), 6.99 (d, 2H), 7.37 (d, 2H). Exchangeable protons not observed.

### Example 255

### 10-tert-Butyl-3-(acetamidomethyl)-7-thia-2,5-diazatricyclo[6.4.0.0^{2,6}]dodeca-1(8),3,5-triene-4-carboxylic acid

The procedure to prepare 3-ethynyl-9,9,11,11-tetramethyl-7-thia-2,5-diazatricyclo[6.4.0.0^{2,6}]dodeca-1(8),3,5-triene-4-carboxylic acid was followed except that ethyl 10-tert-butyl-3-(acetamidomethyl)-7-thia-2,5-diazatricyclo[6.4.0.0^{2,6}]dodeca-1(8),3,5-triene-4-carboxylate was substituted for ethyl 3-ethynyl-9,9,11,11-tetramethyl-7-thia-2,5-diazatricyclo[6.4.0.0^{2,6}]dodeca-1(8),3,5-triene-4-carboxylate and back extraction was by 20% IPA in DCM rather than EtOAc (47% yield); m/z=350.2 (MH)⁺; 1H NMR (500 MHz, Methanol-d₄) δ 0.99 (s, 9H), 1.43-1.58 (m, 1H), 1.58-1.70 (m, 1H), 1.94 (s, 3H), 2.13-2.26 (m, 1H), 2.41-2.63 (m, 1H), 2.67-2.79 (m, 1H), 2.79-2.93 (m, 1H), 3.10-3.24 (m, 1H), 4.86-5.04 (m, 2H). Exchangeable protons not observed.

### Example 256

### 10-tert-Butyl-3-(hydroxymethyl)-7-thia-2,5-diazatricyclo[6.4.0.0^{2,6}]dodeca-1(8),3,5-triene-4-carboxylic acid

The procedure to prepare 3-ethynyl-9,9,11,11-tetramethyl-7-thia-2,5-diazatricyclo[6.4.0.0^{2,6}]dodeca-1(8),3,5-triene-4-carboxylic acid was followed except that ethyl 3-[(acetyloxy)methyl]-10-tert-butyl-7-thia-2,5-diazatricyclo[6.4.0.0^{2,6}]dodeca-1(8),3,5-triene-4-carboxylate was substituted for ethyl 3-ethynyl-9,9,11,11-tetramethyl-7-thia-2,5-diazatricyclo[6.4.0.0^{2,6}]dodeca-1(8),3,5-triene-4-carboxylate and back extraction was by 20% IPA in DCM rather than EtOAc (73% yield); m/z=309.4 (MH)⁺; 1H NMR (500 MHz, Methanol-d4) δ 1.02 (s, 9H), 1.54-1.60 (m, 1H), 1.63-1.70 (m, 1H), 2.18-2.25 (m, 1H), 2.53-2.60 (m, 1H), 2.73-2.83 (m, 1H), 2.86-2.97 (m, 1H), 3.31-3.35 (m, 1H), 5.02-5.12 (m, 1H), 5.22-5.30 (m, 1H). Exchangeable protons not observed.

### Example 257

### 10-tert-Butyl-3-{[methoxy(methyl)amino]methyl}-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 10-tert-butyl-3-{[methoxy(methyl)amino]methyl}-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate and that purification was by FCC on silica, eluent 0-100% DMAW 120 in DCM (92% yield); m/z=352.2 (MH)⁺; 1H NMR (500 MHz, Methanol-d4) δ 1.02 (s, 9H), 1.52-1.58 (m, 1H), 1.64-1.70 (m, 1H), 2.18-2.25 (m, 1H), 2.53-2.61 (m, 1H), 2.64 (s, 3H), 2.75-2.81 (m, 1H), 2.90-2.97 (m, 1H), 3.27 (s, 3H), 3.38-3.45 (m, 1H), 4.42-4.51 (m, 2H). Exchangeable protons not observed.

### Example 258

### 3-Cyclopropyl-9,9,11,11-tetramethyl-7-thia-2,5-diazatricyclo[6.4.0.0^{2,6}]dodeca-1(8),3,5-triene-4-carboxylic acid

The procedure to prepare 3-(4-methoxyphenyl)-9,9,11,11-tetramethyl-7-thia-2,5-diazatricyclo[6.4.0.0^{2,6}]dodeca-1(8),3,5-triene-4-carboxylic acid was used except that ethyl 3-cyclopropyl-9,9,11,11-tetramethyl-7-thia-2,5-diazatricyclo[6.4.0.0^{2,6}]dodeca-1(8),3,5-triene-4-carboxylate was substituted for ethyl 3-(4-methoxyphenyl)-9,9,11,11-tetramethyl-7-thia-2,5-diazatricyclo[6.4.0.0^{2,6}]dodeca-1(8),3,5-triene-4-carboxylate (30% yield); m/z=319.6 (MH)⁺; 1H NMR (500 MHz, Methanol-d4) δ 0.84-0.88 (m, 2H), 1.13-1.17 (m, 2H), 1.19 (s, 6H), 1.39 (s, 6H), 1.75 (s, 2H), 2.04-2.11 (m, 1H), 3.02 (s, 2H). Exchangeable protons not observed.

### Example 259

### 9-Chloro-4-(cyclopentylmethyl)-10-fluoro-7-thia-2,5-diazatricyclo[6.4.0.0^{2,6}]dodeca-1(8),5,9,11-tetraene-4-carboxylic acid

To a stirred solution of tert-butyl 9-chloro-10-fluoro-7-thia-2,5-diazatricyclo[6.4.0.0^{2,6}]dodeca-1(8),5,9,11-tetraene-4-carboxylate (100 mg, 0.304 mmol) in DMF (2 mL) was added sodium 2-methylpropan-2-olate (29.2 mg, 0.304 mmol) followed by (iodomethyl)cyclopentane (40 μL, 0.30 mmol). The reaction mixture was stirred at room temperature for 2 h. More (iodomethyl)cyclopentane (40 μL, 0.30 mmol) was added and the reaction mixture stirred at room temperature for 1 h. The mixture was and partitioned between EtOAc (10 mL) and water (10 mL). The organic layer was reserved and the aqueous layer re-extracted with EtOAc (5 mL). The organic layers were combined, washed with brine (5 mL), dried (magnesium sulfate), filtered and concentrated. To the residue was added 6M HCl_{(aq)} (2 mL) and the resulting suspension stirred for 2 days at room temperature. More 6M HCl_{(aq)} (1 mL) was added and the suspension was heated to 50° C. for 2 h. The reaction mixture was concentrated and the resulting residue was dissolved in DMSO:MeOH (1:1, 1 mL) and purified by automated reverse phase HPLC (low pH method) to afford the title compound as a white powder (6 mg, 6% yield); m/z=355.1 (MH)⁺; 1H NMR (500 MHz, Methanol-d4) δ 1.18-1.28 (m, 2H), 1.51-1.61 (m, 2H), 1.62-1.73 (m, 2H), 1.85-2.01 (m, 3H), 2.15 (d, 2H), 4.17 (d, 1H), 4.68 (d, 1H), 7.23 (dd, 1H), 7.38 (t, 1H). Exchangeable protons not observed.

### Example 260

### 9-Chloro-10-(morpholin-4-yl)-7-thia-2,5-diazatricyclo[6.4.0.0^{2,6}]dodeca-1(8),3,5,9,11-pentaene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 9-chloro-10-(morpholin-4-yl)-7-thia-2,5-diazatricyclo[6.4.0.0^{2,6}]dodeca-1(8),3,5,9,11-pentaene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate. Final purification was by trituration in MeOH (41% yield); m/z=338.0 (MH)⁺; 1H NMR (500 MHz, DMSO-d6) δ 2.98-3.14 (m, 4H), 3.69-3.85 (m, 4H), 7.44 (d, 1H), 8.13 (d, 1H), 8.96 (s, 1H) 12.73 (s, 1H).

### Example 261

### 9,10-Dichloro-11-[2-(morpholin-4-yl)ethoxy]-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 9,10-dichloro-11-[2-(morpholin-4-yl)ethoxy]-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate (61% yield); m/z=416.0 (MH)⁺; 1H NMR (500 MHz, DMSO-d6) δ 3.48-3.76 (m, 4H), 3.76-4.08 (m, 4H), 4.65 (s, 2H), 8.28 (s, 1H), 9.01 (s, 1H), 11.39 (s, 1H), 12.83 (s, 1H). 2 Protons hidden. HCl salt.

### Example 262

### 4-Benzyl-9-chloro-10-fluoro-7-thia-2,5-diazatricyclo[6.4.0.0^{2,6}]dodeca-1(8),5,9,11-tetraene-4-carboxylic acid

To a solution of ethyl 9-chloro-10-fluoro-7-thia-2,5-diazatricyclo[6.4.0.0^{2,6}]dodeca-1(8),5,9,11-tetraene-4-carboxylate (200 mg, 0.665 mmol) in DMF (10 mL) was added (bromomethyl)benzene (118 μL, 0.998 mmol) followed by sodium 2-methylpropan-2-olate (96 mg, 0.998 mmol). The reaction mixture was stirred at room temperature for 2 h. After this time LiOH.H₂O (56 mg, 1.3 mmol) and water (1 mL) were added and stirring was continued for 1 h at 0° C. The mixture was then evaporated to dryness and the resulting residue was partitioned between DCM (20 mL) and 1M HCl_{(aq)} (20 mL). The layers were separated and the aqueous layer extracted with DCM (2×20 mL). The combined organic extracts were dried (MgSO₄), filtered and evaporated to dryness. The residue was purified by automated reverse phase HPLC (low pH method) to afford the title compound as a white solid (98 mg, 41% yield); m/z=363.1 (MH)⁺; 1H NMR (500 MHz, DMSO-d6) δ 3.12 (d, 1H), 3.26 (d, 1H), 3.87 (d, 1H), 4.19 (d, 1H), 6.90 (dd, 1H), 7.16-7.21 (m, 1H), 7.22-7.32 (m, 5H). Exchangeable protons not observed.

### Example 263

### 9-Chloro-10-fluoro-4-(hydroxymethyl)-7-thia-2,5-diazatricyclo[6.4.0.0^{2,6}]dodeca-1(8),5,9,11-tetraene-4-carboxylic acid

To a stirred solution of tert-butyl 9-chloro-10-fluoro-7-thia-2,5-diazatricyclo[6.4.0.0^{2,6}]dodeca-1(8),5,9,11-tetraene-4-carboxylate (30 mg, 0.091 mmol) in DMF (0.5 mL), under nitrogen, was added formaldehyde in water (37% solution, 14 μL, 0.18 mmol). The resulting solution was stirred at room temperature for 1 h. Sodium
2-methylpropan-2-olate (8.8 mg, 0.091 mmol) and more formaldehyde (14 μL, 0.18 mmol) were added and the resulting solution stirred at room temperature for 16 h. More sodium 2-methylpropan-2-olate (8.8 mg, 0.09 mmol) and formaldehyde (14 μL, 0.18 mmol) were added and the reaction mixture was stirred at room temperature for 1 h. The reaction mixture was concentrated and suspended in DMSO:MeOH (1:1, 1 mL). 6M HCl_{(aq)} (2 drops, pH 4) was added and the resulting solution purified via automated reverse phase HPLC (low pH method) to afford the title compound as a white solid (15 mg, 55% yield); m/z=303.0 (MH)⁺; 1H NMR (500 MHz, DMSO-d6) δ 3.59 (d, 1H), 3.74 (d, 1H), 3.96 (d, 1H), 4.24 (d, 1H), 6.99 (dd, 1H), 7.35 (dd, 1H). Exchangeable protons not observed.

### Example 264

### 9-Chloro-10-fluoro-N-methanesulfonyl-7-thia-2,5-diazatricyclo[6.4.0.0^{2,6}]dodeca-1(8),5,9,11-tetraene-4-carboxamide

To a stirred solution of 9-chloro-10-fluoro-7-thia-2,5-diazatricyclo[6.4.0.0^{2,6}]dodeca-1(8),5,9,11-tetraene-4-carboxylic acid (80 mg, 0.29 mmol) in THF (2 mL), at 0° C., under nitrogen, was added oxalyl chloride (154 μL, 1.76 mmol). The resulting solution was stirred at 0° C. for 2 h. Methanesulfonamide (167 mg, 1.76 mmol) was added and the resulting suspension stirred for 3 days at room temperature. The resulting suspension was quenched with water and concentrated under reduced pressure. The residue was suspended in EtOAc (20 mL) and water (20 mL). The mixture was filtered. The filtrate was separated and the aqueous layer washed with EtOAc (10 mL). The organic layer was discarded and the aqueous layer extracted with IPA:CHCl₃ (1:1, 3×10 mL). The combined organic extracts were dried (MgSO₄), filtered and concentrated. The residue was purified by automated reverse phase HPLC (low pH method) to afford the title compound as a pink solid (8 mg, formic acid salt, 7% yield); m/z=350.0 (MH)⁺; 1H NMR (500 MHz, Methanol-d4) δ 3.15 (s, 3H), 4.26-4.33 (m, 2H), 5.08-5.16 (m, 1H), 7.01 (dd, 1H), 7.28 (t, 1H), 8.34 (s, 1H).

### Example 265

### 9-Chloro-10-(piperidin-1-yl)-7-thia-2,5-diazatricyclo[6.4.0.0^{2,6}]dodeca-1(8),3,5,9,11-pentaene-4-carboxylic acid

To a stirred suspension of ethyl 9-chloro-10-(piperidin-1-yl)-7-thia-2,5-diazatricyclo[6.4.0.0^{2,6}]dodeca-1(8),3,5,9,11-pentaene-4-carboxylate (160 mg, 0.440 mmol) in EtOH (2 mL) and water (2 mL) was added LiOH.H₂O (92 mg, 2.2 mmol). The resulting suspension was stirred at 45° C. for 1 h then concentrated under reduced pressure and acidified to pH 1 using 6M HCl_{(aq)}. DCM (10 mL) was added and the resulting precipitate was collected by filtration. The solid was combined with the separated organic layer from the filtrate and the solvent removed under reduced pressure, giving an orange solid which was suspended in MeOH/DMSO (1:1, 3 mL) and filtered. The solid was washed with water and MeOH, to afford the title compound as an orange solid (26 mg, 17%); m/z=336.0 (MH)⁺; 1H NMR (500 MHz, DMSO-d6) δ 1.52-1.60 (m, 2H), 1.66-1.74 (m, 4H), 2.95-3.02 (m, 4H), 7.38 (d, 1H), 8.08 (d, 1H), 8.92 (s, 1H). Exchangeable protons not observed.

### Example 266

### 9-Chloro-10-(3,3-difluoroazetidin-1-yl)-7-thia-2,5-diazatricyclo[6.4.0.0^{2,6}]dodeca-1(8),3,5,9,11-pentaene-4-carboxylic acid

To a solution of ethyl 10-bromo-9-chloro-7-thia-2,5-diazatricyclo[6.4.0.0^{2,6}]dodeca-1(8),3,5,9,11-pentaene-4-carboxylate (250 mg, 0.695 mmol) in 1,4-dioxane (3 mL) in a pressure tube was added Pd₂(dba)₃ (64 mg, 0.070 mmol), Xantphos (40 mg, 0.070 mmol) and Cs₂CO₃ (453 mg, 1.39 mmol). The solution was degassed with nitrogen and then 3,3-difluoroazetidine hydrochloride (180 mg, 1.39 mmol) was added. The tube was sealed and the reaction mixture was stirred at 100° C. for 16 h. The reaction was cooled and poured into EtOAc (25 mL). The resulting solution washed with 1M HCl_{(aq)} (25 mL), water (25 mL) and brine (25 mL). The organic layer was dried (MgSO₄), filtered and concentrated. The residue was purified by FCC on silica (0-100% EtOAc in heptane). The resulting ester was dissolved in EtOH (5 mL) and water (1 mL) and then LiOH.H₂O (13 mg, 0.32 mmol) was added. The mixture was stirred at 50° C. for 1 h then the reaction mixture was evaporated to dryness, taken up in water (10 mL) and acidified to pH 3 using 1M HCl_{(aq)}. The mixture was extracted with DCM (3×25 mL) and the combined organic were dried, filtered and evaporated. The residue was purified by automated reverse-phase HPLC (low pH method) to afford the title compound as a white solid (12 mg, 33% yield); m/z=344.0 (MH)⁺; (500 MHz, DMSO-d6) δ 4.54 (t, 4H), 7.00 (d, 1H), 8.06 (d, 1H), 8.88 (s, 1H).

### Example 267

### 3-(3,3-Difluoroazetidin-1-yl)-9,9,11,11-tetramethyl-7-thia-2,5diazatricyclo[6.4.0.0^{2,6}]dodeca-1(8),3,5-triene-4-carboxylic acid

A solution of ethyl 3-(3,3-difluoroazetidin-1-yl)-9,9,11,11-tetramethyl-7-thia-2,5-diazatricyclo[6.4.0.0^{2,6}]dodeca-1(8),3,5-triene-4-carboxylate (60 mg, 0.15 mmol) and LiOH.H₂O (19 mg, 0.45 mmol) in EtOH:H₂O (1:1, 4 mL) was warmed to 60° C. for 3 h. After this time the reaction mixture was concentrated and the residue was partitioned between EtOAc (20 mL) and 1M NaOH_{(aq)} (10 mL). The organic layer was removed. The aqueous layer was acidified to pH 2-3 using 1M HCl_{(aq)} and extracted using 20% IPA in DCM (20 mL). The organic layer was dried (sodium sulfate), filtered and concentrated to yield the title compound as an off white solid (50 mg, 84% yield); m/z=370.5 (MH)⁺; 1H NMR (500 MHz, Methanol-d4) δ 1.15 (s, 6H), 1.34 (s, 6H), 1.70 (s, 2H), 2.85 (s, 2H), 4.47 (t, 4H). Exchangeable protons not observed.

### Example 268

### 3-{1-[(tert-Butoxy)carbonyl]-1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylic acid

The procedure to prepare 3-(3,3-difluoroazetidin-1-yl)-9,9,11,11-tetramethyl-7-thia-2,5diazatricyclo[6.4.0.0^{2,6}]dodeca-1(8),3,5-triene-4-carboxylic acid was used except that ethyl 3-{1-[(tert-butoxy)carbonyl]-1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylate was substituted for ethyl 3-(3,3-difluoroazetidin-1-yl)-9,9,11,11-tetramethyl-7-thia-2,5diazatricyclo[6.4.0.0^{2,6}]dodeca-1(8),3,5-triene-4-carboxylate (46% yield); m/z=460.2 (MH)⁺; 1H NMR (500 MHz, Methanol-d4) δ 1.09 (s, 6H), 1.36 (s, 6H), 1.50 (s, 9H), 1.70 (s, 2H), 2.48 (s, 4H), 3.67 (s, 2H), 4.10 (s, 2H), 5.86 (s, 1H). Exchangeable protons not observed.

### Example 269

### 9,9,11,11-Tetramethyl-3-(pyrrolidin-1-yl)-7-thia-2,5-diazatricyclo[6.4.0.0^{2,6}]dodeca-1(8),3,5-triene-4-carboxylic acid

The procedure to prepare ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate was used except that pyrrolidine was substituted for morpholine. The work-up procedure was modified so that the reaction mixture was diluted with MeOH and filtered through Celite prior to extraction with 20% IPA/DCM (15 mL). Ester hydrolysis occurred during work-up, the resulting residue was purified by automated reverse-phase HPLC (low pH method) to afford the title compound was as a beige solid (8% yield); m/z=348.2 (MH)⁺; 1H NMR (500 MHz, DMSO-d6) δ 1.08 (s, 6H), 1.30 (s, 6H), 1.65 (s, 2H), 1.86-1.96 (m, 4H), 2.70 (s, 2H), 3.18 (t, 4H), 4.04 (s, 1H).

### Example 270

### 3-({4-[(tert-Butoxy)carbonyl]piperazin-1-yl}methyl)-10-tert-butyl-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 3-({4-[(tert-butoxy)carbonyl]piperazin-1-yl}methyl)-10-tert-butyl-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate and heating was continued for 2 h instead of 90 min (72% yield); m/z=477.4 (MH)⁺; 1H NMR (500 MHz, DMSO-d6) δ 0.94 (s, 9H), 1.39 (s, 9H), 1.50-1.63 (m, 1H), 2.07 (d, 1H), 2.37 (s, 4H), 2.62-2.75 (m, 1H), 2.84-2.97 (m, 1H), 3.27 (s, 5H), 3.95-4.12 (m, 2H). 2 Protons hidden. Exchangeable protons not observed

### Example 271

### 10-tert-Butyl-3-{[4-(2-hydroxyethyl)piperazin-1-yl]methyl}-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 10-tert-butyl-3-{[4-(2-hydroxyethyl)piperazin-11-yl]methyl}-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate, heating was continued for 1 h in place of 90 min and that purification was by FCC on silica, eluent 0-100% DMAW 90 in DCM (20% yield); m/z=421.3 (MH)⁺; 1H NMR (500 MHz, Methanol-d4) δ 1.00 (s, 9H), 1.44-1.59 (m, 1H), 1.59-1.70 (m, 1H), 2.14-2.25 (m, 1H), 2.45-2.59 (m, 1H), 2.67-2.78 (m, 1H), 2.80-2.93 (m, 4H), 2.99-3.26 (m, 5H), 3.75-3.88 (m, 2H), 4.20-4.42 (m, 2H). 3 Protons hidden. Exchangeable protons not observed

### Example 272

### 10-tert-Butyl-3-{[4-(2-methoxyethyl)piperazin-1-yl]methyl}-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 10-tert-butyl-3-{[4-(2-methoxyethyl)piperazin-1-yl]methyl}-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate and heating was continued for 2 h in place of 90 min (61% yield); m/z=435.3 (MH)⁺; 1H NMR (250 MHz, Methanol-d4) δ 1.01 (s, 9H), 1.46-1.75 (m, 2H), 2.21 (d, 1H), 2.44-2.61 (m, 1H), 2.75-2.86 (m, 2H), 2.86-2.95 (m, 10H), 3.28 (s, 1H), 3.36 (s, 3H), 3.63 (s, 2H), 4.14-4.42 (m, 2H). Exchangeable protons not observed.

### Example 273

### 10-tert-Butyl-3-[(4-methylpiperazin-1-yl)methyl]-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 10-tert-butyl-3-[(4-methylpiperazin-1-yl)methyl]-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate (25% yield); m/z=391.2 (MH)⁺; 1H NMR (250 MHz, Methanol-d4) δ 1.02 (s, 9H), 1.49-1.74 (m, 2H), 2.21 (dd, 1H), 2.45-2.62 (m, 1H), 2.72 (s, 3H), 2.74-3.18 (m, 9H), 3.35-3.44 (m, 2H), 4.29 (q, 2H). Exchangeable protons not observed

### Example 274

### 10-tert-Butyl-3-(piperazin-1-ylmethyl)-7-thia-2,5-diazatricyclo[6.4.0.0^{2,6}]dodeca-1(8),3,5-triene-4-carboxylic acid

To a solution of 3-({4-[(tert-butoxy)carbonyl]piperazin-1-yl}methyl)-10-tert-butyl-7-thia-2,5-diazatricyclo[6.4.0.0^{2,6}]dodeca-1(8),3,5-triene-4-carboxylic acid (22 mg, 0.05 mmol) in MeOH (1 mL) at 0° C. was added AcCl (13 μL, 0.20 mmol) with the resulting solution allowed to warm to room temperature over 16 h. The solution was cooled to 0° C. and more AcCl (26 μL, 0.40 mmol) was added. The reaction mixture was allowed to warm to room temperature over 7 h. The resulting precipitate was collected by filtration and washed with MeOH to afford the title compound as a colourless solid (HCl salt; 8 mg, 42% yield); m/z=377.2 (MH)⁺; 1H NMR (500 MHz, DMSO-d6) δ 0.88 (s, 9H), 1.30-1.41 (m, 1H), 1.45-1.57 (m, 1H), 1.99-2.03 (m, 1H), 2.65 (s, 4H), 2.80-2.91 (m, 1H), 3.02 (s, 4H), 4.07 (d, 2H), 8.67 (s, 2H). HCl salt. 2 Protons hidden. Exchangeable protons not observed.

### Example 275

### 3-[(4-Acetylpiperazin-1-yl)methyl]-10-tert-butyl-7-thia-2,5-diazatricyclo[6.4.0.0^{2,6}]dodeca-1(8),3,5-triene-4-carboxylic acid

The procedure to prepare 10-tert-butyl-3-(morpholin-4-ylmethyl)-7-thia-2,5-diazatricyclo[6.4.0.0^{2,6}]dodeca-1(8),3,5-triene-4-carboxylic acid was used except that ethyl 3-[(4-acetylpiperazin-1-yl)methyl]-10-tert-butyl-7-thia-2,5-diazatricyclo[6.4.0.0^{2,6}]dodeca-1(8),3,5-triene-4-carboxylate was substituted for ethyl 10-tert-butyl-3-(morpholin-4-ylmethyl)-7-thia-2,5-diazatricyclo[6.4.0.0^{2,6}]dodeca-1(8),3,5-triene-4-carboxylate (63% yield); m/z=419.3 (MH)⁺; 1H NMR (500 MHz, DMSO-d6) δ 0.94 (s, 9H), 1.35-1.49 (m, 1H), 1.51-1.62 (m, 1H), 1.98 (s, 3H), 2.02-2.12 (m, 1H), 2.37 (s, 2H), 2.43 (s, 2H), 2.47 (s, 1H), 2.65-2.75 (m, 1H), 2.85-2.99 (m, 1H), 3.96-4.13 (m, 4H). 3 Protons hidden. Exchangeable protons not observed.

### Example 276

### 9-Chloro-10-fluoro-3-(4-methylpiperazin-1-yl)-7-thia-2,5-diazatricyclo[6.4.0.0^{2,6}]dodeca-1(8),3,5,9,11-pentaene-4-carboxylic acid

The procedure to prepare 9-chloro-10-(3,3-difluoroazetidin-1-yl)-7-thia-2,5-diazatricyclo[6.4.0.0^{2,6}]dodeca-1(8),3,5,9,11-pentaene-4-carboxylic acid was used except that N-methylpiperizine and ethyl 3-bromo-9-chloro-10-fluoro-7-thia-2,5-diazatricyclo[6.4.0.0^{2,6}]dodeca-1(8),3,5,9,11-pentaene-4-carboxylate were substituted for 3,3-difluoroazetidine hydrochloride and ethyl 10-bromo-9-chloro-7-thia-2,5-diazatricyclo[6.4.0.0^{2,6}]dodeca-1(8),3,5,9,11-pentaene-4-carboxylate. Additionally, ester hydrolysis was carried out by heating for 2 h instead of 1 h (14% yield); m/z=369.1 (MH)⁺; 1H NMR (500 MHz, DMSO-d6) δ 2.31-2.41 (m, 5H), 2.85 (d, 2H), 2.93 (d, 2H), 3.61 (t, 2H), 7.71 (t, 1H), 8.27 (dd, 1H). Exchangeable protons not observed.

### Example 277

### 9-Chloro-10-fluoro-3-{[2-(pyrrolidin-1-yl)ethyl]amino}-7-thia-2,5-diazatricyclo[6.4.0.0^{2,6}]dodeca-1(8),3,5,9,11-pentaene-4-carboxylic acid

To a solution of ethyl 9-chloro-10-fluoro-3-{[2-(pyrrolidin-1-yl)ethyl]amino}-7-thia-2,5-diazatricyclo[6.4.0.0^{2,6}]dodeca-1(8),3,5,9,11-pentaene-4-carboxylate (40 mg, 0.1 mmol) in EtOH (1 mL) and water (0.5 mL) was added LiOH.H₂O (13 mg, 0.29 mmol). The resulting mixture was stirred at 50° C. for 1 h. Further LiOH.H₂O (13 mg, 0.29 mmol) was added and the reaction mixture was stirred at 50° C. for 4 h. After this time the reaction mixture was evaporated to dryness, taken-up in water (3 mL) and acidified with 1M HCl_{(aq)} to pH 2. The solution was evaporated to dryness and purified by automated reverse phase HPLC (low pH method) to afford the title compound as a white solid (46% yield); m/z=383.1 (MH)⁺; 1H NMR (500 MHz, DMSO-d6) δ 1.93 (s, 4H), 3.52 (q, 4H), 5.71 (t, 1H), 7.66 (t, 1H), 8.02 (dd, 1H). 3 Protons hidden. Exchangeable protons not observed

### Example 278

### N-(Benzenesulfonyl)-9-chloro-10-fluoro-7-thia-2,5-diazatricyclo[6.4.0.0^{2,6}]dodeca-1(8),5,9,11-tetraene-4-carboxamide

To a solution of ethyl 9-chloro-10-fluoro-7-thia-2,5-diazatricyclo[6.4.0.0^{2,6}]dodeca-1(8),5,9,11-tetraene-4-carboxylate (90 mg, 0.3 mmol) in THF (3 mL) was added benzenesulfonamide (52 mg, 0.33 mmol) followed by 1,4-diazabicyclo[2.2.2]octane-trimethylaluminum (1:2) (77 mg, 0.30 mmol). The reaction mixture was flushed with nitrogen and stirred in a sealed tube at 80° C. for 2 h. The reaction was then quenched with water (2 mL) at 4° C. and evaporated to dryness. The resulting residue was partitioned between EtOAc (30 mL) and 1M HCl_{(aq)} (30 mL). The precipitate present in the aqueous layer was collected by filtration and dried in vacuo to give the title compound as a white solid (66% yield); m/z=412.1 (MH)⁺; 1H NMR (500 MHz, DMSO-d6) δ 4.41-4.60 (m, 2H), 5.36 (dd, 1H), 7.49 (dd, 1H), 7.57-7.67 (m, 3H), 7.72 (t, 1H), 7.97 (d, 2H). Exchangeable protons not observed

### Example 279

### (4R)-9-Chloro-10-fluoro-4-methyl-7-thia-2,5-diazatricyclo[6.4.0.0^{2,6}]dodeca-1(8),5,9,11-tetraene-4-carboxylic acid and (4S)-9-chloro-10-fluoro-4-methyl-7-thia-2,5-diazatricyclo[6.4.0.0^{2,6}]dodeca-1(8),5,9,11-tetraene-4-carboxylic acid (Configurations Arbitrarily Assigned)

Racemic ethyl 9-chloro-10-fluoro-7-thia-2,5-diazatricyclo[6.4.0.0^{2,6}]dodeca-1(8),5,9,11-tetraene-4-carboxylate was prepared as previously described and was successfully resolved using SFC on a Chiralpak AD-H column (25 cm; 90:10 heptane:EtOH+0.2% formic acid at 18 mL/min) to afford the title compounds as light pink solids (6% and 5% yield respectively); m/z=287.0 (MH)⁺; 1H NMR (500 MHz, DMSO-d6) δ 7.33 (t, 1H), 6.92 (dd, 1H), 4.26 (d, 1H), 3.72 (d, 1H), 1.47 (s, 3H). Exchangeable protons not observed; and m/z=287.0 (MH)⁺; 1H NMR (500 MHz, DMSO-d6) δ 1.48 (s, 3H), 3.73 (d, 1H), 4.27 (d, 1H), 6.93 (dd, 1H), 7.34 (dd, 1H). Exchangeable protons not observed.

### Example 280

### 3-(Dimethylamino)-9,9,11,11-tetramethyl-7-thia-2,5-diazatricyclo[6.4.0.0^{2,6}]dodeca-1(8),3,5-triene-4-carboxylic acid

A suspension of ethyl 3-(dimethylamino)-9,9,11,11-tetramethyl-7-thia-2,5-diazatricyclo[6.4.0.0^{2,6}]dodeca-1(8),3,5-triene-4-carboxylate (23 mg, 0.06 mmol) and LiOH.H₂O (5 mg, 0.12 mmol) in EtOH/H₂O [1:1 (v/v); 0.6 mL] was heated at 60° C. for 90 min. After this time, the reaction mixture was acidified with 1M HCl_{(aq)} and extracted with EtOAc. The organic layer was isolated, washed with brine, dried (MgSO₄), filtered and concentrated. The crude product was triturated with diethyl ether to afford the title compound as an off-white solid (19 mg, 94% yield); m/z=322.2 (MH)⁺; 1H NMR (500 MHz, Methanol-d4) δ 1.16 (s, 6H), 1.37 (s, 6H), 1.73 (s, 2H), 2.83 (s, 2H), 2.86 (s, 6H). Exchangeable protons not observed.

### Example 281

### 3-(Azetidin-3-yl)-9-chloro-10-fluoro-7-thia-2,5-diazatricyclo[6.4.0.0^{2,6}]dodeca-1(8),3,5,9,11-pentaene-4-carboxylic acid

The procedure to prepare 3-(azetidin-3-yl)-9,9,11,11-tetramethyl-7-thia-2,5-diazatricyclo[6.4.0.0^{2,6}]dodeca-1(8),3,5-triene-4-carboxylic acid was used expect that 3-{1-[(tert-butoxy)carbonyl]azetidin-3-yl}-9-chloro-10-fluoro-7-thia-2,5-diazatricyclo[6.4.0.0^{2,6}]dodeca-1(8),3,5,9,11-pentaene-4-carboxylic acid was substituted for 3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylic acid. No further additions of TFA required as the reaction was complete after 80 min (TFA salt, 100% yield); m/z=325.9 (MH)⁺; 1H NMR (500 MHz, DMSO-d6) δ 4.35 (qd, 2H), 4.50 (p, 2H), 5.12 (p, 1H), 7.73 (t, 1H), 8.16 (dd, 1H), 8.55 (s, 1H), 8.89 (s, 1H), 13.16 (s, 1H). TFA salt.

### Example 282

### 9,9,11,11-Tetramethyl-3-(piperazin-1-yl)-7-thia-2,5-diazatricyclo[6.4.0.0^{2,6}]dodeca-1(8),3,5-triene-4-carboxylic acid

The procedure to prepare 3-(dimethylamino)-9,9,11,11-tetramethyl-7-thia-2,5-diazatricyclo[6.4.0.0^{2,6}]dodeca-1(8),3,5-triene-4-carboxylic acid was used except that ethyl 9,9,11,11-tetramethyl-3-(piperazin-1-yl)-7-thia-2,5-diazatricyclo[6.4.0.0^{2,6}]dodeca-1(8),3,5-triene-4-carboxylate was substituted for ethyl 3-(dimethylamino)-9,9,11,11-tetramethyl-7-thia-2,5-diazatricyclo[6.4.0.0^{2,6}]dodeca-1(8),3,5-triene-4-carboxylate. Following acidification the reaction mixture was concentrated, with the residual material triturated with MeOH (36% yield); m/z=363.2 (MH)⁺; 1H NMR (500 MHz, Methanol-d4) δ 1.18 (s, 6H), 1.36 (s, 6H), 1.93 (s, 2H), 2.87 (s, 2H), 3.08-3.14 (m, 2H), 3.92-4.01 (m, 2H), 4.58 (s, 4H). Exchangeable protons not observed.

### Example 283

### 9,9,11,11-Tetramethyl-3-(1,2,3,6-tetrahydropyridin-4-yl)-7-thia-2,5-diazatricyclo[6.4.0.0^{2,6}]dodeca-1(8),3,5-triene-4-carboxylic acid

The procedure to prepare 9,9,11,11-tetramethyl-3-(piperazin-1-yl)-7-thia-2,5-diazatricyclo[6.4.0.0^{2,6}]dodeca-1(8),3,5-triene-4-carboxylic acid was used except that ethyl 3-(1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylate was substituted for ethyl 9,9,11,11-tetramethyl-3-(piperazin-1-yl)-7-thia-2,5-diazatricyclo[6.4.0.0^{2,6}]dodeca-1(8),3,5-triene-4-carboxylate. The residual material was purified by FCC, eluting with 0-100% DMAW 90 in DCM, to afford the title product as a salmon pink solid (90% yield); m/z=360.2 (MH)⁺; 1H NMR (500 MHz, Methanol-d4) δ 1.13 (s, 6H), 1.31 (s, 2H), 1.34 (s, 6H), 1.71 (s, 2H), 2.52 (s, 2H), 3.44 (s, 2H), 3.84 (s, 2H), 5.93 (s, 1H). Exchangeable protons not observed

### Example 284

### 3-(4-Acetylpiperazin-1-yl)-9,9,11,11-tetramethyl-7-thia-2,5-diazatricyclo[6.4.0.0^{2,6}]dodeca-1(8),3,5-triene-4-carboxylic acid

The procedure to prepare 3-(dimethylamino)-9,9,11,11-tetramethyl-7-thia-2,5-diazatricyclo[6.4.0.0^{2,6}]dodeca-1(8),3,5-triene-4-carboxylic acid was used except that ethyl 3-(4-acetylpiperazin-1-yl)-9,9,11,11-tetramethyl-7-thia-2,5-diazatricyclo[6.4.0.0^{2,6}]dodeca-1(8),3,5-triene-4-carboxylate was substituted for ethyl 3-(dimethylamino)-9,9,11,11-tetramethyl-7-thia-2,5-diazatricyclo[6.4.0.0² 6]dodeca-1(8),3,5-triene-4-carboxylate. Trituration was not required (73% yield); m/z=405.3 (MH)⁺; 1H NMR (500 MHz, Methanol-d4) δ 1.08 (s, 6H), 1.30 (s, 6H), 1.66 (s, 2H), 2.06 (s, 3H), 2.78-2.86 (m, 1H), 2.87 (s, 2H), 2.98 (t, 2H), 3.27-3.41 (m, 2H), 3.44-3.52 (m, 1H), 3.85 (d, 1H), 4.43 (d, 1H). Exchangeable protons not observed.

### Example 285

### 9,10-Dichloro-11-[2-(morpholin-4-yl)ethoxy]-7-thia-2,5-diazatricyclo[6.4.0.0^{2,6}]dodeca-1(8),5,9,11-tetraene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except that ethyl 9,10-dichloro-11-[2-(morpholin-4-yl)ethoxy]-7-thia-2,5-diazatricyclo[6.4.0.0^{2,6}]dodeca-1(8),5,9,11-tetraene-4-carboxylate was substituted for ethyl 9,11-dichloro-7-thia-2,5-diazatricyclo[6.4.0.0^{2,6}]dodeca-1(8),3,5,9,11-pentaene-4-carboxylate. Following acidification the aqueous layer was washed with IPA/CHCl₃ (1:1, 3×20 mL) and then the aqueous layer was concentrated. The residue was diluted with water (2.5 mL), sonicated and the title compound collected by filtration (47% yield); m/z=418.1 (MH)⁺; 1H NMR (500 MHz, DMSO-d6) δ 3.61-3.66 (m, 2H), 3.77-4.11 (m, 4H), 4.54 (d, 2H), 4.63 (s, 2H), 5.39 (t, 1H), 7.50 (s, 1H), 11.49 (s, 1H). 4 Protons hidden.

### Example 286

### 9-Chloro-N-[(dimethyl-1,2-oxazol-4-yl)sulfonyl]-10-fluoro-7-thia-2,5-diazatricyclo[6.4.0.0^{2,6}]dodeca-1(8)3,5,9-tetraene-4-carboxamide

The procedure to prepare N-(benzenesulfonyl)-9-chloro-10-fluoro-7-thia-2,5-diazatricyclo[6.4.0.0^{2,6}]dodeca-1(8),5,9,11-tetraene-4-carboxamide was used except for 3,5-dimethyl-1,2-oxazole-4-sulfonamide was used in place of benzenesulfonamide and that the reaction mixture was concentrated and partitioned between EtOAc (15 mL) and water/HCl (pH 2, 15 mL). The organic layer was taken and the aqueous layer extracted with EtOAc (2×10 mL). The combined organic extracts were washed with 1M HCl_{(aq)} (5 mL) and brine (5 mL), dried (MgSO₄), filtered and concentrated. The solvent was removed under reduced pressure and the resulting gum was suspended in DMSO:MeCN (1:1, 2 mL) and filtered. The filtrate was purified by automated reverse-phase HPLC (low pH method) to afford the title compound (43% yield); m/z=431.1 (MH)⁺; 1H NMR (250 MHz, DMSO-d6) δ 2.31 (s, 3H), 2.56 (s, 3H), 4.39 (d, 2H), 5.10 (t, 1H), 7.39 (dd, 1H), 7.50-7.60 (m, 1H). Exchangeable protons not observed

### Example 287

### 9,9,11,11-Tetramethyl-3-(prop-1-en-2-yl)-7-thia-2,5-diazatricyclo[6.4.0.0^{2,6}]dodeca-1(8),3,5-triene-4-carboxylic acid

To a solution of ethyl 9,9,11,11-tetramethyl-3-(prop-1-en-2-yl)-7-thia-2,5-diazatricyclo[6.4.0.0^{2,6}]dodeca-1(8),3,5-triene-4-carboxylate (77% purity, 155 mg, 0.344 mmol) in MeOH (3 mL) and water (1 mL) was added 5M aqueous NaOH solution (0.690 mL, 3.4 mmol) and the solution stirred at 80° C. for 3 h. The MeOH was evaporated and then DCM (30 mL) and water (10 mL) were added. The phases were separated and the organic phase was washed with brine (5 mL), dried (Na₂SO₄), filtered and concentrated. The residue was purified by automated reverse phase HPLC (low pH method) to afford the title compound as a pink solid (32 mg, 29%); m/z=319.1 (MH)⁺; 1H NMR (500 MHz, DMSO-d6) δ 1.05 (s, 6H), 1.31 (s, 6H), 1.65 (s, 2H), 2.10 (s, 3H), 5.13 (s, 1H), 5.46-5.54 (m, 1H). 2 Protons hidden. Exchangeable protons not observed.

### Example 288

### 9,9,11,11-Tetramethyl-3-(4-methylpiperazin-1-yl)-7-thia-2,5-diazatricyclo[6.4.0.0^{2,6}]dodeca-1(8),3,5-triene-4-carboxylic acid

The procedure to prepare 3-(dimethylamino)-9,9,11,11-tetramethyl-7-thia-2,5-diazatricyclo[6.4.0.0^{2,6}]dodeca-1(8),3,5-triene-4-carboxylic acid was used except that ethyl 9,9,11,11-tetramethyl-3-(4-methylpiperazin-1-yl)-7-thia-2,5-diazatricyclo[6.4.0.0^{2,6}]dodeca-1(8),3,5-triene-4-carboxylate was substituted for ethyl 3-(dimethylamino)-9,9,11,11-tetramethyl-7-thia-2,5-diazatricyclo[6.4.0.0² 6]dodeca-1(8),3,5-triene-4-carboxylate (64% yield); m/z=377.2 (MH)⁺; 1H NMR (500 MHz, Methanol-d4) δ 1.19 (s, 6H), 1.36 (s, 6H), 1.72 (s, 2H), 2.75 (s, 3H), 2.85 (t, 2H), 2.88 (s, 2H), 3.06 (d, 2H), 3.27 (d, 2H), 4.02 (t, 2H). Exchangeable protons not observed

### Example 289

### 10-Chloro-9-fluoro-11-[2-(morpholin-4-yl)ethoxy]-7-thia-2,5-diazatricyclo[6.4.0.0^{2,6}]dodeca-1(8),3,5,9,11-pentaene-4-carboxylic acid

To a stirred solution of 6-chloro-7-fluoro-5-[2-(morpholin-4-yl)ethoxy]-1,3-benzothiazol-2-amine (0.250 g, 0.753 mmol) in DME (10 mL) was added ethyl 3-bromo-2-oxopropanoate (734 mg, 3.01 mmol). The reaction was heated at 85° C. for 1.5 h. The solvent was evaporated and the residue purified by FCC (eluent: 10-40% MeOH in EtOAc). The intermediate ester was then dissolved in MeOH (5 mL) and 2M NaOH (5 mL) and the mixture heated at 80° C. for 1 h. The MeOH was evaporated and the aqueous phase was made acidic with 6N HCl_{(aq)}. The solvent was evaporated and the residue purified by automated reverse phase HPLC (low pH method). Further purification was achieved by trituration with MeOH, affording the title compound as a yellow solid (5 mg, 1.5% yield); m/z=400.0 (MH)⁺; 1H NMR (500 MHz, DMSO-d6) δ 3.58-3.63 (m, 4H), 4.34 (t, 2H), 8.07 (s, 1H), 8.98 (s, 1H). 6 Protons hidden. Exchangeable protons not observed.

### Example 290

### 9-Chloro-10-fluoro-3-(1-methylazetidin-3-yl)-7-thia-2,5-diazatricyclo[6.4.0.0^{2,6}]dodeca-1(8),3,5,9,11-pentaene-4-carboxylic acid

The procedure to prepare 9,11-dichloro-7-thia-2,5-diazatricyclo[6.4.0.0^{2,6}]dodeca-1(8),3,5,9,11-pentaene-4-carboxylic acid was used except ethyl 9-chloro-10-fluoro-3-(1-methylazetidin-3-yl)-7-thia-2,5-diazatricyclo[6.4.0.0^{2,6}]dodeca-1(8),3,5,9,11-pentaene-4-carboxylate was substituted for ethyl 9,11-dichloro-7-thia-2,5-diazatricyclo[6.4.0.0^{2,6}]dodeca-1(8),3,5,9,11-pentaene-4-carboxylate (25% yield); m/z=340.0 (MH)⁺; 1H NMR (250 MHz, DMSO-d6) δ 2.96 (s, 3H), 4.41 (t, 2H), 4.61 (t, 2H), 4.95-5.29 (m, 1H), 7.75 (t, 1H), 8.18 (d, 1H). Exchangeable protons not observed.

### Example 291

### 9-Chloro-3-cyclopropyl-10-fluoro-7-thia-2,5-diazatricyclo[6.4.0.0^{2,6}]dodeca-1(8),3,5,9,11-pentaene-4-carboxylic acid

A solution of ethyl 9-chloro-3-cyclopropyl-10-fluoro-7-thia-2,5-diazatricyclo[6.4.0.0^{2,6}]dodeca-1(8),3,5,9,11-pentaene-4-carboxylate (22 mg, 0.050 mmol) and LiOH.H₂O (4.1 mg, 0.10 mmol) in EtOH:H₂O (1:1, 0.5 mL) was warmed to 60° C. for 1 h. After this time the reaction mixture was allowed to cool to room temperature and acidified to pH 3 by the dropwise addition of 1M HCl_{(aq)}. The aqueous layer was extracted with 20% IPA in DCM (×2). The combined organic extracts were washed with brine, dried (MgSO₄), filtered and concentrated. The residue was purified by FCC on silica, eluent: 0-100% DMAW 120 in DCM. Further purification was performed via trituration in MeOH. The resultant carboxylic acid (11 mg, 0.035 mmol) was added to a solution of 2-amino-2-(hydroxymethyl)propane-1,3-diol (4.2 mg, 0.035 mmol) in MeCN/H₂O (10:1; 0.35 mL) and stirred at 60° C. for 2 h. After this time the reaction mixture was diluted with water. The resulting precipitate was removed by filtration and the filtrate was concentrated to afford the title compound as the Tris salt as an off-white solid (12 mg, 62% yield); m/z=311.0 (MH)⁺; 1H NMR (500 MHz, Methanol-d4) δ 0.89 (d, 2H), 1.23-1.29 (m, 2H), 2.18 (ddd, 1H), 3.62 (s, 12H), 7.49 (t, 1H), 8.28 (dd, 1H). Amine:carboxylic acid 2:1. Exchangeable protons not observed.

### Example 292

### 9,9,11,11-Tetramethyl-3-(methylsulfanyl)-7-thia-2,5-diazatricyclo[6.4.0.0^{2,6}]dodeca-1(8),3,5-triene-4-carboxylic acid

The procedure to prepare 10-(benzyloxy)-7-thia-2,5-diazatricyclo[6.4.0.0^{2,6}]dodeca-1(8),3,5,9,11-pentaene-4-carboxylic acid was used except that ethyl 9,9,11,11-tetramethyl-3-(methylsulfanyl)-7-thia-2,5-diazatricyclo[6.4.0.0^{2,6}]dodeca-1(8),3,5-triene-4-carboxylate was substituted for ethyl 10-(benzyloxy)-7-thia-2,5-diazatricyclo[6.4.0.0^{2,6}]dodeca-1(8),3,5,9,11-pentaene-4-carboxylate (35% yield); m/z=325.1 (MH)⁺; 1H NMR (500 MHz, Methanol-d4) δ 1.18 (s, 6H), 1.40 (s, 6H), 1.75 (s, 2H), 2.47 (s, 3H), 3.10 (s, 2H). Exchangeable protons not observed.

### Example 293

### 9-Chloro-10-fluoro-3-(1-methanesulfonylazetidin-3-yl)-7-thia-2,5-diazatricyclo[6.4.0.0^{2,6}]dodeca-1(8),3,5,9,11-pentaene-4-carboxylic acid

To a solution of ethyl 3-(azetidin-3-yl)-9-chloro-10-fluoro-7-thia-2,5-diazatricyclo[6.4.0.0^{2,6}]dodeca-1(8),3,5,9,11-pentaene-4-carboxylate (86% purity, 250 mg, 0.61 mmol) in DCM (10 mL) was added methanesulfonyl chloride (71 μL, 0.91 mmol) followed by triethylamine (168 μL, 1.22 mmol). The resulting mixture was stirred at room temperature for 16 h then washed with water (2×50 mL). The organic layer was evaporated to dryness, the residue was dissolved in EtOH (20 mL) and LiOH.H₂O (92 mg, 2.2 mmol) was added. The mixture was then stirred at 50° C. for 1 h, evaporated to dryness, dissolved in water (20 mL) and acidified to pH 2 using 1M HCl_{(aq)}. The resulting precipitate was collected by filtration and purified by automated reverse phase HPLC (low pH method) to afford the title compound as a white solid (17 mg, 10% yield); m/z=404.0 (MH)⁺; 1H NMR (500 MHz, DMSO-d6) δ 3.10 (s, 3H), 4.36 (d, 4H), 4.99 (p, 1H), 7.68 (t, 1H), 8.25 (dd, 1H). Exchangeable protons not observed.

### Example 294

### 9-Chloro-10-fluoro-N-methanesulfonyl-4-methyl-7-thia-2,5-diazatricyclo[6.4.0.0^{2,6}]dodeca-1(8),5,9,11-tetraene-4-carboxamide

9-Chloro-10-fluoro-4-methyl-7-thia-2,5-diazatricyclo[6.4.0.0^{2,6}]dodeca-1(8),5,9,11-tetraene-4-carboxylic acid (90% purity, 54 mg, 0.15 mmol), methanesulfonamide (18 mg, 0.19 mmol), bis(trimethylaluminum)-1,4-diazabicyclo[2.2.2]octane adduct (40 mg, 0.15 mmol) and THF (2 mL) were added to a pressure tube, under nitrogen. The tube was sealed and the reaction mixture heated to 80° C. for 1 h, cooled, quenched with water and concentrated under reduced pressure. The resulting white gum was suspended in DMSO:MeCN (1:1, 1 mL, containing 3 drops of 1M HCl) and filtered. The filtrate was purified via automated reverse-phase HPLC (low pH method) to afford the title compound as a white solid (11 mg, 19% yield); m/z=364.0 (MH)⁺; 1H NMR (500 MHz, DMSO-d6) δ 1.51 (s, 3H), 3.18 (s, 3H), 3.89 (d, 1H), 4.26 (d, 1H), 7.06 (dd, 1H), 7.38-7.44 (m, 1H). Exchangeable protons not observed.

Structural formulas of Examples 1 to 294 are shown in Table 1.

TABLE 1

| | |
|---|---|
| 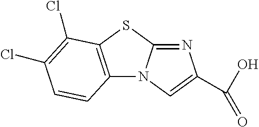 [SMILES: O=C(O)c1cn2c(n1)sc1c(Cl)c(Cl)ccc12] | Ex 1 |
| 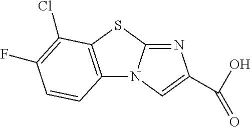 [SMILES: O=C(O)c1cn2c(n1)sc1c(Cl)c(F)ccc12] | Ex 2 |
| 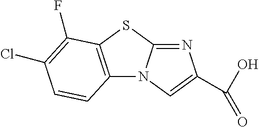 [SMILES: O=C(O)c1cn2c(n1)sc1c(F)c(Cl)ccc12] | Ex 3 |
| 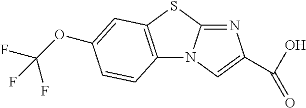 [SMILES: O=C(O)c1cn2c(n1)sc1cc(OC(F)(F)F)ccc12] | Ex 4 |
| 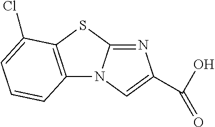 [SMILES: O=C(O)c1cn2c(n1)sc1c(Cl)cccc12] | Ex 5 |
| 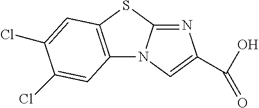 [SMILES: O=C(O)c1cn2c(n1)sc1cc(Cl)c(Cl)cc12] | Ex 6 |
| 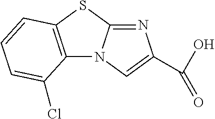 [SMILES: O=C(O)c1cn2c(n1)sc1cccc(Cl)c12] | Ex 7 |
| 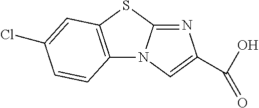 [SMILES: O=C(O)c1cn2c(n1)sc1cc(Cl)ccc12] | Ex 8 |
| 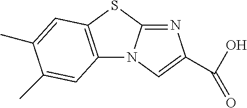 [SMILES: Cc1cc2sc3nc(C(=O)O)cn3c2cc1C] | Ex 9 |
| 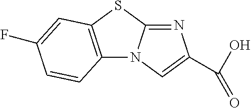 [SMILES: O=C(O)c1cn2c(n1)sc1cc(F)ccc12] | Ex 10 |
| 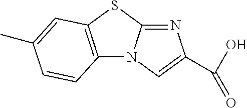 [SMILES: Cc1ccc2c(c1)sc1nc(C(=O)O)cn12] | Ex 11 |
| 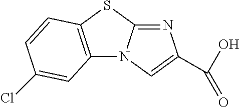 [SMILES: O=C(O)c1cn2c(n1)sc1ccc(Cl)cc12] | Ex 12 |
| 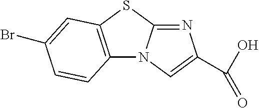 [SMILES: O=C(O)c1cn2c(n1)sc1cc(Br)ccc12] | Ex 13 |
| 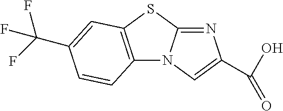 [SMILES: O=C(O)c1cn2c(n1)sc1cc(C(F)(F)F)ccc12] | Ex 14 |
| 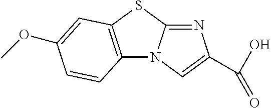 [SMILES: COc1ccc2c(c1)sc1nc(C(=O)O)cn12] | Ex 15 |
| 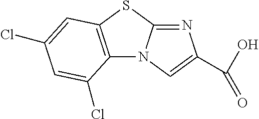 [SMILES: O=C(O)c1cn2c(n1)sc1cc(Cl)cc(Cl)c12] | Ex 16 |
| 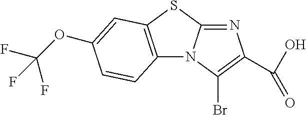 [SMILES: O=C(O)c1nc2sc3cc(OC(F)(F)F)ccc3n2c1Br] | Ex 17 |
| 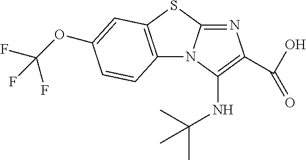 [SMILES: CC(C)(C)Nc1c(C(=O)O)nc2sc3cc(OC(F)(F)F)ccc3n12] | Ex 18 |
| 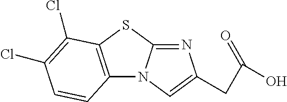 [SMILES: O=C(O)Cc1cn2c(n1)sc1c(Cl)c(Cl)ccc12] | Ex 19 |
| 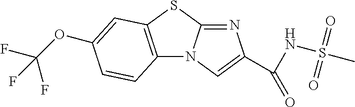 [SMILES: CS(=O)(=O)NC(=O)c1cn2c(n1)sc1cc(OC(F)(F)F)ccc12] | Ex 20 |
| 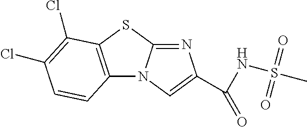 [SMILES: CS(=O)(=O)NC(=O)c1cn2c(n1)sc1c(Cl)c(Cl)ccc12] | Ex 21 |
| 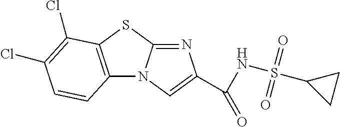 [SMILES: O=C(NS(=O)(=O)C1CC1)c1cn2c(n1)sc1c(Cl)c(Cl)ccc12] | Ex 22 |
| 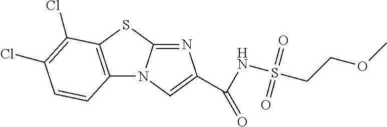 [SMILES: COCCS(=O)(=O)NC(=O)c1cn2c(n1)sc1c(Cl)c(Cl)ccc12] | Ex 23 |
| 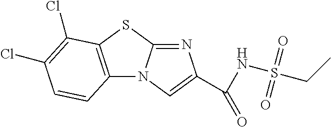 [SMILES: CCS(=O)(=O)NC(=O)c1cn2c(n1)sc1c(Cl)c(Cl)ccc12] | Ex 24 |
| 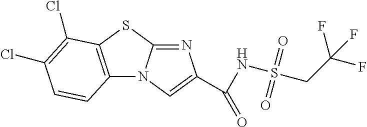 [SMILES: O=C(NS(=O)(=O)CC(F)(F)F)c1cn2c(n1)sc1c(Cl)c(Cl)ccc12] | Ex 25 |
| 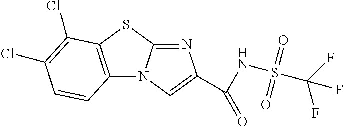 [SMILES: O=C(NS(=O)(=O)C(F)(F)F)c1cn2c(n1)sc1c(Cl)c(Cl)ccc12] | Ex 26 |
| 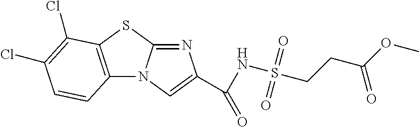 [SMILES: COC(=O)CCS(=O)(=O)NC(=O)c1cn2c(n1)sc1c(Cl)c(Cl)ccc12] | Ex 27 |
| 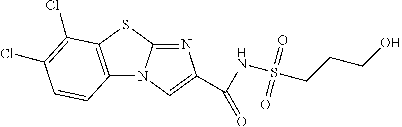 [SMILES: O=C(NS(=O)(=O)CCCO)c1cn2c(n1)sc1c(Cl)c(Cl)ccc12] | Ex 28 |
| 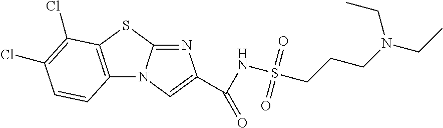 [SMILES: CCN(CC)CCCS(=O)(=O)NC(=O)c1cn2c(n1)sc1c(Cl)c(Cl)ccc12] | Ex 29 |
| 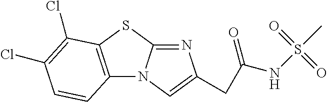 [SMILES: CS(=O)(=O)NC(=O)Cc1cn2c(n1)sc1c(Cl)c(Cl)ccc12] | Ex 30 |
| 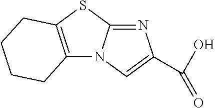 [SMILES: O=C(O)c1cn2c3c(sc2n1)CCCC3] | Ex 31 |
| 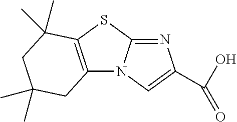 [SMILES: CC1(C)Cc2c(sc3nc(C(=O)O)cn23)C(C)(C)C1] | Ex 32 |
| 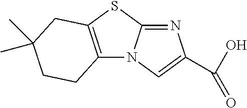 [SMILES: CC1(C)CCc2c(sc3nc(C(=O)O)cn23)C1] | Ex 33 |
| 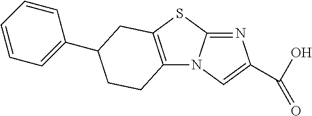 [SMILES: O=C(O)c1cn2c3c(sc2n1)CC(c1ccccc1)CC3] | Ex 34 |
| 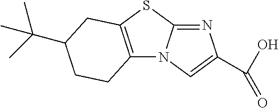 [SMILES: CC(C)(C)C1CCc2c(sc3nc(C(=O)O)cn23)C1] | Ex 35 |
| 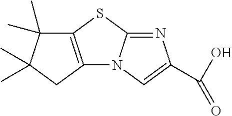 [SMILES: CC1(C)Cc2c(sc3nc(C(=O)O)cn23)C1(C)C] | Ex 36 |
| 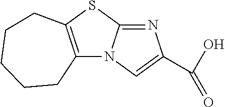 [SMILES: O=C(O)c1cn2c3c(sc2n1)CCCCC3] | Ex 37 |
| 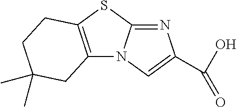 [SMILES: CC1(C)CCc2sc3nc(C(=O)O)cn3c2C1] | Ex 38 |
| 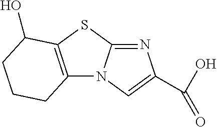 [SMILES: O=C(O)c1cn2c3c(sc2n1)C(O)CCC3] | Ex 39 |
| 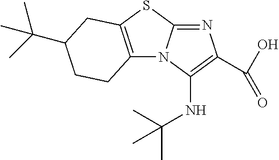 [SMILES: CC(C)(C)Nc1c(C(=O)O)nc2sc3c(n12)CCC(C(C)(C)C)C3] | Ex 40 |
| 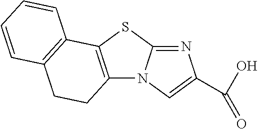 [SMILES: O=C(O)c1cn2c3c(sc2n1)-c1ccccc1CC3] | Ex 41 |
| 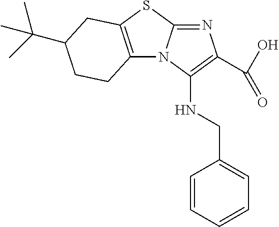 [SMILES: CC(C)(C)C1CCc2c(sc3nc(C(=O)O)c(NCc4ccccc4)n23)C1] | Ex 42 |
| 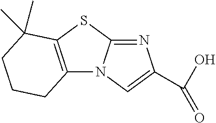 [SMILES: CC1(C)CCCc2c1sc1nc(C(=O)O)cn21] | Ex 43 |
| 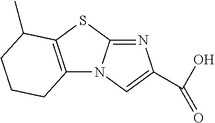 [SMILES: CC1CCCc2c1sc1nc(C(=O)O)cn21] | Ex 44 |
| 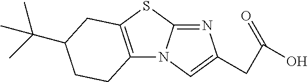 [SMILES: CC(C)(C)C1CCc2c(sc3nc(CC(=O)O)cn23)C1] | Ex 45 |
| 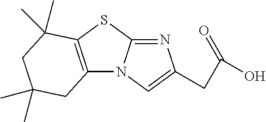 [SMILES: CC1(C)Cc2c(sc3nc(CC(=O)O)cn23)C(C)(C)C1] | Ex 46 |
| 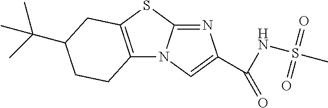 [SMILES: CC(C)(C)C1CCc2c(sc3nc(C(=O)NS(C)(=O)=O)cn23)C1] | Ex 47 |
| 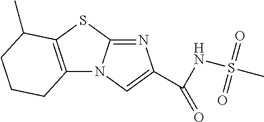 [SMILES: CC1CCCc2c1sc1nc(C(=O)NS(C)(=O)=O)cn21] | Ex 48 |
| 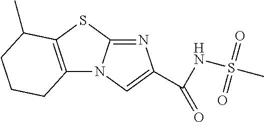 [SMILES: CC1CCCc2c1sc1nc(C(=O)NS(C)(=O)=O)cn21] | Ex 49 |
| 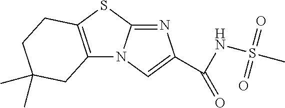 | Ex 50 |
| 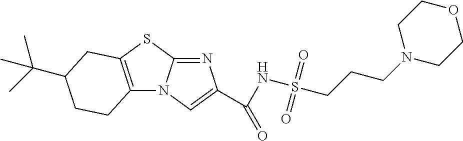 [SMILES: CC(C)(C)C1CCc2c(sc3nc(C(=O)NS(=O)(=O)CCCN4CCOCC4)cn23)C1] | Ex 51 |
| 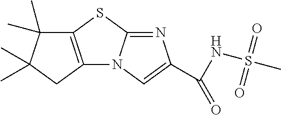 | Ex 52 |
| 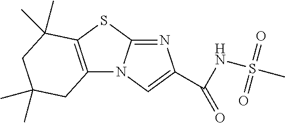 | Ex 53 |
| 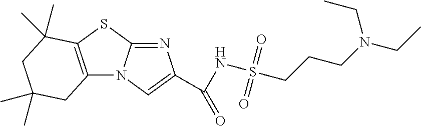 [SMILES: CCN(CC)CCCS(=O)(=O)NC(=O)c1cn2c3c(sc2n1)C(C)(C)CC(C)(C)C3] | Ex 54 |
| 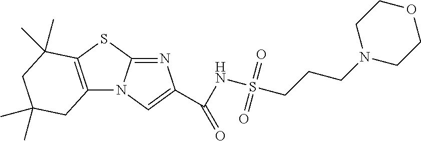 [SMILES: CC1(C)Cc2c(sc3nc(C(=O)NS(=O)(=O)CCCN4CCOCC4)cn23)C(C)(C)C1] | Ex 55 |
| 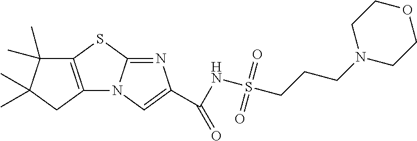 [SMILES: CC1(C)Cc2c(sc3nc(C(=O)NS(=O)(=O)CCCN4CCOCC4)cn23)C1(C)C] | Ex 56 |
| 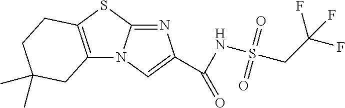 [SMILES: CC1(C)CCc2sc3nc(C(=O)NS(=O)(=O)CC(F)(F)F)cn3c2C1] | Ex 57 |
| 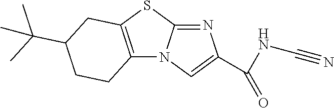 [SMILES: CC(C)(C)C1CCc2c(sc3nc(C(=O)NC#N)cn23)C1] | Ex 58 |
| 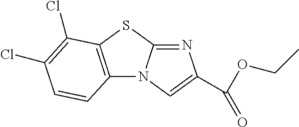 [SMILES: CCOC(=O)c1cn2c(n1)sc1c(Cl)c(Cl)ccc12] | Ex 59 |
| 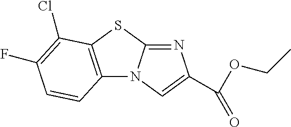 [SMILES: CCOC(=O)c1cn2c(n1)sc1c(Cl)c(F)ccc12] | Ex 60 |
| 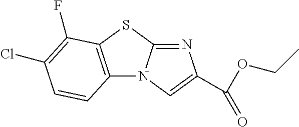 [SMILES: CCOC(=O)c1cn2c(n1)sc1c(F)c(Cl)ccc12] | Ex 61 |
| 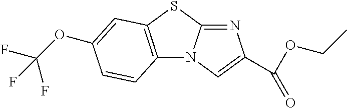 [SMILES: CCOC(=O)c1cn2c(n1)sc1cc(OC(F)(F)F)ccc12] | Ex 62 |
| 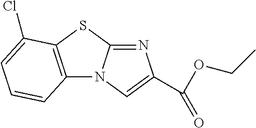 [SMILES: CCOC(=O)c1cn2c(n1)sc1c(Cl)cccc12] | Ex 63 |
| 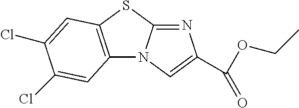 [SMILES: CCOC(=O)c1cn2c(n1)sc1cc(Cl)c(Cl)cc12] | Ex 64 |
| 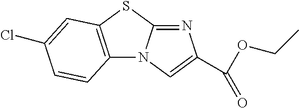 [SMILES: CCOC(=O)c1cn2c(n1)sc1cc(Cl)ccc12] | Ex 65 |
| 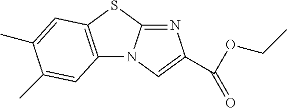 [SMILES: CCOC(=O)c1cn2c(n1)sc1cc(C)c(C)cc12] | Ex 66 |
| 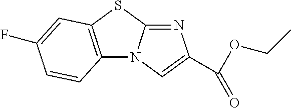 [SMILES: CCOC(=O)c1cn2c(n1)sc1cc(F)ccc12] | Ex 67 |
| 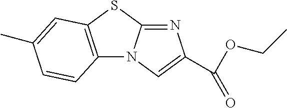 [SMILES: CCOC(=O)c1cn2c(n1)sc1cc(C)ccc12] | Ex 68 |
| 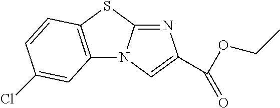 [SMILES: CCOC(=O)c1cn2c(n1)sc1ccc(Cl)cc12] | Ex 69 |
| 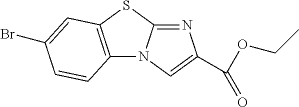 [SMILES: CCOC(=O)c1cn2c(n1)sc1cc(Br)ccc12] | Ex 70 |
| 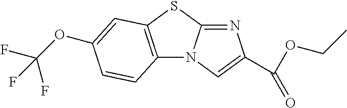 [SMILES: CCOC(=O)c1cn2c(n1)sc1cc(OC(F)(F)F)ccc12] | Ex 71 |
| 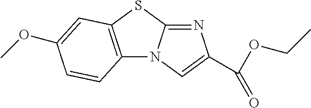 [SMILES: CCOC(=O)c1cn2c(n1)sc1cc(OC)ccc12] | Ex 72 |
| 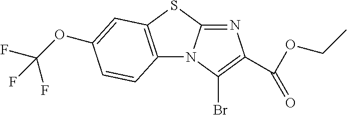 [SMILES: CCOC(=O)c1nc2sc3cc(OC(F)(F)F)ccc3n2c1Br] | Ex 73 |
| 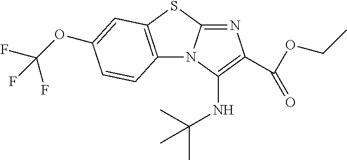 [SMILES: CCOC(=O)c1nc2sc3cc(OC(F)(F)F)ccc3n2c1NC(C)(C)C] | Ex 74 |
| 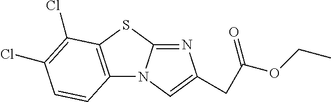 [SMILES: CCOC(=O)Cc1cn2c(n1)sc1c(Cl)c(Cl)ccc12] | Ex 75 |
| 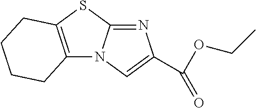 [SMILES: CCOC(=O)c1cn2c3c(sc2n1)CCCC3] | Ex 76 |
| 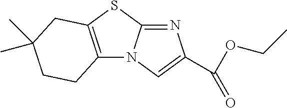 [SMILES: CCOC(=O)c1cn2c3c(sc2n1)CC(C)(C)CC3] | Ex 77 |
| 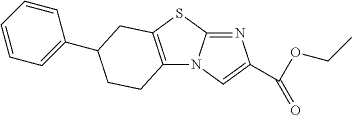 [SMILES: CCOC(=O)c1cn2c3c(sc2n1)CC(c1ccccc1)CC3] | Ex 78 |
| 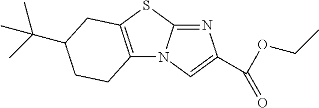 [SMILES: CCOC(=O)c1cn2c3c(sc2n1)CC(C(C)(C)C)CC3] | Ex 79 |
| 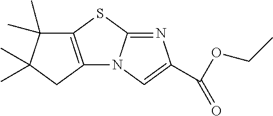 [SMILES: CCOC(=O)c1cn2c3c(sc2n1)C(C)(C)C(C)(C)C3] | Ex 80 |
| 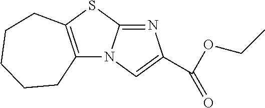 [SMILES: CCOC(=O)c1cn2c3c(sc2n1)CCCCC3] | Ex 81 |
| 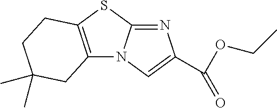 [SMILES: CCOC(=O)c1cn2c3c(sc2n1)CCC(C)(C)C3] | Ex 82 |
| 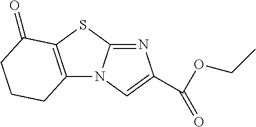 [SMILES: CCOC(=O)c1cn2c3c(sc2n1)C(=O)CCC3] | Ex 83 |
| 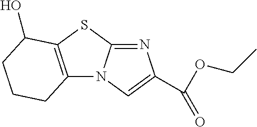 [SMILES: CCOC(=O)c1cn2c3c(sc2n1)C(O)CCC3] | Ex 84 |
| 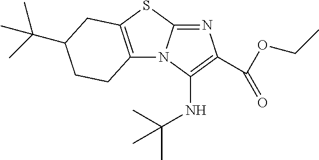 [SMILES: CCOC(=O)c1nc2sc3c(n2c1NC(C)(C)C)CCC(C(C)(C)C)C3] | Ex 85 |
| 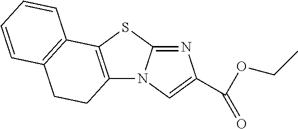 [SMILES: CCOC(=O)c1cn2c3c(sc2n1)-c1ccccc1CC3] | Ex 86 |
| 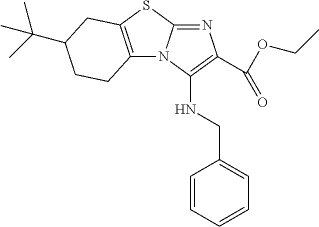 [SMILES: CCOC(=O)c1nc2sc3c(n2c1NCc1ccccc1)CCC(C(C)(C)C)C3] | Ex 87 |
| 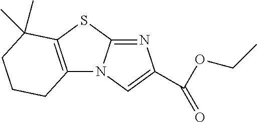 [SMILES: CCOC(=O)c1cn2c3c(sc2n1)C(C)(C)CCC3] | Ex 88 |
| 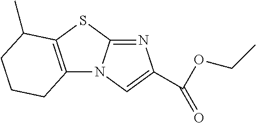 [SMILES: CCOC(=O)c1cn2c3c(sc2n1)C(C)CCC3] | Ex 89 |
| 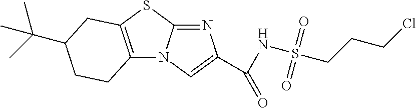 [SMILES: CC(C)(C)C1CCc2c(sc3nc(C(=O)NS(=O)(=O)CCCCl)cn23)C1] | Ex 90 |
| 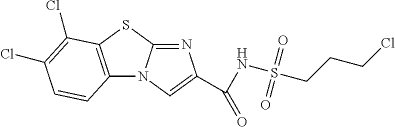 [SMILES: O=C(NS(=O)(=O)CCCCl)c1cn2c(n1)sc1c(Cl)c(Cl)ccc12] | Ex 91 |
| 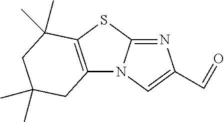 [SMILES: CC1(C)Cc2c(sc3nc(C=O)cn23)C(C)(C)C1] | Ex 92 |
| 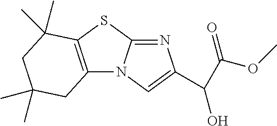 | Ex 93 |
| 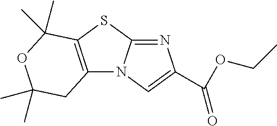 [SMILES: CCOC(=O)c1cn2c3c(sc2n1)C(C)(C)OC(C)(C)C3] | Ex 94 |
| 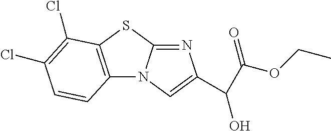 [SMILES: CCOC(=O)C(O)c1cn2c(n1)sc1c(Cl)c(Cl)ccc12] | Ex 95 |
| 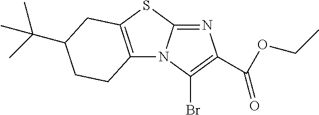 [SMILES: CCOC(=O)c1nc2sc3c(n2c1Br)CCC(C(C)(C)C)C3] | Ex 96 |
| 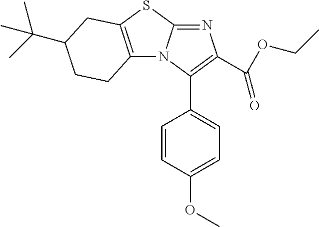 [SMILES: CCOC(=O)c1nc2sc3c(n2c1-c1ccc(OC)cc1)CCC(C(C)(C)C)C3] | Ex 97 |
| 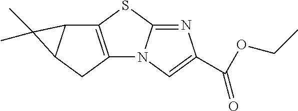 [SMILES: CCOC(=O)c1cn2c3c(sc2n1)C1C(C3)C1(C)C] | Ex 98 |
| 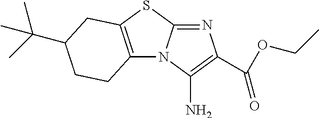 [SMILES: CCOC(=O)c1nc2sc3c(n2c1N)CCC(C(C)(C)C)C3] | Ex 99 |
| 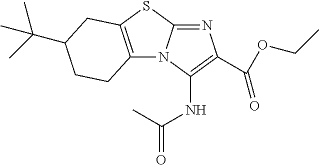 [SMILES: CCOC(=O)c1nc2sc3c(n2c1NC(C)=O)CCC(C(C)(C)C)C3] | Ex 100 |
| 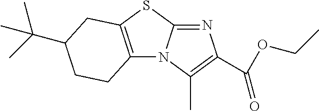 [SMILES: CCOC(=O)c1nc2sc3c(n2c1C)CCC(C(C)(C)C)C3] | Ex 101 |
| 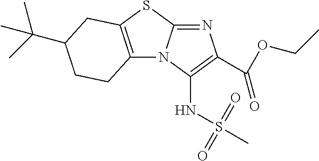 [SMILES: CCOC(=O)c1nc2sc3c(n2c1NS(C)(=O)=O)CCC(C(C)(C)C)C3] | Ex 102 |
| 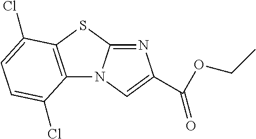 [SMILES: CCOC(=O)c1cn2c(n1)sc1c(Cl)ccc(Cl)c12] | Ex 103 |
| 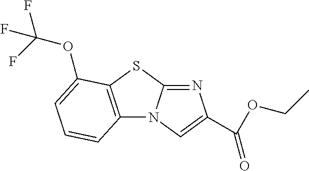 [SMILES: CCOC(=O)c1cn2c(n1)sc1c(OC(F)(F)F)cccc12] | Ex 104 |
| 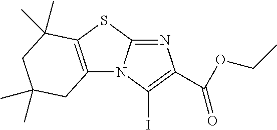 [SMILES: CCOC(=O)c1nc2sc3c(n2c1I)CC(C)(C)CC3(C)C] | Ex 105 |
| 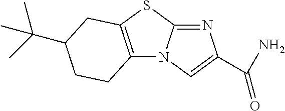 [SMILES: CC(C)(C)C1CCc2c(sc3nc(C(N)=O)cn23)C1] | Ex 106 |
| 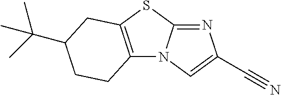 [SMILES: CC(C)(C)C1CCc2c(sc3nc(C#N)cn23)C1] | Ex 107 |
| 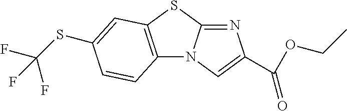 [SMILES: CCOC(=O)c1cn2c(n1)sc1cc(SC(F)(F)F)ccc12] | Ex 108 |
| 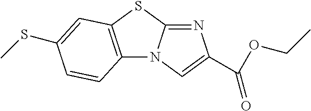 [SMILES: CCOC(=O)c1cn2c(n1)sc1cc(SC)ccc12] | Ex 109 |
| 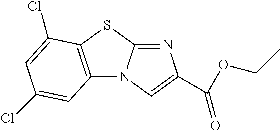 [SMILES: CCOC(=O)c1cn2c(n1)sc1c(Cl)cc(Cl)cc12] | Ex 110 |
| 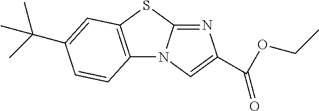 [SMILES: CCOC(=O)c1cn2c(n1)sc1cc(C(C)(C)C)ccc12] | Ex 111 |
| 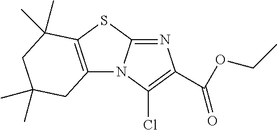 [SMILES: CCOC(=O)c1nc2sc3c(n2c1Cl)CC(C)(C)CC3(C)C] | Ex 112 |
| 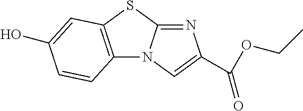 [SMILES: CCOC(=O)c1cn2c(n1)sc1cc(O)ccc12] | Ex 113 |
| 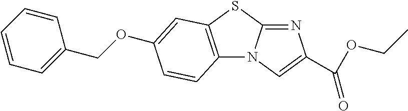 [SMILES: CCOC(=O)c1cn2c(n1)sc1cc(OCc3ccccc3)ccc12] | Ex 114 |
| 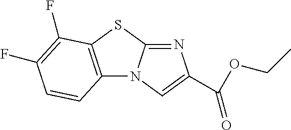 [SMILES: CCOC(=O)c1cn2c(n1)sc1c(F)c(F)ccc12] | Ex 115 |
| 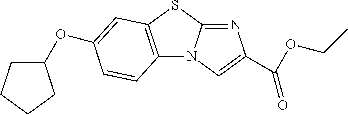 [SMILES: CCOC(=O)c1cn2c(n1)sc1cc(OC3CCCC3)ccc12] | Ex 116 |
| 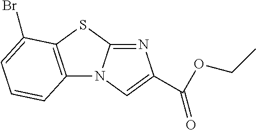 [SMILES: CCOC(=O)c1cn2c(n1)sc1c(Br)cccc12] | Ex 117 |
| 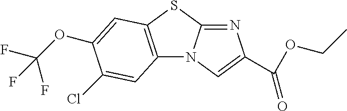 [SMILES: CCOC(=O)c1cn2c(n1)sc1cc(OC(F)(F)F)c(Cl)cc12] | Ex 118 |
| 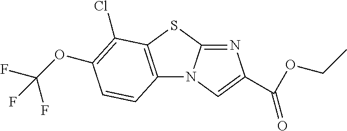 [SMILES: CCOC(=O)c1cn2c(n1)sc1c(Cl)c(OC(F)(F)F)ccc12] | Ex 119 |
| 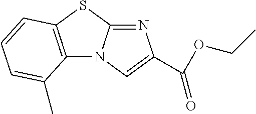 [SMILES: CCOC(=O)c1cn2c(n1)sc1cccc(C)c12] | Ex 120 |
| 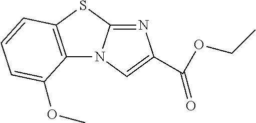 [SMILES: CCOC(=O)c1cn2c(n1)sc1cccc(OC)c12] | Ex 121 |
| 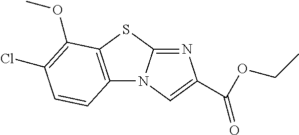 [SMILES: CCOC(=O)c1cn2c(n1)sc1c(OC)c(Cl)ccc12] | Ex 122 |
| 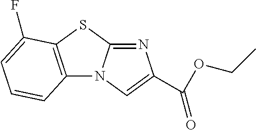 [SMILES: CCOC(=O)c1cn2c(n1)sc1c(F)cccc12] | Ex 123 |
| 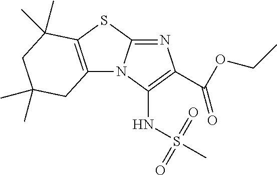 [SMILES: CCOC(=O)c1nc2sc3c(n2c1NS(C)(=O)=O)CC(C)(C)CC3(C)C] | Ex 124 |
| 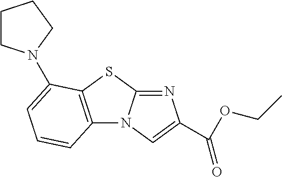 [SMILES: CCOC(=O)c1cn2c(n1)sc1c(N3CCCC3)cccc12] | Ex 125 |
| 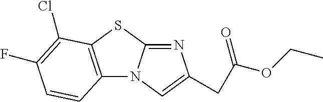 [SMILES: CCOC(=O)Cc1cn2c(n1)sc1c(Cl)c(F)ccc12] | Ex 126 |
| 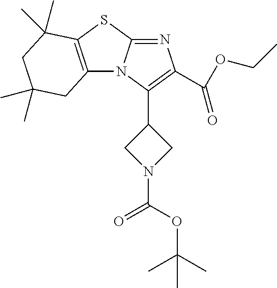 [SMILES: CCOC(=O)c1nc2sc3c(n2c1C1CN(C(=O)OC(C)(C)C)C1)CC(C)(C)CC3(C)C] | Ex 127 |
| 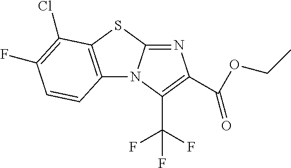 [SMILES: CCOC(=O)c1nc2sc3c(Cl)c(F)ccc3n2c1C(F)(F)F] | Ex 128 |
| 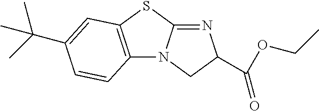 [SMILES: CCOC(=O)C1CN2C(=N1)Sc1cc(C(C)(C)C)ccc12] | Ex 129 |
| 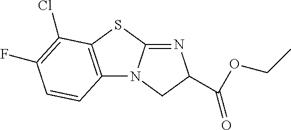 [SMILES: CCOC(=O)C1CN2C(=N1)Sc1c2ccc(F)c1Cl] | Ex 130 |
| 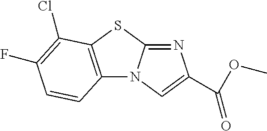 [SMILES: COC(=O)c1cn2c(n1)sc1c(Cl)c(F)ccc12] | Ex 131 |
| 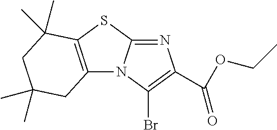 [SMILES: CCOC(=O)c1nc2sc3c(n2c1Br)CC(C)(C)CC3(C)C] | Ex 132 |
| 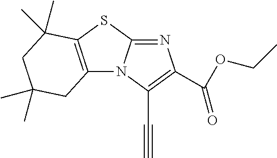 [SMILES: C#Cc1c(C(=O)OCC)nc2sc3c(n12)CC(C)(C)CC3(C)C] | Ex 133 |
| 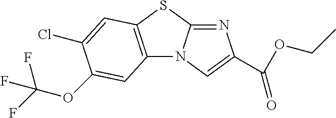 [SMILES: CCOC(=O)c1cn2c(n1)sc1cc(Cl)c(OC(F)(F)F)cc12] | Ex 134 |
| 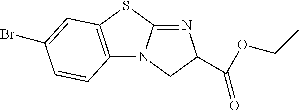 [SMILES: CCOC(=O)C1CN2C(=N1)Sc1cc(Br)ccc12] | Ex 135 |
| 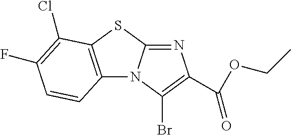 [SMILES: CCOC(=O)c1nc2sc3c(Cl)c(F)ccc3n2c1Br] | Ex 136 |
| 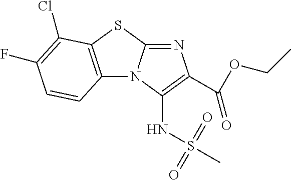 [SMILES: CCOC(=O)c1nc2sc3c(Cl)c(F)ccc3n2c1NS(C)(=O)=O] | Ex 137 |
| 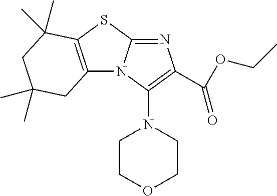 [SMILES: CCOC(=O)c1nc2sc3c(n2c1N1CCOCC1)CC(C)(C)CC3(C)C] | Ex 138 |
| 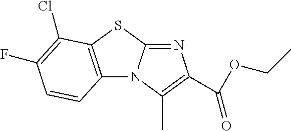 [SMILES: CCOC(=O)c1nc2sc3c(Cl)c(F)ccc3n2c1C] | Ex 139 |
| 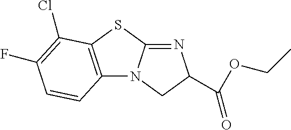 [SMILES: CCOC(=O)C1CN2C(=N1)Sc1c2ccc(F)c1Cl] | Ex 140 |
| 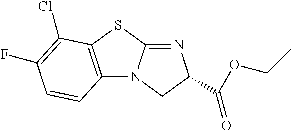 [SMILES: CCOC(=O)[C@@H]1CN2C(=N1)Sc1c2ccc(F)c1Cl] | Ex 141 |
| 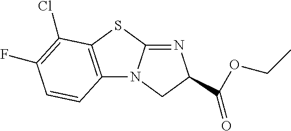 [SMILES: CCOC(=O)[C@H]1CN2C(=N1)Sc1c2ccc(F)c1Cl] | |
| 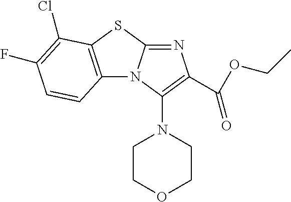 [SMILES: CCOC(=O)c1nc2sc3c(Cl)c(F)ccc3n2c1N1CCOCC1] | Ex 142 |
| 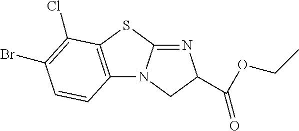 [SMILES: CCOC(=O)C1CN2C(=N1)Sc1c2ccc(Br)c1Cl] | Ex 143 |
| 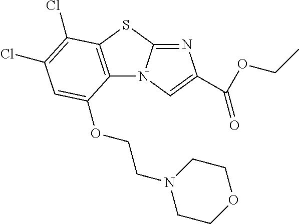 [SMILES: CCOC(=O)c1cn2c(n1)sc1c(Cl)c(Cl)cc(OCCN3CCOCC3)c12] | Ex 144 |
| 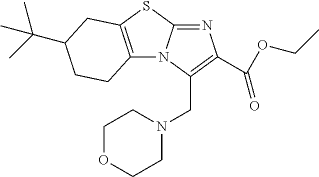 [SMILES: CCOC(=O)c1nc2sc3c(n2c1CN1CCOCC1)CCC(C(C)(C)C)C3] | Ex 145 |
| 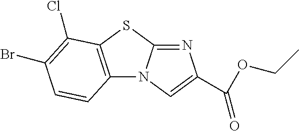 [SMILES: CCOC(=O)c1cn2c(n1)sc1c(Cl)c(Br)ccc12] | Ex 146 |
| 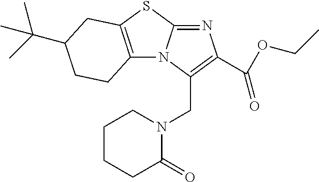 [SMILES: CCOC(=O)c1nc2sc3c(n2c1CN1CCCCC1=O)CCC(C(C)(C)C)C3] | Ex 147 |
| 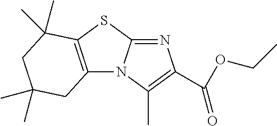 [SMILES: CCOC(=O)c1nc2sc3c(n2c1C)CC(C)(C)CC3(C)C] | Ex 148 |
| 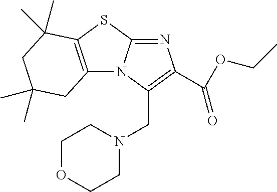 [SMILES: CCOC(=O)c1nc2sc3c(n2c1CN1CCOCC1)CC(C)(C)CC3(C)C] | Ex 149 |
| 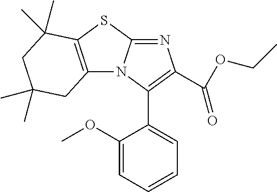 [SMILES: CCOC(=O)c1nc2sc3c(n2c1-c1ccccc1OC)CC(C)(C)CC3(C)C] | Ex 150 |
| 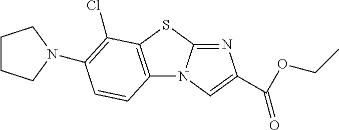 [SMILES: CCOC(=O)c1cn2c(n1)sc1c(Cl)c(N3CCCC3)ccc12] | Ex 151 |
| 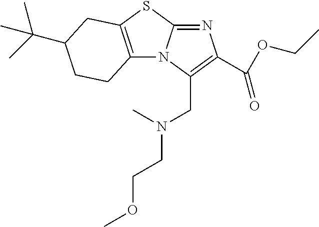 [SMILES: CCOC(=O)c1nc2sc3c(n2c1CN(C)CCOC)CCC(C(C)(C)C)C3] | Ex 152 |
| 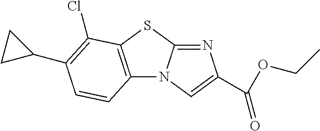 [SMILES: CCOC(=O)c1cn2c(n1)sc1c(Cl)c(C3CC3)ccc12] | Ex 153 |
| 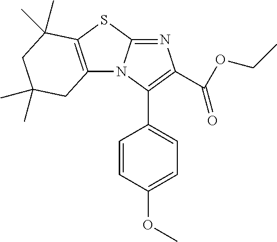 [SMILES: CCOC(=O)c1nc2sc3c(n2c1-c1ccc(OC)cc1)CC(C)(C)CC3(C)C] | Ex 154 |
| 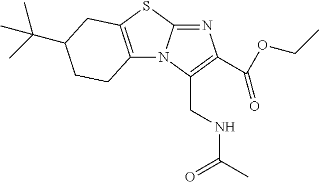 [SMILES: CCOC(=O)c1nc2sc3c(n2c1CNC(C)=O)CCC(C(C)(C)C)C3] | Ex 155 |
| 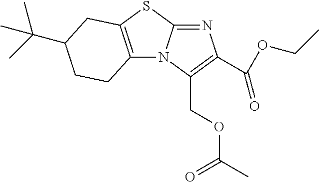 [SMILES: CCOC(=O)c1nc2sc3c(n2c1COC(C)=O)CCC(C(C)(C)C)C3] | Ex 156 |
| 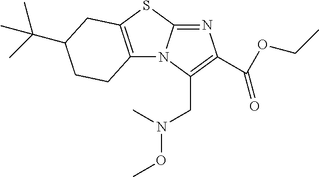 [SMILES: CCOC(=O)c1nc2sc3c(n2c1CN(C)OC)CCC(C(C)(C)C)C3] | Ex 157 |
| 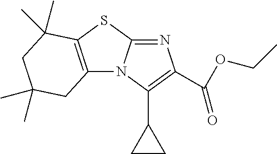 [SMILES: CCOC(=O)c1nc2sc3c(n2c1C1CC1)CC(C)(C)CC3(C)C] | Ex 158 |
| 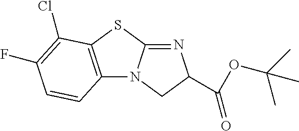 [SMILES: CC(C)(C)OC(=O)C1CN2C(=N1)Sc1c2ccc(F)c1Cl] | Ex 159 |
| 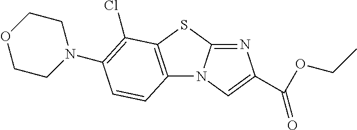 [SMILES: CCOC(=O)c1cn2c(n1)sc1c(Cl)c(N3CCOCC3)ccc12] | Ex 160 |
| 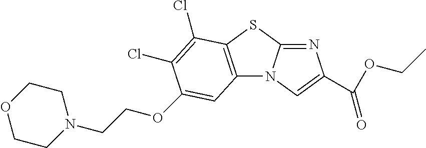 [SMILES: CCOC(=O)c1cn2c(n1)sc1c(Cl)c(Cl)c(OCCN3CCOCC3)cc12] | Ex 161 |
| 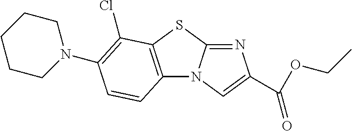 [SMILES: CCOC(=O)c1cn2c(n1)sc1c(Cl)c(N3CCCCC3)ccc12] | Ex 162 |
| 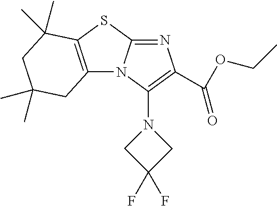 [SMILES: CCOC(=O)c1nc2sc3c(n2c1N1CC(F)(F)C1)CC(C)(C)CC3(C)C] | Ex 163 |
| 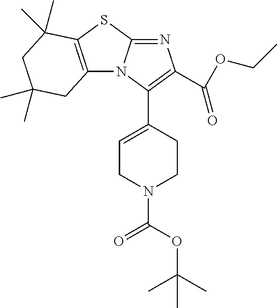 [SMILES: CCOC(=O)c1nc2sc3c(n2c1C1=CCN(C(=O)OC(C)(C)C)CC1)CC(C)(C)CC3(C)C] | Ex 164 |
| 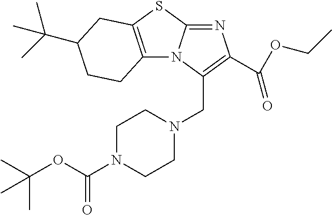 [SMILES: CCOC(=O)c1nc2sc3c(n2c1CN1CCN(C(=O)OC(C)(C)C)CC1)CCC(C(C)(C)C)C3] | Ex 165 |
| 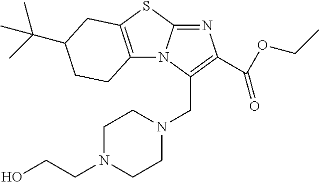 [SMILES: CCOC(=O)c1nc2sc3c(n2c1CN1CCN(CCO)CC1)CCC(C(C)(C)C)C3] | Ex 166 |
| 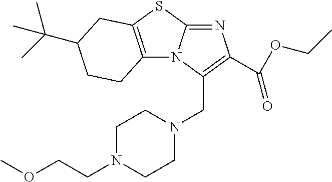 [SMILES: CCOC(=O)c1nc2sc3c(n2c1CN1CCN(CCOC)CC1)CCC(C(C)(C)C)C3] | Ex 167 |
| 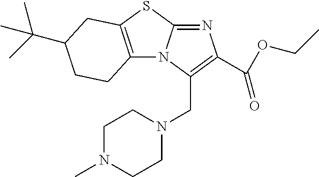 [SMILES: CCOC(=O)c1nc2sc3c(n2c1CN1CCN(C)CC1)CCC(C(C)(C)C)C3] | Ex 168 |
| 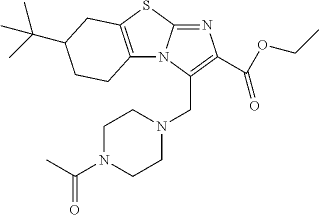 [SMILES: CCOC(=O)c1nc2sc3c(n2c1CN1CCN(C(C)=O)CC1)CCC(C(C)(C)C)C3] | Ex 169 |
| 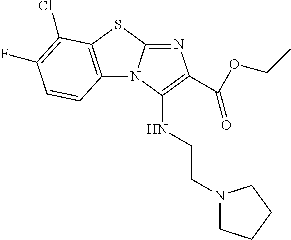 [SMILES: CCOC(=O)c1nc2sc3c(Cl)c(F)ccc3n2c1NCCN1CCCC1] | Ex 170 |
| 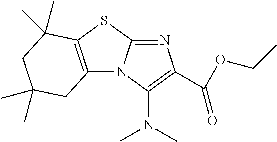 [SMILES: CCOC(=O)c1nc2sc3c(n2c1N(C)C)CC(C)(C)CC3(C)C] | Ex 171 |
| 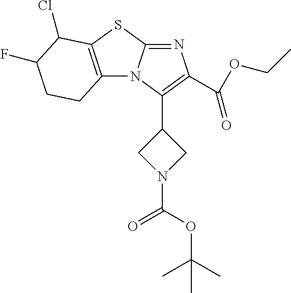 [SMILES: CCOC(=O)c1nc2sc3c(n2c1C1CN(C(=O)OC(C)(C)C)C1)CCC(F)C3Cl] | Ex 172 |
| 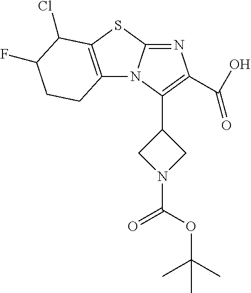 [SMILES: CC(C)(C)OC(=O)N1CC(c2c(C(=O)O)nc3sc4c(n23)CCC(F)C4Cl)C1] | Ex 173 |
| 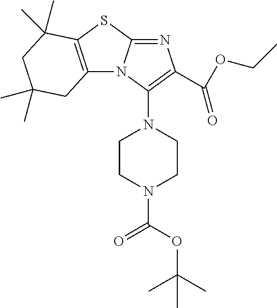 [SMILES: CCOC(=O)c1nc2sc3c(n2c1N1CCN(C(=O)OC(C)(C)C)CC1)CC(C)(C)CC3(C)C] | Ex 174 |
| 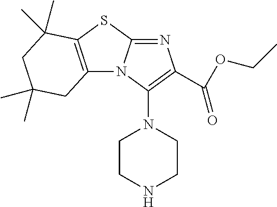 [SMILES: CCOC(=O)c1nc2sc3c(n2c1N1CCNCC1)CC(C)(C)CC3(C)C] | Ex 175 |
| 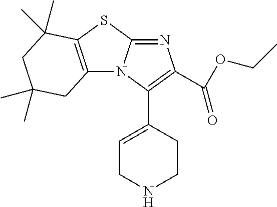 [SMILES: CCOC(=O)c1nc2sc3c(n2c1C1=CCNCC1)CC(C)(C)CC3(C)C] | Ex 176 |
| 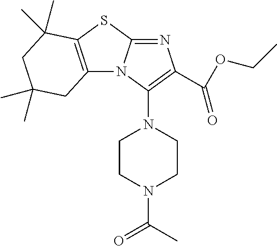 [SMILES: CCOC(=O)c1nc2sc3c(n2c1N1CCN(C(C)=O)CC1)CC(C)(C)CC3(C)C] | Ex 177 |
| 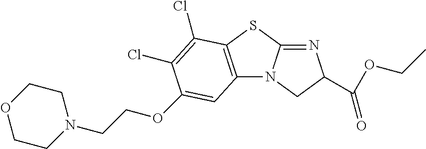 [SMILES: CCOC(=O)C1CN2C(=N1)Sc1c2cc(OCCN2CCOCC2)c(Cl)c1Cl] | Ex 178 |
| 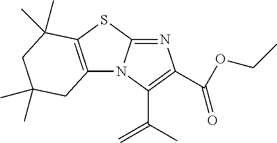 [SMILES: C=C(C)c1c(C(=O)OCC)nc2sc3c(n12)CC(C)(C)CC3(C)C] | Ex 179 |
| 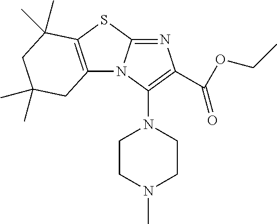 [SMILES: CCOC(=O)c1nc2sc3c(n2c1N1CCN(C)CC1)CC(C)(C)CC3(C)C] | Ex 180 |
| 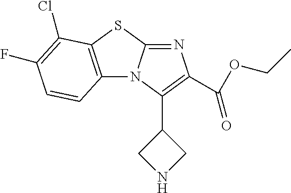 [SMILES: CCOC(=O)c1nc2sc3c(Cl)c(F)ccc3n2c1C1CNC1] | Ex 181 |
| 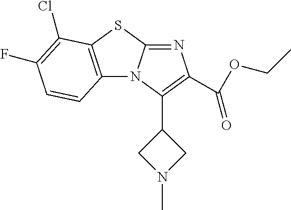 [SMILES: CCOC(=O)c1nc2sc3c(Cl)c(F)ccc3n2c1C1CN(C)C1] | Ex 182 |
| 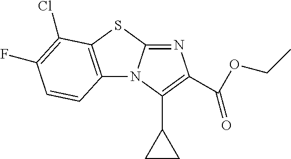 [SMILES: CCOC(=O)c1nc2sc3c(Cl)c(F)ccc3n2c1C1CC1] | Ex 183 |
| 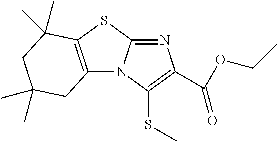 [SMILES: CCOC(=O)c1nc2sc3c(n2c1SC)CC(C)(C)CC3(C)C] | Ex 184 |
| 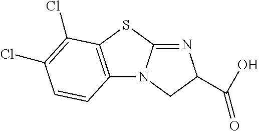 [SMILES: O=C(O)C1CN2C(=N1)Sc1c2ccc(Cl)c1Cl] | Ex 185 |
| 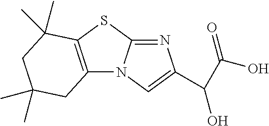 [SMILES: CC1(C)Cc2c(sc3nc(C(O)C(=O)O)cn23)C(C)(C)C1] | Ex 186 |
| 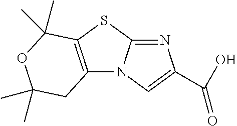 [SMILES: CC1(C)Cc2c(sc3nc(C(=O)O)cn23)C(C)(C)O1] | Ex 187 |
| 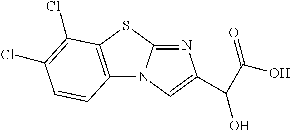 [SMILES: O=C(O)C(O)c1cn2c(n1)sc1c(Cl)c(Cl)ccc12] | Ex 188 |
| 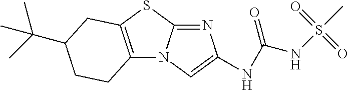 [SMILES: CC(C)(C)C1CCc2c(sc3nc(NC(=O)NS(C)(=O)=O)cn23)C1] | Ex 189 |
| 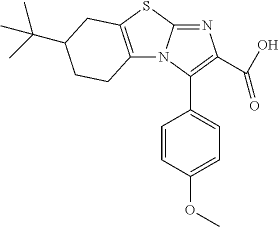 [SMILES: COc1ccc(-c2c(C(=O)O)nc3sc4c(n23)CCC(C(C)(C)C)C4)cc1] | Ex 190 |
| 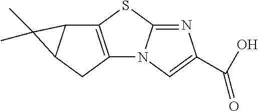 [SMILES: CC1(C)C2Cc3c(sc4nc(C(=O)O)cn34)C21] | Ex 191 |
| 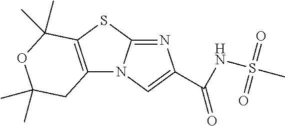 | Ex 192 |
| 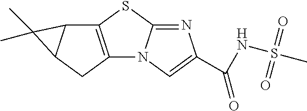 | Ex 193 |
| 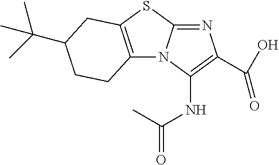 [SMILES: CC(=O)Nc1c(C(=O)O)nc2sc3c(n12)CCC(C(C)(C)C)C3] | Ex 194 |
| 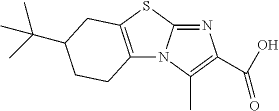 [SMILES: Cc1c(C(=O)O)nc2sc3c(n12)CCC(C(C)(C)C)C3] | Ex 195 |
| 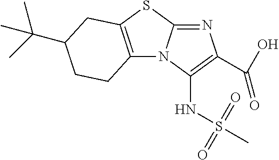 [SMILES: CC(C)(C)C1CCc2c(sc3nc(C(=O)O)c(NS(C)(=O)=O)n23)C1] | Ex 196 |
| 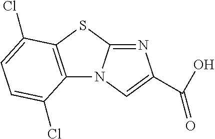 [SMILES: O=C(O)c1cn2c(n1)sc1c(Cl)ccc(Cl)c12] | Ex 197 |
| 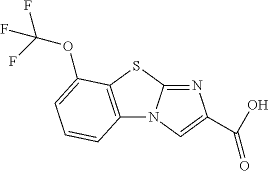 [SMILES: O=C(O)c1cn2c(n1)sc1c(OC(F)(F)F)cccc12] | Ex 198 |
| 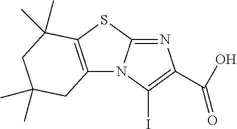 [SMILES: CC1(C)Cc2c(sc3nc(C(=O)O)c(I)n23)C(C)(C)C1] | Ex 199 |
| 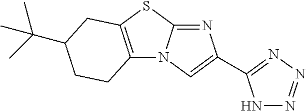 [SMILES: CC(C)(C)C1CCc2c(sc3nc(-c4nnn[nH]4)cn23)C1] | Ex 200 |
| 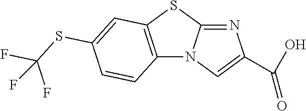 [SMILES: O=C(O)c1cn2c(n1)sc1cc(SC(F)(F)F)ccc12] | Ex 201 |
| 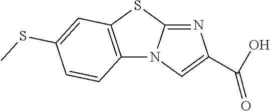 [SMILES: CSc1ccc2c(c1)sc1nc(C(=O)O)cn12] | Ex 202 |
| 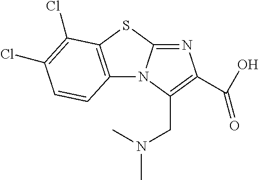 [SMILES: CN(C)Cc1c(C(=O)O)nc2sc3c(Cl)c(Cl)ccc3n12] | Ex 203 |
| 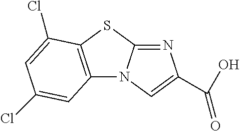 [SMILES: O=C(O)c1cn2c(n1)sc1c(Cl)cc(Cl)cc12] | Ex 204 |
| 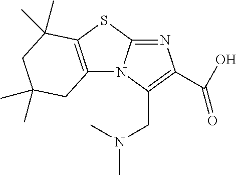 [SMILES: CN(C)Cc1c(C(=O)O)nc2sc3c(n12)CC(C)(C)CC3(C)C] | Ex 205 |
| 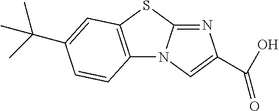 [SMILES: CC(C)(C)c1ccc2c(c1)sc1nc(C(=O)O)cn12] | Ex 206 |
| 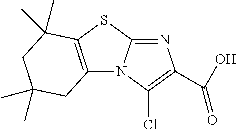 [SMILES: CC1(C)Cc2c(sc3nc(C(=O)O)c(Cl)n23)C(C)(C)C1] | Ex 207 |
| 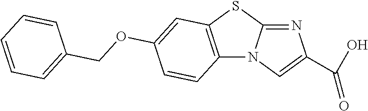 [SMILES: O=C(O)c1cn2c(n1)sc1cc(OCc3ccccc3)ccc12] | Ex 208 |
| 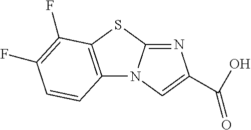 [SMILES: O=C(O)c1cn2c(n1)sc1c(F)c(F)ccc12] | Ex 209 |
| 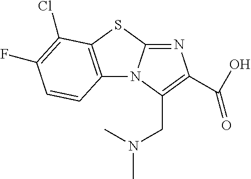 [SMILES: CN(C)Cc1c(C(=O)O)nc2sc3c(Cl)c(F)ccc3n12] | Ex 210 |
| 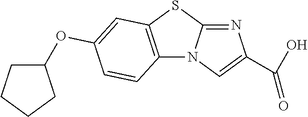 [SMILES: O=C(O)c1cn2c(n1)sc1cc(OC3CCCC3)ccc12] | Ex 211 |
| 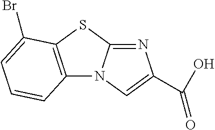 [SMILES: O=C(O)c1cn2c(n1)sc1c(Br)cccc12] | Ex 212 |
| 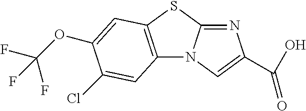 [SMILES: O=C(O)c1cn2c(n1)sc1cc(OC(F)(F)F)c(Cl)cc12] | Ex 213 |
| 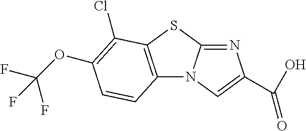 [SMILES: O=C(O)c1cn2c(n1)sc1c(Cl)c(OC(F)(F)F)ccc12] | Ex 214 |
| 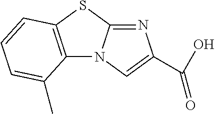 [SMILES: Cc1cccc2sc3nc(C(=O)O)cn3c12] | Ex 215 |
| 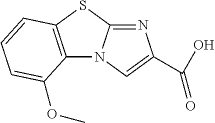 [SMILES: COc1cccc2sc3nc(C(=O)O)cn3c12] | Ex 216 |
| 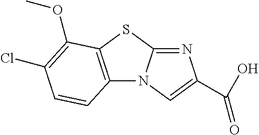 [SMILES: COc1c(Cl)ccc2c1sc1nc(C(=O)O)cn12] | Ex 217 |
| 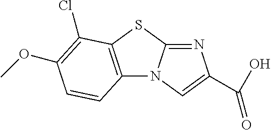 [SMILES: COc1ccc2c(sc3nc(C(=O)O)cn32)c1Cl] | Ex 218 |
| 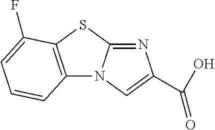 [SMILES: O=C(O)c1cn2c(n1)sc1c(F)cccc12] | Ex 219 |
| 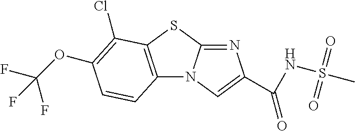 [SMILES: CS(=O)(=O)NC(=O)c1cn2c(n1)sc1c(Cl)c(OC(F)(F)F)ccc12] | Ex 220 |
| 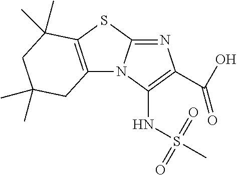 [SMILES: CC1(C)Cc2c(sc3nc(C(=O)O)c(NS(C)(=O)=O)n23)C(C)(C)C1] | Ex 221 |
| 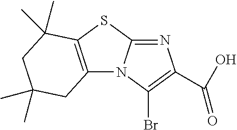 [SMILES: CC1(C)Cc2c(sc3nc(C(=O)O)c(Br)n23)C(C)(C)C1] | Ex 222 |
| 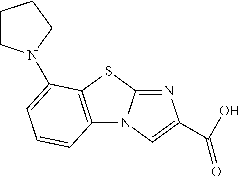 [SMILES: O=C(O)c1cn2c(n1)sc1c(N3CCCC3)cccc12] | Ex 223 |
| 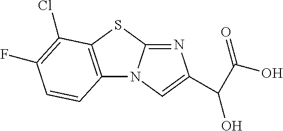 [SMILES: O=C(O)C(O)c1cn2c(n1)sc1c(Cl)c(F)ccc12] | Ex 224 |
| 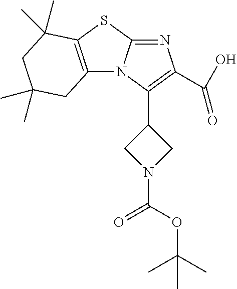 [SMILES: CC1(C)Cc2c(sc3nc(C(=O)O)c(C4CN(C(=O)OC(C)(C)C)C4)n23)C(C)(C)C1] | Ex 225 |
| 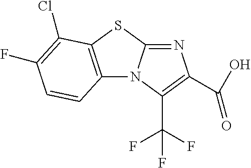 [SMILES: O=C(O)c1nc2sc3c(Cl)c(F)ccc3n2c1C(F)(F)F] | Ex 226 |
| 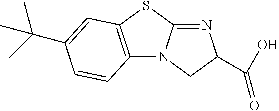 [SMILES: CC(C)(C)c1ccc2c(c1)SC1=NC(C(=O)O)CN12] | Ex 227 |
| 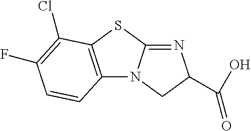 [SMILES: O=C(O)C1CN2C(=N1)Sc1c2ccc(F)c1Cl] | Ex 228 |
| 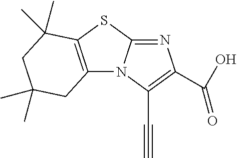 [SMILES: C#Cc1c(C(=O)O)nc2sc3c(n12)CC(C)(C)CC3(C)C] | Ex 229 |
| 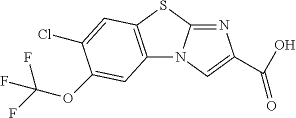 [SMILES: O=C(O)c1cn2c(n1)sc1cc(Cl)c(OC(F)(F)F)cc12] | Ex 230 |
| 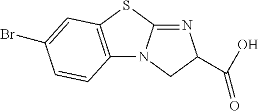 [SMILES: O=C(O)C1CN2C(=N1)Sc1cc(Br)ccc12] | Ex 231 |
| 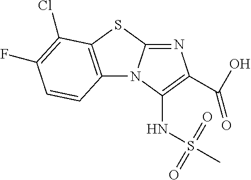 [SMILES: CS(=O)(=O)Nc1c(C(=O)O)nc2sc3c(Cl)c(F)ccc3n12] | Ex 232 |
| 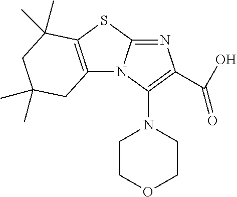 [SMILES: CC1(C)Cc2c(sc3nc(C(=O)O)c(N4CCOCC4)n23)C(C)(C)C1] | Ex 233 |
| 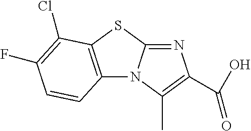 [SMILES: Cc1c(C(=O)O)nc2sc3c(Cl)c(F)ccc3n12] | Ex 234 |
| 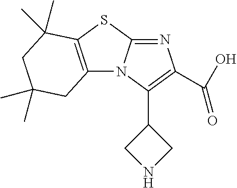 [SMILES: CC1(C)Cc2c(sc3nc(C(=O)O)c(C4CNC4)n23)C(C)(C)C1] | Ex 235 |
| 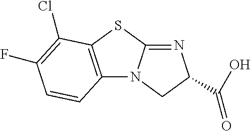 [SMILES: O=C(O)[C@@H]1CN2C(=N1)Sc1c2ccc(F)c1Cl] | Ex 236 |
| 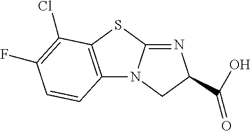 [SMILES: O=C(O)[C@H]1CN2C(=N1)Sc1c2ccc(F)c1Cl] | Ex 237 |
| 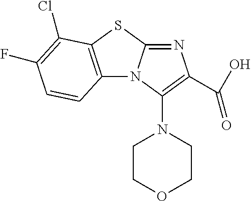 [SMILES: O=C(O)c1nc2sc3c(Cl)c(F)ccc3n2c1N1CCOCC1] | Ex 238 |
| 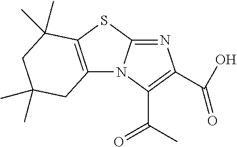 [SMILES: CC(=O)c1c(C(=O)O)nc2sc3c(n12)CC(C)(C)CC3(C)C] | Ex 239 |
| 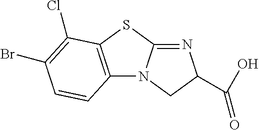 [SMILES: O=C(O)C1CN2C(=N1)Sc1c2ccc(Br)c1Cl] | Ex 240 |
| 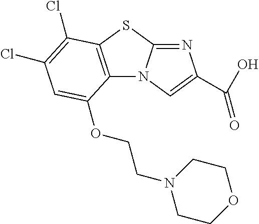 [SMILES: O=C(O)c1cn2c(n1)sc1c(Cl)c(Cl)cc(OCCN3CCOCC3)c12] | Ex 241 |
| 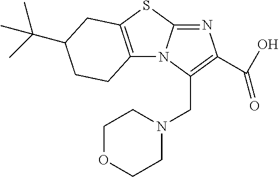 [SMILES: CC(C)(C)C1CCc2c(sc3nc(C(=O)O)c(CN4CCOCC4)n23)C1] | Ex 242 |
| 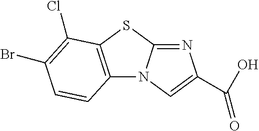 [SMILES: O=C(O)c1cn2c(n1)sc1c(Cl)c(Br)ccc12] | Ex 243 |
| 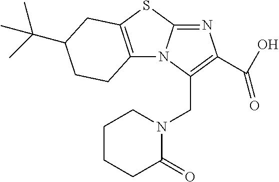 [SMILES: CC(C)(C)C1CCc2c(sc3nc(C(=O)O)c(CN4CCCCC4=O)n23)C1] | Ex 244 |
| 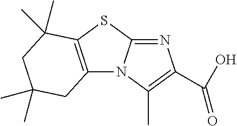 [SMILES: Cc1c(C(=O)O)nc2sc3c(n12)CC(C)(C)CC3(C)C] | Ex 245 |
| 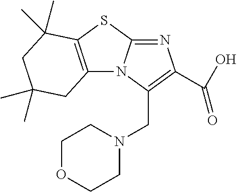 [SMILES: CC1(C)Cc2c(sc3nc(C(=O)O)c(CN4CCOCC4)n23)C(C)(C)C1] | Ex 246 |
| 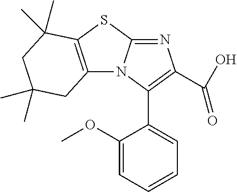 [SMILES: COc1ccccc1-c1c(C(=O)O)nc2sc3c(n12)CC(C)(C)CC3(C)C] | Ex 247 |
| 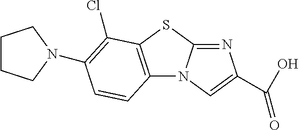 [SMILES: O=C(O)c1cn2c(n1)sc1c(Cl)c(N3CCCC3)ccc12] | Ex 248 |
| 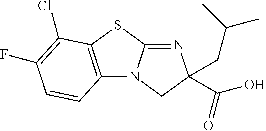 [SMILES: CC(C)CC1(C(=O)O)CN2C(=N1)Sc1c2ccc(F)c1Cl] | Ex 249 |
| 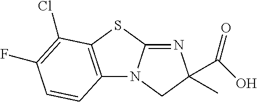 [SMILES: CC1(C(=O)O)CN2C(=N1)Sc1c2ccc(F)c1Cl] | Ex 250 |
| 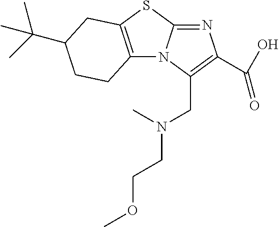 [SMILES: COCCN(C)Cc1c(C(=O)O)nc2sc3c(n12)CCC(C(C)(C)C)C3] | Ex 251 |
| 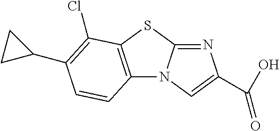 [SMILES: O=C(O)c1cn2c(n1)sc1c(Cl)c(C3CC3)ccc12] | Ex 252 |
| 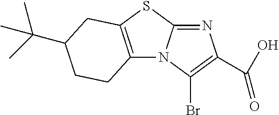 [SMILES: CC(C)(C)C1CCc2c(sc3nc(C(=O)O)c(Br)n23)C1] | Ex 253 |
| 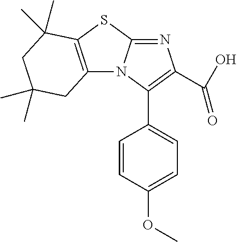 [SMILES: COc1ccc(-c2c(C(=O)O)nc3sc4c(n23)CC(C)(C)CC4(C)C)cc1] | Ex 254 |
| 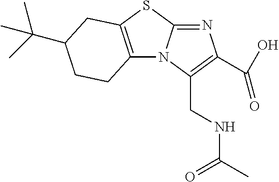 [SMILES: CC(=O)NCc1c(C(=O)O)nc2sc3c(n12)CCC(C(C)(C)C)C3] | Ex 255 |
| 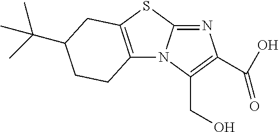 [SMILES: CC(C)(C)C1CCc2c(sc3nc(C(=O)O)c(CO)n23)C1] | Ex 256 |
| 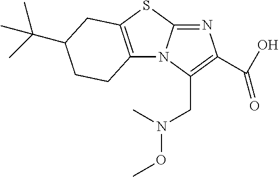 [SMILES: CON(C)Cc1c(C(=O)O)nc2sc3c(n12)CCC(C(C)(C)C)C3] | Ex 257 |
| 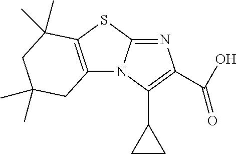 [SMILES: CC1(C)Cc2c(sc3nc(C(=O)O)c(C4CC4)n23)C(C)(C)C1] | Ex 258 |
| 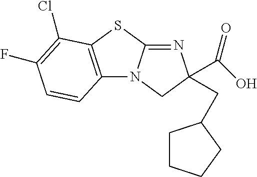 [SMILES: O=C(O)C1(CC2CCCC2)CN2C(=N1)Sc1c2ccc(F)c1Cl] | Ex 259 |
| 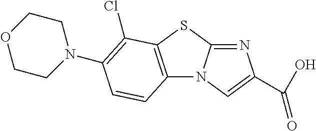 [SMILES: O=C(O)c1cn2c(n1)sc1c(Cl)c(N3CCOCC3)ccc12] | Ex 260 |
| 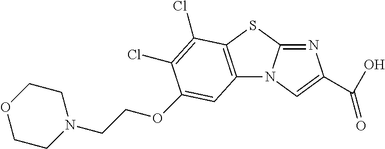 [SMILES: O=C(O)c1cn2c(n1)sc1c(Cl)c(Cl)c(OCCN3CCOCC3)cc12] | Ex 261 |
| 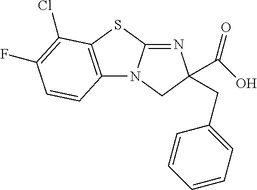 [SMILES: O=C(O)C1(Cc2ccccc2)CN2C(=N1)Sc1c2ccc(F)c1Cl] | Ex 262 |
| 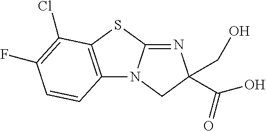 [SMILES: O=C(O)C1(CO)CN2C(=N1)Sc1c2ccc(F)c1Cl] | Ex 263 |
| 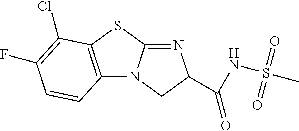 [SMILES: CS(=O)(=O)NC(=O)C1CN2C(=N1)Sc1c2ccc(F)c1Cl] | Ex 264 |
| 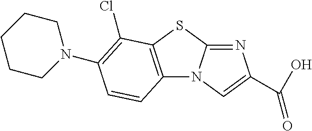 [SMILES: O=C(O)c1cn2c(n1)sc1c(Cl)c(N3CCCCC3)ccc12] | Ex 265 |
| 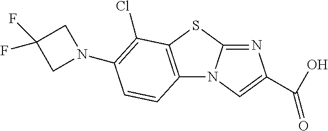 [SMILES: O=C(O)c1cn2c(n1)sc1c(Cl)c(N3CC(F)(F)C3)ccc12] | Ex 266 |
| 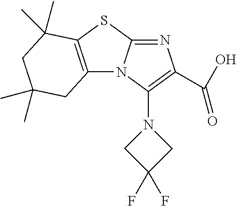 [SMILES: CC1(C)Cc2c(sc3nc(C(=O)O)c(N4CC(F)(F)C4)n23)C(C)(C)C1] | Ex 267 |
| 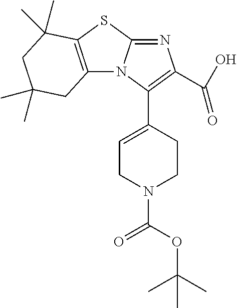 [SMILES: CC1(C)Cc2c(sc3nc(C(=O)O)c(C4=CCN(C(=O)OC(C)(C)C)CC4)n23)C(C)(C)C1] | Ex 268 |
| 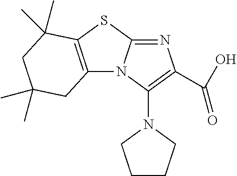 [SMILES: CC1(C)Cc2c(sc3nc(C(=O)O)c(N4CCCC4)n23)C(C)(C)C1] | Ex 269 |
| 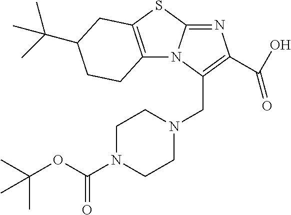 [SMILES: CC(C)(C)OC(=O)N1CCN(Cc2c(C(=O)O)nc3sc4c(n23)CCC(C(C)(C)C)C4)CC1] | Ex 270 |
| 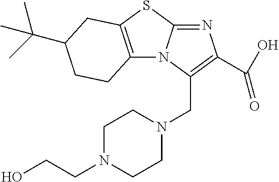 [SMILES: CC(C)(C)C1CCc2c(sc3nc(C(=O)O)c(CN4CCN(CCO)CC4)n23)C1] | Ex 271 |
| 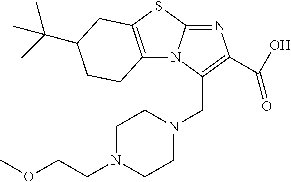 [SMILES: COCCN1CCN(Cc2c(C(=O)O)nc3sc4c(n23)CCC(C(C)(C)C)C4)CC1] | Ex 272 |
| 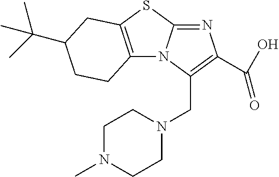 [SMILES: CN1CCN(Cc2c(C(=O)O)nc3sc4c(n23)CCC(C(C)(C)C)C4)CC1] | Ex 273 |
| 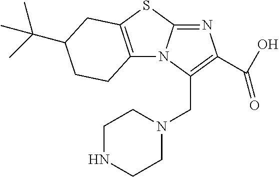 [SMILES: CC(C)(C)C1CCc2c(sc3nc(C(=O)O)c(CN4CCNCC4)n23)C1] | Ex 274 |
| 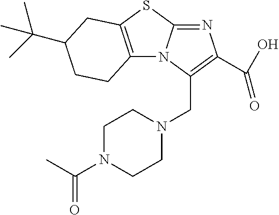 [SMILES: CC(=O)N1CCN(Cc2c(C(=O)O)nc3sc4c(n23)CCC(C(C)(C)C)C4)CC1] | Ex 275 |
| 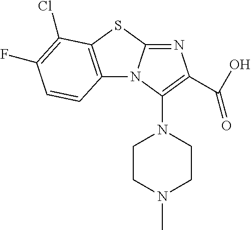 [SMILES: CN1CCN(c2c(C(=O)O)nc3sc4c(Cl)c(F)ccc4n23)CC1] | Ex 276 |
| 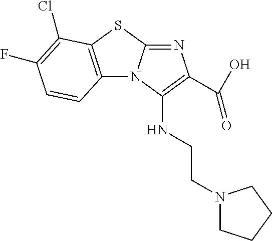 [SMILES: O=C(O)c1nc2sc3c(Cl)c(F)ccc3n2c1NCCN1CCCC1] | Ex 277 |
| 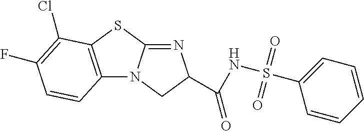 [SMILES: O=C(NS(=O)(=O)c1ccccc1)C1CN2C(=N1)Sc1c2ccc(F)c1Cl] | Ex 278 |
| 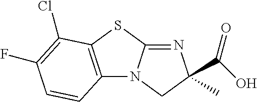 [SMILES: C[C@]1(C(=O)O)CN2C(=N1)Sc1c2ccc(F)c1Cl] | Ex 279 |
| 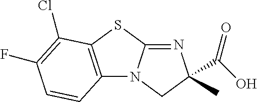 [SMILES: C[C@@]1(C(=O)O)CN2C(=N1)Sc1c2ccc(F)c1Cl] | |
| 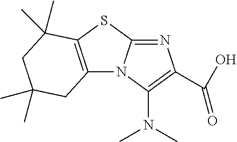 [SMILES: CN(C)c1c(C(=O)O)nc2sc3c(n12)CC(C)(C)CC3(C)C] | Ex 280 |
| 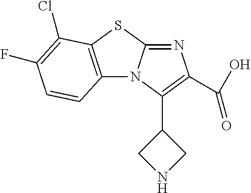 [SMILES: O=C(O)c1nc2sc3c(Cl)c(F)ccc3n2c1C1CNC1] | Ex 281 |
| 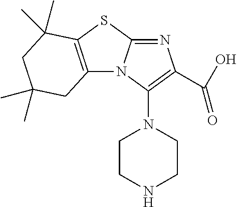 [SMILES: CC1(C)Cc2c(sc3nc(C(=O)O)c(N4CCNCC4)n23)C(C)(C)C1] | Ex 282 |
| 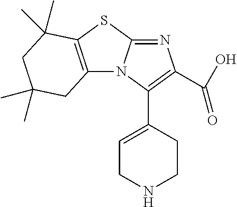 [SMILES: CC1(C)Cc2c(sc3nc(C(=O)O)c(C4=CCNCC4)n23)C(C)(C)C1] | Ex 283 |
| 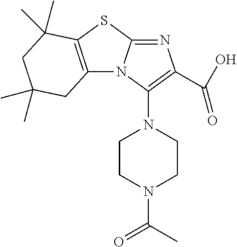 [SMILES: CC(=O)N1CCN(c2c(C(=O)O)nc3sc4c(n23)CC(C)(C)CC4(C)C)CC1] | Ex 284 |
| 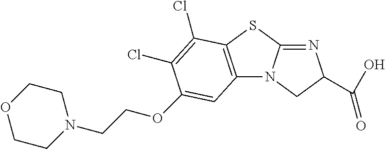 [SMILES: O=C(O)C1CN2C(=N1)Sc1c2cc(OCCN2CCOCC2)c(Cl)c1Cl] | Ex 285 |
| 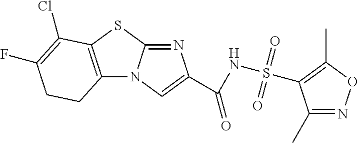 [SMILES: Cc1noc(C)c1S(=O)(=O)NC(=O)c1cn2c3c(sc2n1)C(Cl)=C(F)CC3] | Ex 286 |
| 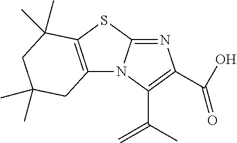 [SMILES: C=C(C)c1c(C(=O)O)nc2sc3c(n12)CC(C)(C)CC3(C)C] | Ex 287 |
| 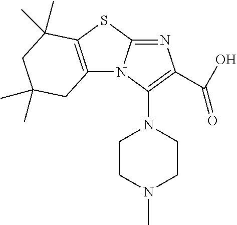 [SMILES: CN1CCN(c2c(C(=O)O)nc3sc4c(n23)CC(C)(C)CC4(C)C)CC1] | Ex 288 |
| 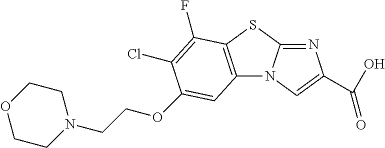 [SMILES: O=C(O)c1cn2c(n1)sc1c(F)c(Cl)c(OCCN3CCOCC3)cc12] | Ex 289 |
| 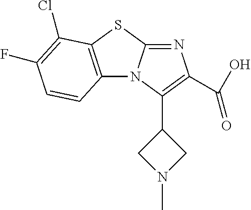 [SMILES: CN1CC(c2c(C(=O)O)nc3sc4c(Cl)c(F)ccc4n23)C1] | Ex 290 |
| 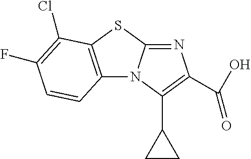 [SMILES: O=C(O)c1nc2sc3c(Cl)c(F)ccc3n2c1C1CC1] | Ex 291 |
| 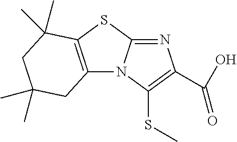 [SMILES: CSc1c(C(=O)O)nc2sc3c(n12)CC(C)(C)CC3(C)C] | Ex 292 |
| 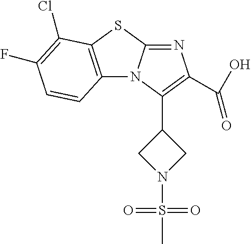 [SMILES: CS(=O)(=O)N1CC(c2c(C(=O)O)nc3sc4c(Cl)c(F)ccc4n23)C1] | Ex 293 |
| 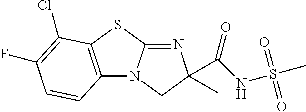 [SMILES: CC1(C(=O)NS(C)(=O)=O)CN2C(=N1)Sc1c2ccc(F)c1Cl] | Ex 294 |

### BIOLOGICAL ASSAYS

Biological Reagents Prepared and Purified for S100A9 Related Assays

Recombinant Human S100A9 Wild Type

Cultivation:

Expression of rhS100A9 wt was performed by shake flask cultivations of the working cell bank BL21(DE3)/pET1120 (pLR757) with 0.5 mM IPTG induction. Cell pellets were frozen.

Purification of Inclusion Bodies:

The E. coli pellets were thawed at RT with 150 mL Lysis buffer (50 mM Tris/HCl, 1 mM EDTA, 25% Saccarose, pH 8.0) and sonicated 3×15 s under ice in a beaker. Thereafter 10 μL of 1 M MgCl₂ (10 mM end conc.)/mL pellet solution, 1 μL 1 M MnCl₂ (1 mM end conc.)/mL pellet solution and 1 μL 10 mg/mL DNase I (10 μg/mL end conc.)/mL pellet solution were added. After 30 min of incubation in RT a detergent buffer (20 mM Tris/HCl, pH 7.5, 2 mM EDTA, 1% Nonidet P-40) with protease inhibitor (Complete Mini Protease Inhibitors, Roche), 1-2 tablets/25 mL was added in a 1:1 volume ratio. The solution was centrifuged at 14,000×g, 5° C., for 20 min. The pellet was resuspended with 90 mL 0.5% Triton X-100, 1 mM EDTA for sonication 3×15 s and was spinned down again. This wash and sonication procedure was repeated for additionally 5 times.

Resuspension and Folding:

Milli-Q water was used in all solutions and dialysis steps. The final pellet was resuspended in 100 mL of 8 M urea, 40 mM DTT in 500 mM NaH₂PO₄ buffer, pH 1.8. When the solution was clear it was centrifuged at 20,000×g, 5° C. for 25 min. The supernatant containing the resuspended inclusion bodies was set to pH 2 with the 500 mM phosphate buffer, pH 1.8.

First dialysis of the supernatant was against 5 L 50 mM NaH₂PO₄ buffer, 1.5 mM DTT, pH 2 for 6 h. Second dialysis against 5 L 10 mM Na-acetate buffer, 150 mM NaCl, 1.5 mM DTT, pH 4 for 15 h. Third dialysis against 5 L 10 mM Na-acetate buffer, 150 mM NaCl, 1.5 mM DTT, pH 4 for 8 h. Fourth dialysis against 5 L 20 mM Tris/HCl, 150 mM NaCl, 1.5 mM DTT, pH 7.2 for 16 h. Fifth dialysis against 5 L 20 mM Tris/HCl, 1 mM EDTA, 1 mM EGTA, 1.5 mM DTT, pH 8.5 for 6 h. Centrifugation was done at 22,000×g, 5° C. for 30 min.

Purification by Chromatography:

All chromatography columns and resins were purchased from GE HealthCare, Sweden. DTT was added to a final concentration of 1.5 mM. An anion-exchange chromatography on a HiPrep Q FF 16/10 column was run at a flow-rate of 1.5 mL/min using a 0-1 M NaCl gradient in 20 mM Tris, 1 mM EDTA, 1 mM EGTA, 1.5 mM DTT, pH 8.5 for elution of proteins. The same buffer, without NaCl, was used for equilibration and washing before elution. The pooled fractions containing rhS100A9 wt were concentrated to 1.5 mL using Centriprep YM-3 (Amicon, USA). The size-exclusion chromatography on a Superdex 75 16/790 column was run at a flow-rate of 0.5 mL/min using a HBS-N buffer (10 mM Hepes, 150 mM NaCl, pH 7.4) supplemented with 10 mM DTT. A PD-10 was run for buffer exchange to 10 mM Hepes, 150 mM NaCl, pH 7.5.

Biacore Binding Assays

The Ca²⁺ and Zn²⁺ dependent interaction of S100A9 with its target receptors—e.g. RAGE, TLR4/MD2 and EMMPRIN—was studied using surface plasmon resonance (SPR) technology (Björk et al. 2009). Briefly, S100A9 was injected over RAGE, TLR4/MD2 or EMMPRIN, immobilized via primary amines on a Biacore sensor chip, in the presence of physiological concentrations of Ca²⁺ and Zn²⁺ allowing label-free and real-time analysis of these interactions. Recombinant human RAGE and EMMPRIN, both fused with human IgG1Fc, and TLR4/MD2 were all purchased from R&D Systems. Obviously, the assay can be reversed in the way that S100A9 is immobilized and RAGE, TLR4/MD2 or EMMPRIN is injected. The person of ordinary skill in the art will be able to perform essentially the same assay directed to the interaction of S100A9 and TLR4/MD2 or EMMPRIN.

The assay showed the inhibitory effect of studied inventive compounds on protein-protein interactions between S100A9 and RAGE, TLR4/MD2 or EMMPRIN, respectively.

Inhibition Assay, Biot-hS100A9:hRAGE-Fc

Principle.

The AlphaScreen (Amplified Luminescent Proximity Homogeneous Assay) contains two types of beads, Alpha Donor beads and Acceptor beads (PerkinElmer). Upon laser excitation at 680 nm a photosensitizer in the Donor bead converts ambient oxygen to a more excited singlet state. The singlet oxygen molecule diffuses (maximum 200 nm) to react with a thioxene derivative in the Acceptor bead and generates a chemiluminescence reaction. Fluorophores in the Acceptor bead subsequently emit light at 520-620 nm which can be detected in the EnVision® Multilabel plate Reader (PerkinElmer). The beads are light sensitive and all work with the beads is performed under subdued light conditions or using green filters on light sources (Roscolux Chroma Green #389, Rosco).

In the AlphaScreen Inhibition Assay described here, protein A (Staphylococcus aureus) conjugated Acceptor beads are used together with streptavidin coated Donor beads (Perkin Elmer 6760617M). The Acceptor beads are pre-incubated with Fc-tagged recombinant human RAGE (rhRAGE-Fc) allowing binding of the rhRAGE-Fc to protein A on the beads. Biotinylated human S100A9 (biot-hS100A9) is pre-incubated with the low molecular test compounds. The pre-mixes are then added to the wells of a micro-plate and incubated allowing interaction between biot-hS100A9 and rhRAGE-Fc. Subsequent addition of streptavidin coated Donor beads causes binding of the streptavidin to the biotinylated hS100A9. After an additional incubation the signal is measured.

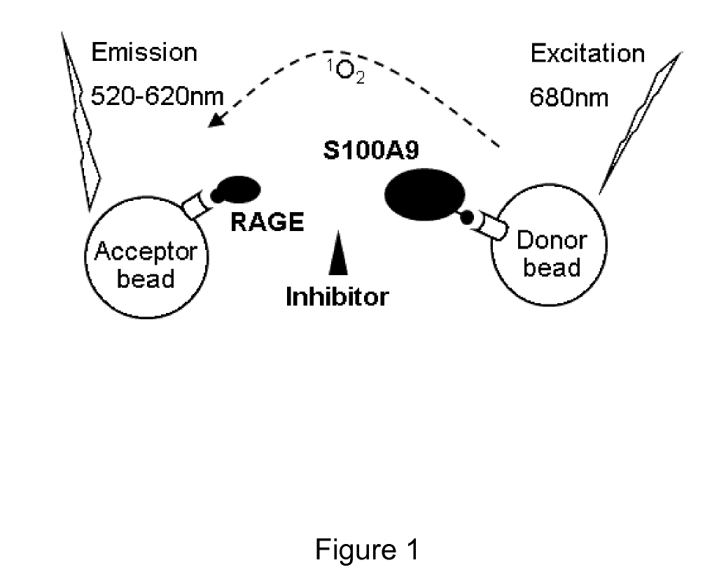

Without inhibitory compounds, the interaction of biot-hS100A9 to rhRAGE-Fc will bring the Acceptor and Donor beads in close proximity thus generating a high signal. With an inhibitor present the complex will not form resulting in a decreased signal. The assay is illustrated in FIG. 1.

Chemicals and Reagents.
AlphaScreen® General IgG (Protein A) Detection Kit, (PerkinElmer 6760617M)
   HBS-P buffer (GE Healthcare, BR-1003-68)
   HBS-N buffer (GE Healthcare, BR-1003-69)
   CaCl₂ in HBS-P
   ZnCl₂ in Milli-Q water
   DMSO
Biotinylated hS100A9, (biotinylated via cystein by EZ-link Iodoacetyl-PEG2-Biotin reagent (Pierce no. 21334), in HBS-N
   rhRAGE-Fc (R&D Systems, 1145-RG-50), in HBS-P

Procedure.

The AlphaScreen assay method is used for screening of the inhibitory effect of different compound samples at fixed concentrations or for IC50 determination by varying the compound concentrations. Samples of test compounds and references are prepared from solutions in DMSO. Relevant reference inhibitors and DMSO are used as controls for defined inhibition and non-inhibition, respectively in the assay. The percent inhibition in assay for test compounds and references are calculated by comparing their obtained assay signals with the signal values for the control with only DMSO (no compound).

Assay concentration of biotinylated hS100A9 and rhRAGE-Fc are batch dependent, and are determined and defined by separate cross-titration experiments using this AlphaScreen inhibition method to verify the optimal setup regarding signal strength and achievement of a defined inhibition with relevant reference compounds. The Final assay concentrations of Acceptor and Donor beads are 20 μg/mL.

Experimental Set Up for Screening, Preparation of Solutions and Beads.

Assay buffer is prepared by adding CaCl₂ and ZnCl₂ to HBS-P and is used freshly prepared in the experiment.

Biotin-hS100A9 solution for the experiment is prepared by dilution of appropriate amount of stock solution biot-hS100A9 in assay buffer (with CaCl₂ and ZnCl₂) and incubation in room temperature for 30 minutes.

rhRAGE-Fc solution for the experiment is prepared by dilution appropriate amount of rhRAGE-Fc stock in assay buffer.

Protein A Acceptor beads are diluted in assay buffer and are added to an equal volume of the prepared diluted rhRAGE-Fc solution. The beads are light sensitive. The vial is covered with aluminum foil and incubated at room temperature in the dark until biot-hS100A9+compound incubation is finished (see below).

Streptavidin-coated Donor beads are diluted in assay buffer. The beads are very light sensitive. The vial is covered with aluminum foil and incubated at room temperature in the dark until use (see below).

Dilution of Samples and Incubation with Biot-hS100A9

Samples of test compounds, appropriate references and DMSO control are diluted in assay buffer. The diluted test compounds, references and DMSO control are added to wells on a Greiner micro titer 96 well plate (PP, u-bottom (no. 650201)) and appropriate amount of diluted biot-hS100A9 solution are added to each well with samples (final DMSO conc.≤1.25% (v/v)). The plate is covered with a plate seal and is incubated in the dark on an orbital plate shaker for 1 h at room temperature.

Incubation of Biot-hS100A9+Compound Samples and rhRAGE-Fc-Acceptor Beads in Optiplate

When the biot-hS100A9+compound incubation is finished the solutions are transferred to Optiplate (Optiplate 384 white, Perkin Elmer no. 6007299) and rhRAGE-Fc—Acceptor bead solution is added to each well (use green filtered light). The plate is covered with a plate seal and incubated in the dark in a plate incubator at 25° C. nominally for 40 minutes.

Incubation of Biot-hS100A9+Compound Samples and rhRAGE-Fc-Acceptor and Donor Beads in Optiplate

After incubation Donor bead solution is added to each well (use green filtered light). The plate is covered with a plate seal and incubated in the dark in a plate incubator at 25° C. nominally. After 50 minutes, the plate is incubated (in the dark) on the bench next to the EnVision® instrument for 10 minutes, for temperature equilibrium.

Reading of Optiplate in EnVision® Multilabel Plate Reader

The plate seal is removed and the plate is placed in the EnVision® for 5 minutes before reading.

Calculations Percent (%) inhibition for each sample (test compound or reference) is calculated using the formula: 1-(Signal sample/Signal DMSO)×100%. The IC50 values for a number of compounds of the invention in the S100A9-RAGE inhibition assay are listed in Table 2.

TABLE 2

| Ex. | IC50 μM |
|---|---|
| 1 | 0.13 |
| 2 | 0.25 |
| 3 | 0.35 |
| 4 | 1.16 |
| 5 | 1.30 |
| 7 | 2.16 |
| 8 | 2.70 |
| 10 | 2.97 |
| 11 | 5.02 |
| 12 | 5.61 |
| 13 | 1.30 |
| 14 | 2.14 |
| 16 | 1.59 |
| 17 | 3.41 |
| 18 | 1.03 |
| 19 | 0.72 |
| 20 | 2.21 |
| 21 | 0.18 |
| 22 | 0.28 |
| 23 | 0.32 |
| 24 | 0.34 |
| 25 | 0.18 |
| 26 | 0.42 |
| 27 | 0.07 |
| 28 | 0.17 |
| 29 | 0.46 |
| 30 | 2.91 |
| 31 | 2.39 |
| 32 | 0.32 |
| 33 | 0.83 |
| 34 | 1.50 |
| 35 | 0.45 |
| 36 | 0.36 |
| 37 | 1.05 |
| 38 | 0.83 |
| 39 | 2.98 |
| 40 | 0.46 |
| 41 | 0.36 |
| 42 | 1.04 |
| 43 | 1.24 |
| 44 | 0.84 |
| 45 | 3.10 |
| 46 | 4.94 |
| 47 | 0.34 |
| 48 | 1.20 |
| 49 | 1.01 |
| 50 | 1.04 |
| 51 | 0.49 |
| 52 | 0.44 |
| 53 | 0.48 |
| 54 | 1.36 |
| 55 | 0.55 |
| 57 | 0.74 |
| 58 | 0.64 |
| 185 | 0.45 |
| 188 | 2.19 |
| 190 | 1.68 |
| 195 | 0.24 |
| 196 | 0.49 |
| 197 | 1.44 |
| 198 | 1.56 |
| 199 | 0.53 |
| 201 | 1.47 |
| 203 | 0.78 |
| 204 | 0.94 |
| 206 | 0.96 |
| 207 | 0.78 |
| 208 | 2.26 |
| 209 | 1.02 |
| 212 | 0.70 |
| 214 | 0.64 |
| 217 | 1.09 |
| 218 | 1.58 |
| 220 | 0.77 |
| 222 | 0.69 |
| 225 | 0.31 |
| 228 | 0.26 |
| 229 | 0.74 |
| 230 | 0.98 |
| 232 | 0.20 |
| 233 | 0.43 |
| 234 | 0.09 |
| 235 | 1.68 |
| 236 | 1.30 |
| 238 | 0.20 |
| 237 | 0.68 |
| 240 | 0.43 |
| 241 | 0.69 |
| 242 | 1.39 |
| 243 | 0.21 |
| 244 | 1.13 |
| 245 | 0.27 |
| 246 | 0.60 |
| 247 | 1.53 |
| 248 | 1.00 |
| 249 | 0.69 |
| 250 | 0.67 |
| 252 | 0.29 |
| 253 | 1.68 |
| 254 | 1.15 |
| 255 | 1.15 |
| 256 | 0.50 |
| 257 | 0.89 |
| 258 | 0.51 |
| 261 | 0.55 |
| 262 | 0.77 |
| 263 | 1.06 |
| 266 | 0.95 |
| 272 | 1.77 |
| 273 | 1.49 |
| 275 | 1.09 |
| 276 | 0.39 |
| 277 | 1.05 |
| 278 | 0.17 |
| 279a | 0.17 |
| 280 | 0.42 |
| 279b | 0.39 |
| 281 | 1.80 |
| 284 | 0.70 |
| 285 | 1.27 |
| 287 | 0.76 |
| 288 | 1.06 |
| 289 | 0.82 |
| 290 | 0.94 |
| 291 | 0.10 |
| 292 | 0.29 |
| 293 | 0.50 |
| 294 | 0.78 |

### REFERENCE LIST

Abignente et al, Farmaco (1981), 36(10), 893-904
Acharyya S et al., Cell 2012, 150(1), 165-7
Ager et al, J Med Chem (1988), 31(6), 1098-1115
Arai K et al., Curr Cancer Drug Targets 2008, 8(4): 243-52
Bhardwaj R S et al., Eur J Immunol 1992, 22:1891-97
Björk P et al., PLoS Biol. 2009, 7(4):e97
Blackburn et al, Bioorganic & Medicinal Chemistry Letters (2010), 20(16), 4795-4799
Cesaro A et al., PLoS One 2012, 7:e45478.
Chang K A et al., Neurodegener Dis 2012, 10(1-4):27-9
Cheng P et al., J Exp Med 2008, 205(10), 2235-49
Clements-Jewery et al, J Med Chem (1988), 31(6), 1220-1226
Deane R et al., J Clin Invest 2013. 122(4):1377-92
Foell D et al., J Leukoc Biol 2007, 81:28-37
Foell D et al., Arthritis Rheum 2004, 50, 3762-3771
Ghavami S et al., J Leukoc Biol 2008, 83(6), 1484-92
Grandolini et al, Farmaco (1993), 48(1), 31-43
Ha T et al., PLoS one 2010, 5(1):e8840
Herath et al, Organic Letters (2010), 12(3), 412-415
Hibino T et al., Cancer Res. 2013 Jan. 1; 73(1):172-83
Hiratsuka S et al., Nat Cell Biol. 8(12), 1369-75 (2006)
Marenholz I et al., Biochemical and Biophysical Research Comm. 322 (2004) 1111-1122
Palagiano et al, Eur J Med Chem (1995), 30(11), 901-909
Palagiano et al, Farmaco (1996), 51(7), 483-491
Patel et al, Bioorganic & Medicinal Chemistry Letters (2009), 19(17), 5182-5185
Riva M et al., Immunology 2012, 137:172-182
Ryckman C et al., J. Immunol. 170, 3233-42 (2003)
Shepherd C E et al., Neurobiol Aging 2006, 27:1554-1563
Sinha P et al., J Immunol 2008, 181:4666-4675
Srikrishna G et al., J Innate Immun 2012, 4:31-40
U.S. Pat. No. 4,137,320 (Enzo Tedeschi)
van Lent P et al., Arthritis & Rheumatism 2012, 64(5), 1466-76
Vu et al, J Med Chem (2009), 52(5), 1275-1283
Wang L et al., J Immunol 2013, 190:794-804
WO 02/069965 (Transtech Pharma Inc)
WO2006008556 (Istituto Di Ricerche Di Biologia Molecolare)

1. A compound of formula (I) 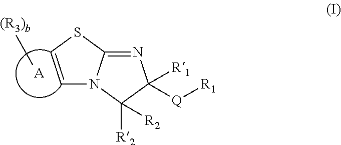
or a pharmaceutically acceptable salt thereof, wherein
b is an integer of from 0 to 4;
ring A is a 5- to 7-membered, aromatic or non-aromatic carbocycle or heterocycle;
Q is a direct bond, CH₂, CH(OH) or NH;
R₁ is R₄C(O), cyano, or tetrazolyl;
R₄ is H, R₅₀, or NHR₆;
R₅ is H or C1-C6 alkyl;
R₆ is H, cyano, C1-C6 alkyl, or R₇S(O)₂;
R₇ is C1-C6 alkyl, C3-C6 cycloalkyl, R₈(CH₂)_{y}, or 5- or 6-membered aryl or heteroaryl, said aryl or heteroaryl optionally being substituted by one or more moieties independently selected from C1-C6 alkyl,
R₈ is R₉O, R₁₀R₁₁N or R₁₂OC(O);
R₉ is H or C1-C6 alkyl;
R₁₀ and R₁₁ are independently selected from H and C1-C6 alkyl, or R₁₀ and R₁₁, together with the nitrogen atom to which they are both attached, form a 4- to 6-membered ring;
R₁₂ is H or C1-C6 alkyl;
y is an integer from 1 to 4;
R₂ is H, C1-C6 alkyl, C2-C6 alkenyl, C3-C6 cycloalkyl, halogen, cyano, R₁₃R₁₄N(CH₂)_{d}, R₁₅O(CH₂)ₑ, R₁₆S(CH₂)_{f}, R₁₇C(O)(CH₂)_{g}, 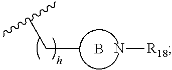
or
phenyl, optionally substituted by R₁₉O(CH₂)ᵢ;
R₁₃ is H, C1-C6 alkyl, R₂₀C(O), R₂₁S(O)₂, R₂₂O(CH₂)ⱼ, R₂₃R₂₄N(CH₂)ₖ, or benzyl, and
R₁₄ is H or C1-C6 alkyl; or
R₁₃ and R₁₄, together with the nitrogen atom to which they are both attached, form a 4- to 6-membered ring, said ring optionally being substituted by one or more substituents independently selected from oxo group, halogen, C1-C6 alkyl, R₂₅C(O), R₂₆OC(O), and R₂₇O(CH₂)ₘ;
R₁₅ is H, C1-C6 alkyl or R₂₈C(O);
R₁₆ and R₁₇ are selected from H and C1-C6 alkyl;
R₁₈ is H, C1-C6 alkyl, R₂₉OC(O)(CH₂)ₙ, or R₃₀S(O)₂(CH₂)ₚ;
R₁₉, R₂₀, R₂₁, R₂₂, R₂₃, R₂₄, R₂₅, R₂₆, R₂₇, R₂₈, R₂₉ and R₃₀ are selected from H and C1-C6 alkyl;
ring B is 4- to 6-membered, and saturated or unsaturated;
d, e, f, g, h, i, j, k, m, n, and p are integers of from 0 to 4;
R′₁ and R′₂ together form a bond; or
R′₁ is H, C1-C6 alkyl, C3-C6 carbocyclyl-(CH₂)_{q}, or R₃₁O(CH₂)ᵣ; and R′₂ is H;
R₃₁ is H or C1-C6 alkyl;
q and r are integers of from 0 to 4; and
each R₃ is independently selected from C1-C6 alkyl, C3-C6 carbocyclyl, halogen, oxo, R₃₂O, R₃₃S, and R₃₄R₃₅N;
R₃₂ is H, C1-C6 alkyl, C3-C6 carbocyclyl-(CH₂)ₛ, or R₃₆R₃₇N(CH₂)ₜ;
R₃₃ is H or C1-C6 alkyl;
R₃₄ and R₃₅ are independently selected from H and C1-C6 alkyl; or R₃₄ and R₃₅, together with the nitrogen atom to which they are both attached, form a 4- to 6-membered ring optionally substituted by one or more halogen;
R₃₆ and R₃₇ are independently selected from H and C1-C6 alkyl, or R₃₆ and R₃₇, together with the nitrogen atom to which they are both attached, form a 4- to 6-membered ring; optionally substituted by one or more halogen;
s and t are integers of from 0 to 4;
and
two R₃ attached to adjacent atoms of ring A, together with the atoms to which they are attached, may form a 3- to 6 membered ring, said ring being optionally substituted by one or more C1-C6 alkyl;
any alkyl, alkenyl and cycloalkyl is optionally substituted by one or more F;
provided that the compound is not
9,10-dichloro-7-thia-2,5-diazatricyclo[6.4.0.0^{2,6}]dodeca-1(8),3,5,9,11-pentaene-4-carboxylic acid,
10-(trifluoromethoxy)-7-thia-2,5-diazatricyclo[6.4.0.0^{2,6}]dodeca-1(8),3,5,9,11-pentaene-4-carboxylic acid,
9-chloro-7-thia-2,5-diazatricyclo[6.4.0.0^{2,6}]dodeca-1(8),3,5,9,11-pentaene-4-carboxylic acid,
10,11-dichloro-7-thia-2,5-diazatricyclo[6.4.0.0^{2,6}]dodeca-1(8),3,5,9,11-pentaene-4-carboxylic acid,
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-fluoro-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
10,12-dichloro-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5-triene-4-carboxylic acid,
10-tert-butyl-7-thia-2,5-diazatricyclo[6.4.0.0^{2,6}]dodeca-1(8),3,5-triene-4-carboxylic acid,
11-chloro-7-thia-2,5-diazatricyclo[6.4.0.0^{2,6}]dodeca-1(8),3,5,9,11-pentaene-4-carboxylic acid,
9-oxo-7-thia-2,5-diazatricyclo[6.4.0.0^{2,6}]dodeca-1(8),3,5-triene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
ethyl 2-{7-thia-2,5-diazatricyclo[6.4.0.0^{2,6}]dodeca-1(8),3,5,9,11-pentaen-4-yl}acetate,
ethyl 2-{10,11-dimethyl-7-thia-2,5-diazatricyclo[6.4.0.0^{2,6}]dodeca-1(8),3,5,9,11-pentaen-4-yl}acetate,
ethyl 2-{10-chloro-7-thia-2,5-diazatricyclo[6.4.0.0^{2,6}]dodeca-1(8),3,5,9,11-pentaen-4-yl}acetate,
ethyl 2-{11-methoxy-7-thia-2,5-diazatricyclo[6.4.0.0^{2,6}]dodeca-1(8),3,5,9,11-pentaen-4-yl}acetate,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
ethyl 7-thia-2,5-diazatricyclo[6.4.0.0^{2,6}]dodeca-1(8),3,5,9,11-pentaene-4-carboxylate,
ethyl 10,11-dimethyl-7-thia-2,5-diazatricyclo[6.4.0.0^{2,6}]dodeca-1(8),3,5,9,11-pentaene-4-carboxylate,
ethyl 10-methoxy-7-thia-2,5-diazatricyclo[6.4.0.0^{2,6}]dodeca-1(8),3,5,9,11-pentaene-4-carboxylate,
ethyl 12-methoxy-7-thia-2,5-diazatricyclo[6.4.0.0^{2,6}]dodeca-1(8),3,5,9,11-pentaene-4-carboxylate,
ethyl 10-ethyl-7-thia-2,5-diazatricyclo[6.4.0.0^{2,6}]dodeca-1(8),3,5,9,11-pentaene-4-carboxylate,
methyl 7-thia-2,5-diazatricyclo[6.4.0.0^{2,6}]dodeca-1(8),3,5,9,11-pentaene-4-carboxylate,
propyl 7-thia-2,5-diazatricyclo[6.4.0.0^{2,6}]dodeca-1(8),3,5,9,11-pentaene-4-carboxylate,
isopropyl 7-thia-2,5-diazatricyclo[6.4.0.0^{2,6}]dodeca-1(8),3,5,9,11-pentaene-4-carboxylate,
7-thia-2,5-diazatricyclo[6.4.0.0^{2,6}]dodeca-1(8),3,5,9,11-pentaene-4-carbaldehyde,
10-ethyl-7-thia-2,5-diazatricyclo[6.4.0.02,6]dodeca-1(8),3,5,9,11-pentaene-4-carbaldehyde,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1(10),2,4,6,8,12,14-heptaen-13-yl}acetic acid,
ethyl 16-thia-11,14-diazatetracyclo[8.6.0.0^{2,7}.0^{11,15}]hexadeca-1(10),2,4,6,8,12,14-heptaene-13-carboxylate,
ethyl 2-{16-thia-11,14-diazatetracyclo[8.6.0.0^{2,7}.0^{11,15}]hexadeca-1(10),2,4,6,8,12,14-heptaen-13-yl}acetate,
ethyl 10-(trifluoromethyl)-7-thia-2,5-diazatricyclo[6.4.0.0^{2,6}]dodeca-1(8),3,5,9,11-pentaene-4-carboxylate,
ethyl 10-chloro-7-thia-2,5-diazatricyclo[6.4.0.0^{2,6}]dodeca-1(8),3,5,9,11-pentaene-4-carboxylate,
ethyl 10-bromo-7-thia-2,5-diazatricyclo[6.4.0.0^{2,6}]dodeca-1(8),3,5,9,11-pentaene-4-carboxylate,
ethyl 10-methyl-7-thia-2,5-diazatricyclo[6.4.0.0^{2,6}]dodeca-1(8),3,5,9,11-pentaene-4-carboxylate,
ethyl 12-methyl-7-thia-2,5-diazatricyclo[6.4.0.0^{2,6}]dodeca-1(8),3,5,9,11-pentaene-4-carboxylate,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid,
2-{12-methyl-7-thia-2,5-diazatricyclo[6.4.0.0^{2,6}]dodeca-1(8),3,5,9,11-pentaen-4-yl}acetic acid,
ethyl 2-{10-trifluoromethyl-7-thia-2,5-diazatricyclo[6.4.0.0^{2,6}]dodeca-1(8),3,5,9,11-pentaen-4-yl}acetate,
ethyl 2-{10-bromo-7-thia-2,5-diazatricyclo[6.4.0.0^{2,6}]dodeca-1(8),3,5,9,11-pentaen-4-yl}acetate,
ethyl 2-{10-methyl-7-thia-2,5-diazatricyclo[6.4.0.0^{2,6}]dodeca-1(8),3,5,9,11-pentaen-4-yl}acetate, or
ethyl 2-{10-methyl-7-thia-2,5-diazatricyclo[6.4.0.0^{2,6}]dodeca-1(8),3,5,9,11-pentaen-4-yl}acetate.

2. The compound of claim 1, or a pharmaceutically acceptable salt thereof, wherein ring A is a 5- to 7-membered, aromatic or non-aromatic carbocycle.

3. The compound of claim 1, or a pharmaceutically acceptable salt thereof, wherein ring A is 6-membered.

4. The compound of claim 1, or a pharmaceutically acceptable salt thereof, wherein ring A is benzene.

5. The compound of claim 1, or a pharmaceutically acceptable salt thereof, wherein Q is a direct bond.

6. The compound of claim 1, or a pharmaceutically acceptable salt thereof, wherein R₁ is R₄C(O).

7. The compound of claim 1, or a pharmaceutically acceptable salt thereof, wherein R₄ is R₅₀ or NHR₆.

8. The compound of claim 1, or a pharmaceutically acceptable salt thereof, wherein R₄ is R₅O.

9. The compound of claim 1, or a pharmaceutically acceptable salt thereof, wherein R₂ is H, R₁₃R₁₄N(CH₂)_{d} or

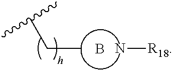

10. The compound of claim 1, or a pharmaceutically acceptable salt thereof, wherein R₂ is H or R₁₃R₁₄N(CH₂)_{d}.

11. The compound of claim 1, or a pharmaceutically acceptable salt thereof, wherein R₂ is H.

12. The compound of claim 1, or a pharmaceutically acceptable salt thereof, wherein R′₁ and R′₂ together form a bond.

13. A compound according to claim 1, selected from
9-chloro-10-fluoro-7-thia-2,5-diazatricyclo[6.4.0.0^{2,6}]dodeca-1(8),3,5,9,11-pentaene-4-carboxylic acid,
10-chloro-9-fluoro-7-thia-2,5-diazatricyclo[6.4.0.0^{2,6}]dodeca-1(8),3,5,9,11-pentaene-4-carboxylic acid,
11-chloro-7-thia-2,5-diazatricyclo[6.4.0.0^{2,6}]dodeca-1(8),3,5,9,11-pentaene-4-carboxylic acid,
3-bromo-10-(trifluoromethoxy)-7-thia-2,5-diazatricyclo[6.4.0.0^{2,6}]dodeca-1(8),3,5,9,11-pentaene-4-carboxylic acid,
3-(tert-butylamino)-10-(trifluoromethoxy)-7-thia-2,5-diazatricyclo[6.4.0.0^{2,6}]dodeca-1(8),3,5,9,11-pentaene-4-carboxylic acid,
2-{9,10-dichloro-7-thia-2,5-diazatricyclo[6.4.0.0^{2,6}]dodeca-1(8),3,5,9,11-pentaen-4-yl}acetic acid,
N-methanesulfonyl-10-(trifluoromethoxy)-7-thia-2,5-diazatricyclo[6.4.0.0^{2,6}]dodeca-1(8),3,5,9,11-pentaene-4-carboxamide,
9,10-dichloro-N-methanesulfonyl-7-thia-2,5-diazatricyclo[6.4.0.0^{2,6}]dodeca-1(8),3,5,9,11-pentaene-4-carboxamide,
9,10-dichloro-N-(cyclopropanesulfonyl)-7-thia-2,5-diazatricyclo[6.4.0.0^{2,6}]dodeca-1(8),3,5,9,11-pentaene-4-carboxamide,
9,10-dichloro-N-(2-methoxyethanesulfonyl)-7-thia-2,5-diazatricyclo[6.4.0.0^{2,6}]dodeca-1(8),3,5,9,11-pentaene-4-carboxamide,
9,10-dichloro-N-(ethanesulfonyl)-7-thia-2,5-diazatricyclo[6.4.0.0^{2,6}]dodeca-1(8),3,5,9,11-pentaene-4-carboxamide,
9,10-dichloro-N-(2,2,2-trifluoroethanesulfonyl)-7-thia-2,5-diazatricyclo[6.4.0.0^{2,6}]dodeca-1(8),3,5,9,11-pentaene-4-carboxamide,
9,10-dichloro-N-trifluoromethanesulfonyl-7-thia-2,5-diazatricyclo[6.4.0.0^{2,6}]dodeca-1(8),3,5,9,11-pentaene-4-carboxamide,
methyl 3-[({9,10-dichloro-7-thia-2,5-diazatricyclo[6.4.0.0^{2,6}]dodeca-1(8),3,5,9,11-pentaen-4-yl}formamido)sulfonyl]propanoate
9,10-dichloro-N-(3-hydroxypropanesulfonyl)-7-thia-2,5-diazatricyclo[6.4.0.0^{2,6}]dodeca-1(8),3,5,9,11-pentaene-4-carboxamide,
9,10-dichloro-N-[3-(diethylamino)propanesulfonyl]-7-thia-2,5-diazatricyclo[6.4.0.0^{2,6}]dodeca-1(8),3,5,9,11-pentaene-4-carboxamide,
2-{9,10-dichloro-7-thia-2,5-diazatricyclo[6.4.0.0^{2,6}]dodeca-1(8),3,5,9,11-pentaen-4-yl}-N-methanesulfonylacetamide,
9,9,11,11-tetramethyl-7-thia-2,5-diazatricyclo[6.4.0.0^{2,6}]dodeca-1(8),3,5-triene-4-carboxylic acid,
10,10-dimethyl-7-thia-2,5-diazatricyclo[6.4.0.0^{2,6}]dodeca-1(8),3,5-triene-4-carboxylic acid,
10-phenyl-7-thia-2,5-diazatricyclo[6.4.0.0^{2,6}]dodeca-1(8),3,5-triene-4-carboxylic acid,
4,4,5,5-tetramethyl-7-thia-1,9-diazatricyclo[6.3.0.0^{2,6}]undeca-2(6),8,10-triene-10-carboxylic acid,
7-thia-2,5-diazatricyclo[6.5.0.0^{2,6}]trideca-1(8),3,5-triene-4-carboxylic acid,
11,11-dimethyl-7-thia-2,5-diazatricyclo[6.4.0.0^{2,6}]dodeca-1(8),3,5-triene-4-carboxylic acid,
9-hydroxy-7-thia-2,5-diazatricyclo[6.4.0.0^{2,6}]dodeca-1(8),3,5-triene-4-carboxylic acid,
10-tert-butyl-3-(tert-butylamino)-7-thia-2,5-diazatricyclo[6.4.0.0^{2,6}]dodeca-1(8),3,5-triene-4-carboxylic acid,
16-thia-11,14-diazatetracyclo[8.6.0.0^{2,7}.0^{11,15}]hexadeca-1(10),2(7),3,5,12,14-hexaene-13-carboxylic acid,
3-(benzylamino)-10-tert-butyl-7-thia-2,5-diazatricyclo[6.4.0.0^{2,6}]dodeca-1(8),3,5-triene-4-carboxylic acid,
9,9-dimethyl-7-thia-2,5-diazatricyclo[6.4.0.0^{2,6}]dodeca-1(8),3,5-triene-4-carboxylic acid,
9-methyl-7-thia-2,5-diazatricyclo[6.4.0.0^{2,6}]dodeca-1(8),3,5-triene-4-carboxylic acid,
2-{10-tert-butyl-7-thia-2,5-diazatricyclo[6.4.0.0^{2,6}]dodeca-1(8),3,5-trien-4-yl}acetic acid,
2-{9,9,11,11-tetramethyl-7-thia-2,5-diazatricyclo[6.4.0.0^{2,6}]dodeca-1(8),3,5-trien-4-yl}acetic acid,
10-tert-butyl-N-methanesulfonyl-7-thia-2,5-diazatricyclo[6.4.0.0^{2,6}]dodeca-1(8), 3,5-triene-4-carboxamide,
N-methanesulfonyl-9-methyl-7-thia-2,5-diazatricyclo[6.4.0.0^{2,6}]dodeca-1(8),3,5-triene-4-carboxamide,
N-methanesulfonyl-11,11-dimethyl-7-thia-2,5-diazatricyclo[6.4.0.0^{2,6}]dodeca-1(8),3,5-triene-4-carboxamide,
10-tert-butyl-N-[3-(morpholin-4-yl)propanesulfonyl]-7-thia-2,5-diazatricyclo[6.4.0.0^{2,6}]dodeca-1(8),3,5-triene-4-carboxamide,
N-methanesulfonyl-4,4,5,5-tetramethyl-7-thia-1,9-diazatricyclo[6.3.0.0^{2,6}]undeca-2(6),8,10-triene-10-carboxamide,
N-methanesulfonyl-9,9,11,11-tetramethyl-7-thia-2,5-diazatricyclo[6.4.0.0^{2,6}]dodeca-1(8),3,5-triene-4-carboxamide,
N-[3-(diethylamino)propanesulfonyl]-9,9,11,11-tetramethyl-7-thia-2,5-diazatricyclo[6.4.0.0^{2,6}]dodeca-1(8),3,5-triene-4-carboxamide,
9,9,11,11-tetramethyl-N-[3-(morpholin-4-yl)propanesulfonyl]-7-thia-2,5-diazatricyclo[6.4.0.0^{2,6}]dodeca-1(8),3,5-triene-4-carboxamide,
4,4,5,5-tetramethyl-N-[3-(morpholin-4-yl)propanesulfonyl]-7-thia-1,9-diazatricyclo[6.3.0.0^{2,6}]undeca-2(6),8,10-triene-10-carboxamide,
11,11-dimethyl-N-(2,2,2-trifluoroethanesulfonyl)-7-thia-2,5-diazatricyclo[6.4.0.0^{2,6}]dodeca-1(8),3,5-triene-4-carboxamide,
10-tert-butyl-N-cyano-7-thia-2,5-diazatricyclo[6.4.0.0^{2,6}]dodeca-1(8),3,5-triene-4-carboxamide,
ethyl 9,10-dichloro-7-thia-2,5-diazatricyclo[6.4.0.0^{2,6}]dodeca-1(8),3,5,9,11-pentaene-4-carboxylate,
ethyl 9-chloro-10-fluoro-7-thia-2,5-diazatricyclo[6.4.0.0^{2,6}]dodeca-1(8),3,5,9,11-pentaene-4-carboxylate,
ethyl 10-chloro-9-fluoro-7-thia-2,5-diazatricyclo[6.4.0.0^{2,6}]dodeca-1(8),3,5,9,11-pentaene-4-carboxylate,
ethyl 10-(trifluoromethoxy)-7-thia-2,5-diazatricyclo[6.4.0.0^{2,6}]dodeca-1(8),3,5,9,11-pentaene-4-carboxylate,
ethyl 9-chloro-7-thia-2,5-diazatricyclo[6.4.0.0^{2,6}]dodeca-1(8),3,5,9,11-pentaene-4-carboxylate,
ethyl 10,11-dichloro-7-thia-2,5-diazatricyclo[6.4.0.0^{2,6}]dodeca-1(8),3,5,9,11-pentaene-4-carboxylate,
ethyl 10-fluoro-7-thia-2,5-diazatricyclo[6.4.0.0^{2,6}]dodeca-1(8),3,5,9,11-pentaene-4-carboxylate,
ethyl 11-chloro-7-thia-2,5-diazatricyclo[6.4.0.0^{2,6}]dodeca-1(8),3,5,9,11-pentaene-4-carboxylate,
ethyl 10-trifluoromethoxy-7-thia-2,5-diazatricyclo[6.4.0.0^{2,6}]dodeca-1(8),3,5,9,11-pentaene-4-carboxylate,
ethyl 3-bromo-10-(trifluoromethoxy)-7-thia-2,5-diazatricyclo[6.4.0.0^{2,6}]dodeca-1(8),3,5,9,11-pentaene-4-carboxylate,
ethyl 3-(tert-butylamino)-10-(trifluoromethoxy)-7-thia-2,5-diazatricyclo[6.4.0.0^{2,6}]dodeca-1(8),3,5,9,11-pentaene-4-carboxylate,
ethyl 2-{9,10-dichloro-7-thia-2,5-diazatricyclo[6.4.0.0^{2,6}]dodeca-1(8),3,5,9,11-pentaen-4-
ethyl 7-thia-2,5-diazatricyclo[6.4.0.0^{2,6}]dodeca-1(8),3,5-triene-4-carboxylate,
ethyl 10,10-dimethyl-7-thia-2,5-diazatricyclo[6.4.0.0^{2,6}]dodeca-1(8),3,5-triene-4-carboxylate,
ethyl 10-phenyl-7-thia-2,5-diazatricyclo[6.4.0.0^{2,6}]dodeca-1(8),3,5-triene-4-carboxylate,
ethyl 10-tert-butyl-7-thia-2,5-diazatricyclo[6.4.0.0^{2,6}]dodeca-1(8),3,5-triene-4-carboxylate,
ethyl 4,4,5,5-tetramethyl-7-thia-1,9-diazatricyclo[6.3.0.0^{2,6}]undeca-2(6),8,10-triene-10-carboxylate,
ethyl 7-thia-2,5-diazatricyclo[6.5.0.0^{2,6}]trideca-1(8), 3,5-triene-4-carboxylate,
ethyl 11,11-dimethyl-7-thia-2,5-diazatricyclo[6.4.0.0^{2,6}]dodeca-1(8),3,5-triene-4-carboxylate,
ethyl 9-oxo-7-thia-2,5-diazatricyclo[6.4.0.0^{2,6}]dodeca-1(8),3,5-triene-4-carboxylate,
ethyl 9-hydroxy-7-thia-2,5-diazatricyclo[6.4.0.0^{2,6}]dodeca-1(8), 3,5-triene-4-carboxylate,
ethyl 10-tert-butyl-3-(tert-butylamino)-7-thia-2,5-diazatricyclo[6.4.0.0^{2,6}]dodeca-1(8), 3,5-triene-4-carboxylate,
ethyl 16-thia-11,14-diazatetracyclo[8.6.0.0^{2,7}.0^{11,15}]hexadeca-1(10),2(7),3,5,12,14-hexaene-13-carboxylate,
ethyl 3-(benzylamino)-10-tert-butyl-7-thia-2,5-diazatricyclo[6.4.0.0^{2,6}]dodeca-1(8),3,5-triene-4-carboxylate,
ethyl 9,9-dimethyl-7-thia-2,5-diazatricyclo[6.4.0.0^{2,6}]dodeca-1(8),3,5-triene-4-carboxylate,
ethyl 9-methyl-7-thia-2,5-diazatricyclo[6.4.0.0^{2,6}]dodeca-1(8),3,5-triene-4-carboxylate,
10-tert-butyl-N-(3-chloropropanesulfonyl)-7-thia-2,5-diazatricyclo[6.4.0.0^{2,6}]dodeca-1(8),3,5-triene-4-carboxamide,
9,10-dichloro-N-(3-chloropropanesulfonyl)-7-thia-2,5-diazatricyclo[6.4.0.0^{2,6}]dodeca-1(8),3,5,9,11-pentaene-4-carboxamide,
9,9,11,11-tetramethyl-7-thia-2,5-diazatricyclo[6.4.0.0^{2,6}]dodeca-1(8),3,5-triene-4-carbaldehyde
methyl 2-hydroxy-2-{9,9,11,11-tetramethyl-7-thia-2,5-diazatricyclo[6.4.0.0^{2,6}]dodeca-1(8),3,5-trien-4-yl}acetate,
ethyl 9,9,11,11-tetramethyl-10-oxa-7-thia-2,5-diazatricyclo[6.4.0.0^{2,6}]dodeca-1(8),3,5-triene-4-carboxylate,
ethyl 2-{9,10-dichloro-7-thia-2,5-diazatricyclo[6.4.0.0^{2,6}]dodeca-1(8),3,5,9,11-pentaen-4-yl}-2-hydroxyacetate,
ethyl 3-bromo-10-tert-butyl-7-thia-2,5-diazatricyclo[6.4.0.0^{2,6}]dodeca-1(8),3,5-triene-4-carboxylate,
ethyl 10-tert-butyl-3-(4-methoxyphenyl)-7-thia-2,5-diazatricyclo[6.4.0.0^{2,6}]dodeca-1(8),3,5-triene-4-carboxylate,
ethyl 10,10-dimethyl-7-thia-2,5-diazatetracyclo[6.4.0.0^{2,6}.0^{9,11}]dodeca-1(8),3,5-triene-4-carboxylate,
ethyl 3-amino-10-tert-butyl-7-thia-2,5-diazatricyclo[6.4.0.0^{2,6}]dodeca-1(8),3,5-triene-4-carboxylate,
ethyl 10-tert-butyl-3-acetamido-7-thia-2,5-diazatricyclo[6.4.0.0^{2,6}]dodeca-1(8),3,5-triene-4-carboxylate,
ethyl 10-tert-butyl-3-methyl-7-thia-2,5-diazatricyclo[6.4.0.0^{2,6}]dodeca-1(8), 3,5-triene-4-carboxylate,
ethyl 10-tert-butyl-3-methanesulfonamido-7-thia-2,5-diazatricyclo[6.4.0.0^{2,6}]dodeca-1(8),3,5-triene-4-carboxylate,
ethyl 9,12-dichloro-7-thia-2,5-diazatricyclo[6.4.0.0^{2,6}]dodeca-1(8),3,5,9,11-pentaene-4-carboxylate,
[ethyl 9-(trifluoromethoxy)-7-thia-2,5-diazatricyclo[6.4.0.0^{2,6}]dodeca-1(8),3,5,9,11-pentaene-4-carboxylate,
ethyl 3-iodo-9,9,11,11-tetramethyl-7-thia-2,5-diazatricyclo[6.4.0.0^{2,6}]dodeca-1(8),3,5-triene-4-carboxylate,
10-tert-butyl-7-thia-2,5-diazatricyclo[6.4.0.0^{2,6}]dodeca-1(8),3,5-triene-4-carboxamide,
10-tert-butyl-7-thia-2,5-diazatricyclo[6.4.0.0^{2,6}]dodeca-1(8),3,5-triene-4-carbonitrile,
ethyl 10-[(trifluoromethyl)sulfanyl]-7-thia-2,5-diazatricyclo[6.4.0.0^{2,6}]dodeca-1(8),3,5,9,11-pentaene-4-carboxylate,
ethyl 10-(methylsulfanyl)-7-thia-2,5-diazatricyclo[6.4.0.0^{2,6}]dodeca-1(8),3,5,9,11-pentaene-4-carboxylate,
ethyl 9,11-dichloro-7-thia-2,5-diazatricyclo[6.4.0.0^{2,6}]dodeca-1(8),3,5,9,11-pentaene-4-carboxylate,
ethyl 10-tert-butyl-7-thia-2,5-diazatricyclo[6.4.0.0^{2,6}]dodeca-1(8),3,5,9,11-pentaene-4-carboxylate,
ethyl 3-chloro-9,9,11,11-tetramethyl-7-thia-2,5-diazatricyclo[6.4.0.0^{2,6}]dodeca-1(8),3,5-triene-4-carboxylate,
ethyl 10-hydroxy-7-thia-2,5-diazatricyclo[6.4.0.0^{2,6}]dodeca-1(8),3,5,9,11-pentaene-4-carboxylate,
ethyl 10-(benzyloxy)-7-thia-2,5-diazatricyclo[6.4.0.0^{2,6}]dodeca-1(8),3,5,9,11-pentaene-4-carboxylate,
ethyl 9,10-difluoro-7-thia-2,5-diazatricyclo[6.4.0.0^{2,6}]dodeca-1(8),3,5,9,11-pentaene-4-carboxylate,
ethyl 10-(cyclopentyloxy)-7-thia-2,5-diazatricyclo[6.4.0.0^{2,6}]dodeca-1(8),3,5,9,11-pentaene-4-carboxylate,
ethyl 9-bromo-7-thia-2,5-diazatricyclo[6.4.0.0^{2,6}]dodeca-1(8),3,5,9,11-pentaene-4-carboxylate,
ethyl 11-chloro-10-(trifluoromethoxy)-7-thia-2,5-diazatricyclo[6.4.0.0^{2,6}]dodeca-1(8),3,5,9,11-pentaene-4-carboxylate,
ethyl 9-chloro-10-(trifluoromethoxy)-7-thia-2,5-diazatricyclo[6.4.0.0^{2,6}]dodeca-1(8),3,5,9,11-pentaene-4-carboxylate,
ethyl 10-chloro-9-methoxy-7-thia-2,5-diazatricyclo[6.4.0.0^{2,6}]dodeca-1(8),3,5,9,11-pentaene-4-carboxylate,
ethyl 9-fluoro-7-thia-2,5-diazatricyclo[6.4.0.0^{2,6}]dodeca-1(8),3,5,9,11-pentaene-4-carboxylate,
ethyl 3-methanesulfonamido-9,9,11,11-tetramethyl-7-thia-2,5-diazatricyclo[6.4.0.0^{2,6}]dodeca1(8),3,5-triene-4-carboxylate,
ethyl 9-(pyrrolidin-1-yl)-7-thia-2,5-diazatricyclo[6.4.0.0^{2,6}]dodeca-1(8),3,5,9,11-pentaene-4-carboxylate,
ethyl 2-{9-chloro-10-fluoro-7-thia-2,5-diazatricyclo[6.4.0.0^{2,6}]dodeca-1(8),3,5,9,11-pentaen-4-yl}acetate,
ethyl 3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylate,
ethyl 9-chloro-10-fluoro-3-(trifluoromethyl)-7-thia-2,5-diazatricyclo[6.4.0.0^{2,6}]dodeca-1(8),3,5,9,11-pentaene-4-carboxylate,
ethyl 10-tert-butyl-7-thia-2,5-diazatricyclo[6.4.0.0^{2,6}]dodeca-1(8),5,9,11-tetraene-4-carboxylate,
ethyl 9-chloro-10-fluoro-7-thia-2,5-diazatricyclo[6.4.0.0^{2,6}]dodeca-1(8),5,9,11-tetraene-4-carboxylate,
methyl 9-chloro-10-fluoro-7-thia-2,5-diazatricyclo[6.4.0.0^{2,6}]dodeca-1(8),3,5,9,11-pentaene-4-carboxylate,
ethyl 3-bromo-9,9,11,11-tetramethyl-7-thia-2,5-diazatricyclo[6.4.0.0^{2,6}]dodeca-1(8),3,5-triene-4-carboxylate,
ethyl 3-ethynyl-9,9,11,11-tetramethyl-7-thia-2,5-diazatricyclo[6.4.0.0^{2,6}]dodeca-1(8),3,5-triene-4-carboxylate,
ethyl 10-chloro-11-(trifluoromethoxy)-7-thia-2,5-diazatricyclo[6.4.0.0^{2,6}]dodeca-1(8),3,5,9,11-pentaene-4-carboxylate,
ethyl 10-bromo-7-thia-2,5-diazatricyclo[6.4.0.0^{2,6}]dodeca-1(8),5,9,11-tetraene-4-carboxylate,
ethyl 3-bromo-9-chloro-10-fluoro-7-thia-2,5-diazatricyclo[6.4.0.0^{2,6}]dodeca-1(8),3,5,9,11-pentaene-4-carboxylate,
ethyl 9-chloro-10-fluoro-3-methanesulfonamido-7-thia-2,5-diazatricyclo[6.4.0.0^{2,6}]dodeca-1(8),3,5,9,11-pentaene-4-carboxylate,
ethyl 9,9,11,11-tetramethyl-3-(morpholin-4-yl)-7-thia-2,5-diazatricyclo[6.4.0.0^{2,6}]dodeca-1(8),3,5-triene-4-carboxylate,
ethyl 9-chloro-10-fluoro-3-methyl-7-thia-2,5-diazatricyclo[6.4.0.0^{2,6}]dodeca-1(8),3,5,9,11-pentaene-4-carboxylate,
ethyl 9-chloro-10-fluoro-7-thia-2,5-diazatricyclo[6.4.0.0^{2,6}]dodeca-1(8),5,9,11-tetraene-4-carboxylate,
ethyl (4S)-9-chloro-10-fluoro-7-thia-2,5-diazatricyclo[6.4.0.0^{2,6}]dodeca-1(8),5,9,11-tetraene-4-carboxylate,
ethyl (4R)-9-chloro-10-fluoro-7-thia-2,5-diazatricyclo[6.4.0.0^{2,6}]dodeca-1(8),5,9,11-tetraene-4-carboxylate,
ethyl 9-chloro-10-fluoro-3-(morpholin-4-yl)-7-thia-2,5-diazatricyclo[6.4.0.0^{2,6}]dodeca-1(8),3,5,9,11-pentaene-4-carboxylate,
ethyl 10-bromo-9-chloro-7-thia-2,5-diazatricyclo[6.4.0.0^{2,6}]dodeca-1(8),5,9,11-tetraene-4-carboxylate,
ethyl 9,10-dichloro-12-[2-(morpholin-4-yl)ethoxy]-7-thia-2,5-diazatricyclo[6.4.0.0^{2,6}]dodeca-1(8),3,5,9,11-pentaene-4-carboxylate,
ethyl 10-tert-butyl-3-(morpholin-4-ylmethyl)-7-thia-2,5-diazatricyclo[6.4.0.0^{2,6}]dodeca-1(8),3,5-triene-4-carboxylate,
ethyl 10-bromo-9-chloro-7-thia-2,5-diazatricyclo[6.4.0.0^{2,6}]dodeca-1(8),3,5,9,11-pentaene-4-carboxylate,
ethyl 10-tert-butyl-3-[(2-oxopiperidin-1-yl)methyl]-7-thia-2,5-diazatricyclo[6.4.0.0^{2,6}]dodeca-1(8),3,5-triene-4-carboxylate,
ethyl 3,9,9,11,11-pentamethyl-7-thia-2,5-diazatricyclo[6.4.0.0^{2,6}]dodeca-1(8),3,5-triene-4-carboxylate,
ethyl 9,9,11,11-tetramethyl-3-(morpholin-4-ylmethyl)-7-thia-2,5-diazatricyclo[6.4.0.0^{2,6}]dodeca-1(8),3,5-triene-4-carboxylate,
ethyl 3-(2-methoxyphenyl)-9,9,11,11-tetramethyl-7-thia-2,5-diazatricyclo[6.4.0.0^{2,6}]dodeca-1(8),3,5-triene-4-carboxylate,
ethyl 9-chloro-10-(pyrrolidin-1-yl)-7-thia-2,5-diazatricyclo[6.4.0.0^{2,6}]dodeca-1(8),3,5,9,11-pentaene-4-carboxylate,
ethyl 10-tert-butyl-3-{[(2-methoxyethyl)(methyl)amino]methyl}-7-thia-2,5-diazatricyclo[6.4.0.0^{2,6}]dodeca-1(8),3,5-triene-4-carboxylate,
ethyl 9-chloro-10-cyclopropyl-7-thia-2,5-diazatricyclo[6.4.0.0^{2,6}]dodeca-1(8),3,5,9,11-pentaene-4-carboxylate,
ethyl 3-(4-methoxyphenyl)-9,9,11,11-tetramethyl-7-thia-2,5-diazatricyclo[6.4.0.0^{2,6}]dodeca-1(8),3,5-triene-4-carboxylate,
ethyl 10-tert-butyl-3-(acetamidomethyl)-7-thia-2,5-diazatricyclo[6.4.0.0^{2,6}]dodeca-1(8),3,5-triene-4-carboxylate,
ethyl 3-[(acetyloxy)methyl]-10-tert-butyl-7-thia-2,5-diazatricyclo[6.4.0.0^{2,6}]dodeca-1(8),3,5-triene-4-carboxylate,
ethyl 10-tert-butyl-3-{[methoxy(methyl)amino]methyl}-7-thia-2,5-diazatricyclo[6.4.0.0^{2,6}]dodeca-1(8),3,5-triene-4-carboxylate,
ethyl 3-cyclopropyl-9,9,11,11-tetramethyl-7-thia-2,5-diazatricyclo[6.4.0.0^{2,6}]dodeca-1(8),3,5-triene-4-carboxylate,
tert-butyl 9-chloro-10-fluoro-7-thia-2,5-diazatricyclo[6.4.0.0^{2,6}]dodeca-1(8),5,9,11-tetraene-4-carboxylate,
ethyl 9-chloro-10-(morpholin-4-yl)-7-thia-2,5-diazatricyclo[6.4.0.0^{2,6}]dodeca-1(8),3,5,9,11-pentaene-4-carboxylate,
ethyl 9,10-dichloro-11-[2-(morpholin-4-yl)ethoxy]-7-thia-2,5-diazatricyclo[6.4.0.0^{2,6}]dodeca-1(8),3,5,9,11-pentaene-4-carboxylate,
ethyl 3-({4-[(tert-butoxy)carbonyl]piperazin-1-yl}methyl)-10-tert-butyl-7-thia-2,5-diazatricyclo[6.4.0.0^{2,6}]dodeca-1(8),3,5-triene-4-carboxylate,
ethyl 10-tert-butyl-3-{[4-(2-hydroxyethyl)piperazin-1-yl]methyl}-7-thia-2,5-diazatricyclo[6.4.0.0^{2,6}]dodeca-1(8),3,5-triene-4-carboxylate,
ethyl 10-tert-butyl-3-{[4-(2-m ethoxyethyl)piperazin-1-yl]methyl}-7-thia-2,5-diazatricyclo[6.4.0.0^{2,6}]dodeca-1(8),3,5-triene-4-carboxylate,
ethyl 10-tert-butyl-3-[(4-methyl piperazin-1-yl)methyl]-7-thia-2,5-diazatricyclo[6.4.0.0^{2,6}]dodeca-1(8),3,5-triene-4-carboxylate,
ethyl 3-[(4-acetylpiperazin-1-yl)methyl]-10-tert-butyl-7-thia-2,5-diazatricyclo[6.4.0.0^{2,6}]dodeca-1(8),3,5-triene-4-carboxylate,
ethyl 9-chloro-10-fluoro-3-{[2-(pyrrolidin-1-yl)ethyl]amino}-7-thia-2,5-diazatricyclo[6.4.0.0^{2,6}]dodeca-1(8),3,5,9,11-pentaene-4-carboxylate,
ethyl 3-(dimethylamino)-9,9,11,11-tetramethyl-7-thia-2,5-diazatricyclo[6.4.0.0^{2,6}]dodeca-1(8),3,5-triene-4-carboxylate,
ethyl 3-{1-[(tert-butoxy)carbonyl]azetidin-3-yl}-9-chloro-10-fluoro-7-thia-2,5-diazatricyclo[6.4.0.0^{2,6}]dodeca-1(8),3,5-triene-4-carboxylate,
3-{1-[(tert-butoxy)carbonyl]azetidin-3-yl}-9-chloro-10-fluoro-7-thia-2,5-diazatricyclo[6.4.0.0^{2,6}]dodeca-1(8),3,5-triene-4-carboxylic acid,
ethyl 3-{4-[(tert-butoxy)carbonyl]piperazin-1-yl}-9,9,11,11-tetramethyl-7-thia-2,5-diazatricyclo[6.4.0.0^{2,6}]dodeca-1(8),3,5-triene-4-carboxylate,
ethyl 9,9,11,11-tetramethyl-3-(piperazin-1-yl)-7-thia-2,5-diazatricyclo[6.4.0.0^{2,6}]dodeca-1(8),3,5-triene-4-carboxylate,
ethyl 3-(1,2,3,6-tetrahydropyridin-4-yl)-9,9,11,11-tetramethyl-7-thia-2,5-diazatricyclo[6.4.0.0^{2,6}]dodeca-1(8),3,5-triene-4-carboxylate,
ethyl 3-(4-acetylpiperazin-1-yl)-9,9,11,11-tetramethyl-7-thia-2,5-diazatricyclo[6.4.0.0^{2,6}]dodeca-1(8),3,5-triene-4-carboxylate,
ethyl 9,10-dichloro-11-[2-(morpholin-4-yl)ethoxy]-7-thia-2,5-diazatricyclo[6.4.0.0^{2,6}]dodeca-1(8),5,9,11-tetraene-4-carboxylate,
ethyl 9,9,11,11-tetramethyl-3-(prop-1-en-2-yl)-7-thia-2,5-diazatricyclo[6.4.0.0^{2,6}]dodeca-1(8),3,5-triene-4-carboxylate,
ethyl 9,9,11,11-tetramethyl-3-(4-methylpiperazin-1-yl)-7-thia-2,5-diazatricyclo[6.4.0.0^{2,6}]dodeca-1(8),3,5-triene-4-carboxylate,
ethyl 3-(azetidin-3-yl)-9-chloro-10-fluoro-7-thia-2,5-diazatricyclo[6.4.0.0^{2,6}]dodeca-1(8),3,5,9,11-pentaene-4-carboxylate,
ethyl 9-chloro-10-fluoro-3-(1-methylazetidin-3-yl)-7-thia-2,5-diazatricyclo[6.4.0.0^{2,6}]dodeca-1(8),3,5,9,11-pentaene-4-carboxylate,
ethyl 9-chloro-3-cyclopropyl-10-fluoro-7-thia-2,5-diazatricyclo[6.4.0.0^{2,6}]dodeca-1(8),3,5,9,11-pentaene-4-carboxylate,
ethyl 9,9,11,11-tetramethyl-3-(methylsulfanyl)-7-thia-2,5-diazatricyclo[6.4.0.0^{2,6}]dodeca-1(8),3,5-triene-4-carboxylate,
9,10-dichloro-7-thia-2,5-diazatricyclo[6.4.0.0^{2,6}]dodeca-1(8),5,9,11-tetraene-4-carboxylic acid,
2-hydroxy-2-{9,9,11,11-tetramethyl-7-thia-2,5-diazatricyclo[6.4.0.0^{2,6}]dodeca-1(8),3,5-trien-4-yl}acetic acid,
9,9,11,11-tetramethyl-10-oxa-7-thia-2,5-diazatricyclo[6.4.0.0^{2,6}]dodeca-1(8),3,5-triene-4-carboxylic acid,
2-{9,10-dichloro-7-thia-2,5-diazatricyclo[6.4.0.0^{2,6}]dodeca-1(8),3,5,9,11-pentaen-4-yl}-2-hydroxyacetic acid,
1-{10-tert-butyl-7-thia-2,5-diazatricyclo[6.4.0.0^{2,6}]dodeca-1(8),3,5-trien-4-yl}-3-methanesulfonylurea,
10-tert-butyl-3-(4-methoxyphenyl)-7-thia-2,5-diazatricyclo[6.4.0.0^{2,6}]dodeca-1(8),3,5-triene-4-carboxylic acid,
10,10-dimethyl-7-thia-2,5-diazatetracyclo[6.4.0.0^{2,6}.0^{9,11}]dodeca-1(8),3,5-triene-4-carboxylic acid,
N-methanesulfonyl-9,9,11,11-tetramethyl-10-oxa-7-thia-2,5-diazatricyclo[6.4.0.0^{2,6}]dodeca-1(8),3,5-triene-4-carboxamide,
N-methanesulfonyl-10,10-dimethyl-7-thia-2,5-diazatetracyclo[6.4.0.0^{2,6}.0^{9,11}]dodeca-1(8),3,5-triene-4-carboxamide,
10-tert-butyl-3-acetamido-7-thia-2,5-diazatricyclo[6.4.0.0^{2,6}]dodeca-1(8),3,5-triene-4-carboxylic acid,
10-tert-butyl-3-methyl-7-thia-2,5-diazatricyclo[6.4.0.0^{2,6}]dodeca-1(8),3,5-triene-4-carboxylic acid,
10-tert-butyl-3-methanesulfonamido-7-thia-2,5-diazatricyclo[6.4.0.0^{2,6}]dodeca-1(8),3,5-triene-4-carboxylic acid,
9,12-dichloro-7-thia-2,5-diazatricyclo[6.4.0.0^{2,6}]dodeca-1(8),3,5,9,11-pentaene-4-carboxylic acid,
9-(trifluoromethoxy)-7-thia-2,5-diazatricyclo[6.4.0.0^{2,6}]dodeca-1(8),3,5,9,11-pentaene-4-carboxylic acid,
3-Iodo-9,9,11,11-tetramethyl-7-thia-2,5-diazatricyclo[6.4.0.0^{2,6}]dodeca-1(8),3,5-triene-4-carboxylic acid,
10-tert-butyl-4-(1H-1,2,3,4-tetrazol-5-yl)-7-thia-2,5-diazatricyclo[6.4.0.0^{2,6}]dodeca-1(8),3,5-triene
10-[(trifluoromethyl)sulfanyl]-7-thia-2,5-diazatricyclo[6.4.0.0^{2,6}]dodeca-1(8),3,5,9,11-pentaene-4-carboxylic acid,
10-(methylsulfanyl)-7-thia-2,5-diazatricyclo[6.4.0.0^{2,6}]dodeca-1(8),3,5,9,11-pentaene-4-carboxylic acid,
9,10-dichloro-3-[(dimethylamino)methyl]-7-thia-2,5-diazatricyclo[6.4.0.0^{2,6}]dodeca-1(8),3,5,9,11-pentaene-4-carboxylic acid,
9,11-dichloro-7-thia-2,5-diazatricyclo[6.4.0.0^{2,6}]dodeca-1(8),3,5,9,11-pentaene-4-carboxylic acid,
3-[(dimethylamino)methyl]-9,9,11,11-tetramethyl-7-thia-2,5-diazatricyclo[6.4.0.0^{2,6}]dodeca-1(8),3,5-triene-4-carboxylic acid,
10-tert-butyl-7-thia-2,5-diazatricyclo[6.4.0.0^{2,6}]dodeca-1(8),3,5,9,11-pentaene-4-carboxylic acid,
3-chloro-9,9,11,11-tetramethyl-7-thia-2,5-diazatricyclo[6.4.0.0^{2,6}]dodeca-1(8),3,5-triene-4-carboxylic acid,
10-(benzyloxy)-7-thia-2,5-diazatricyclo[6.4.0.0^{2,6}]dodeca-1(8),3,5,9,11-pentaene-4-carboxylic acid,
9,10-difluoro-7-thia-2,5-diazatricyclo[6.4.0.0^{2,6}]dodeca-1(8),3,5,9,11-pentaene-4-carboxylic acid,
9-chloro-3-[(dimethylamino)methyl]-10-fluoro-7-thia-2,5-diazatricyclo[6.4.0.0^{2,6}]dodeca-1(8),3,5,9,11-pentaene-4-carboxylic acid,
10-(cyclopentyloxy)-7-thia-2,5-diazatricyclo[6.4.0.0^{2,6}]dodeca-1(8),3,5,9,11-pentaene-4-carboxylic acid,
9-bromo-7-thia-2,5-diazatricyclo[6.4.0.0^{2,6}]dodeca-1(8),3,5,9,11-pentaene-4-carboxylic acid,
11-chloro-10-(trifluoromethoxy)-7-thia-2,5-diazatricyclo[6.4.0.0^{2,6}]dodeca-1(8),3,5,9,11-pentaene-4-carboxylic acid,
9-chloro-10-(trifluoromethoxy)-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
10-chloro-9-methoxy-7-thia-2,5-diazatricyclo[6.4.0.0^{2,6}]dodeca-1(8),3,5,9,11-pentaene-4-carboxylic acid,
9-chloro-10-methoxy-7-thia-2,5-diazatricyclo[6.4.0.0^{2,6}]dodeca-1(8),3,5,9,11-pentaene-4-carboxylic acid,
9-fluoro-7-thia-2,5-diazatricyclo[6.4.0.0^{2,6}]dodeca-1(8),3,5,9,11-pentaene-4-carboxylic acid,
9-chloro-N-methanesulfonyl-10-(trifluoromethoxy)-7-thia-2,5-diazatricyclo[6.4.0.0^{2,6}]dodeca-1(8),3,5,9,11-pentaene-4-carboxamide,
3-methanesulfonamido-9,9,11,11-tetramethyl-7-thia-2,5-diazatricyclo[6.4.0.0^{2,6}]dodeca-1(8),3,5-triene-4-carboxylic acid,
3-bromo-9,9,11,11-tetramethyl-7-thia-2,5-diazatricyclo[6.4.0.0^{2,6}]dodeca-1(8),3,5-triene-4-carboxylic acid,
9-(Pyrrolidin-1-yl)-7-thia-2,5-diazatricyclo[6.4.0.0^{2,6}]dodeca-1(8),3,5,9,11-pentaene-4-carboxylic acid,
2-{9-chloro-10-fluoro-7-thia-2,5-diazatricyclo[6.4.0.0^{2,6}]dodeca-1(8),3,5,9,11-pentaen-4-yl}-2-hydroxyacetic acid,
3-{1-[(tert-butoxy)carbonyl]azetidin-3-yl}-9,9,11,11-tetramethyl-7-thia-2,5-diazatricyclo[6.4.0.0^{2,6}]dodeca-1(8),3,5-triene-4-carboxylic acid,
9-chloro-10-fluoro-3-(trifluoromethyl)-7-thia-2,5-diazatricyclo[6.4.0.0^{2,6}]dodeca-1(8),3,5,9,11-pentaene-4-carboxylic acid,
10-tert-butyl-7-thia-2,5-diazatricyclo[6.4.0.0^{2,6}]dodeca-1(8),5,9,11-tetraene-4-carboxylic acid,
9-chloro-10-fluoro-7-thia-2,5-diazatricyclo[6.4.0.0^{2,6}]dodeca-1(8),5,9,11-tetraene-4-carboxylic acid,
3-ethynyl-9,9,11,11-tetramethyl-7-thia-2,5-diazatricyclo[6.4.0.0^{2,6}]dodeca-1(8),3,5-triene-4-carboxylic acid,
10-chloro-11-(trifluoromethoxy)-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),5,9,11-tetraene-4-carboxylic acid,
9-chloro-10-fluoro-3-methanesulfonamido-7-thia-2,5-diazatricyclo[6.4.0.0^{2,6}]dodeca-1(8),3,5,9,11-pentaene-4-carboxylic acid,
9,9,11,11-tetramethyl-3-(morpholin-4-yl)-7-thia-2,5-diazatricyclo[6.4.0.0^{2,6}]dodeca-1(8),3,5-triene-4-carboxylic acid,
9-chloro-10-fluoro-3-methyl-7-thia-2,5-diazatricyclo[6.4.0.0^{2,6}]dodeca-1(8),3,5,9,11-pentaene-4-carboxylic acid,
3-(Azetidin-3-yl)-9,9,11,11-tetramethyl-7-thia-2,5-diazatricyclo[6.4.0.0^{2,6}]dodeca-1(8),3,5-triene-4-carboxylic acid,
(4S)-9-chloro-10-fluoro-7-thia-2,5-diazatricyclo[6.4.0.0^{2,6}]dodeca-1(8),5,9,11-tetraene-4-carboxylic acid, (configuration arbitrarily assigned)
(4R)-9-chloro-10-fluoro-7-thia-2,5-diazatricyclo[6.4.0.0^{2,6}]dodeca-1(8),5,9,11-tetraene-4-carboxylic acid, (configuration arbitrarily assigned)
9-chloro-10-fluoro-3-(morpholin-4-yl)-7-thia-2,5-diazatricyclo[6.4.0.0^{2,6}]dodeca-1(8),3,5,9,11-pentaene-4-carboxylic acid,
3-acetyl-9,9,11,11-tetramethyl-7-thia-2,5-diazatricyclo[6.4.0.0^{2,6}]dodeca-1(8),3,5-triene-4-carboxylic acid,
10-bromo-9-chloro-7-thia-2,5-diazatricyclo[6.4.0.0^{2,6}]dodeca-1(8),5,9,11-tetraene-4-carboxylic acid,
9,10-dichloro-12-[2-(morpholin-4-yl)ethoxy]-7-thia-2,5-diazatricyclo[6.4.0.0^{2,6}]dodeca-1(8),3,5,9,11-pentaene-4-carboxylic acid,
10-tert-butyl-3-(morpholin-4-ylmethyl)-7-thia-2,5-diazatricyclo[6.4.0.0^{2,6}]dodeca-1(8),3,5-triene-4-carboxylic acid,
10-bromo-9-chloro-7-thia-2,5-diazatricyclo[6.4.0.0^{2,6}]dodeca-1(8),3,5,9,11-pentaene-4-carboxylic acid,
10-tert-butyl-3-[(2-oxopiperidin-1-yl)methyl]-7-thia-2,5-diazatricyclo[6.4.0.0^{2,6}]dodeca-1(8),3,5-triene-4-carboxylic acid,
3,9,9,11,11-Pentamethyl-7-thia-2,5-diazatricyclo[6.4.0.0^{2,6}]dodeca-1(8),3,5-triene-4-carboxylic acid,
9,9,11,11-tetramethyl-3-(morpholin-4-ylmethyl)-7-thia-2,5-diazatricyclo[6.4.0.0^{2,6}]dodeca-1(8),3,5-triene-4-carboxylic acid,
3-(2-methoxyphenyl)-9,9,11,11-tetramethyl-7-thia-2,5-diazatricyclo[6.4.0.0^{2,6}]dodeca-1(8),3,5-triene-4-carboxylic acid,
9-chloro-10-(pyrrolidin-1-yl)-7-thia-2,5-diazatricyclo[6.4.0.0^{2,6}]dodeca-1(8),3,5,9,11-pentaene-4-carboxylic acid,
9-chloro-10-fluoro-4-(2-methylpropyl)-7-thia-2,5-diazatricyclo[6.4.0.0^{2,6}]dodeca-1(8),5,9,11-tetraene-4-carboxylic acid,
9-chloro-10-fluoro-4-methyl-7-thia-2,5-diazatricyclo[6.4.0.0^{2,6}]dodeca-1(8),5,9,11-tetraene-4-carboxylic acid,
10-tert-butyl-3-{[(2-methoxyethyl)(methyl)amino]methyl}-7-thia-2,5-diazatricyclo[6.4.0.0^{2,6}]dodeca-1(8),3,5-triene-4-carboxylic acid,
9-chloro-10-cyclopropyl-7-thia-2,5-diazatricyclo[6.4.0.0^{2,6}]dodeca-1(8),3,5,9,11-pentaene-4-carboxylic acid, 3-bromo-10-tert-butyl-7-thia-2,5-diazatricyclo[6.4.0.0^{2,6}]dodeca-1(8),3,5-triene-4-carboxylic acid,
3-(4-methoxyphenyl)-9,9,11,11-tetramethyl-7-thia-2,5-diazatricyclo[6.4.0.0^{2,6}]dodeca-1(8),3,5-triene-4-carboxylic acid,
10-tert-butyl-3-(acetamidomethyl)-7-thia-2,5-diazatricyclo[6.4.0.0^{2,6}]dodeca-1(8),3,5-triene-4-carboxylic acid,
10-tert-butyl-3-(hydroxymethyl)-7-thia-2,5-diazatricyclo[6.4.0.0^{2,6}]dodeca-1(8),3,5-triene-4-carboxylic acid,
10-tert-butyl-3-{[methoxy(methyl)amino]methyl}-7-thia-2,5-diazatricyclo[6.4.0.0^{2,6}]dodeca-1(8),3,5-triene-4-carboxylic acid,
3-cyclopropyl-9,9,11,11-tetramethyl-7-thia-2,5-diazatricyclo[6.4.0.0^{2,6}]dodeca-1(8),3,5-triene-4-carboxylic acid,
9-chloro-4-(cyclopentylmethyl)-10-fluoro-7-thia-2,5-diazatricyclo[6.4.0.0^{2,6}]dodeca-1(8),5,9,11-tetraene-4-carboxylic acid,
9-chloro-10-(morpholin-4-yl)-7-thia-2,5-diazatricyclo[6.4.0.0^{2,6}]dodeca-1(8),3,5,9,11-pentaene-4-carboxylic acid,
9,10-dichloro-11-[2-(morpholin-4-yl)ethoxy]-7-thia-2,5-diazatricyclo[6.4.0.0^{2,6}]dodeca-1(8),3,5,9,11-pentaene-4-carboxylic acid,
4-benzyl-9-chloro-10-fluoro-7-thia-2,5-diazatricyclo[6.4.0.0^{2,6}]dodeca-1(8),5,9,11-tetraene-4-carboxylic acid,
9-chloro-10-fluoro-4-(hydroxymethyl)-7-thia-2,5-diazatricyclo[6.4.0.0^{2,6}]dodeca-1(8),5,9,11-tetraene-4-carboxylic acid,
9-chloro-10-fluoro-N-methanesulfonyl-7-thia-2,5-diazatricyclo[6.4.0.0^{2,6}]dodeca-1(8),5,9,11-tetraene-4-carboxamide,
9-chloro-10-(piperidin-1-yl)-7-thia-2,5-diazatricyclo[6.4.0.0^{2,6}]dodeca-1(8),3,5,9,11-pentaene-4-carboxylic acid,
9-chloro-10-(3,3-difluoroazetidin-1-yl)-7-thia-2,5-diazatricyclo[6.4.0.0^{2,6}]dodeca-1(8),3,5,9,11-pentaene-4-carboxylic acid,
3-(3,3-difluoroazetidin-1-yl)-9,9,11,11-tetramethyl-7-thia-2,5diazatricyclo[6.4.0.0^{2,6}]dodeca-1(8),3,5-triene-4-carboxylic acid,
3-{1-[(tert-butoxy)carbonyl]-1,2,3,6-tetrahydropyridin-4-yl}-9,9,11,11-tetramethyl-7-thia-2,5-diazatricyclo[6.4.0.0^{2,6}]dodeca-1(8),3,5-triene-4-carboxylic acid,
9,9,11,11-tetramethyl-3-(pyrrolidin-1-yl)-7-thia-2,5-diazatricyclo[6.4.0.0^{2,6}]dodeca-1(8),3,5-triene-4-carboxylic acid,
3-({4-[(tert-butoxy)carbonyl]piperazin-1-yl}methyl)-10-tert-butyl-7-thia-2,5-diazatricyclo[6.4.0.0^{2,6}]dodeca-1(8),3,5-triene-4-carboxylic acid,
10-tert-butyl-3-{[4-(2-hydroxyethyl)piperazin-1-yl]methyl}-7-thia-2,5-diazatricyclo[6.4.0.0^{2,6}]dodeca-1(8),3,5-triene-4-carboxylic acid,
10-tert-butyl-3-{[4-(2-methoxyethyl)piperazin-1-yl]methyl}-7-thia-2,5-diazatricyclo[6.4.0.0^{2,6}]dodeca-1(8),3,5-triene-4-carboxylic acid,
10-tert-butyl-3-[(4-methylpiperazin-1-yl)methyl]-7-thia-2,5-diazatricyclo[6.4.0.0^{2,6}]dodeca-1(8),3,5-triene-4-carboxylic acid,
10-tert-butyl-3-(piperazin-1-ylmethyl)-7-thia-2,5-diazatricyclo[6.4.0.0^{2,6}]dodeca-1(8),3,5-triene-4-carboxylic acid,
3-[(4-acetylpiperazin-1-yl)methyl]-10-tert-butyl-7-thia-2,5-diazatricyclo[6.4.0.0^{2,6}]dodeca-1(8),3,5-triene-4-carboxylic acid,
9-chloro-10-fluoro-3-(4-methylpiperazin-1-yl)-7-thia-2,5-diazatricyclo[6.4.0.0^{2,6}]dodeca-1(8),3,5,9,11-pentaene-4-carboxylic acid,
9-chloro-10-fluoro-3-{[2-(pyrrolidin-1-yl)ethyl]amino}-7-thia-2,5-diazatricyclo[6.4.0.0^{2,6}]dodeca-1(8),3,5,9,11-pentaene-4-carboxylic acid,
N-(benzenesulfonyl)-9-chloro-10-fluoro-7-thia-2,5-diazatricyclo[6.4.0.0^{2,6}]dodeca-1(8),5,9,11-tetraene-4-carboxamide,
(4R)-9-chloro-10-fluoro-4-methyl-7-thia-2,5-diazatricyclo[6.4.0.0^{2,6}]dodeca-1(8),5,9,11-tetraene-4-carboxylic acid,
(4S)-9-chloro-10-fluoro-4-methyl-7-thia-2,5-diazatricyclo[6.4.0.0^{2,6}]dodeca-1(8),5,9,11-tetraene-4-carboxylic acid,
3-(dimethylamino)-9,9,9,11,11-tetramethyl-7-thia-2,5-diazatricyclo[6.4.0.0^{2,6}]dodeca-1(8),3,5-triene-4-carboxylic acid,
3-(azetidin-3-yl)-9-chloro-10-fluoro-7-thia-2,5-diazatricyclo[6.4.0.0^{2,6}]dodeca-1(8),3,5,9,11-pentaene-4-carboxylic acid,
9,9,11,11-tetramethyl-3-(piperazin-1-yl)-7-thia-2,5-diazatricyclo[6.4.0.0^{2,6}]dodeca-1(8),3,5-triene-4-carboxylic acid,
9,9,11,11-tetramethyl-3-(1,2,3,6-tetrahydropyridin-4-yl)-7-thia-2,5-diazatricyclo[6.4.0.0^{2,6}]dodeca-1(8),3,5-triene-4-carboxylic acid,
3-(4-acetylpiperazin-1-yl)-9,9,11,11-tetramethyl-7-thia-2,5-diazatricyclo[6.4.0.0^{2,6}]dodeca-1(8),3,5-triene-4-carboxylic acid,
9,10-dichloro-11-[2-(morpholin-4-yl)ethoxy]-7-thia-2,5-diazatricyclo[6.4.0.0^{2,6}]dodeca-1(8),5,9,11-tetraene-4-carboxylic acid,
9-chloro-N-[(dimethyl-1,2-oxazol-4-yl)sulfonyl]-10-fluoro-7-thia-2,5-diazatricyclo[6.4.0.0^{2,6}]dodeca-1(8)3,5,9-tetraene-4-carboxamide,
9,9,11,11-tetramethyl-3-(prop-1-en-2-yl)-7-thia-2,5-diazatricyclo[6.4.0.0^{2,6}]dodeca-1(8),3,5-triene-4-carboxylic acid,
9,9,11,11-tetramethyl-3-(4-methylpiperazin-1-yl)-7-thia-2,5-diazatricyclo[6.4.0.0^{2,6}]dodeca-1(8),3,5-triene-4-carboxylic acid,
10-chloro-9-fluoro-11-[2-(morpholin-4-yl)ethoxy]-7-thia-2,5-diazatricyclo[6.4.0.0^{2,6}]dodeca-1(8),3,5,9,11-pentaene-4-carboxylic acid,
9-chloro-10-fluoro-3-(1-methylazetidin-3-yl)-7-thia-2,5-diazatricyclo[6.4.0.0^{2,6}]dodeca-1(8),3,5,9,11-pentaene-4-carboxylic acid,
9-chloro-3-cyclopropyl-10-fluoro-7-thia-2,5-diazatricyclo[6.4.0.0^{2,6}]dodeca-1(8),3,5,9,11-pentaene-4-carboxylic acid,
9,9,11,11-tetramethyl-3-(methylsulfanyl)-7-thia-2,5-diazatricyclo[6.4.0.0^{2,6}]dodeca-1(8),3,5-triene-4-carboxylic acid,
9-chloro-10-fluoro-3-(1-methanesulfonylazetidin-3-yl)-7-thia-2,5-diazatricyclo[6.4.0.0^{2,6}]dodeca-1(8),3,5,9,11-pentaene-4-carboxylic acid, and
9-chloro-10-fluoro-N-methanesulfonyl-4-methyl-7-thia-2,5-diazatricyclo[6.4.0.0^{2,6}]dodeca-1(8),5,9,11-tetraene-4-carboxamide,
or a pharmaceutically acceptable salt thereof.

14. (canceled)

15. A pharmaceutical composition comprising a compound of formula (I) 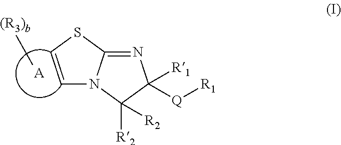
or a pharmaceutically acceptable salt thereof, wherein
b is an integer of from 0 to 4;
ring A is a 5- to 7-membered, aromatic or non-aromatic carbocycle or heterocycle;
Q is a direct bond, CH₂, CH(OH) or NH;
R₁ is R₄C(O), cyano, or tetrazolyl;
R₄ is H, R₅O, or NHR₆;
R₅ is H or C1-C6 alkyl;
R₆ is H, cyano, C1-C6 alkyl, or R₇S(O)₂;
R₇ is C1-C6 alkyl, C3-C6 cycloalkyl, R₈(CH₂)_{y}, or 5- or 6-membered aryl or heteroaryl, said aryl or heteroaryl optionally being substituted by one or more moieties independently selected from C1-C6 alkyl,
R₈ is R₉O, R₁₀R₁₁N or R₁₂OC(O);
R₉ is H or C1-C6 alkyl;
R₁₀ and R₁₁ are independently selected from H and C1-C6 alkyl, or R₁₀ and R₁₁, together with the nitrogen atom to which they are both attached, form a 4- to 6-membered ring;
R₁₂ is H or C1-C6 alkyl;
y is an integer from 1 to 4;
R₂ is H, C1-C6 alkyl, C2-C6 alkenyl, C3-C6 cycloalkyl, halogen, cyano, R₁₃R₁₄N(CH₂)_{d}, R₁₅O(CH₂)ₑ, R₁₆S(CH₂)_{f}, R₁₇C(O)(CH₂)_{g}, 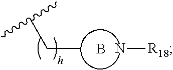
or
phenyl, optionally substituted by R₁₉O(CH₂)ᵢ;
R₁₃ is H, C1-C6 alkyl, R₂₀C(O), R₂₁S(O)₂, R₂₂O(CH₂)ⱼ, R₂₃R₂₄N(CH₂)ₖ, or benzyl, and
R₁₄ is H or C1-C6 alkyl; or
R₁₃ and R₁₄, together with the nitrogen atom to which they are both attached, form a 4- to 6-membered ring, said ring optionally being substituted by one or more substituents independently selected from oxo, halogen, C1-C6 alkyl, R₂₅C(O), R₂₆OC(O), and R₂₇O(CH₂)ₘ;
R₁₅ is H, C1-C6 alkyl or R₂₈C(O);
R₁₆ and R₁₇ are selected from H and C1-C6 alkyl;
R₁₈ is H, C1-C6 alkyl, R₂₉OC(O)(CH₂)ₙ, or R₃₀S(O)₂(CH₂)ₚ;
R₁₉, R₂₀, R₂₁, R₂₂, R₂₅, R₂₆, R₂₇, R₂₈, R₂₉ and R₃₀ are selected from H and C1-C6 alkyl;
R₂₃ and R₂₄ are independently selected from H and C1-C6 alkyl; or R₂₃ and R₂₄, together with the nitrogen atom to which they are both attached, form a 4- to 6-membered ring;
ring B is 4- to 6-membered, and saturated or unsaturated;
d, e, f, g, h, i, j, k, m, n, and p are integers of from 0 to 4;
R′₁ and R′₂ together form a bond; or
R′₁ is H, C1-C6 alkyl, C3-C6 carbocyclyl-(CH₂)_{q}, or R₃₁O(CH₂)ᵣ; and R′₂ is H;
R₃₁ is H or C1-C6 alkyl;
q and r are integers of from 0 to 4; and
each R₃ is independently selected from C1-C6 alkyl, C3-C6 carbocyclyl, halogen, oxo, R₃₂O, R₃₃S, and R₃₄R₃₅N;
R₃₂ is H, C1-C6 alkyl, C3-C6 carbocyclyl-(CH₂)ₛ, or R₃₆R₃₇N(CH₂)ₜ;
R₃₃ is H or C1-C6 alkyl;
R₃₄ and R₃₅ are independently selected from H and C1-C6 alkyl; or R₃₄ and R₃₅, together with the nitrogen atom to which they are both attached, form a 4- to 6-membered ring optionally substituted by one or more halogen;
R₃₆ and R₃₇ are independently selected from H and C1-C6 alkyl, or R₃₆ and R₃₇, together with the nitrogen atom to which they are both attached, form a 4- to 6-membered ring; optionally substituted by one or more halogen;
s and t are integers of from 0 to 4;
and
two R₃ attached to adjacent atoms of ring A, together with the atoms to which they are attached, may form a 3- to 6 membered ring, said ring being optionally substituted by one or more C1-C6 alkyl;
any alkyl, alkenyl and cycloalkyl is optionally substituted by one or more F, and optionally a pharmaceutically acceptable excipient;
provided that the compound is not
12-chloro-7-thia-2,5-diazatricyclo[6.4.0.0^{2,6}]dodeca-1(8),3,5,9,11-pentaene-4-carboxylic acid,
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
ethyl 10-ethyl-7-thia-2,5-diazatricyclo[6.4.0.0^{2,6}]dodeca-1(8),3,5,9,11-pentaene-4-carboxylate,
methyl 7-thia-2,5-diazatricyclo[6.4.0.0^{2,6}]dodeca-1(8),3,5,9,11-pentaene-4-carboxylate,
propyl 7-thia-2,5-diazatricyclo[6.4.0.0^{2,6}]dodeca-1(8),3,5,9,11-pentaene-4-carboxylate,
isopropyl 7-thia-2,5-diazatricyclo[6.4.0.0^{2,6}]dodeca-1(8),3,5,9,11-pentaene-4-carboxylate,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1(10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

16. A pharmaceutical composition comprising a compound according to claim 1, or a compound selected from
9,10-dichloro-7-thia-2,5-diazatricyclo[6.4.0.0^{2,6}]dodeca-1(8),3,5,9,11-pentaene-4-carboxylic acid,
10-(trifluoromethoxy)-7-thia-2,5-diazatricyclo[6.4.0.0^{2,6}]dodeca-1(8),3,5,9,11-pentaene-4-carboxylic acid,
9-chloro-7-thia-2,5-diazatricyclo[6.4.0.0^{2,6}]dodeca-1(8),3,5,9,11-pentaene-4-carboxylic acid,
10,11-dichloro-7-thia-2,5-diazatricyclo[6.4.0.0^{2,6}]dodeca-1(8),3,5,9,11-pentaene-4-carboxylic acid,
10-fluoro-7-thia-2,5-diazatricyclo[6.4.0.0^{2,6}]dodeca-1(8),3,5,9,11-pentaene-4-carboxylic acid,
10,12-dichloro-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5-triene-4-carboxylic acid,
10-tert-butyl-7-thia-2,5-diazatricyclo[6.4.0.0^{2,6}]dodeca-1(8),3,5-triene-4-carboxylic acid,
ethyl 10,11-dimethyl-7-thia-2,5-diazatricyclo[6.4.0.0^{2,6}]dodeca-1(8),3,5,9,11-pentaene-4-carboxylate,
ethyl 10-methoxy-7-thia-2,5-diazatricyclo[6.4.0.0^{2,6}]dodeca-1(8),3,5,9,11-pentaene-4-carboxylate,
ethyl 12-methoxy-7-thia-2,5-diazatricyclo[6.4.0.0^{2,6}]dodeca-1(8),3,5,9,11-pentaene-4-carboxylate,
ethyl 10-chloro-7-thia-2,5-diazatricyclo[6.4.0.0^{2,6}]dodeca-1(8),3,5,9,11-pentaene-4-carboxylate,
ethyl 10-methyl-7-thia-2,5-diazatricyclo[6.4.0.0^{2,6}]dodeca-1(8),3,5,9,11-pentaene-4-carboxylate,
ethyl 10-bromo-7-thia-2,5-diazatricyclo[6.4.0.0^{2,6}]dodeca-1(8),3,5,9,11-pentaene-4-carboxylate, and
ethyl 12-methyl-7-thia-2,5-diazatricyclo[6.4.0.0^{2,6}]dodeca-1(8),3,5,9,11-pentaene-4-carboxylate,
or a pharmaceutically acceptable salt thereof, and optionally a pharmaceutically acceptable excipient.

17-25. (canceled)

26. A method of treatment of cancer, an inflammatory disorder, an autoimmunity disorder or a neurodegenerative disorder by administering, to a mammal in need thereof, a compound of formula (I) 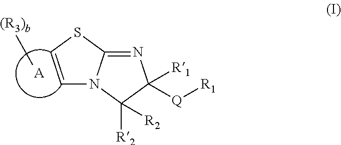
or a pharmaceutically acceptable salt thereof, wherein
b is an integer of from 0 to 4;
ring A is a 5- to 7-membered, aromatic or non-aromatic carbocycle or heterocycle;
Q is a direct bond, CH₂, CH(OH) or NH;
R₁ is R₄C(O), cyano, or tetrazolyl;
R₄ is H, R₅O, or NHR₆;
R₅ is H or C1-C6 alkyl;
R₆ is H, cyano, C1-C6 alkyl, or R₇S(O)₂;
R₇ is C1-C6 alkyl, C3-C6 cycloalkyl, R₈(CH₂)_{y}, or 5- or 6-membered aryl or heteroaryl, said aryl or heteroaryl optionally being substituted by one or more moieties independently selected from C1-C6 alkyl,
R₈ is R₉O, R₁₀R₁₁N or R₁₂OC(O);
R₉ is H or C1-C6 alkyl;
R₁₀ and R₁₁ are independently selected from H and C1-C6 alkyl, or R₁₀ and R₁₁, together with the nitrogen atom to which they are both attached, form a 4- to 6-membered ring;
R₁₂ is H or C1-C6 alkyl;
y is an integer from 1 to 4;
R₂ is H, C1-C6 alkyl, C2-C6 alkenyl, C3-C6 cycloalkyl, halogen, cyano, R₁₃R₁₄N(CH₂)_{d}, R₁₅O(CH₂)ₑ, R₁₆S(CH₂)_{f}, R₁₇C(O)(CH₂)_{g}, 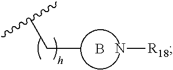
or
phenyl, optionally substituted by R₁₉O(CH₂)ᵢ;
R₁₃ is H, C1-C6 alkyl, R₂₀C(O), R₂₁S(O)₂, R₂₂O(CH₂)ⱼ, R₂₃R₂₄N(CH₂)ₖ, or benzyl, and
R₁₄ is H or C1-C6 alkyl; or
R₁₃ and R₁₄, together with the nitrogen atom to which they are both attached, form a 4- to 6-membered ring, said ring optionally being substituted by one or more substituents independently selected from oxo, halogen, C1-C6 alkyl, R₂₅C(O), R₂₆OC(O), and R₂₇O(CH₂)ₘ;
R₁₅ is H, C1-C6 alkyl or R₂₈C(O);
R₁₆ and R₁₇ are selected from H and C1-C6 alkyl;
R₁₈ is H, C1-C6 alkyl, R₂₉OC(O)(CH₂)ₙ, or R₃₀S(O)₂(CH₂)ₚ;
R₁₉, R₂₀, R₂₁, R₂₂, R₂₅, R₂₆, R₂₇, R₂₈, R₂₉ and R₃₀ are selected from H and C1-C6 alkyl;
R₂₃ and R₂₄ are independently selected from H and C1-C6 alkyl; or R₂₃ and R₂₄, together with the nitrogen atom to which they are both attached, form a 4- to 6-membered ring;
ring B is 4- to 6-membered, and saturated or unsaturated;
d, e, f, g, h, i, j, k, m, n, and p are integers of from 0 to 4;
R′₁ and R′₂ together form a bond; or
R′₁ is H, C1-C6 alkyl, C3-C6 carbocyclyl-(CH₂)_{q}, or R₃₁O(CH₂)ᵣ; and R′₂ is H;
R₃₁ is H or C1-C6 alkyl;
q and r are integers of from 0 to 4; and
each R₃ is independently selected from C1-C6 alkyl, C3-C6 carbocyclyl, halogen, oxo, R₃₂O, R₃₃S, and R₃₄R₃₅N;
R₃₂ is H, C1-C6 alkyl, C3-C6 carbocyclyl-(CH₂)ₛ, or R₃₆R₃₇N(CH₂)ₜ;
R₃₃ is H or C1-C6 alkyl;
R₃₄ and R₃₅ are independently selected from H and C1-C6 alkyl; or R₃₄ and R₃₅, together with the nitrogen atom to which they are both attached, form a 4- to 6-membered ring optionally substituted by one or more halogen;
R₃₆ and R₃₇ are independently selected from H and C1-C6 alkyl, or R₃₆ and R₃₇, together with the nitrogen atom to which they are both attached, form a 4- to 6-membered ring; optionally substituted by one or more halogen;
s and t are integers of from 0 to 4;
and
two R₃ attached to adjacent atoms of ring A, together with the atoms to which they are attached, may form a 3- to 6 membered ring, said ring being optionally substituted by one or more C1-C6 alkyl;
any alkyl, alkenyl and cycloalkyl is optionally substituted by one or more F;
provided that the compound is not
10-chloro-7-thia-2,5-diazatricyclo[6.4.0.0^{2,6}]dodeca-1(8),3,5,9,11-pentaene-4-carboxylic acid,
10,11-dimethyl-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid,
10-(trifluoromethyl)-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
7-thia-2,5-diazatricyclo[6.4.0.0^{2,6}]dodeca-1(8),3,5,9,11-pentaene-4-carboxylic acid,
12-methoxy-7-thia-2,5-diazatricyclo[6.4.0.0^{2,6}]dodeca-1(8),3,5,9,11-pentaene-4-carboxylic acid,
12-methyl-7-thia-2,5-diazatricyclo[6.4.0.0^{2,6}]dodeca-1(8),3,5,9,11-pentaene-4-carboxylic acid,
2-{7-thia-2,5-diazatricyclo[6.4.0.0^{2,6}]dodeca-1(8),3,5,9,11-pentaen-4-yl}acetic acid,
2-{10,11-dimethyl-7-thia-2,5-diazatricyclo[6.4.0.0^{2,6}]dodeca-1(8),3,5,9,11-pentaen-4-yl}acetic acid,
2-{10-chloro-7-thia-2,5-diazatricyclo[6.4.0.0^{2,6}]dodeca-1(8),3,5,9,11-pentaen-4-yl}acetic acid,
2-{11-methoxy-7-thia-2,5-diazatricyclo[6.4.0.0^{2,6}]dodeca-1(8),3,5,9,11-pentaen-4-yl}acetic acid,
16-thia-11,14-diazatetracyclo[8.6.0.0^{2,7}.0^{11,15}]hexadeca-1(10),2,4,6,8,12,14-heptaene-13-carboxylic acid,
2-{16-thia-11,14-diazatetracyclo[8.6.0.0^{2,7}.0^{11,15}]hexadeca-1(10),2,4,6,8,12,14-heptaen-13-yl}acetic acid,
2-{10-trifluoromethyl-7-thia-2,5-diazatricyclo[6.4.0.0^{2,6}]dodeca-1(8),3,5,9,11-pentaen-4-yl}acetic acid,
2-{10-bromo-7-thia-2,5-diazatricyclo[6.4.0.0^{2,6}]dodeca-1(8),3,5,9,11-pentaen-4-yl}acetic acid,
2-{10-methyl-7-thia-2,5-diazatricyclo[6.4.0.0^{2,6}]dodeca-1(8),3,5,9,11-pentaen-4-yl}acetic acid, or
2-{12-methyl-7-thia-2,5-diazatricyclo[6.4.0.0^{2,6}]dodeca-1(8),3,5,9,11-pentaen-4-yl}acetic acid.

27. The method according to claim 26, wherein the disorder is cancer, an autoimmunity disorder or a neurodegenerative disorder, and wherein the compound is as defined in claim 26 or is selected from
10-chloro-7-thia-2,5-diazatricyclo[6.4.0.0^{2,6}]dodeca-1(8),3,5,9,11-pentaene-4-carboxylic acid,
10-methyl-7-thia-2,5-diazatricyclo[6.4.0.0^{2,6}]dodeca-1(8),3,5,9,11-pentaene-4-carboxylic acid,
10-bromo-7-thia-2,5-diazatricyclo[6.4.0.0^{2,6}]dodeca-1(8),3,5,9,11-pentaene-4-carboxylic acid, and
10-(trifluoromethyl)-7-thia-2,5-diazatricyclo[6.4.0.0^{2,6}]dodeca-1(8),3,5,9,11-pentaene-4-carboxylic acid,
or a pharmaceutically acceptable salt thereof.

28. The method according to claim 27, wherein the disorder is cancer or a neurodegenerative disorder, and wherein the compound is as defined in claim 27 or is a compound selected from
10,11-dimethyl-7-thia-2,5-diazatricyclo[6.4.0.0^{2,6}]dodeca-1(8),3,5,9,11-pentaene-4-carboxylic acid,
10-methoxy-7-thia-2,5-diazatricyclo[6.4.0.0^{2,6}]dodeca-1(8),3,5,9,11-pentaene-4-carboxylic acid,
or a pharmaceutically acceptable salt thereof.
